(19) United States
(12) Patent Application Publication
(10) Pub. No.: US 2010/0255026 A1
(43) Pub. Date: Oct. 7, 2010

(54) Methods and compositions relating to anthrax spore glycoproteins as vaccines

(76) Inventors: Michael Jason Stump, Lexington, SC (US); Erin Pauline Worthy, Lexington, SC (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/545,051

(22) Filed: Oct. 6, 2006

Related U.S. Application Data
(60) Provisional application No. 60/724,306, filed on Oct. 6, 2005.

(51) Int. Cl. A61K 39/07; A61P 31/04

(52) U.S. Cl. 424/197.11

(57) ABSTRACT

Disclosed are methods for preparing an anthrax spore glycoprotein complex vaccine. Also, disclosed compositions of an anthrax vaccine including a spore glycoprotein complex as the active agent. In certain embodiments, the vaccines are sufficient to protect against infection from Bacillus anthracis and some forms of Bacillus cereus that cause an infections such as inhalation anthrax and the like

### STATEMENT OF RELATED APPLICATIONS

The present application claims priority to U.S. Provisional Patent Application 60/724,306, filed Oct. 6, 2005, and entitled “Novel Anthrax Spore Vaccine.”

### FIELD OF THE INVENTION

The present invention relates methods and compositions relating to anthrax spore glycoproteins as vaccines.

### BACKGROUND

Anthrax was previously known as woolsorters' disease as human infection had usually resulted from contact with infected animals or animal products such as hides or wool. The events of Sep. 11, 2001 and the subsequent anthrax outbreaks highlighted the more recent use of this bacterium for biological warfare and terrorism. Louis Pasteur produced the first anthrax vaccine in 1881 using a heat attenuated strain. The current U.S. licensed human anthrax vaccine, BIOTHRAX™ or Anthrax Vaccine Adsorbed (AVA) produced by BioPort Corporation (Lansing, Mich.), consists of aluminum hydroxide-adsorbed supernatant material from fermentor cultures of a toxigenic, non-encapsulated strain of B. anthracis.

Only toxin components have thus far been shown to confer protective immunity against anthrax (Mahlandt, B. G., et al. 1966. J Immunol 96:727-33). For example, protective antigen (PA) is an essential component of an anthrax vaccine (Grabenstein, J. D. 2003, Immunol. Allergy Clin. North Am., 23(4):713-30). Anti-PA antibody specific immunity may include anti-spore activity and thus, may have a role in impeding the early stages of infection with B. anthracis spores (Welkos, S. et al., 2001, Microbiology 147:1677-85). The current U.S. licensed human anthrax vaccine, primarily consists of protective antigen (PA) and undefined quantities of Lethal Factor (LF) and Edema Factor (EF), from fermentor cultures of a toxigenic, non-encapsulated strain of B. anthracis. Human vaccination with BIOTHRAX™ may require six immunizations followed by annual boosters (2002, Anthrax Vaccine Adsorbed (BioThrax™) Product Insert, BioPort Corporation; Friedlander, A. M., et al., 1999, Jama 282:2104-6). Using this vaccine, about 1 percent systemic and 3.6 percent local adverse events in humans have been reported (Pittman, P. R. et al., 2001, Vaccine 20:972-8).

There have been many attempts to improve the safety profile and immunogenicity of the anthrax vaccine using PA as an antigen, including the formulation of PA in adjuvants (Ivins, B. E. et al., 1992, Infect. Immun., 60:662-8; Kenney, R. T., et al., 2004. J. Infect. Dis., 190:774-82, Epub 2004 Jul. 13) (Matyas, G. R., et al., 2004, Infect. Immun., 72:1181-3), conjugating capsular poly-gamma-d-glutamic acid (PGA) to PA (Rhie, G. E. et al., 2003. Proc. Natl. Acad. Sci., USA 100:10925-30), the use of purified PA (Singh, Y. et al., 1998. Infect. Immun., 66:3447-8) and C-domain 4 of PA (PA-D4), (Flick-Smith, H. C. et al., 2002, Infect. Immun., 70:1653-6), the development of PA-based DNA vaccines (Gu, M. L. et al., 1999, Vaccine 17:340-4; Riemenschneider, J. et al., 2003, Vaccine 21:4071-80), and expression of PA in adenovirus, Salmonella typhimurium, Bacillus subtilis, vaccinia viral vector, and venezuelan equine encephalitis virus (Coulson, N. M. et al., 1994, Vaccine, 12:1395-401; Garmory, H. S. et al., 2003, Infect. Immun., 71:3831-6; Iacono-Connors, L. C. et al., 1991, Infect. Immun., 59:1961-5; Ivins, B. E., and S. L. Welkos, 1986, Infect. Immun., 54:537-42; Lee, J. S. et al., 2003., Infect. Immun., 71:1491-6; Tan, Y. et al. 2003, Hum. Gene Ther., 14:1673-82). Anthrax protective antigen (PA) is the major antigen in the current licensed anthrax vaccine BIOTHRAX™. The c-terminal domain 4 (PA-D4, residues 596-735) of PA appears to be responsible for binding cellular receptor, the anthrax toxin receptor (ATR), and may contain the dominant protective epitopes of PA (Flick-Smith, H. C. et al., 2002, Infect. Immun. 70:1653-6; Little, S. F. et al. 1996, Microbiology 142:707-15). Previous research indicated that immunization with plasmid expression vectors in a combination of PA and N-terminal region truncated LF (residues 10-254 of the mature protein) may provide better protection than PA alone (Galloway, D., et al. 2004, Vaccine, 22:1604-8; Price, B. M. et al., 2001, Infect. Immun., 69:4509-15).

The highly fatal nature of pulmonary anthrax, the ease of production and storage of the spores of B. anthracis, and the ability of spores to survive in the environment after an attack, make B. anthracis attractive as an agent in biowarfare and bioterrorism. Because the window of opportunity for effective antibiotic treatment is so small, vaccination may be the best defense against pulmonary anthrax. The current vaccine against anthrax is a crude culture supernatant from a non-encapsulated strain of B. anthracis that contains protective antigen (PA) generated by the vegetative cell. This vaccine may provide protection against the pulmonary form of anthrax in rhesus macaques and rabbits, but protection in guinea pigs is variable (Fellows et al., 2001). Furthermore, the current vaccine which utilizes PA can only be expected to afford protection against the natural agent, and would not be expected to provide protection against engineered forms of the organism. The selection of B. anthracis as a biological weapon is due not only to the toxic properties of the bacterium, but also because it provides an easily produced, stably maintained, delivery vehicle. It is possible to introduce other toxins, such as botulism toxin or shiga toxin, into this bacterium. Such engineered B. anthracis spores could then deliver not only the anthrax toxin, but also the additional toxins introduced into the spore. The current vaccine (which utilizes PA) would not be effective against such engineered organisms because it provides no protection against the foreign toxins. For these reasons, antitoxin immunity alone may not be a long-term solution.

While the currently available vaccines are an improvement over the use of a heat-attenuated anthrax strain, there is still a need for an improved vaccine. For example, the currently available vaccines are characterized by a lack of standardization, and a relatively high expense of production. Additionally, human vaccination with BIOTHRAX™ requires six immunizations followed by annual boosters (see e.g., the Anthrax Vaccine Adsorbed BIOTHRAX™ Product Insert, BioPort Corporation, 2002; Friedlander, A. M., et al., 1999, JAMA 282:2104-6). Further underscoring the need for development of new, improved anthrax vaccines are the reported 1% systemic and 3.6% local adverse events in humans (Pittman, P. R. et al., 2001, Vaccine 20:972-8).

Thus, there is a need to provide methods and systems for the isolation of proteins complexes from the surface of microorganisms, where such complexes may be antigenic. There is also a need to develop vaccines that may be used to defend against various biowarfare agents as well as other disease agents such as HIV.

### SUMMARY OF THE INVENTION

Embodiments of the present invention comprise methods and compositions relating to isolation of glycoprotein complexes from anthrax and other microbiological agents for use as vaccines. The present invention may be embodied in a variety of ways.

In one embodiment, the present invention comprises a method for isolation of glycoproteins on the exosporium or surface of a microorganism that may be used in a vaccine. In an embodiment, the microorganism may be Bacillus anthracis or an anthrax-like bacterim. In an embodiment, the method may comprise the step of isolating at least one glycoprotein from an extract of the exosporium of the bacterium by absorption of the extract to a sugar-binding agent. In an embodiment, the sugar binding agent is lectin. Or, other agents such as proteins, lipids, sugars and other antibodies that can combine with sugars, and that are known to interact with specific sugars found in glyoproteins may be used to capture proteins and other glycoprotein complexes.

In another embodiment, the present invention comprises a composition comprising at least one glycoprotein isolated from the exosporium or surface of a microorganism, where the glycoprotein comprises at least one lectin-binding sugar. In an embodiment, exosporium is from an Bacillus anthracis spore. In an embodiment, the composition may comprise a pharmaceutical carrier. In certain embodiments the glycoprotein is isolated as a complex comprising at least one of an oligosaccharide, a lipid, or a phospholipid.

In certain embodiments, the compositions of the present invention provide an anthrax vaccine that is protective against all strains Bacillus anthracis or associated diseases, and other anthrax-like infections including, but not limited to, Bacillus cereus G9241.

### BRIEF DESCRIPTION OF THE DRAWINGS

The present invention may be better understood by reference to the following non-limiting drawings. The patent or application file contains at least one drawing executed in color. Copies of this patent or patent application publication with color drawing(s) will be provided by the Office upon request and payment of the necessary fee.

FIG. 1 illustrates a schematic presentation of the exosporium of the Bacillus anthracis spore in accordance with an embodiment of the present invention.

FIG. 2 illustrates a flow-chart presentation of a method for the isolation of glycoproteins from the exosporium of the Bacillus anthracis spore in accordance with an embodiment of the present invention.

FIG. 3 illustrates an embodiment of protein distribution of Bacillus anthracis spores before and after lectin treatment run by one-dimensional gel electrophoresis in accordance with an embodiment of the present invention.

FIG. 4 illustrates glycoprotein staining of urea extracted spores before lectin treatment run by two dimensional gel electorphoresis in accordance with an embodiment of the present invention.

FIG. 5 illustrates a MALDI TOF MS characterization of a single glycoprotein band (EA1 1D) (band 1 of the gel of FIG. 3) in accordance with an embodiment of the present invention.

### DETAILED DESCRIPTION

Definitions

The following definitions may be used to understand the description herein. Unless defined otherwise, all technical and scientific terms used herein have the same meaning as commonly understood by one of ordinary skill in the art.

The term “a” or “an” as used herein may refer to more than one object unless the context clearly indicates otherwise. The term “or” is used interchangeably with the term “and/or” unless the context clearly indicates otherwise.

“Polypeptide” and “protein” are used interchangeably herein to describe protein molecules that may comprise either partial or full-length proteins. As used herein, a “polypeptide domain” comprises a region along a polypeptide that comprises an independent unit. Domains may be defined in terms of structure, sequence and/or biological activity. In one embodiment, a polypeptide domain may comprise a region of a protein that folds in a manner that is substantially independent from the rest of the protein. Domains may be identified using domain databases such as, but not limited to PFAM, PRODOM, PROSITE, BLOCKS, PRINTS, SBASE, ISREC PROFILES, SAMRT, and PROCLASS. As used herein, the term “glycoprotein” refers to any protein that is glycosylated.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues. DNA molecules may be identified by their nucleic acid sequences, which are generally presented in the 5′ to 3′ direction (as the coding strand), where the 5′ and 3′ indicate the linkages formed between the 5′-hydroxyl group of one nucleotide and the 3′-hydroxyl group of the next nucleotide. For a coding strand presented in the 5′-3′ direction, its complement (or non-coding strand) is the DNA strand which hybridizes to that sequence according to Watson-Crick base pairing. Thus, as used herein, the complement of a nucleic acid is the same as the “reverse complement” and describes the nucleic acid that in its natural form, would be based paired with the nucleic acid in question.

As used herein, “primers” are a subset of oligonucleotides that can hybridize with a target nucleic acid such that an enzymatic reactions, that uses the primers as a substrate, at least in part, can occur. A primer can be made from any combination of nucleotides or nucleotide derivatives or analogs available in the art which do not interfere with the enzymatic manipulation. “Probes” are oligonucleotide molecules capable of interacting with a target nucleic acid, typically in a sequence specific manner, for example through hybridization. Typically a probe can be made from any combination of nucleotides or nucleotide derivatives or analogs available in the art.

The term “vector” refers to a nucleic acid molecule that may be used to transport a second nucleic acid molecule into a cell. In one embodiment, the vector allows for replication of DNA sequences inserted into the vector. The vector may comprise a promoter to enhance expression of the nucleic acid molecule in at least some host cells. Vectors may replicate autonomously (extrachromasomal) or may be integrated into a host cell chromosome. In one embodiment, the vector may comprise an expression vector capable of producing a protein derived from at least part of a nucleic acid sequence inserted into the vector.

The term “percent identical” or “percent identity” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, Adv. Appl. Math., 1981, 2:482; Needleman and Wunsch, 1970, J. Mol. Biol., 48:443); Pearson and Lipman, 1988, Proc. Natl. Acad. Sci. USA, 85:2444) or by computerized versions of these algorithms (Wisconsin Genetics Software Package Release 7.0, Genetics Computer Group, 575 Science Drive, Madison, Wisc.) publicly available as BLAST and FASTA. Also, ENTREZ, available through the National Institutes of Health, Bethesda Md., may be used for sequence comparison. In one embodiment, percent identity of two sequences may be determined using GCG with a gap weight of 1, such that each amino acid gap is weighted as if it were a single amino acid or nucleotide mismatch between the two sequences.

An “effective amount” as used herein means the amount of an agent that is effective for producing a desired effect. Where the agent is being used to achieve a insecticidal effect, the actual dose which comprises the effective amount may depend upon the route of administration, and the formulation being used.

As used herein, an “immune response” refers to reaction of the body as a whole to the presence of an antigen which includes making antibodies, developing immunity, developing hypersensitivity to the antigen, and developing tolerance. Therefore, an immune response to an antigen also includes the development in a subject of a humoral and/or cellular immune response to the antigen of interest. A “humoral immune response” is mediated by antibodies produced by plasma cells. A “cellular immune response” is one mediated by T lymphocytes and/or other white blood cells. Spores can germinate within macrophages, so immunization to a spore can cause the development of opsonizing antibodies. Cell mediated immunity can compensate by causing macrophage activation and increased spore death. Humoral immunity to spore components can also cause immunity, and this effect may be augmented by cell mediated immunity. As used herein, “antibody titers” are defined as the highest dilution in post-immune sera that resulted in equal absorbance value of pre-immune samples for each subject.

As used herein, the term “antigen” refers to any agent, (e.g., any substance, compound, molecule, protein or other moiety) that is recognized by an antibody and/or can elicit an immune response in an individual. As used herein, the term “adjuvant” refers to any agent (e.g., any substance, compound, molecule, protein or other moiety) that can increase the immune response of an antigen.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain may also have regularly spaced intrachain disulfide bridges. Each heavy chain may have at one end a variable domain V_{H} followed by a number of constant domains. Each light chain may have a variable domain at one end V_{L} and a constant domain at its other end; the constant domain of the light chain may be aligned with the first constant domain of the heavy chain, and the light chain variable domain may be aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. There are similar class for other species (e.g., mouse). The heavy chain constant domains that correspond to the different classes of immunoglobulins are called alpha, delta, epsilon, gamma, and mu, respectively.

The term “variable” is used herein to describe certain portions of the variable antibody domains that differ in sequence among antibodies and are used in the binding and specificity of each particular antibody for its particular antigen. However, the variability is not usually evenly distributed through the variable domains of antibodies, but is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which can form loops connecting, and in some cases forming part of, the b-sheet structure. The CDRs in each chain may be held together in close proximity by the FR regions and, with the CDRs from the other chain, contribute to the formation of the antigen binding site of antibodies (see Kabat E. A. et al., 1987, “Sequences of Proteins of Immunological Interest,” National Institutes of Health, Bethesda, Md.). The constant domains are not involved directly in binding an antibody to an antigen, but may exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are included in this definition. For example, fragments of antibodies which maintain EFn binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)). Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Also, as used herein, “humanized forms of antibodies” are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin.

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

As used herein, the term “anthrax” refers to any strain of Bacillus anthracis either in vegatative or spore form. As used herein, the terms “anthrax-like” or “anthrax-like infections” or “anthrax-like diseases” refer to any strain of Bacillus cereus or other related Bacillus strain, and diseases similar to that of inhalation, gastrointestinal, or cutaneous anthrax. As used herein, the term “spore surface” refers to the exosporium, spore coat, and the outer layer of the cortex. Specifically, B. cereus ATCC 10987, B. cereus ATCC 10987, B. cereus G9241 have been known to cause anthrax-like response in recent studies. (Rask et al., 2004, Nucleic Acids Res. 32(3):977-88; Han et al., 2006; J. Bacteriology, 188 (9): 3382-90; Hoffmaster et al., 2006, J Clin. Microbiol., 44: 3352-60).

As used herein, the term “complexed,” “complex,” or “complexes” means anything that is bound together by either covalent or non-covalent interactions. For example, the glycoprotein BclA complex is BclA and any other proteins, lipids, phospholipids, polysaccharides or glycoproteins bound to BclA.

### Methods and Compositions Relating to Anthrax Spore Glycoproteins as Vaccines

Embodiments of the present invention comprise methods and compositions relating to the isolation anthrax spore glycoproteins and glycoprotein complexes as vaccines. The present invention may be embodied in a variety of ways.

In one embodiment, the present invention comprises a method for isolation of glycoproteins on the exosporium of a microorganism that may be used in a vaccine. In am embodiment, the microorganism may be a bacterium. In an embodiment, the bacterium may be Bacillus anthracis or an anthrax-like bacterium. In an embodiment, the method may comprise the step of isolating at least one glycoprotein from an extract of the exosporium of the bacterium by absorption of the extract to a sugar-binding agent. In an embodiment, the sugar binding agent is lectin. Or, other agents, such as proteins, lipids, sugars and other antibodies that are known to interact with specific sugars found in glyoproteins may be used to capture glycoproteins or glycoprotein complexes.

In an embodiment, the method comprises a step wherein the glycoprotein is isolated as part of a complex comprising at least one other molecule, wherein the at least one other molecule comprises a protein, an oligosaccharide, a lipid, or a phospholipid. For example, the complex may be isolated from the exosporium using at least one of size-exclusion chromatography or electro-elution. Or other size selection method may be used. Also, in an embodiment, at least one other molecule of the complex is identified. In an embodiment, the methods used to identify the glycoprotein and/or other molecule may include MS-TOF, protein sequencing or other similar methods such as Matrix-assisted laser desorption/ionization (MALDI), Time-of-flight (TOF) mass spectrometry (MS), Electrospray-ionization (ESI) Ion Trap (IT) MS, Matrix-assisted laser desorption/ionization (MALDI) Fourier transform ion cyclotron resonance (FT-ICR) MS, Electrospray ionization (ESI) Fourier transform ion cyclotron resonance (FT-ICR) MS.

For example, in one embodiment, the present invention comprises a method for isolation of glycoproteins on the exosporium of the Bacillus anthracis spore that may be used in a vaccine. In an embodiment, the method may comprise the step of isolating at least one glycoprotein from an extract of the exosporium of the Bacillus anthracis spore by absorption of proteins in the extract to lectin. In certain embodiments, the glycoprotein is isolated as a complex comprising at least one of an oligosaccharide, a lipid, or a phospholipid.

In an embodiment, the glycoprotein is isolated as part of a complex comprising at least one other molecule, wherein the at least one other molecule comprises a protein, an oligosaccharide, a lipid, or a phospholipid. For example, the complex may be isolated from the exosporium using at least one of size-exclusion chromatography or electro-elution. Or other size selection method may be used. Also, in an embodiment, at least one other molecule of the complex is identified. In an embodiment, the methods used to identify the glycoprotein and/or other molecule may include MS-TOF, protein sequencing or other similar methods such as Matrix-assisted laser desorption/ionization (MALDI), Time-of-flight (TOF) mass spectrometry (MS), Electrospray-ionization (ESI) Ion Trap (IT) MS, Matrix-assisted laser desorption/ionization (MALDI) Fourier transform ion cyclotron resonance (FT-ICR) MS, Electrospray ionization (ESI) Fourier transform ion cyclotron resonance (FT-ICR) MS.

In an embodiment, the complex comprises at least one of the following proteins from Bacillus anthracis: CotS, CotJA, CotJB, CotJC, CotM, CotH, CotC, CotAlpha, CotF, CotD, CotZ, Cot(Putative 1, 2, 3, 4), CotHypoAlpha, CotE, CotF(Related), BclA, EA1, EA2, srtA (Sortase A), SSPH1, SSPH2, SSPI, SSPK, SSPN, SSPO, TLP, SSPB, SSPalpha/beta1, SSPalpha/beta2, SSPalpha/beta3, SSPalpha/beta4, SASP-2, SSPF, SASP-1, SSPE(SSPgamma), ExsB, cspA, cspB-1, cspB-2, cspC, cspD, cspE, NDK, NupC-1, NupC-2, NupC-3, NupC-4, NupC-5, NupC-6, NupC-7, PnuC, Alanine racemase, Alanine dehydrogenase, Nucleoside hydrolase, BxpB, ExsFA, or ExsFB.

In another embodiment, the complex is isolated from a Bacillus subtilis spore. Thus, in an embodiment, the complex comprises at least one of the following proteins from Bacillus subtilis: CotA, CotB, CotC, CotD, CotE, CotF, CotG, CotH, CotJA, CotJB, CotJC, CotM, CotR, CotSA, CotS, CotT, CotV, CotW, CotY, CotZ, GerPA, GerPB, GerPC, GerPD, GerPE, GerPF, YaaH, YabG, YrbA (SafA), CotQ (YvdP), CotU (YnzH), CotI (YtaA), YckK, YdhD, YhdA, YhdE, YirY, YisY, YodI, YopQ, YdeP/YpeB, YpzA, YusA, YwqH, YxeF, CspD, Hsb, PhoA, SleB, SspA, SspE, YhcN, YrbB, CggR, CoxA, CwlJ, SpoIVA, SpoVM, SpoVID, YhbA, CSI5, CspB, CspC, CspD, DHBA, FABI, RL10, SRFAD, SAS1, SAS2, SASG, SSPA, SSPB, SSPC, SSPD, SSPE, SSPF, SSPG, SSPH, SSPI, SSPJ, SSPK, SSPL, SSPM, SSPN, SSPO, SSPP, TLP, SSPG-1, or SSPG-2.

In another embodiment, the complex is isolated from a Bacillus cereus spore. Thus, in an embodiment, the complex comprises at least one of the following proteins from Bacillus cereus: ExsA, ExsB, ExsC, ExsD, ExsE, ExsG, ExsH, ExsY, ExsJ, ExsF, YrbB, or NadA.

In another embodiment, the present invention comprises a composition comprising at least one glycoprotein from the exosporium of the Bacillus anthracis spore, where the glycoprotein comprises at least one lectin-binding sugar. In certain embodiments the glycoprotein is isolated as a complex comprising at least one of an oligosaccharide, a lipid, or a phospholipid. In an embodiment, the composition may comprise a pharmaceutically acceptable carrier.

Pharmaceutically acceptable carriers may comprise any of the standard pharmaceutically accepted carriers known in the art. In one embodiment, the pharmaceutical carrier may be a liquid and the protein or nucleic acid construct of the present invention may be in the form of a solution. In another embodiment, the pharmaceutically acceptable carrier may be a solid in the form of a powder, a lyophilized powder, or a tablet. Or, the pharmaceutical carrier may be a gel, suppository, or cream. In alternate embodiments, the carrier may comprise a liposome, a microcapsule, a polymer encapsulated cell, or a virus. Thus, the term pharmaceutically acceptable carrier encompasses, but is not limited to, any of the standard pharmaceutically accepted carriers, such as water, alcohols, phosphate buffered saline solution, sugars (e.g., sucrose or mannitol), oils or emulsions such as oil/water emulsions or a triglyceride emulsion, various types of wetting agents, tablets, coated tablets and capsules.

In an embodiment, the complex comprises at least one of the following proteins from Bacillus anthracis: CotS, CotJA, CotJB, CotJC, CotM, CotH, CotC, CotAlpha, CotF, CotD, CotZ, Cot(Putative 1, 2, 3, 4), CotHypoAlpha, CotE, CotF(Related), BclA, EA1, EA2, srtA (Sortase A), SSPH1, SSPH2, SSPI, SSPK, SSPN, SSPO, TLP, SSPB, SSPalpha/beta1, SSPalpha/beta2, SSPalpha/beta3, SSPalpha/beta4, SASP-2, SSPF, SASP-1, SSPE(SSPgamma), ExsB, cspA, cspB-1, cspB-2, cspC, cspD, cspE, NDK, NupC-1, NupC-2, NupC-3, NupC-4, NupC-5, NupC-6, NupC-7, PnuC, Alanine racemase, Alanine dehydrogenase, Nucleoside hydrolase, BxpB, ExsFA, or ExsFB.

In another embodiment, the complex is isolated from a Bacillus subtilis spore. Thus, in an embodiment, the complex comprises at least one of the following proteins from Bacillus subtilis: CotA, CotB, CotC, CotD, CotE, CotF, CotG, CotH, CotJA, CotJB, CotJC, CotM, CotR, CotSA, CotS, CotT, CotV, CotW, CotY, CotZ, GerPA, GerPB, GerPC, GerPD, GerPE, GerPF, YaaH, YabG, YrbA (SafA), CotQ (YvdP), CotU (YnzH), CotI (YtaA), YckK, YdhD, YhdA, YhdE, YirY, YisY, YodI, YopQ, YdeP/YpeB, YpzA, YusA, YwqH, YxeF, CspD, Hsb, PhoA, SleB, SspA, SspE, YhcN, YrbB, CggR, CoxA, CwlJ, SpoIVA, SpoVM, SpoVID, YhbA, CSI5, CspB, CspC, CspD, DHBA, FABI, RL10, SRFAD, SAS1, SAS2, SASG, SSPA, SSPB, SSPC, SSPD, SSPE, SSPF, SSPG, SSPH, SSPI, SSPJ, SSPK, SSPL, SSPM, SSPN, SSPO, SSPP, TLP, SSPG-1, or SSPG-2.

In another embodiment, the complex is isolated from a Bacillus cereus spore. Thus, in an embodiment, the complex comprises at least one of the following proteins from Bacillus cereus: ExsA, ExsB, ExsC, ExsD, ExsE, ExsG, ExsH, ExsY, ExsJ, ExsF, YrbB, or NadA.

In an embodiment, the method comprises a step wherein the glycoprotein is isolated as part of a complex comprising at least one other molecule, wherein the at least one other molecule comprises a protein, an oligosaccharide, a lipid, or a phospholipid. For example, the complex may be isolated from the exosporium using at least one of size-exclusion chromatography or electro-elution. Or other size selection method may be used. Also, in an embodiment, at least one other molecule of the complex is identified. In an embodiment, the methods used to identify the glycoprotein and/or other molecule may include MS-TOF, protein sequencing or other similar methods such as Matrix-assisted laser desorption/ionization (MALDI), Time-of-flight (TOF) mass spectrometry (MS), Electrospray-ionization (ESI) Ion Trap (IT) MS, Matrix-assisted laser desorption/ionization (MALDI) Fourier transform ion cyclotron resonance (FT-ICR) MS, Electrospray ionization (ESI) Fourier transform ion cyclotron resonance (FT-ICR) MS.

In yet other embodiments, the present invention comprises compositions comprising a complex isolated from the exosporium of the Bacillus anthracis spore comprising at least one of a polypeptide, glycoprotein, lipid, phospholipid, or oligosaccharide wherein the polypeptide, glycoprotein, lipid, phospholipids, or oligosaccharide comprises an antigen, and/or wherein the at least one polypeptide, glycoprotein, lipid, phospholipid, or oligosaccharide is capable of producing a cellular or a humoral immune response. In an embodiment, the composition may comprise a pharmaceutically acceptable carrier.

In an embodiment, the complex comprises at least one of the following proteins from Bacillus anthracis: CotS, CotJA, CotJB, CotJC, CotM, CotH, CotC, CotAlpha, CotF, CotD, CotZ, Cot(Putative 1, 2, 3, 4), CotHypoAlpha, CotE, CotF(Related), BclA, EA1, EA2, srtA (Sortase A), SSPH1, SSPH2, SSPI, SSPK, SSPN, SSPO, TLP, SSPB, SSPalpha/beta1, SSPalpha/beta2, SSPalpha/beta3, SSPalpha/beta4, SASP-2, SSPF, SASP-1, SSPE(SSPgamma), ExsB, cspA, cspB-1, cspB-2, cspC, cspD, cspE, NDK, NupC-1, NupC-2, NupC-3, NupC-4, NupC-5, NupC-6, NupC-7, PnuC, Alanine racemase, Alanine dehydrogenase, Nucleoside hydrolase, BxpB, ExsFA, or ExsFB.

In another embodiment, the complex is isolated from a Bacillus subtilis spore. Thus, in an embodiment, the complex comprises at least one of the following proteins from Bacillus subtilis: CotA, CotB, CotC, CotD, CotE, CotF, CotG, CotH, CotJA, CotJB, CotJC, CotM, CotR, CotSA, CotS, CotT, CotV, CotW, CotY, CotZ, GerPA, GerPB, GerPC, GerPD, GerPE, GerPF, YaaH, YabG, YrbA (SafA), CotQ (YvdP), CotU (YnzH), CotI (YtaA), YckK, YdhD, YhdA, YhdE, YirY, YisY, YodI, YopQ, YdeP/YpeB, YpzA, YusA, YwqH, YxeF, CspD, Hsb, PhoA, SleB, SspA, SspE, YhcN, YrbB, CggR, CoxA, CwIJ, SpoIVA, SpoVM, SpoVID, YhbA, CSI5, CspB, CspC, CspD, DHBA, FABI, RL10, SRFAD, SAS1, SAS2, SASG, SSPA, SSPB, SSPC, SSPD, SSPE, SSPF, SSPG, SSPH, SSPI, SSPJ, SSPK, SSPL, SSPM, SSPN, SSPO, SSPP, TLP, SSPG-1, or SSPG-2.

In another embodiment, the complex is isolated from a Bacillus cereus spore. Thus, in an embodiment, the complex comprises at least one of the following proteins from Bacillus cereus: ExsA, ExsB, ExsC, ExsD, ExsE, ExsG, ExsH, ExsY, ExsJ, ExsF, YrbB, or NadA.

In an embodiment, the glycoprotein is isolated as part of a complex comprising at least to one other molecule, wherein the at least one other molecule comprises a protein, an oligosaccharide, a lipid, or a phospholipid. For example, the complex may be isolated from the exosporium using at least one of size-exclusion chromatography or electro-elution. Or other size selection method may be used. Also, in an embodiment, at least one other molecule of the complex is identified. In an embodiment, the methods used to identify the glycoprotein and/or other molecule may include MS-TOF, protein sequencing or other similar methods such as Matrix-assisted laser desorption/ionization (MALDI), Time-of-flight (TOF) mass spectrometry (MS), Electrospray-ionization (ESI) Ion Trap (IT) MS, Matrix-assisted laser desorption/ionization (MALDI) Fourier transform ion cyclotron resonance (FT-ICR) MS, Electrospray ionization (ESI) Fourier transform ion cyclotron resonance (FT-ICR) MS.

In an embodiment, the microorganism from which the glycoprotein or glycoprotein complex is isolated may comprise an Anthrax bacterium. Or, other the microorganism may comprise any one of the microorganisms listed in Table 1.

TABLE 1

| Pathogen or | | | | |
|---|---|---|---|---|
| Toxin | Lectin | Carbohydrate or Ligand | Year | Citation |
| Escherichia coli | 17 kDa | Man | 1987 | FEBS Letters, vol. 217, |
| | | | | no. 2, pp. 145-157, |
| | | | | 1987 |
| Escherichia coli | 18 kDa | Gal | 2001 | Arch Biochem Biophys |
| | | | | 2001 Jun. 1; 390(1): 109-18 |
| Escherichia coli | 18 kDa | Gal | 2001 | Arch Biochem Biophys |
| | | | | 2001 Jun. 1; 390(1): 109-18 |
| Streptococcus | 18-kDa | Gal(a1-4)Gal | 1996 | Infection and |
| suis | | | | Immunity. 1996 September |
| | | | | 64(9): 3659-65 |
| Escherichia coli | 20-kDa | GlcNAc | 1996 | Infect. Immun., 1996 |
| | subunits | | | January; 64(1): 332-42 |
| Burkholderia | 22-kDa | Gal(a1-4)Gal | 1996 | Infection and |
| cepacia | | | | Immunity, vol. 64, no. |
| | | | | 4, pp. 1420-1425, 1996 |
| Pasteurella | 68-kDa | GlcNAc | 2000 | Glycobiology, 2000, |
| haemolytica | | | | Vol. 10, No. 1 31-37 |
| Pasteurella | 68-kDa | NeuAc | 2000 | Glycobiology, 2000, |
| haemolytica | | | | Vol. 10, No. 1 31-37 |
| Clostridium | B subunit | Gal(b1-3)[NeuAc(a2- | 1998 | Microbial Pathogenesis. |
| botulinum type B | | 3)]GalNAc(b1-4)Gal(b1- | | 1998 August 25(2): 91-9 |
| | | 4)[NeuAc(a2-3)Glc(b1-1)Cer | | |
| Shiga toxin | B subunit | Gal(a1-3)Gal(b1-4)Glc | 1986 | The Journal of |
| | | | | Experimental Medicine. |
| | | | | 1986 Jun. 1 163(6): |
| | | | | 1391-404 |
| Shiga toxin | B subunit | Gal(a1-3)Gal(b1- | 1986 | The Journal of |
| | | 4)GlcNAc | | Experimental Medicine. |
| | | | | 1986 Jun. 1 163(6): |
| | | | | 1391-404 |
| Shiga toxin | B subunit | GlcNAc(b1-4)GlcNAc | 1986 | The Journal of |
| | | | | Experimental Medicine. |
| | | | | 1986 Jun. 1 163(6): |
| | | | | 1391-404 |
| Ricin toxin | B- | (b1-3)Gal | 2004 | Journal of |
| | subunit | | | Immunology. 2004; |
| | | | | 172: 6836-6845 |
| Ricin toxin | B- | (b1-4)Gal | 2004 | Journal of |
| | subunit | | | Immunology. 2004; |
| | | | | 172: 6836-6845 |
| Cholera toxin | B- | Gal(b1-3)GalNAc(b1- | 2004 | Biochemical and |
| (Vibrio cholerae) | subunit; | 4)[NeuAc(a2-3)]Gal(b1- | | Biophysical Research |
| | pentameric | 4)Glc(b1-1) | | Communications. 2004 |
| | | | | Aug. 13; vol. 321, no. 1: |
| | | | | 192-196 |
| Cholera toxin | B- | NeuAc(a2-3)[Gal(b1- | 2004 | Biochemical and |
| (Vibrio cholerae) | subunit; | 3)GalNAc(b1-4)]Gal(b1- | | Biophysical Research |
| | pentameric | 4)Glc(b1-1) | | Communications. 2004 |
| | | | | Aug. 13; vol. 321, no. 1: |
| | | | | 192-196 |
| Helicobacter | BabA | Fuc(a1-2)[Gal(a1- | 2004 | Science. 2004 Jul. 23; |
| pylori | | 3)Gal(b1- | | Vol 305: 519-22 |
| | | 3)]GlcNAc[Fuc(a1-4)] | | |
| Helicobacter | BabA | Fuc(a1-2)[GalNAc(a1- | 2004 | Science. 2004 Jul. 23; |
| pylori | | 3)Gal(b1-3)]Fuc(a1- | | Vol 305: 519-22 |
| | | 4)[GlcNAc] | | |
| Helicobacter | BabA | Fuc(a1-2)[GalNAc(a1- | 2004 | Science. 2004 Jul. 23; |
| pylori | | 3)Gal(b1-3)]GlcNAc | | Vol 305: 519-22 |
| Helicobacter | BabA | Fuc(a1-2)[GalNAc(a1- | 2004 | Science. 2004 Jul. 23; |
| pylori | | 3)Gal(b1- | | Vol 305: 519-22 |
| | | 3)]GlcNAc[Fuc(a1-4)] | | |
| Helicobacter | BabA | Fuc(a1-2)Gal(b1- | 2004 | Science. 2004 Jul. 23; |
| pylori | | 3)Fuc(a1-4)[GlcNAc] | | Vol 305: 519-22 |
| Helicobacter | BabA | Fuc(a1-2)Gal(b1- | 2004 | Science. 2004 Jul. 23; |
| pylori | | 3)GlcNAc | | Vol 305: 519-22 |
| Helicobacter | BabA | Gal(a1-3)Gal(b1- | 2004 | Science. 2004 Jul. 23; |
| pylori | | 3)[Fuc(a1-2)]Fuc(a1- | | Vol 305: 519-22 |
| | | 4)[GlcNAc] | | |
| Helicobacter | BabA | Gal(a1-3)Gal(b1- | 2004 | Science. 2004 Jul. 23; |
| pylori | | 3)[Fuc(a1- | | Vol 305: 519-22 |
| | | 2)]GlcNAc[Fuc(a1-4)] | | |
| Helicobacter | BabA | GalNAc(a1-3)Gal(b1- | 2004 | Science. 2004 Jul. 23; |
| pylori | | 3)[Fuc(a1-2)]Fuc(a1- | | Vol 305: 519-22 |
| | | 4)[GlcNAc] | | |
| Helicobacter | BabA | GalNAc(a1-3)Gal(b1- | 2004 | Science. 2004 Jul. 23; |
| pylori | | 3)[Fuc(a1-2)]GlcNAc | | Vol 305: 519-22 |
| Helicobacter | BabA | GalNAc(a1-3)Gal(b1- | 2004 | Science. 2004 Jul. 23; |
| pylori | | 3)[Fuc(a1- | | Vol 305: 519-22 |
| | | 2)]GlcNAc[Fuc(a1-4)] | | |
| Escherichia coli | CfaB | GalNAc(b1-4)[NeuGc(a1- | 2000 | Int J Med Microbiol. |
| | | 3)]Gal(b1-4)Glc(b1-1)Cer | | 2000 March; 290(1): 27- |
| | | | | 35. Review |
| Escherichia coli | CfaB | NeuGc(a1-3)[GalNAc(b1- | 2000 | Int J Med Microbiol. |
| | | 4)]Gal(b1-4)Glc(b1-1)Cer | | 2000 March; 290(1): 27- |
| | | | | 35. Review |
| Escherichia coli | Class I G | Gal(a1-4)Gal | 1998 | Journal of |
| | | | | Microbiological |
| | | | | Methods. Vol. 34, no. |
| | | | | 1, pp. 23-29. 1 Sep. |
| | | | | 1998 |
| Escherichia coli | Class II | Gal(a1-4)Gal | 1998 | Journal of |
| | G | | | Microbiological |
| | | | | Methods. Vol. 34, no. |
| | | | | 1, pp. 23-29. 1 Sep. |
| | | | | 1998 |
| Escherichia coli | Class III | Gal(a1-4)Gal | 1998 | Journal of |
| | G | | | Microbiological |
| | | | | Methods. Vol. 34, no. |
| | | | | 1, pp. 23-29. 1 Sep. |
| | | | | 1998 |
| Escherichia coli | CS3 | GalNAc(b1-4)Gal | 1995 | Infection and |
| | | | | Immunity, vol. 63, no. |
| | | | | 2, pp. 640-646, 1995 |
| Pseudomonas | exoenzyme | Gal(b1-3)GalNAc(b1- | 1997 | Gene. 1997 Jun. 11; |
| aeruginosa | S | 4)Gal(b1-4)Glc(b1-1)Cer | | 192(1): 99-108 |
| Pseudomonas | exoenzyme | GalNAc(b1-4)Gal(b1- | 1997 | Gene. 1997 Jun. 11; |
| aeruginosa | S | 4)Glc(b1-1)Cer | | 192(1): 99-108 |
| Escherichia coli | F | Gal(a1-4)Gal | 1998 | Journal of |
| | | | | Microbiological |
| | | | | Methods. Vol. 34, no. |
| | | | | 1, pp. 23-29. 1 Sep. |
| | | | | 1998 |
| Escherichia coli | FaeG | Fuc | 2000 | Int J Med Microbiol. |
| | | | | 2000 March; 290(1): 27- |
| | | | | 35. Review |
| Escherichia coli | FaeG | Gal(b | 2000 | Int J Med Microbiol. |
| | | | | 2000 March; 290(1): 27- |
| | | | | 35. Review |
| Escherichia coli | FaeG | Gal(b1-3)Gal | 2000 | Int J Med Microbiol. |
| | | | | 2000 March; 290(1): 27- |
| | | | | 35. Review |
| Escherichia coli | FaeG | GalNAc | 2000 | Int J Med Microbiol. |
| | | | | 2000 March; 290(1): 27- |
| | | | | 35. Review |
| Escherichia coli | FaeG | GlcNAc | 2000 | Int J Med Microbiol. |
| | | | | 2000 March; 290(1): 27- |
| | | | | 35. Review |
| Escherichia coli | FanC | NeuGc(a1-3)Gal(b1- | 2000 | Int J Med Microbiol. |
| | | 4)Glc(b1-1)Cer | | 2000 March; 290(1): 27- |
| | | | | 35. Review |
| Bordetella | FHA | Gal(b1-3)GlcNAc(b1- | 1993 | Infection and |
| pertussis | | 3)Gal(b1-4)Glc(b1-1)Cer | | Immunity. 1993 July; |
| | | | | 61(7): 2780-5 |
| Escherichia coli | FimH | Man | 1999 | Emerg Infect Dis. 1999 |
| | | | | May-Jun; 5(3): 395-403. |
| | | | | Review |
| Escherichia coli | FimH | Man | 1999 | J. Bacteriol., Feb. |
| | | | | 15, 1999; 181(4): 1059- |
| | | | | 1071 |
| Escherichia coli | FimH | Man | 2002 | Molecular |
| | | | | Microbiology, 2002 |
| | | | | May, 44(4): 903-15 |
| Escherichia coli | FimH | Man | 2003 | Med Sci Monit. 2003 |
| | | | | March; 9(3): RA76-82 |
| Escherichia coli | FocH | Gal | 2000 | Int J Med Microbiol. |
| | | | | 2000 March; 290(1): 27-35 |
| Escherichia coli | FocH | GalNAc | 2000 | Int J Med Microbiol. |
| | | | | 2000 March; 290(1): 27-35 |
| Human | gp120 | Gal(b1-1)Cer | 1993 | PNAS of the United |
| Immunodeficiency | | | | States of America. 1993 |
| Virus | | | | Apr. 1; 90(7): 2700-4 |
| Entamoeba | Heavy | Gal | 1999 | Infection and |
| histolytica | (170-kDa) | | | Immunity. Vol. 65, no. |
| | subunit | | | 5, pp. 2096-2102. May 1999 |
| Entamoeba | Heavy | GalNAc | 1999 | Infection and |
| histolytica | (170-kDa) | | | Immunity. Vol. 65, no. |
| | subunit | | | 5, pp. 2096-2102. May 1999 |
| Influenza | hemagglutinin | Gal(a1-3)Gal(b1- | 2003 | Biochem Pharmacol. |
| | | 4)GlcNAc(b1- | | 2003 Mar. 1; 65(5): 699- |
| | | 6)[NeuAc(a2-3)Gal(b1- | | 707. Review |
| | | 4)Glc(b1-3)]Gal(b1- | | |
| | | 4)GlcNAc(b1-3)Gal(b1- | | |
| | | 4)Glc(b1-1) | | |
| Influenza | hemagglutinin | NeuAc(a2-3)[NeuAc(a2- | 2003 | Biochem Pharmacol. |
| | | 3)Gal(b1-3)GalNAc(b1- | | 2003 Mar. 1; 65(5): 699- |
| | | 4)]Gal(b1-3)Glc(b1-1) | | 707. Review |
| Influenza | hemagglutinin | NeuAc(a2-3)Gal(b1- | 2003 | Biochem Pharmacol. |
| | | 3)GalNAc(b1- | | 2003 Mar. 1; 65(5): 699- |
| | | 4)[NeuAc(a2-3)]Gal(b1- | | 707. Review |
| | | 3)Glc(b1-1) | | |
| Influenza | hemagglutinin | NeuAc(a2-3)Gal(b1- | 2003 | Biochem Pharmacol. |
| | | 4)Glc(b1-1) | | 2003 Mar. 1; 65(5): 699- |
| | | | | 707. Review |
| Influenza | hemagglutinin | NeuAc(a2-3)Gal(b1- | 2003 | Biochem Pharmacol. |
| | | 4)Glc(b1-3)[Gal(a1- | | 2003 Mar. 1; 65(5): 699- |
| | | 3)Gal(b1-4)GlcNAc(b1- | | 707. Review |
| | | 6)]Gal(b1-4)GlcNAc(b1- | | |
| | | 3)Gal(b1-4)Glc(b1-1) | | |
| Influenza | hemagglutinin | NeuAc(a2-3)Gal(b1- | 2003 | Biochem Pharmacol. |
| | | 4)GlcNAc(b1-4)Gal(b1- | | 2003 Mar. 1; 65(5): 699- |
| | | 4)Glc(b1-1) | | 707. Review |
| Influenza | hemagglutinin | NeuAc(a2-3)Gal(b1- | 2003 | Biochem Pharmacol. |
| | | 4)GlcNAc(b1-4)Gal(b1- | | 2003 Mar. 1; 65(5): 699- |
| | | 4)GlcNAc(b1-3)Gal(b1- | | 707. Review |
| | | 4)Glc(b1-1) | | |
| Influenza | hemagglutinin | NeuAc(a2-6)Gal(b1- | 2003 | Biochem Pharmacol. |
| | | 4)GlcNAc(b1-4)Gal(b1- | | 2003 Mar. 1; 65(5): 699- |
| | | 4)Glc(b1-1) | | 707. Review |
| Influenza | hemagglutinin | NeuGc(a2-3)Gal(b1- | 2003 | Biochem Pharmacol. |
| | | 4)Glc(b1-1) | | 2003 Mar. 1; 65(5): 699- |
| | | | | 707. Review |
| Rota virus | hemagglutinin | NeuAc | 1990 | Journal of Virology. |
| | | | | 1990 October; 64(10): |
| | | | | 4830-5 |
| Entamoeba | Light (35- | Gal | 1999 | Infection and |
| histolytica | or 31-kDa) | | | Immunity. Vol. 65, no. |
| | subunit | | | 5, pp. 2096-2102. May |
| | | | | 1999 |
| Entamoeba | Light (35- | GalNAc | 1999 | Infection and |
| histolytica | or 31-kDa) | | | Immunity. Vol. 65, no. |
| | subunit | | | 5, pp. 2096-2102. May |
| | | | | 1999 |
| Proteus mirabilis | MrpII | Gal(a1-4)Gal | 2000 | Int J Med Microbiol. |
| | | | | 2000 March; 290(1): 27- |
| | | | | 35. Review |
| Escherichia coli | P | NeuAc(a2-3)Gal(b1- | 1998 | Infect. Immun., Aug. |
| | | 3)[NeuAc(a2- | | 1, 1998; 66(8): 3856- |
| | | 6)]GalNAc(b1-3)Gal(a1- | | 3861 |
| | | 4)Gal(b1-4)Glc(b1-1)Cer | | |
| Escherichia coli | P | NeuAc(a2-3)Gal(b1- | 1998 | Infect. Immun., Aug. |
| | | 3)GalNAc(b1-3)Gal(a1- | | 1, 1998; 66(8): 3856- |
| | | 4)Gal(b1-4)Glc(b1-1)Cer | | 3861 |
| Escherichia coli | P | NeuAc(a2-6)[NeuAc(a2- | 1998 | Infect. Immun., Aug. |
| | | 3)Gal(b1-3)]GalNAc(b1- | | 1, 1998; 66(8): 3856- |
| | | 3)Gal(a1-4)Gal(b1- | | 3861 |
| | | 4)Glc(b1-1)Cer | | |
| Pseudomonas | PA-IIL | Fuc | 2004 | Microbes and Infection. |
| aeruginosa | | | | 2004 February; 6(2): 221-8 |
| Pseudomonas | PA-IIL | Man | 2004 | Microbes and Infection. |
| aeruginosa | | | | 2004 February; 6(2): 221-8 |
| Pseudomonas | PA-IL | Gal | 2004 | Microbes and Infection. |
| aeruginosa | | | | 2004 February; 6(2): 221-8 |
| Escherichia coli | PapG | Gal(a1-4)Gal | 1995 | Current Opinion in |
| | | | | Structural Biology, vol. |
| | | | | 5, no. 5, pp. 622-635, |
| | | | | 1995 |
| Escherichia coli | PapG | Gal(a1-4)Gal | 1999 | Emerg Infect Dis. 1999 |
| | | | | May-Jun; 5(3): 395-403. |
| | | | | Review |
| Escherichia coli | PapG | Gal(a1-4)Gal | 1999 | Infect. Immun., |
| | | | | Nov. 1, 1999; |
| | | | | 67(11): 6161-6163 |
| Escherichia coli | PapG | Gal(a1-4)Gal | 1999 | J. Bacteriol., Feb. |
| | | | | 15, 1999; 181(4): 1059- |
| | | | | 1071 |
| Escherichia coli | PapG | Gal(a1-4)Gal | 2000 | Int J Med Microbiol. |
| | | | | 2000 March; 290(1): 27- |
| | | | | 35. Review |
| Escherichia coli | PapG | Gal(a1-4)Gal | 2003 | Med Sci Monit. 2003 |
| | | | | March; 9(3): RA76-82 |
| Escherichia coli | PapG | Gal(a1-4)Gal(b | 1996 | Bioorganic & medicinal |
| | | | | chemistry, 1996 November, |
| | | | | 4(11): 1809-17 |
| Escherichia coli | PapGII | GalNAc(b1-3)Gal(a1- | 2001 | EMBO Rep., Jul. 1, |
| | | 4)Gal(b1-4)Glc(b1-1)Cer | | 2001; 2(7): 621-627 |
| Escherichia coli | PapGIII | GalNAc(a1-3)GalNAc(a1- | 2001 | EMBO Rep., Jul. 1, |
| | | 3)Gal(a1-4)Gal(b1-4)Cer | | 2001; 2(7): 621-627 |
| Escherichia coli | PapGJ96 | Gal(a1-4)Gal | 1998 | Journal of |
| | | | | Microbiological |
| | | | | Methods. Vol. 34, no. |
| | | | | 1, pp. 23-29. 1 Sep. |
| | | | | 1998 |
| Yersinia pestis | pH 6 | Gal(b1-1)Cer | 1998 | Infection and |
| | | | | Immunity. 1998 September; |
| | | | | 66(9): 4545-8 |
| Yersinia pestis | pH 6 | Gal(b1-3)GalNAc(b1- | 1998 | Infection and |
| | | 4)Gal(b1-4)Glc(b1-1)Cer | | Immunity. 1998 September; |
| | | | | 66(9): 4545-8 |
| Yersinia pestis | pH 6 | Gal(b1-4)Glc(b1-1)Cer | 1998 | Infection and |
| | | | | Immunity. 1998 September; |
| | | | | 66(9): 4545-8 |
| Yersinia pestis | pH 6 | GalNAc(b1-4)Gal(b1- | 1998 | Infection and |
| | | 4)Glc(b1-1)Cer | | Immunity. 1998 September; |
| | | | | 66(9): 4545-8 |
| Pseudomonas | pilin | Gal(b1-3)GalNAc(b1- | 1997 | Gene. 1997 Jun. 11; |
| aeruginosa | subunit | 4)Gal(b1-4)Glc(b1-1)Cer | | 192(1): 99-108 |
| Pseudomonas | pilin | GalNAc(b1-4)Gal | 1997 | Gene. 1997 Jun. 11; |
| aeruginosa | subunit | | | 192(1): 99-108 |
| Pseudomonas | pilin | GalNAc(b1-4)Gal(b1- | 1997 | Gene. 1997 Jun. 11; |
| aeruginosa | subunit | 4)Glc(b1-1)Cer | | 192(1): 99-108 |
| Streptococcus | PN | Gal(a1-4)Gal | 1995 | The Journal of |
| suis | | | | biological chemistry. |
| | | | | Dec. 1, 1995. v. 270 |
| | | | | (48) p. 28874-28878 |
| Streptococcus | PO | Gal(a1-4)Gal | 1995 | The Journal of |
| suis | | | | biological chemistry. |
| | | | | Dec. 1, 1995. v. 270 |
| | | | | (48) p. 28874-28878 |
| Escherichia coli | PrsG | Gal(a1-4)Gal | 1999 | Emerg Infect Dis. 1999 |
| | | | | May-Jun; 5(3): 395-403. |
| | | | | Review |
| Escherichia coli | PrsG | Gal(a1-4)Gal | 2000 | Int J Med Microbiol. |
| | | | | 2000 March; 290(1): 27- |
| | | | | 35. Review |
| Escherichia coli | PrsG | Gal(a1-4)Gal | 2003 | Med Sci Monit. 2003 |
| | | | | March; 9(3): RA76-82 |
| Pertussis toxin | S2 | Gal(b1-3)GlcNAc(b1- | 1992 | PNAS United States of |
| (Bordetella | subunit | 3)Gal(b1-4)Glc(b1-1)Cer | | America. 1992 Jan. 1; |
| pertussis) | | | | 89(1): 118-22 |
| Pertussis toxin | S3 | Gal(b1-3)GalNAc(b1- | 1992 | PNAS United States of |
| (Bordetella | subunit | 4)Gal(b1-4)Glc(b1-1)Cer | | America. 1992 Jan. 1; |
| pertussis) | | | | 89(1): 118-22 |
| Escherichia coli | SafS | NeuAc(a2-3)Gal | 2003 | Med Sci Monit. 2003 |
| | | | | March; 9(3): RA76-82 |
| Escherichia coli | SfaS | NeuAc(a2-3)Gal | 1999 | Emerg Infect Dis. 1999 |
| | | | | May-Jun; 5(3): 395-403 |
| Escherichia coli | SfaS | NeuAc(a2-3)Gal | 2000 | Int J Med Microbiol. |
| | | | | 2000 March; 290(1): 27-35 |
| Entamoeba | transmembrane | | 2004 | Infection and |
| histolytica | heavy subunit | | | Immunity. 2004 vol. |
| | (Hgl; 170 kDa) | Gal | | 72, no. 9: 5349-5357 |
| | disulfide | | | |
| Entamoeba | transmembrane | GlcNAc | 2004 | Infection and |
| histolytica | heavy subunit | | | Immunity. 2004 vol. |
| | (Hgl; 170 kDa) | | | 72, no. 9: 5349-5357 |
| | disulfide | | | |
| Rotavirus | Virus Spike | NeuAc | 1997 | Journal of Virology, |
| | Protein VP4 | | | vol. 71, no. 9, pp. 6749- |
| | | | | 6756, September 1997 |

In an embodiment, the composition may comprise a vaccine. In certain embodiments, the compositions of the present invention provide an anthrax vaccine that is protective against all strains Bacillus anthracis, and other anthrax-like infections including, but not limited to, Bacillus cereus G9241. The vaccines may comprise a purified antigen, wherein the antigen comprises the any one of the polypeptides disclosed herein. In an embodiment, the antigen may comprise a complex of at least one glycoprotein isolated from the exosporium of a Bacillus anthracis spore. In certain embodiments, the vaccine may comprise a combination vaccine, where the combination vaccine comprises a purified antigen isolated from the exosporium of a Bacillus anthracis spore, and another Bacillus anthracis antigen, such as protective antigen (PA), the lethal factor (LF) protein, edema factor (EF), and the like.

In certain embodiments of the methods or compositions of the present invention, the complex comprises an isolated molecule comprising at least one of the nucleic acid sequences or at least one of the amino acid sequences, as set forth in SEQ ID NOs: 1-379. Or, the complex may comprise a nucleic acid molecule having 95%-99% identity to the nucleic acid sequences, or a protein or polypeptide having 95%-99% identity amino acid sequences, as set forth in SEQ ID NOs: 1-379. In other embodiments, the complex may comprise a nucleic acid molecule having 90%-99% identity to the nucleic acid sequences, or a protein or polypeptide having 90%-99% identity amino acid sequences, as set forth in SEQ ID NOs: 1-379. In other embodiments, the complex may comprise a nucleic acid molecule having 85%-99% identity to the nucleic acid sequences, or a protein or polypeptide having 85%-99% identity amino acid sequences as set forth in SEQ ID NOs: 1-379. In yet other embodiments, the complex may comprise a nucleic acid molecule having 80%-99% identity to the nucleic acid sequences, or a protein or polypeptide having 80%-99% identity amino acid sequences as set forth in SEQ ID NOs: 1-379. For example, the complex may comprise a fragment and/or homologue of a protein encoded by at least one of the nucleic acid and/or amino acid sequences, respectively, as set forth in SEQ ID NOs: 1-379, wherein the homologue comprises conservative amino acid substitutions and the fragment comprises the portion of the polypeptide that is antigenic. The present invention also comprises fragments of nucleic acid sequences that comprise at least 15 consecutive nucleic acid sequences for the nucleic acid sequences included in the sequences as set forth in SEQ ID NOs: 1-379. In yet another embodiment, the present invention also comprises fragments of nucleic acid sequences that comprise at least 15 consecutive nucleic acid sequences for the complement of nucleic acid sequences included in the sequences as set forth in SEQ ID NOs: 1-379. In an embodiment, the glycoprotein comprises an amino acid sequence having at least 80% homology to at least one of the amino acid sequences as set forth in SEQ ID. NO: 44, SEQ ID. NO 46, SEQ ID. NO 48, SEQ ID. NO 50, SEQ ID. NO 52, SEQ ID. NO 54, SEQ ID. NO 56, SEQ ID. NO 58, SEQ ID. NO 60, SEQ ID. NO 62, SEQ ID. NO 64, SEQ ID. NO 70, or SEQ ID. NO 72. For example, in an embodiment, the present invention comprises an isolated nucleic acid molecule encoding a lectin-binding glycoprotein isolated from the exosporium of the Bacillus anthracis spore comprising a nucleic acid sequence as set forth in SEQ ID NO: 43, SEQ ID. NO: 45, SEQ ID. NO: 47, SEQ ID. NO: 49, SEQ ID. NO: 51, SEQ ID. NO: 53, SEQ ID. NO: 55, SEQ ID. NO: 57, SEQ ID. NO: 59, SEQ ID. NO: 61, SEQ ID. NO: 63, SEQ ID. NO: 69, or SEQ ID. NO: 71.

In an embodiment, the present invention also comprises vectors, wherein the vectors comprise recombinant DNA constructs comprising any of the nucleic acids disclosed herein. Also, the present invention may comprise cells comprising vectors that comprise recombinant DNA constructs comprising any of the nucleic acids disclosed herein.

In yet another embodiment, the present invention comprises methods of using these compositions for vaccination against anthrax infection and anthrax-like infections such as Bacillus cereus G9241. For example, in an embodiment, the compositions of the present invention can be used, either alone or in combination, as an antigen for eliciting protective immunity against anthrax. In an embodiment, the composition can be used with an adjuvant to help elicit an immune response.

The present invention also provides methods of preventing or treating anthrax infection. In another embodiment, the present invention comprises a method of treating or preventing anthrax infection, anthrax-like diseases, or other diseases of interest in a subject, comprising administering to the subject a composition comprising at least one glycoprotein from the exosporium of the Bacillus anthracis spore. Thus, in an embodiment, the present invention comprises a method of producing an immune response to Bacillus anthracis in a subject comprising administering to the subject the composition comprising a composition comprising at least one glycoprotein on the exosporium of the Bacillus anthracis spore, where the glycoprotein comprises at least one lectin-binding sugar. In an embodiment, the immune response is a cellular immune response. Alternatively or additionally, the immune response is a humoral immune response. In yet another embodiment, the present invention comprises a method of producing an immune response to Bacillus anthracis in a subject comprising administering to the subject any of the nucleic acids disclosed herein, whereby the nucleic acid of the composition can be expressed, for example, wherein the immune response is a cellular or humoral immune response.

The subjects treated with the vaccines and compositions of the present invention can be any mammal, such as a mouse, a primate, a human, a bovine, an ovine, an ungulate, or an equine. The compositions and/or vaccines of the present invention can be administered in any manner standard to vaccine administration. In an embodiment, administration is by injection. In another embodiment, administration may be by nasal inhalation.

The compositions and vaccines disclosed herein can be used individually, or in combination with other components of a spore from anthrax or an anthrax-like bacterium. Or, the compositions and vaccines may be used in combination with vaccines used to treat anthrax infection such as vaccines comprising protective antigen (PA), LF or EF (Pezard, C. et al. 1995, Infect. Immun., 63:1369-72) vaccine. Furthermore, the vaccines disclosed herein may include the use of an adjuvant. Also, other B. anthracis antigens can may be used (Brossier, F., and M. Mock, 2001, Toxicol., 39:1747-55; Cohen, S et al., 2000, Infect Immun 68:4549-58).

### Anthrax and Other Anthrax Like Infections

Anthrax is a highly fatal disease primarily of cattle, sheep and goats caused by the Gram-positive, endospore-producing, rod-shaped bacterium Bacillus anthracis. B. anthracis, like the other members of the genus Bacillus, can shift to a developmental pathway, sporulation, when growth conditions become unfavorable. The result of the sporulation process is the production of an endospore, a metabolically inert form of the cell which is refractive to numerous environmental insults including desiccation and heat. The spores produced by Bacillus species can persist in soil for long periods of time and are found worldwide.

Humans are also susceptible to infections by B. anthracis. Infections can occur in one of three forms. Entry of spores through abrasions in the skin results in the production of a lesion referred to as a malignant pustule, which is the hallmark of the cutaneous form of anthrax. This form is the most common form of “natural” human anthrax, has a low mortality rate, and responds well to antibiotic treatment. Ingestion of anthrax contaminated meat gives rise to the gastrointestinal form of the disease. This type of the disease is rare in the United States, although cases were reported in Minnesota in the year 2000 (Morbid. Mortal. Weekly Report, 2000, 49:813-816). This form of the disease has a higher mortality rate, approximately 40% in untreated cases. The most lethal form of human anthrax is the pulmonary form. Inhaled spores are deposited in the lungs and are engulfed by the alveolar macrophages (Ross, J. M., 1957, J. Pathol. Bacteriol, 73:485-494). The spores are then transported to the regional lymph nodes, germinating inside the macrophages en route (Ross, 1957; Guidi-Rontani, C., M., et al., 1999, Mol. Microbiol. 31:9-17). The early symptoms of pulmonary anthrax are nondescript influenza-like symptoms. The patient's condition deteriorates rapidly after the onset of symptoms and death often occurs within a few days. The mortality rate is high, 98% or greater, even with antibiotic therapy. Pulmonary anthrax is thus the primary concern in a bioterrorism attack. Recently, a strain of Bacillus cereus G9241 has been shown to cause a disease similar to inhalation anthrax (Hoffmaster, A. R., et al., 2004, Proc. Natl. Acad. Sci., USA, 101: 8449-8454). In mice, B. cereus G9241 is 100% lethal (Hoffmaster et al., 2004). Other strains of cereus have shown some of the virulence factors of B. anthracis such as B. cereus ATCC 10987 (Rask et al., 2004; Han et al., 2006, and Hoffmaster et al., 2006). It may be possible to combat infection from anthrax and anthrax like diseases with a single vaccine.

The spore is the infectious form of B. anthracis. The outside of the spore is characterized by the presence of an external exosporium that consists of a basal layer surrounded by an external nap of hair-like projections (Hoffmaster et al., 2004; Hachisuka, Y., et al., 1966, J. Bacteriol. 91:2382-2384; Kramer, M. J., and I. L. Roth, 1968, Can J. Microbiol. 14:1297-1299). Upon entry of spores in the lung, the spores are rapidly taken up by macrophages where they germinate. In the vegetative form (multiplicative form) the spore exosporium and coat layers are replaced by a poly-D-glutamic acid capsule and S (surface) layers.

The fate of macrophage engulfed spores has been examined (Dixon, T. C., et al., 2000, Cell. Microbiol., 2:453-463; Guidi-Rontani, C., et al., 1999, Mol. Microbiol. 31:9-17; Guidi-Rontani, C., et al., 2001, Molec. Microbiol. 42:931-938). When spores of B. anthracis attach to the surface of macrophages, they may be rapidly phagocytized. There can be a tight interaction between the exosporium and the phagolysosomal membrane; however, newly vegetative bacilli may escape from the phagosomes of cultured macrophages and replicate within the cytoplasm of the cells. Release of bacteria from the macrophage occurs 4-6 hours after phagocytosis of the spores. The principal virulence factors of B. anthracis are encoded on plasmids. One plasmid (pXO1) carries the toxin genes while a second plasmid (pXO2) encodes the polyglutamic acid capsule biosynthetic apparatus.

In certain embodiments, the methods and compositions of the present invention may also be used to develop vaccines for other anthrax-like bacteria or microorganisms of interest. Spores of anthrax-like infections are similar to those of B. anthracis spores. For example, Bacillus cereus has been shown to have an exosporium that contains glycoproteins, oligosaccharides, and other sugars. Also, the B. cereus G9241 vegetative cell can resemble an anthrax vegatative cell because both contain a capsule, although the B. cereus G9241 capsule is not coded for the pXO2 plasmid of B. anthracis, but appears to be encoded for by a pBC218 cluster (Hoffmaster et al., 2004). Several of the anthrax toxins encoded for on the pXO1 plasmid may have similar counterparts in B. cereus G9241 encoded for on pBC218 including AtxA (toxin regulator), lethal factor, and protective antigen (PA). There is evidence that the PA found in B. cereus G9241 may be functional, because 27 out of 33 amino acids important to the functionality of the PA are identical in B. anthracis Ames strain and B. cereus G9241.

Antibodies reactive with the surface of spores of B. anthracis spores may affect the interactions of the spore with host cells and/or the environment. For example, spore surface reactive antibodies may enhance phagocytosis of the spores by murine peritoneal macrophages, and may inhibit spore germination in vitro. The first spore-surface protein, termed BclA (Bacillus, collagen-like protein) has been recently described in B. anthracis. The poly-D-glutamic acid capsule is not present in the spore, thus surface proteins, including BclA, constitute the surface layer. Mass spectrometry has been utilized to look for other spore-specific constituents of B. anthracis.

The spore is characterized by the presence of 3-O-methyl rhamnose, rhamnose and galactosamine. This carbohydrate is found only in the spores and is not synthesized by vegetatively growing cells. B. thuringiensis and B. cereus are closely related genetically to B. anthracis and the exosporium of both contain a glycoprotein whose major carbohydrate constituent is rhamnose, while the B. thuringiensis protein additionally contains galactosamine. Another sugar monomer is present in the B. thuringienisis exosporium, which can be 3-O-methyl rhamnose or 2-O-methyl rhamnose, identified previously as spore sugars.

1. Preparation of Compositions

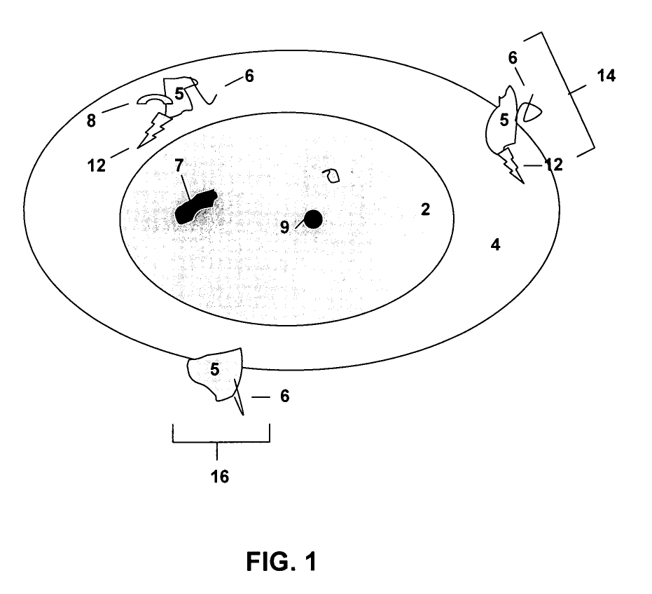

In an embodiment, glycoproteins on the exosporium of the B. anthracis spore may be complexed to other proteins, glycoproteins, oligosaccharides, lipids, or phospholipids. A diagrammatic representation of a B. anthracis bacterium (or other microorganisms) 2 surround by a exosporium 4 is provided in FIG. 1. Thus, it can be seen that the spore may comprise a variety of glycoproteins or lippopolysaccharides 5, complexed with other biomolecules such as sugars or oligosaccharides 6, peptides 8, lipids 12 and the like. Also, in an embodiment, at least some of these complexes 14, 16 are antigenic, such that isolation of the antigenic epitopes may be used to create an anti-anthrax vaccine. Thus, as discussed herein, it has been found that vaccines comprising only toxin proteins 7,9 (e.g., PA; LF) isolated from the actual bacterium are not completely effective against inhalation anthrax. By adding spore-based antigens to a vaccine, embodiments of the compositions of the present invention can provide improved immunity to anthrax and anthrax-based diseases (or to other disease of interest).

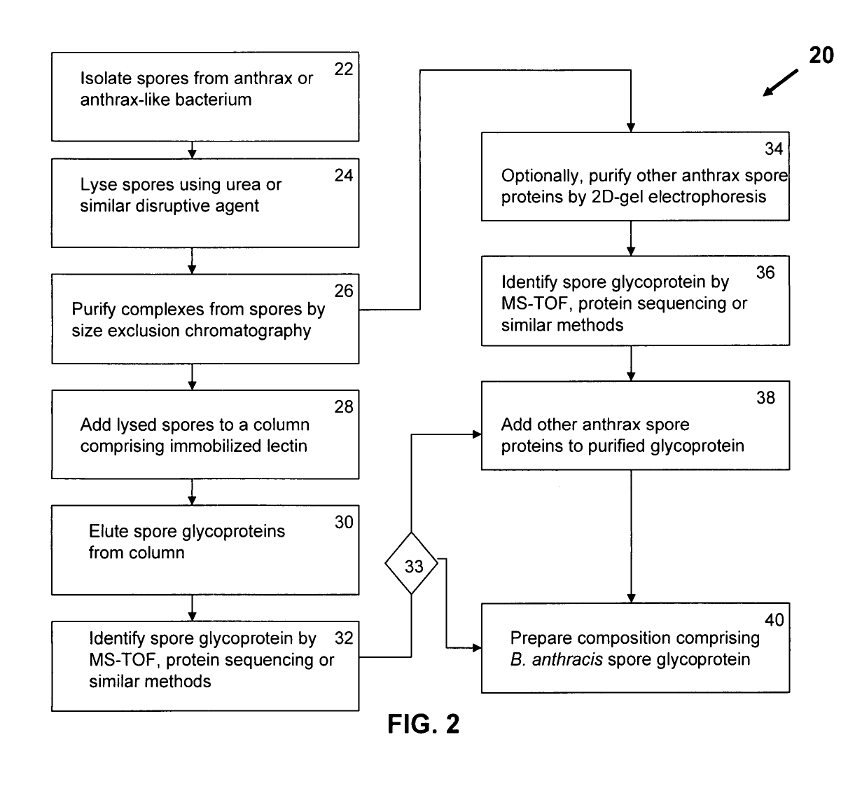

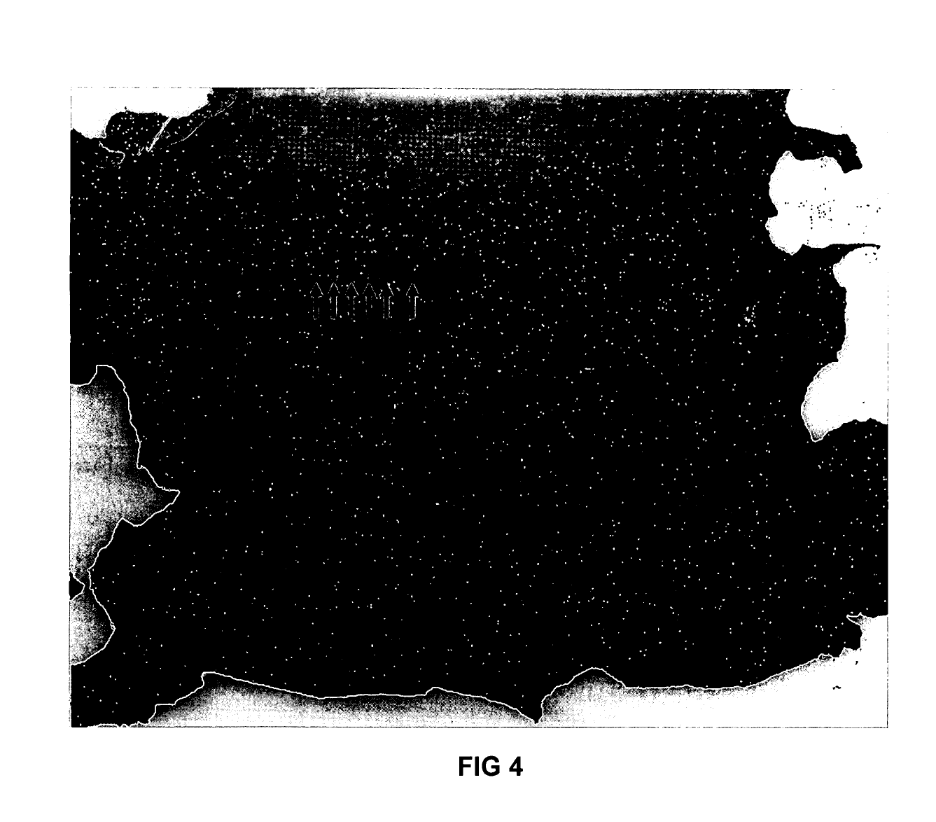

FIG. 2 provides a schematic representation of a method of the present invention. The method may comprise two parts which may be performed individually, or in combination as shown in FIG. 2. As shown in FIG. 2, in an embodiment, the present invention provides a method for purifying glycoproteins and other molecules from the B. anthracis spore. In an embodiment, the method may comprise a first step of isolating spores from B. anthracis, or another anthrax-like bacterium (or microorganism of interest) 22. Isolation of the spores may be performed centrifugation as described in Example 11 herein or other methods known in the art such as high performance liquid chromatography (HPLC). An example of isolated B. anthracis spores as isolated by 2D-gel electrophoresis is shown in FIG. 4 (arrows point to the white spores). Next the method may comprise lysing the spores using urea, sonication, bead beatting, French press, or some other means 24. Lysing the spores may be performed by taking a pure (about 95-100% purity) spore solution (B. anthracis spores plus PBS or water) and performing a urea extract or some other lysis procedure such as sonicating herein or using methods known in the art.

At this point an optional step of purifying complexes from the spores by size-exclusion chromatography or HPLC 26 may be performed.

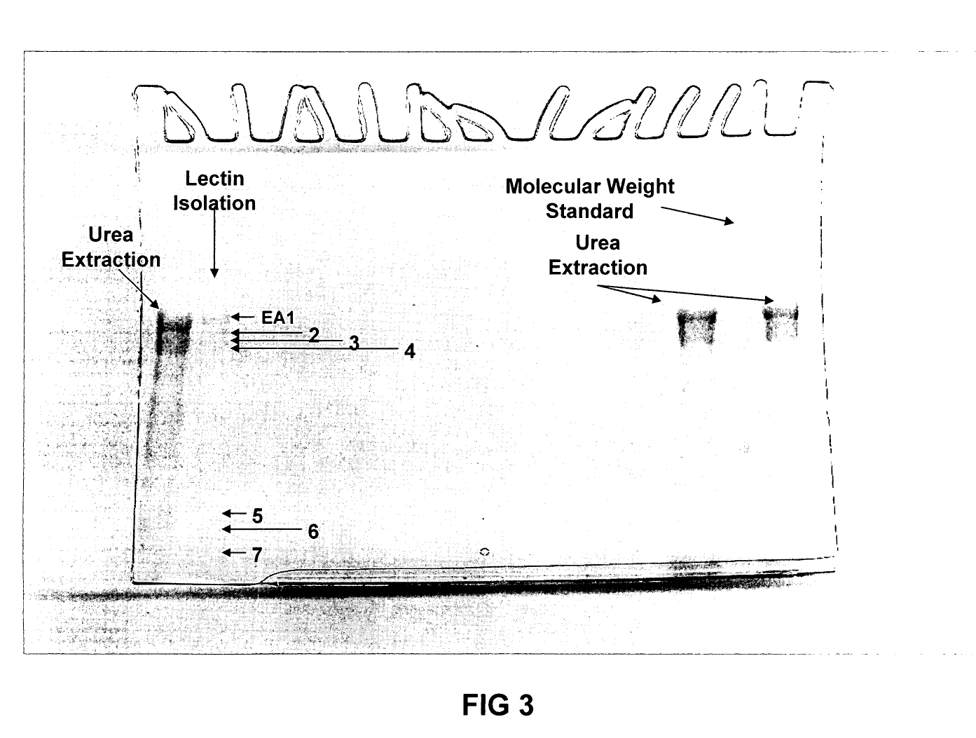

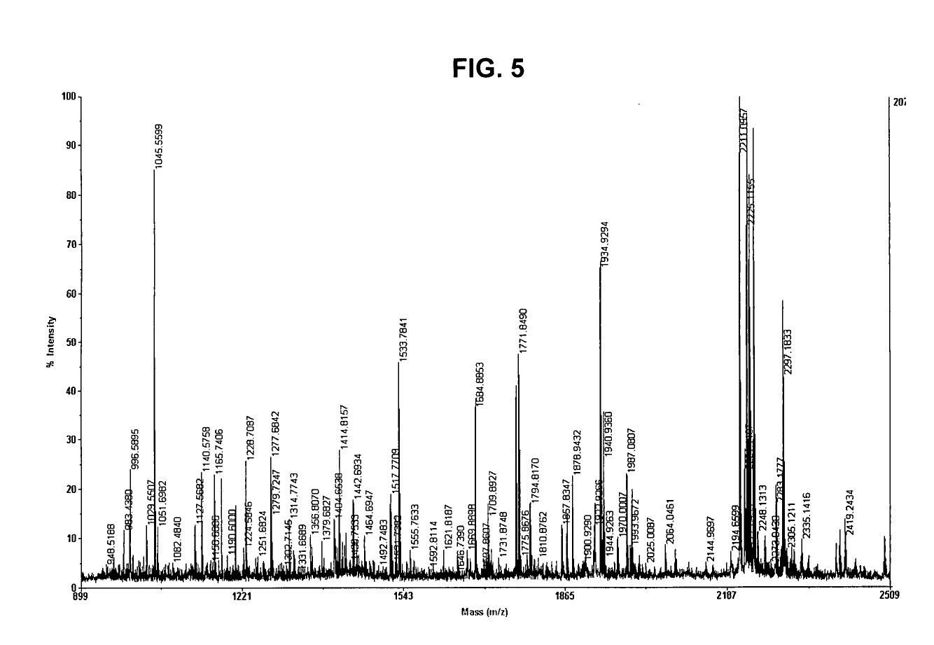

Next, the lysed spores, or size-selected fraction may be applied to a column to purify glycoproteins contained in the complexes. In an embodiment, lectin is used to purify glycoprotein complexes from the spore mixture 28. Lectins are sugar binding proteins that can recognize and bind to the carbohydrate portion of a glycoprotein. The lectin can then be released from the glycoprotein by washing the lectin with another sugar that has a stronger affinity for the lectin than the B. anthracis glycoprotein 30. An example showing a subset of B. anthracis proteins purified by lectin-binding is shown in FIG. 3. Thus, it can be seen that upon extraction with lectin, a subset of the proteins (e.g., EA1, and new proteins 1, 2, 3, 4, 5, 6, and 7) seen in the urea extracted spore are isolated. At this point, the eluted glycoprotein may be identified by time of flight mass spectrometry (MS-TOF), protein sequencing or other similar methods 32. For example, FIG. 5 shows results for MALDI TOF MS of the EA1 band seen on the gel of FIG. 3. As described herein, the glycoprotein complexes can be used as a vaccine for immunity against anthrax infection or any anthrax like diseases or as a diagnostic tool for detection of Bacillus anthracis, any other anthrax like spores or where another microorganism of interest.

In an embodiment, electroelution may be used to delete specific proteins from the lectin-purified complexes. Alternatively, electroelution of urea extracted or other lysed spores may be used to add proteins to the lectin complexed mixture 34 (FIG. 2). For electroelution, one or two dimensional SDS (sodium dodecyl sulfate) PAGE (polyacrylamide gel electrophoresis) or native gel electrophoresis of the isolated spore proteins may be performed. The gel may then be stained, and the spot of interest cut out, and destained. Next, an electrical charge is ran through the isolated portion of the gel containing the protein of interest to elute the protein from the gel. Other techniques, such as size exclusion chromatography or HPLC may be used to remove proteins, glycoproteins, lipids, phospholipids, or oligosaccharides outside the molecular weights of interest. The eluted protein may be captured on a filter, or in a vessel such as a tube or filter tube, and analyzed by MS-TOF, protein sequencing or other similar methods such s MALDI TOF-TOF, ESI-IT, MADLIFT-ICR or ESI FT-ICR MS 36.

In an embodiment, only specific glycoproteins isolated from the lectin column and correlating with the spots of interest on a one or two dimensional SDS or native gel are used to make the compositions of the present invention (e.g., a vaccine) 33, 40 (FIG. 2). Alternatively, proteins isolated from the spore complex may be added back to the purified glycoprotein complex(es) and used to make a composition of the present invention. 33, 38, 40 (FIG. 2).

FIG. 3, panels A and B, shows a representation of the type of results that may be obtained upon upon isolating B. anthracis spore proteins by lectin treatment. Thus, in an embodiment, the profile of proteins in the sample may be characterized by one or two-dimensional (2D) gel electrophoresis. In an embodiment the samples are separated in one dimension on the basis of charge along a gradient of increasing pH, as in 2D gel electrophoresis an in the other dimension on the basis of size. It can be seen that the profile of proteins isolated from the B. anthracis spore comprises substantially fewer proteins after lectin treatment (FIG. 3B) than before lectin treatment (FIG. 3A).

2. Vaccines

In an embodiment, the compositions of the present invention comprise a vaccine. Several basic strategies may be used to make vaccines against viral and bacterial infections. U.S. Patent applications disclosing vaccines to anthrax and anthrax like infections are 20030118591, 2004/0009178, 2004/0009945, 2002/0142002; these patent applications are incorporated by reference herein with respect to material related to anthrax vaccines and the materials used to make anthrax vaccines. The anthrax vaccine containing the protective antigen (PA) component of the tripartite anthrax toxin (AVA) is not fully protective in animal studies. Indeed, a conjugate vaccine, additionally targeting the poly-D-glutamic acid capsule (PGA), which surrounds and protects the vegetative cell from killing by complement mediated killing (Rhie et al., 2003; Schneerson et al., 2003), has been sought after. However, such a vaccine would target the vegetative cell and lethal toxin, but not the initial interaction of the macrophage with the spore.

The vaccines disclosed herein may be composed of lectin-purified glycoprotein complexes isolated from B. anthracis spores. In an embodiment, the vaccines are used in combination with other components isolated from the anthrax bacterium and/or spore such as protective antigen or LF antigen. Or capsule components may be included. Furthermore, the vaccine may use lectin-purified glycoprotein complexes isolated from the B. anthracis spores in whole or in part, including complexes that may contain deglycosylated forms, fusion proteins, or missing or deleted subunits of the glycoprotein complex. In an embodiment, fragments of a B. anthracis lectin binding glycoprotein can be combined with PA fragments. For example, fragments of a B. anthracis lectin binding glycoprotein complex can be combined with PA fragments. Or, fragments of a B. anthracis lectin binding glycoprotein complexes can be combined with other spore associated antigens such as extractable antigen 1 (EA1), Serum Amyloid P Component (SAP) or capsular poly-gamma-d-glutamic acid (PGA). In another embodiment, the present-invention relates to an anthrax vaccine comprising one or more replicon particles derived from one or more replicons encoding one or more B. anthracis proteins or polypeptides.

In an embodiment, the vaccines of the present invention comprise an adjuvant to increase the humoral and/or cellular immune response. In an embodiment, the adjuvant is one that is approved by the Food and Drug Administration such as aluminum hydroxide and aluminum phosphate. Or the Ribi adjuvant can be employed.

3. Vaccine Administration

The peptides, compositions, vaccines or antibodies disclosed herein can be administered by any mode of administration capable of delivering a desired dosage to a desired location for a desired biological effect which are known to those of ordinary skill in the art. Routes or modes include, for example, oral administration, parenteral administration (e.g., intravenously, by intramuscular injection, by intraperitoneal injection), or by subcutaneous administration. In an embodiment, the vaccine is prepared for subcutaneous or intramuscular injection. The vaccine may be formulated in such a way as to render it deliverable to a mucosal membrane without the peptides being broken down before providing systemic or mucosal immunity, such as, orally, inhalationally, intranasally, or rectally. The amount of active compound administered will, of course, be dependent, for example, on the subject being treated, the subject's weight, the manner of administration and the judgment of the prescribing physician. Immunogenic amounts can be determined by standard procedures. An “immunogenic amount” is an amount of the protein sufficient to evoke an immune response in the subject to which the vaccine is administered. An amount of from about 10² to 10⁷ micrograms per kilogram dose is suitable, with more or less used depending upon the age and species of the subject being treated.

Depending on the intended mode of administration, the compositions or vaccines may be in the form of solid, semi solid or liquid dosage forms, such as, for example, tablets, suppositories, pills, capsules, powders, liquids, suspensions, or the like, preferably in unit dosage form suitable for single administration of a precise dosage. The compositions or vaccines may include, as noted above, an effective amount of the selected immunogens in combination with a pharmaceutically acceptable carrier and, in addition, may include other medicinal agents, pharmaceutical agents, carriers, adjuvants, diluents, etc. Exemplary pharmaceutical carriers include sterile pyrogen-free water and sterile pyrogen-free physiological saline solution.

Parental administration can involve the use of a slow release or sustained release system, such that a constant level of dosage is maintained. See, e.g., U.S. Pat. No. 3,710,795, which is incorporated by reference herein. A system using slow release or sustained release may be used with oral administration as well. The vaccine or composition can be administered in liposomes, encapsulated, or otherwise protected or formulated for slower or sustained release. The antibody level following the first exposure to a vaccine antigen referred to as primary antibody response may consist primarily of IgM, and may be of brief duration and low intensity, so as to be inadequate for effective protection. The antibody level following the second and subsequent antigenic challenges, or secondary antibody response, may appear more quickly and persists for a longer period, attain a higher titer, and consists predominantly of IgG. The shorter latent period is generally due to antigen-sensitive cells, called memory cells, already present at the time of repeat exposure.

In an embodiment, the vaccine is provided as an adenovirus vector. In an embodiment, the adenovirus-based vaccine can be administrated by different routes to achieve immunization such as intramuscular injection (parentally), intranasal administration or oral administration. The intranasal immunization with this type of vaccine may be preferred to elicit more potent mucosal immunity against the pathogen, in this case, anthrax spores. In an embodiment, intranasal administration may be provided for protection against inhalation anthrax caused by aerosol dismissed anthrax spore propagated by a bioterrorism attack.

Anthrax vaccines as currently administered can function with six immunizations over a period of 18 months followed by annual boosters. In an embodiment, the vaccines of the present invention may be provided with 1, 2, 3, 4, or 5 immunizations to provide protective immunity with optional boosters. Examples of suitable immunization schedules include, but are not limited to: (i) 0, 1 months and 6 months, (ii) 0, 7 days and 1 month, (iii) 0 and 1 month, (iv) 0 and 6 months, or other schedules sufficient to elicit the desired immune responses expected to confer protective immunity, or reduce disease symptoms, or reduce severity of disease.

In an embodiment, the vaccine of the present invention may provide at least one of anti-glycoprotein complex IgG antibody titers, anti-glycoprotein complex IgG1 antibody titers, anti-glycoprotein complex IgG2a antibody titers. In alternate embodiments, antibody titers of 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 10500, 11000, 11500, and 12000 by 2, 4, 6, 8, 10, 12, 14, 16, 18, and 20 weeks post-immunization following 1, 2, 3, 4, 5, or more immunizations are achieved. In an embodiment, booster inoculations are used to maintain effective immunization. Boosters can be given every 1, 2, 3, 4, 6, 8, 12 years following prior inoculation, for example.

In an embodiment, the vaccine may comprise a nucleic acid that encode for an immunogenic anthrax protein or polypeptide isolated by the methods of the present invention. For example, in an embodiment, a nucleic acid comprising a nucleic acid sequence included in the sequences as set forth in SEQ ID NOs: 1-379 may be used in a vaccine of the present invention.

When DNA (or RNA corresponding to the DNA sequence) is used as a vaccine, the DNA (or RNA) can be administered directly using techniques such as delivery on gold beads (gene gun), delivery by liposomes, or direct injection, among other methods known to people in the art. Any one or more constructs or DNA or RNA can be use in any combination effective to elicit an immunogenic response in a subject. Generally, the nucleic acid vaccine administered may be in an amount of about 1-5 μg of nucleic acid per dose and will depend on the subject to be treated, capacity of the subject's immune system to develop the desired immune response, and the degree of protection desired. Precise amounts of the vaccine to be administered may depend on the judgment of the practitioner and may be peculiar to each subject and antigen.

4. Assays for Assessing the Immune Response

Embodiments of the present invention also provide assays for assessing an immune response to the components isolated from the endosporium of B. anthracis.

The assays may comprise in vivo assays, such as assays to measure antibody responses and delayed type hypersensitivity responses. In an embodiment, the assay to measure antibody responses primarily may measure B-cell function as well as B-cell/T-cell interactions. In another embodiment, the delayed type hypersensitivity response assay may measure T-cell immunity. For the antibody response assay, antibody titers in the blood may be compared following an antigenic challenge. These levels can be quantitated according to the type of antibody, as for example, IgG, IgG1, IgG2, IgM, or IgD. Also, the development of immune systems may be assessed by determining levels of antibodies and lymphocytes in the blood without antigenic stimulation. An agglutination assay to test the highest dilution of antibodies that can still bind to B. anthracis spores or any other strain of anthrax may be used.

The assays may also comprise in vitro assays. The in vitro assays may comprise determining the ability of cells to divide, or to provide help for other cells to divide, or to release lymophokines and other factors, express markers of activation, and lyse target cells. Lymphocytes in mice and man can be compared in vitro assays. In an embodiment, the lymphocytes from similar sources such as peripheral blood cells, spleenocytes, or lymph node cells, are compared. It is possible, however, to compare lymphocytes from different sources as in the non-limiting example of peripheral blood cells in humans and splenocytes in mice. For the in vitro assay, cells may be purified (e.g., B-cells, T-cells, and macrophages) or left in their natural state (e.g., splenocytes or lymph node cells). Purification may be by any method that gives the desired results. The cells can be tested in vitro for their ability to proliferate using mitogens or specific antigens. Mitogens can specifically test the ability of-either T-cells to divide as in the non-limiting examples of concanavalin A and T-cell receptor antibodies, or B-cells to divide as in the non-limiting example of phytohemagglutinin. The ability of cells to divide in the presence of specific antigens can be determined using a mixed lymphocyte reaction, MLR, assay. Supernatant from the cultured cells can be tested to quantitate the ability of the cells to secrete specific lymphokines. The cells can be removed from culture and tested for their ability to express activation antigens. This can be done by any method that is suitable as in the non-limiting example of using antibodies or ligands to which bind the activation antigen as well as probes that bind the RNA coding for the activation antigen.

Also, in an embodiment, phenotypic cell assays can be performed to determine the frequency of certain cell types. Peripheral blood cell counts may be performed to determine the number of lymphocytes or macrophages in the blood. Antibodies can be used to screen peripheral blood lymphocytes to determine the percent of cells expressing a certain antigen as in the non-limiting example of determining CD4 cell counts and CD4/CD8 ratios.

In certain embodiments, transformed host cells can be used to analyze the effectiveness of drugs and agents which inhibit anthrax or B. anthracis proteins, such as host proteins or chemically derived agents or other proteins which may interact with B. anthracis proteins of the present invention to inhibit its function. A method for testing the effectiveness of an anti-anthrax drug or anti-anthrax like diseases drug or agent can for example be the rat anthrax toxin assay (Ivins et al. 1986, Infec. Immun. 52, 454-458; and Ezzell et al., Infect. Immun., 1984, 45:761-767) or a skin test in rabbits for assaying antiserum against anthrax toxin (Belton and Henderson, 1956, Br. J. Exp. Path. 37, 156-160).

5. Generation of Antibodies

Other embodiments of the present invention comprise generation of antibodies that specifically recognize a lectin-binding glycoprotein isolated from the endosporium of the B. anthracis spore alone, or in combination with other B. anthracis components. In an embodiment, the antibody preparation, whether polyclonal, monoclonal, chimeric, human, humanized, or non-human can recognize and target the variants and fragments a lectin-binding glycoprotein complex isolated from the B. anthracis spore alone, or in combination with other B. anthracis components. Antibodies that specifically recognize non-native variants or fragments of any of the lectin-binding glycoprotein complexes isolated from the endosporium of the B. anthracis spore alone, or in combination with other B. anthracis components could, for example, be used to purify recombinant fragments lectin-binding glycoprotein complexes isolated from the endosporium of the B. anthracis spore and variants of such proteins. Such antibodies could also be used as “passive vaccines” for the direct immunotherapeutic targeting of Bacillus anthracis in vivo. Also disclosed are methods of using said antibodies to detect anthrax spores or spore fragments, either in vitro or in vivo, for research or diagnostic use.

In an embodiment, the antibodies provided herein are capable of neutralizing anthrax spores and spores of other closely related species to anthrax. The provided antibodies can be delivered directly, such as through needle injection, for example, to treat anthrax or anthrax-like infections. The provided antibodies can be delivered non-invasively, such as intranasally, to treat inhalation anthrax or anthrax-like diseases.

In an embodiment, the antibodies may be encapsulated, for example into lipsomes, microspheres, or other transfection enhancement agents, for improved delivery into the cells to maximize the treatment efficiency. In an embodiment, the DNA sequences encoding the provided antibodies, or their fragments such as Fab fragments, may be cloned into genetic vectors, such as plasmid or viral vectors, and delivered into the hosts for endogenous expression of the antibodies for treatment of anthrax or anthrax-like diseases.

In an embodiment, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., 1986, Nature, 321:522-525; Riechmann et al., 1988, Nature, 332:323-327; and Presta, Curr. Op. Struct. Biol., 1992, 2:593-596.

Methods for humanizing non-human antibodies known in the art may be used to humanize the antibodies of the present invention. Generally, a humanized antibody has one or more amino acid residues introduced into it from a source that is non-human. These non-human amino acid residues are often referred to as “import” residues, which are typically taken from an “import” variable domain. Humanization can be essentially performed by substituting rodent CDRs or CDR sequences for the corresponding sequences of a human antibody (see e.g., Jones et al., 1986, Nature, 321:522-525; Riechmann et al., 1988, Nature, 332:323-327; Verhoeyen et al., 1988, Science, 239:1534-1536. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies may be highly important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., 1993, J. Immunol., 151:2296; Chothia et al., 1987, J. Mol. Biol., 196:901. Another method uses a particular framework derived from the consensus sequence of all human antibodies of a particular subgroup of light or heavy chains. The same framework may be used for several different humanized antibodies (Carter et al., Proc. Natl. Acad. Sci. USA, 1992, 89:4285; Presta et al., J. Immunol., 1993, 151:2623).

In an embodiment, the antibodies are humanized with retention of high affinity for the antigen and other favorable biological properties. To achieve this goal the humanized antibodies may be prepared by analysis of the parental sequences and various conceptual humanized products using three dimensional models of the parental and humanized sequences. Computerized comparison of these displays to publicly available three dimensional immunoglobulin models permits analysis of the likely role of the residues in the functioning of the candidate immunoglobulin sequence, i.e., the analysis of residues that influence the ability of the candidate immunoglobulin to bind its antigen. In this way, the human framework (FR) residues can be selected and combined from the consensus and import sequence so that the desired antibody characteristic, such as increased affinity for the target antigen(s), is achieved. In general, the CDR residues are directly and most substantially involved in influencing antigen binding (see e.g., WO 94/04679).

In an embodiment, transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region J_{H} gene in chimeric and germ-line mutant mice results in complete inhibition of endogenous antibody production. Transfer of the human germ-line immunoglobulin gene array in such germ-line mutant mice can result in the production of human antibodies upon antigen challenge (see, e.g., Jakobovits et al., 1993, Proc. Natl. Acad. Sci. USA, 90:2551-2555; Jakobovits et al., 1993, Nature, 362:255-258; Bruggemann et al., 1993, Year in Immunology, 7:33).

In yet another embodiment, human antibodies may also be produced in phage display libraries (Hoogenboom et al., 1991, J. Mol. Biol., 227:381; Marks et al., 1991, J. Mol. Biol., 222:581. In another embodiment, the antibodies are monoclonal antibodies (see e.g., Cole et al., 1985, Monoclonal Antibodies and Cancer Therapy, Alan R. Liss, p. 77; Boerner et al., 1991, J. Immunol., 147(1):86-95. For example, the present invention may comprise hybridoma cells that produce monoclonal antibodies. Monoclonal antibodies may be prepared using hybridoma methods (see e.g., Kohler and Milstein, 1975, Nature, 256:495; or Harlow and Lane, 1988, Antibodies, A Laboratory Manual, Cold Spring Harbor Publications, New York). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises a composition comprising at least one glycoprotein on the exosporium of the Bacillus anthracis spore where the glycoprotein comprises at least one lectin-binding sugar.

Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of the anthrax spores expressed as a fusion protein with human IgG 1 is injected into the host animal according to methods known in the art (e.g., Kilpatrick K E, et al., 1998, Hybridoma, December 17(6):569-76; Kilpatrick K E et al., 2000, Hybridoma, August, 19(4):297-302) and as described in the examples.

In yet another embodiment, the antigen may be expressed in baculovirus. The advantages to the baculovirus system include ease of generation, high levels of expression, and post-translational modifications that are highly similar to those seen in mammalian systems. The antigen is produced by inserting a gene encoding the B. anthracis antigenic protein so as to be operably linked to a signal sequence such that the antigen is displayed on the surface of the virion. This method allows immunization with whole virus, eliminating the need for purification of target antigens.

In an embodiment, peripheral blood lymphocytes (“PBLs”) are used in methods of producing monoclonal antibodies if cells of human origin are desired. In an alternate embodiment, spleen cells or lymph node cells may be used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines may be transformed mammalian cells, including myeloma cells of rodent, bovine, equine, and human origin. In an embodiment, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, 1984, J. Immunol., 133:3001; Brodeur et al., 1987, “Monoclonal Antibody Production Techniques and Applications” Marcel Dekker, Inc., New York, pp. 51-63). The culture medium in which the hybridoma cells are cultured can then be assayed for the presence of monoclonal antibodies directed against the B. anthracis antigen.

In an embodiment, the binding specificity of monoclonal antibodies produced by the hybridoma cells may be determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for anthrax spores and anthrax-like other species.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348; U.S. Pat. No. 4,342,566; and Harlow and Lane, Antibodies, 1988, A Laboratory Manual, Cold Spring Harbor Publications, New York. Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen. The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

In other embodiments, an isolated immunogenically specific paratope or fragment of the antibody is also provided. A specific immunogenic epitope of the antibody can be isolated from the whole antibody by chemical or mechanical disruption of the molecule. The purified fragments thus obtained may then be tested to determine their immunogenicity and specificity by the methods described herein. Immunoreactive paratopes of the antibody, optionally, are synthesized directly. An immunoreactive fragment is defined as an amino acid sequence of at least about two to five consecutive amino acids derived from the antibody amino acid sequence.

In another embodiment, the antibodies of the present invention may be made by linking two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide may be independently synthesized in vivo as described above. Once isolated, these independent peptides or polypeptides may be linked to form an antibody or fragment thereof via similar peptide condensation reactions.

For example, in an embodiment, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al., 1994, Science, 266:776-779). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al., 1992, FEBS Lett. 307:97-101; Clark-Lewis I et al., 1994, J. Biol. Chem., 269:16075); Clark-Lewis I. et al., 1991, Biochemistry, 30:3128; Rajarathnam K et al., 1994, Biochemistry 33:6623-30).

Alternatively, unprotected peptide segments may be chemically linked where the bond formed between the peptide segments as a result of the chemical ligation is an unnatural (non-peptide) bond (Schnolzer, M et al., 1992, Science, 256:221). This technique has been used to synthesize analogs of protein domains as well as large amounts of relatively pure proteins with full biological activity (deLisle Milton R C et al., 1992, Techniques in Protein Chemistry IV. Academic Press, New York, pp. 257-267).

Also disclosed are fragments of antibodies which have bioactivity. The polypeptide fragments can be recombinant proteins obtained by cloning nucleic acids encoding a glycoprotein of the B. anthracis spore polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with anthrax spores or spores of other closely related species. Amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule, or the immunoglobulin molecule, and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragment of the B. anthracis spore polypeptide, whether attached to other sequences or not, include insertions, deletions, substitutions, or other selected modifications of particular regions or specific amino acids residues, provided the activity of the fragment is not significantly altered or impaired compared to the non-modified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al., 1982, Nucl. Acids Res. 10:6487-500). A variety of immunoassay formats may be used to select antibodies that selectively bind with a particular protein, variant, or fragment. For example, solid-phase ELISA immunoassays are routinely used to select antibodies selectively immunoreactive with a protein, protein variant, or fragment thereof (Harlow and Lane, 1988).

In yet another embodiment, the present invention comprises an antibody reagent kit comprising containers of the monoclonal antibody to at least one of the sugar complexed components of the Bacillus anthracis spore where the complex comprises at least one lectin-binding sugar or fragment thereof and one or more reagents for detecting binding of the antibody or fragment thereof to at least one of the sugar complexed components on the Bacillus anthracis spore where the glycoprotein comprises at least one lectin-binding sugar. The reagents can include, for example, fluorescent tags, enzymatic tags, or other tags. The reagents can also include secondary or tertiary antibodies or reagents for enzymatic reactions, wherein the enzymatic reactions produce a product that can be visualized.

6. Functional Nucleic Acids

In an embodiment, the compositions of the present invention comprise a functional nucleic acid as a therapeutic agent for the treatment or prevention of anthrax, anthrax-like infections or other diseases of interest. Functional nucleic acids are nucleic acid molecules that have a specific function, such as binding a target molecule or catalyzing a specific reaction. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. In an embodiment, the functional nucleic acid of the present invention can interact with the mRNA encoding for at least one glycoprotein on the exosporium of the Bacillus anthracis spore where the glycoprotein comprises at least one lectin-binding sugar. In yet another embodiment the functional nucleic acid of the present invention can interact with at least one glycoprotein on the exosporium of the Bacillus anthracis spore where the glycoprotein comprises at least one lectin-binding sugar. Or, the functional nucleic acid of the present invention may interact with the genomic DNA encoding for at least one glycoprotein on the exosporium of the Bacillus anthracis spore where the glycoprotein comprises at least one lectin-binding sugar. The functional nucleic acids may be designed to interact with other B. anthracis nucleic acids based on sequence homology between the target molecule and the functional nucleic acid molecule. In other embodiments, the specific recognition between the functional nucleic acid molecule and the target molecule is not based on sequence homology between the functional nucleic acid molecule and the target molecule, but rather is based on the formation of tertiary structure that allows specific recognition to take place.

In an embodiment, the functional nucleic acid may comprise an antisense nucleic acid. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule may be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods may include in vitro selection experiments and DNA modification studies using DMS and DEPC. In alternate embodiments, antisense molecules bind the target molecule with a dissociation constant (k_{d}) less than or equal to 10⁻⁶, 10⁻⁸, 10⁻¹⁰, or 10⁻¹² M. A representative sample of methods and techniques which aid in the design and use of antisense molecules can be found in the following U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

In another embodiment, the functional nucleic acid may comprise an aptamer. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophylline (U.S. Pat. No. 5,580,737), as well as large molecules, such as reverse transcriptase (U.S. Pat. No. 5,786,462) and thrombin (U.S. Pat. No. 5,543,293). In an embodiment, the aptamers of the present invention can bind very tightly to the target molecule with a dissociation constant (k_{d}) of less than 10⁻¹² M. In alternate embodiments, the aptamers may bind the target molecule with a k_{d} less than 10⁻⁶, 10⁻⁸, 10⁻¹⁰, or 10⁻¹² M. The aptamers of the present invention can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). In alternate embodiments, the aptamer may have a k_{d} with the target molecule at least 10, 100, 1000, 10,000, or 100,000 fold lower than the k_{d} with a background binding molecule such as serum albumin. Representative examples of how to make and use aptamers to bind a variety of different target molecules can be found in the following non-limiting list of U.S. Pat. Nos. 5,476,766, 5,503,978, 5,631,146, 5,731,424 , 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

In another embodiment, the composition may comprise a ribozyme. Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes (e.g., U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, and international patent applications WO 9858058, WO 9858057, and WO 9718312) hairpin ribozymes (e.g., U.S. Pat. Nos. 5,631,115, 5,646,031, 5,683,902, 5,712,384, 5,856,188, 5,866,701, 5,869,339, and 6,022,962), and tetrahymena ribozymes (e.g., U.S. Pat. Nos. 5,595,873 and 5,652,107). There are also a number of ribozymes that are not found in natural systems, but which have been engineered to catalyze specific reactions de novo (e.g., U.S. Pat. Nos. 5,580,967, 5,688,670, 5,807,718, and 5,910,408). In an embodiment, the ribozyme may cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

In another embodiment, the composition may comprise a triplex forming nucleic acid. Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. In alternate embodiments, the triplex forming molecules bind the target molecule with a k_{d} less than 10⁻⁶, 10⁻⁸, 10⁻¹⁰, or 10⁻¹²M. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

In another embodiment, the composition may comprise an external guide sequences (EGSs). External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)). Similarly, eukaryotic EGS/RNAse P-directed cleavage of RNA can be utilized to cleave desired targets within eukaryotic cells. (Yuan et al., Proc. Natl. Acad. Sci. USA, 1992, 89:8006-8010; WO 93/22434; WO 95/24489; Yuan and Altman, EMBO J., 1995, 14:159-168, and Carrara et al., Proc. Natl. Acad. Sci. (USA), 1995, 92:2627-2631. Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

7. Peptides

In an embodiment, the composition and/or vaccine of the present invention may comprise a polypeptide fragment of at least one glycoprotein on the exosporium of the Bacillus anthracis spore where the glycoprotein comprises at least one lectin-binding sugar. The peptide can be an antigen or the antigen bound to a carrier or a mixture of bound or unbound antigens. The peptide can then be used in a method of preventing anthrax infection or anthrax-like infections. For example, in an embodiment, the peptide may be useful as a vaccine.

Immunogenic amounts of the antigen can be determined using standard procedures. Briefly, various concentrations of a putative specific immunoreactive peptides or polypeptides may be prepared, administered to an animal, such as a human, and the immunological response (e.g., the production of antibodies or cell-mediated response) of an animal to each concentration determined. The pharmaceutically acceptable carrier in the vaccine can comprise saline or other suitable carriers (Arnon, R. (Ed.), 1987, Synthetic Vaccines 1:83-92, CRC Press, Inc., Boca Raton, Fla.). An adjuvant can also be a part of the carrier of the vaccine, in which case it can be selected by standard criteria based on the antigen used, the mode of administration and the subject (Arnon, 1987). Methods of administration can be by oral or sublingual means, or by injection, depending on the particular vaccine used and the subject to whom it is administered.

In an embodiment, the protein comprising at least one glycoprotein on the exosporium of the Bacillus anthracis spore where the glycoprotein comprises at least one lectin-binding sugar may comprise a variant. Spore-specific sugars (rhamnose, 3-O-methyl rhamnose and galactosamine) not found in vegetative cells of B. anthracis that are distinct from the spore sugars found in related organisms have been found (Fox et al., 1993; Wunschel et al., 1994). It has been directly demonstrated that the anthrax spore is surrounded by carbohydrate.

In an embodiment, the peptide may comprise a Bcl-like peptide. For example, the glycoprotein BclA has a region of tandem repeats as are found in collagen (Bacillus, collagen-like protein anthracis) which consists of approximately 90% carbohydrate (Sylvester et al., 2002). BclA is localized to the exosporium nap as demonstrated by monoclonal antibody labeling (Sylvester et al, 2002). The spore-specific sugars were subsequently demonstrated to be components of a glycoprotein BclA (Daubenspeck et al., 2004). The operon coding for BclA synthesis was found, and a second glycoprotein ExsH having tandem repeats was demonstrated to be present in B. cereus and B. thuringiensis (Garcia Patronne, and Tandecarz, 1995; Todd et al., 2003).

The peptide backbone of BclA has a predicted molecular weight (MW) of approximately 39-kDa, but the intact protein migrates with an apparent mass of >250-kDa, for the Sterne strain, which is consistent with the protein being heavily glycosylated. There is considerable size heterogeneity among the BclA proteins due to different numbers of GPT repeats and [GPT]₅GDTGTT repeats in the protein. The latter 21 amino acid repeat has been named “the BclA repeat”. These repeats are the primary anchor point for rhamnose-oligosaccharides within BclA (Sylvestre et al., 2003).

In addition to the known glycoproteins on the exosporium of the Bacillus anthracis spore, where the glycoprotein comprises at least one lectin-binding sugar, there are protein variants which may also function in the disclosed methods and compositions. In certain embodiments, the variants are substitutional, insertional, truncational or deletional variants.

Protein variants and derivatives are well understood to those of skill in the art and in can involve amino acid sequence modifications. For example, amino acid sequence modifications typically fall into one or more of four classes: substitutional, insertional, truncational or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, are made by fusing a polypeptide sufficiently large to confer immunogenicity to the target sequence by cross-linking in vitro or by recombinant cell culture transformed with DNA encoding the fusion. Truncations are characterized by the removal of amino acids from the C-terminus or N-terminus of the full length protein. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, truncations, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the types of substitutions shown in Table 2 and are referred to as conservative substitutions.

TABLE 2
Amino Acid Substitutions

| | Exemplary Conservative |
|---|---|
| Original | Substitutions, others |
| Residue | are known in the art. |
| Ala | Ser |
| Arg | Lys, Gln |
| Asn | Gln; His |
| Asp | Glu |
| Cys | Aer |
| Gln | Asn, Lys |
| Glu | Asp |
| Gly | Pro |
| His | Asn; Gln |
| Ile | Leu; Val |
| Leu | Ile; Val |
| Lys; | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein. Substitutional or deletional mutagenesis may be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

The polypeptides of the present invention may include post-translational modifications. In an embodiment, certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 (1983)), acetylation of the N-terminal amine and, in some instances, amidation of the C-terminal carboxyl.

In an embodiment, the variants and derivatives of the disclosed proteins is through defining the variants and derivatives in terms of homology/identity to specific known sequences. Those of skill in the art readily understand how to determine the homology and/or percent identity of two proteins. For example, the homology can be calculated after aligning the two sequences so that the homology is at its highest level. Another way of calculating homology can be performed by published algorithms. Optimal alignment of sequences for comparison may be conducted by the local homology algorithm of Smith and Waterman, 1981, Adv. Appl. Math. 2: 482, by the homology alignment algorithm of Needleman and Wunsch (1970, J. MoL Biol. 48: 443 (1970)), by the search for similarity method of Pearson and Lipman, (Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wisc.), or by inspection. The same types of homology can be obtained for nucleic acids (Zuker, M., 1989, Science 244:48-52; Jaeger et al., 1989, Proc. Natl. Acad. Sci. USA, 86:7706-7710; Jaeger et al., 1989, Methods Enzymol., 183:281-306) which are herein incorporated by reference for at least material related to nucleic acid alignment. In an embodiment, the description of conservative mutations and homology can be combined together in any combination, such as embodiments that have at least 80% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, certain of the nucleic acid sequences sequences of SEQ ID NO: 1-379 can encode for specific protein sequences as set forth in the sequences of SEQ ID NO: 1-379.

In an embodiment, amino acid and peptide analogs can be incorporated into the disclosed compositions. For example, there are numerous D amino acids or amino acids which have a different functional substituent than the amino acids shown in Table 1. In an embodiment, the peptides may comprise the opposite stereo isomers of naturally occurring peptides, as well as the stereo isomers of peptide analogs. These amino acids can readily be incorporated into polypeptide chains by charging tRNA molecules with the amino acid of choice and engineering genetic constructs that utilize amber codons to insert the analog amino acid into a peptide chain in a site specific way (Thorson et al., 1991, Methods in Molec. Biol. 77:43-73; Zoller, 1992, Current Opinion in Biotechnology, 3:348-354; Ibba, 1995, Biotechnology & Genetic Engineering Reviews 13:197-216; Cahill et al., 1989, TIBS, 14(10):400-403; Benner, 1994, TIBS Tech, 12:158-163; Ibba and Hennecke, 1994, Bio/technology, 12:678-682; all of which are herein incorporated by reference at least for material related to amino acid analogs).

In an embodiment, the compounds of the present invention may include molecules that resemble peptides, but which are not connected via a natural peptide linkage. For example, linkages for amino acids or amino acid analogs can include [(CH₂NH)—], [—(CH₂S)—], [—(CH₂—CF₂)—], [—(CH═CH)—] [(cis and trans)], [—(COCH₂)—], [—(CH(OH)CH₂)—], and [—(CHH₂SO)—] (Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) [—(CH₂NH)—, (CH₂CH₂)—]; Spatola et al. Life Sci 38:1243-1249 (1986) [—(CH H₂)—(S)]; Hann J. Chem. Soc Perkin Trans. I 307-314 (1982) [—(CH—CH)—, cis and trans]; Almquist et al. J. Med. Chem. 23:1392-1398 (1980) [—(COCH₂)—]; Jennings-White et al. Tetrahedron Lett 23:2533 (1982) [—(COCH₂)—]; Szelke et al. European Appin, EP 45665 CA (1982): 97:39405 (1982) [—(CH(OH)CH₂)—]; Holladay et al. Tetrahedron. Lett 24:4401-4404 (1983) [—(C(OH)CH₂)—]; and Hruby Life Sci 31:189-199 (1982) [—(CH₂)—(S)—]; each of which is incorporated herein by reference. A particularly preferred non-peptide linkage is —[—(CH₂NH)—]. It is understood that peptide analogs can have more than one atom between the bond atoms, such as b-alanine, g-aminobutyric acid, and the like. Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch, 1992, Ann. Rev. Biochem. 61:387).

8. Nucleic Acids

As vaccines can consist of nucleic acids, there are a variety of molecules disclosed herein that are nucleic acid based, including the nucleic acids that encode for at least one glycoprotein from an extract of the exosporium of the Bacillus anthracis spore by absorption of the extract to lectin as well as any other proteins disclosed herein and variants and fragments of such polypeptides and/or proteins. In an embodiment, the nucleic acids used in the vaccines of the present invention may comprise nucleotides, nucleotide analogs, or nucleotide substitutes. Non-limiting examples of these and other molecules are discussed herein.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). It is understood for example that when a vector is expressed in a cell the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

In certain embodiments, the nucleotide vaccines of the present invention may comprise at least one of a nucleotide analog, a nucleotide substitute, or a conjugated nucleotide. A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. Other types of molecules may be linked to nucleic acid molecules to form conjugates. Conjugates can be chemically linked to the nucleotide or nucleotide analogs. Such conjugates include but are not limited to lipid moieties such as a cholesterol moiety. (Letsinger et al., 1989, Proc. Natl. Acad. Sci. USA, 86, 6553-6556). A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

Embodiments of the present invention also comprise oligonucleotides that are capable of interacting as either primers or probes with genes that encode for the glycoproteins and polypeptides associated with the glycoproteins of the complexes found in the B. anthracis spore as described herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

In an embodiment, the compositions are formulated for delivery to a cell, either in vivo or in vitro. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered by a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA (Wolff, J. A., et al., 1990, Science, 247, 1465-1468; Wolff, J. A., 1991, Nature, 352, 815-818). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

In an embodiment, the present invention may comprise the use of transfer vectors to deliver genes into cells (e.g., a plasmid), or as part of a general strategy to deliver genes, e.g., as part of recombinant retrovirus or adenovirus (Ram et al., 1993, Cancer Res. 53:83-88). As used herein, plasmid or viral vectors are agents that transport the nucleic acid of interest into a cell without degradation. The transfer vectors may comprise a promoter yielding expression of the gene of interest in the cells into which it is delivered. In some embodiments the vectors are derived from either a virus or a retrovirus. Viral vectors that may be used to deliver the DNA constructs of the present invention to cells may comprise Adenovirus, Adeno-associated virus, Herpes virus, Vaccinia virus, Polio virus, AIDS virus, neuronal trophic virus, Sindbis and other RNA viruses, including these viruses with the HIV backbone. Also included are any viral families which share the properties of these viruses which make them suitable for use as vectors. For example, retroviruses, including Murine Maloney Leukemia virus, MMLV, and retroviruses that express the desirable properties of MMLV as a vector may be used to deliver the DNA constructs of the present invention to cells. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. In an embodiment, a viral vector which has been engineered so as to suppress the immune response of the host organism, elicited by the viral antigens may be used such as vectors that carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase Ill transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

In an embodiment, a retrovirus is used to deliver the nucleic acid molecules of the present invention to a cell. A retrovirus is an animal virus belonging to the virus family of Retroviridae, including any types, subfamilies, genus, or tropisms. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

In an embodiment, an adenovirus vector is used to deliver the nucleic acid molecules of the present invention to cells. Replication-incompetent adenoviruses are currently available efficient gene transfer vehicles for both in vitro and in vivo deliveries (Lukashok, S. A., and M. S. Horwitz. 1998. Current Clinical Topics in Infectious Diseases 18:286-305). Adenovirus-vectored recombinant vaccines expressing a wide array of antigens have been constructed and protective immunities against different pathogens have been demonstrated in animal models (Lubeck, M. D., et al. 1997. Nat Med 3:651-8) (Shi, Z., et al., 2001, J Virol 75:11474-82; Shiver, J. W., et al., 2002, Nature 415:331-5; Tan, Y., et al., 2003, Hum Gene Ther 14:1673-82).

The construction of replication-defective adenoviruses has been described (Berkner et al., J. Virology, 1987, 61:1213-1220; Massie et al., 1986, Mol. Cell. Biol. 6:2872-2883; Haj-Ahmad et al., 1986, J. Virology 57:267-274; Davidson et al., 1987, J. Virology 61:1226-1239; Zhang, 1993, BioTechniques 15:868-872). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, 1993, J. Clin. Invest. 92:1580-1586; Kirshenbaum, 1993, J. Clin. Invest. 92:381-387; Roessler, 1993, J. Clin. Invest. 92:1085-1092; Moullier, 1993, Nature Genetics 4:154-159; La Salle, Science, 1993, 259:988-990; Gomez-Foix, 1992, J. Biol. Chem. 267:25129-25134; Rich, 1993, Human Gene Therapy 4:461-476; Zabner, 1994, Nature Genetics 6:75-83; Guzman, 1993, Circulation Research 73:1201-1207; Bout, 1994, Human Gene Therapy 5:3-10; Zabner, 1993, Cell 75:207-216; Caillaud, 1993, Eur. J. Neuroscience 5:1287-1291; and Ragot, 1993, J. Gen. Virology 74:501-507). Recombinant adenoviruses achieve gene transduction by binding to specific cell surface receptors, after which the virus is internalized by receptor-mediated endocytosis, in the same manner as wild type or replication-defective adenovirus (Chardonnet and Dales, 1970, Virology 40:462-477); Brown and Burlingham, 1973, J. Virology 12:386-396); Svensson and Persson, 1985, J. Virology 55:442-449); Seth, et al., 1984, J. Virol. 51:650-655); Seth, et al., 1984, Mol. Cell. Biol. 4:1528-1533); Varga et al., 1991, J. Virology 65:6061-6070); Wickham et al., 1993, Cell 73:309-319).

The viral vector can be one based on an adenovirus which has had the E1 gene removed. The E1 gene is necessary for viral replication and expression. However, E1-deleted viruses can be to propagated in cell lines that provide E1 in trans, such as 293 cells (Graham and Prevec, 1995, Mol. Biotechnol. 3:207-220). In another embodiment, both the E1 and E3 genes are removed from the adenovirus genome. The E3 region is involved in blocking the immune response to the infected cell.

In yet another embodiment, alternative serotype adenoviral vectors, such as human Ad35 or Ad7 to which the majority of human populations have very low pre-existing immunity could be used (31, 46). Also, adenoviral vectors derived from animals such as ovine and chimpanzee adenoviruses could also be used as alternative vaccine delivery vectors (Farina, S. F. et al. J Virol 75:11603-13; Hofmann, C. et al. 1999. J Virol 73:6930-6).

iii. Adeno-Associated Viral Vectors

In an embodiment, an Adeno-associated viral vector is used to deliver the nucleic acid molecules of the present invention to cells. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B 19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector. In certain embodiments, the inserted genes in viral and retroviral vectors will contain promoters, and/or enhancers to help control the expression of the desired gene product.

iv. Large Payload Viral Vectors

In yet another embodiment, a large payload viral vector, such as a herpes virus vector, is used to deliver the nucleic acid molecules of the present invention to cells. Molecular genetic experiments with large human herpesviruses have provided a means whereby large heterologous DNA fragments can be cloned, propagated and established in cells permissive for infection with herpesviruses (Sun et al., 1994, Nature genetics 8: 33-41; Cotter and Robertson, 1999, Curr. Opin. Mol. Ther., 5: 633-644). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes. In other embodiments, replicating and host-restricted non-replicating vaccinia virus vectors may also be used.

v. Non-Nucleic Acid Based Systems

The nucleic acid molecules of the present invention can be delivered to the target cells in a variety of ways. For example, in certain embodiments, the compositions may be delivered through electroporation, or through lipofection, or through calcium phosphate precipitation. The delivery mechanism chosen will depend in part on the type of cell targeted and whether the delivery is occurring in vivo or in vitro.

Thus, the compositions can comprise, in addition to the disclosed viruses or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract (see, e.g., Brigham et al., 1989, Am. J. Resp. Cell. Mol. Biol. 1:95-100); Feigner et al., 1987, Proc. Natl. Acad. Sci USA 84:7413-7417); U.S. Pat. No. 4,897,355). Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wisc.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., 1991, Bioconjugate Chem., 2:447-451; Bagshawe, K. D., 1989, Br. J. Cancer, 60:275-281; Bagshawe, et al., 1988, Br. J. Cancer, 58:700-703; Senter, et al., 1993, Bioconjugate Chem., 4:3-9; Battelli, et al., 1992, Cancer Immunol. Immunother., 35:421-425; Pietersz and McKenzie, 1992, Immunolog. Reviews, 129:57-80); and Roffler, et al., 1991, Biochem. Pharmacol, 42:2062-2065). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo (Hughes et al., 1989, Cancer Research, 49:6214-6220; and Litzinger and Huang, 1992, Biochimica et Biophysica Acta, 1104:179-187). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, 1991, DNA and Cell Biology 10:6, 399-409).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

In an embodiment, the nucleic acid molecules can be administered in a pharmaceutically acceptable carrier and can be delivered to the subjects' cells in vivo and/or ex vivo by a variety of mechanisms well known in the art (e.g., uptake of naked DNA, liposome fusion, intramuscular injection of DNA via a gene gun, endocytosis and the like). If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

(e) Expression Systems

In an embodiment, the nucleic acids that are delivered to cells may contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

In certain embodiments, promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

As used herein, an enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

In certain embodiments, the promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Also, in certain embodiments, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(f) Markers

In certain embodiments, the viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

10. Methods of Making the Compositions

The compositions disclosed herein and the compositions necessary to perform the disclosed methods can be made using any method known to those of skill in the art for that particular reagent or compound unless otherwise specifically noted. It is also understood that basic recombinant biotechnology methods can be used to produce the nucleic acids and proteins disclosed herein.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition, Cold Spring Harbor Laboratory Press, Cold Spring Harbor, N.Y., 1989, Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B; Ikuta et al., 1984, Ann. Rev. Biochem. 53:323-356, describing a phosphotriester and phosphite-triester methods; and Narang et al., 1980, Methods Enzymol., 65:610-620; describing a phosphotriester method). Protein nucleic acid molecules can be made using known methods (e.g., Nielsen et al., 1994, Bioconjug. Chem. 5:3-7).

2. Peptide Synthesis

One method of producing a protein for use as in a B. anthracis vaccine, such as those included in the sequences of SEQ ID NO: 1-379 is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A, 1992, Synthetic Peptides: A User Guide. W.H. Freeman and Co., N.Y., 1992; Bodansky M and Trost B., Ed., 1993, Principles of Peptide Synthesis. Springer-Verlag Inc., NY. Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen Let al., 1991, Biochemistry, 30:4151). Alternatively, native chemical ligation of synthetic peptides can be utilized to synthetically construct large peptides or polypeptides from shorter peptide fragments. This method consists of a two step chemical reaction (Dawson et al., 1994, Synthesis of Proteins by Native Chemical Ligation. Science, 266:776-779). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al., 1992, FEBS Lett. 307:97-101; Clark-Lewis I et al., 1994, J. Biol. Chem., 269:16075; Clark-Lewis I et al., 1991, Biochemistry, 30:3128; Rajarathnam K et al., 1994, Biochemistry 33:6623-30).

Alternatively, unprotected peptide segments are chemically linked where the bond formed between the peptide segments as a result of the chemical ligation is an unnatural (non-peptide) bond (Schnolzer, M et al. , 1992, Science, 256:221). This technique has been used to synthesize analogs of protein domains as well as large amounts of relatively pure proteins with full biological activity (deLisle Milton R C et al., 1992, Techniques in Protein Chemistry IV. Academic Press, New York, pp. 257-267).

3. Processes for Making the Compositions

In an embodiment, the spore surface glycoproteins complexes are produced after urea extracted or lysed spores are lectin purified. In an embodiment, the preparation comprises proteins, glycoproteins, oligosaccharides, lipids, or phospholipids that are produced by lysing the spore by urea extract or another means of lysis such as sonication but not limited to the above listed techniques. In an embodiment, the composition may comprise proteins, glycoproteins, polysaccharides, lipids, or phospholipids isolated by electro-elution or size exclusion chromatography after the spores have been lysed.

Embodiments of the present invention also comprise processes for making the compositions as well as making the intermediates leading to the compositions, and where reference to a particular sequence occurs, this is understood as exemplary only. In an embodiment, the protein used in the vaccine comprises a sequence that is encoded by one of the nucleic acid sequences having the sequence as set forth in any one of the nucleic acid sequences of sequences 1-379. There are a variety of methods that can be used for making these compositions, such as synthetic chemical methods and standard molecular biology methods. It is understood that the methods of making these and the other disclosed compositions are specifically disclosed. For example, in an embodiment, the protein or polypeptide of interest is generated by linking in an operative way a sequence that is encoded by one of the nucleic acid sequences having the sequence as set forth in any one of the nucleic acid sequences of sequences 1-379 to a sequence controlling the expression of the nucleic acid. In an embodiment, the nucleic acid sequence may comprise at least 80%, or at least 90%, or at least 95%, or at least 99% sequence identity to one of the nucleic acid sequences having the sequence as set forth in any one of the nucleic acid sequences of sequences 1-379. Or, a sequence that hybridizes under stringent hybridization conditions to one of the nucleic acid sequences having the sequence as set forth in any one of the nucleic acid sequences of sequences 1-379 may be used. For example, in an embodiment, the present invention comprises an isolated nucleic acid molecule encoding a lectin-binding glycoprotein isolated from the exosporium of the Bacillus anthracis spore comprising a nucleic acid sequence as set forth in SEQ ID NO: 43, SEQ ID. NO: 45, SEQ ID. NO: 47, SEQ ID. NO: 49, SEQ ID. NO: 51, SEQ ID. NO: 53, SEQ ID. NO: 55, SEQ ID. NO: 57, SEQ ID. NO: 59, SEQ ID. NO: 61, SEQ ID. NO: 63, SEQ ID. NO: 69, or SEQ ID. NO: 71.

The polypeptide encoded by the nucleic acid construct may comprise one of the polypeptide sequences having the sequence as set forth in any one of the amino acid sequences of sequences 1-379, or a fragment of such a protein, or a protein having conservative amino acid substitutions. In an embodiment, the amino acid sequence has at least 80% homology to at least one of the amino acid sequences as set forth in SEQ ID. NO: 44, SEQ ID. NO: 46, SEQ ID. NO: 48, SEQ ID. NO: 50, SEQ ID. NO: 52, SEQ ID. NO: 54, SEQ ID. NO: 56, SEQ ID. NO: 58, SEQ ID. NO: 60, SEQ ID. NO: 62, SEQ ID. NO: 64, SEQ ID. NO: 70, or SEQ ID. NO: 72.

In yet another embodiment, the present invention comprises genetically modified animals produced by the process of transfecting a cell within the animal with any of the nucleic acid molecules disclosed herein. The animal may be a mammal. In alternate embodiments, the mammal may be a mouse, rat, rabbit, cow, sheep, pig, or primate. Alternatively, a genetically modified animal may be made by adding to the animal any of the cells disclosed herein.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Ultra-Structural Demonstration of a Glycoprotein Nap Surrounding the Exosporium

To the buffer-washed spore pellets, one milliliter (ml) of a 25% glutaraldehyde, 0.1 M sodium cacodylate solution is supplemented with ruthenium red (1 mg/ml) and incubated for one hr at 37° C. Each pellet will is washed in sodium phosphate buffer and fixed for 3 hr at room temp. in 2% osmium tetroxide in 0.1 M sodium cacodylate solution containing ruthenium red. A negative control is treated identically, but ruthenium red was omitted from these two steps. Spores can be washed in buffer and embedded in 3% agar. Dehydration involves sequential treatment with 25%, 50%, 75%, 95%, and 100% ethanol. Afterwards, cells may be placed sequentially in propylene oxide, propylene oxide/polybed 812, and pure polybed 812. Polymerization is carried out at 60° C. Then sections are cut and stained with a 2% uranyl acetate solution for 40 min at 37° C., followed by Hanaichi lead citrate for 2 min. Spores are observed by transmission electron microscopy.

For ultra-structural observation of B. anthracis spores, upon staining with uranyl acetate and osmium tetroxide, the external basement membrane of the exosporium may be readily visible separated from the underlying coat layers. After additional ruthenium red staining, the external nap is readily demonstrable. It will be demonstrated, using immuno-gold labeling that the peptide portion of BclA is expressed on the exosporium surface. Furthermore, exosporium nap additionally is rich in carbohydrate. The standard procedures to purify spores involve renografin gradients

### Example 2

### Analysis of Glycoproteins, Proteins, Lipids, and Phospholipids using Gel Electrophoresis, Glycoprotein Staining and Matrix Assisted-Time-of-Flight Mass Spectrometry (MALDI-TOF MS)

B. anthracis spores (50 mg wet weight) were extracted with a urea buffer (50 mM Tris-HCl, pH 10, 8 M urea, 2% 2-mercaptoethanol) for 15 min at 90° C. The extracted spores were centrifuged at 13,000 g for 10 min at room temp. The supernatant was removed and stored for protein analysis. Spore protein extract was combined with loading buffer (35:1) and loaded onto IPG strips (pH 3-10) using the multiphor II electrophoresis system or other appropriate piece of equipment. Next, the strips are rehydrated for focusing at 23,000 Vh for 24 hours. Then, the strips were equilibrated immediately in SDS equilibrium buffer (50 mM Tris-HCl, pH 8.8, 6M Urea, 30% (v/v) glycerol, 2% (w/v) SDS, bromophenol blue, trace) for 15 minutes at room temperature. Afterwards the strips were equilibrated in a second solution of DTT (10 mg/mL; 65 mM) for 15 minutes at room temperature. The equilibrated strips were loaded on to a 4-15% gradient polyacrylamide gels and electrophoresed in Tris-glycine-SDS buffer. The gels are stained with ProtoBlue safe with identify protein spots.

To perform the electro-elution, the gel spots are cut out with a scalpel and destained in water or another appropriate destaining buffer. Next, the gel slices are placed in sample tubes (Millipore) and placed in a electro-eluter (Millipore) with the appropriate molecular weight cut off filter. For example, EA1 runs on a gel at approximately 100 kDa so a 100 kDa molecular weight filter would be used to capture the protein and still allow the degassed Tris-glycine buffer to run through. The protein samples are electro-eluted at 100 Vh for 22-24 hours depending upon the specific protein being electro-eluted (smaller proteins require less time). Finally, the protein samples are washed in their filter with ddH₂O three times and centrifuged at 5,000 rpm for 5 minute intervals until the desired volume is reached.

The proteins were then treated with Zip tips (Michron BioResources, Auburn, Calif.) to remove the SDS and tris-glycine from the glycoprotein solution. Next, an appropriate enzyme at the appropriate conditions is used to break apart the protein or chew off the carbohydrate component of a glycoprotein. For example, EA1 can be digested using Trypsin for 3 hours at room temperature. Next, the samples are Zip Tiped again to remove any salt or detergent contamination; SDS interferes with MALDI ionization and crystallization while high concentrations of Tris and glycine in the MALDI preparation interfere with absorbance of laser energy by the matrix. The purified samples were mixed with the MALDI matrix (1:1 v/v solution of α-cyanno hydroxycinnamic acid (20 mg/ml in 7:3 v/v acetonitrile:0.1% trifuoroacetic acid) and 2,5-dihydroxy benzoic acid (20 mg/ml in 7:3 v/v acetonitrile:5% formic acid), (31). The molecular weight (MW) of the intact protein will be determined using a Applied Biosystems 4700 Protein Analyzer MALDI TOF mass spectrometer (Applied Biosystems, Foster City, Calif.) equipped with a 20 Hz nitrogen laser and a reflectron.

For example, EA1 was identified by MALDI TOF MS analysis and can be seen as an intensely stained band, <100 kDa band, on gel electrophoresis, See FIG. 3. There are at least 7 other visible proteins that appeared after staining and will be analyzed by MALDI TOF MS. Using MS analysis the following masses were recorded, 983.4373, 1014.571, 1029.5479, 1140.5757, 1179.5699, 1206.5680, 1223.5785, 1228.7073, 1277.6838, 1356.8062, 1359.7783, 1405.7643, 1414.8136, 1424.7617, 1515.8846, 1517.7678, 1526.8829, 1533.7843, 1684.8827, 1709.8922, 1765.9010, 1771.8489, 1857.8329, 1878.9424, 1901.8921, 1934.9288, 1996.9645, 2063.0415, 2230.1863, and 2497.2002 for the gel band corresponding to the <100 kDa band. Imputing these values into Protein Prospector and searching the entire Swiss-Prot database for all species a MOWSE Score of 7.39×10¹⁴ was obtained for P94217, which corresponds to S-layer protein EAI precursor for B. anthracis. With a MOWSE Score this high the probability that this is any other protein is almost zero. Additionally, 46.1% coverage of the protein was achieved with a mean ppm error of only 6.3. Furthermore, MS/MS spectra were taken of each mass above to further support the sequence of each peptide analyzed.

### Example 3

### Lysed Spores, Gel Electrophoresis, and Electro-Elution to Isolated Specific Proteins, Glycoprotein, Oligosaccarides, Lipids, or Phospholipids

B. anthracis spores (50 mg wet weight) were extracted with a urea buffer (50 mM Tris-HCl, pH 10, 8 M urea, 2% 2-mercaptoethanol) for 15 min at 90° C. The extracted spores were centrifuged at 13,000 g for 10 min at room temp. The supernatant was removed and stored for protein analysis. 35:1 of spore protein extract was combined with loading buffer and loaded onto IPG strips (pH 3-10) using the multiphor II electrophoresis system or other appropriate piece of equipment. Next, the strips are rehydrated for focusing at 23,000 Vh for 24 hours. Then, the strips were equilibrated immediately in SDS equilibrium buffer (50 mM Tris-HCl, pH 8.8, 6M Urea, 30% (v/v) glycerol, 2% (w/v) SDS, bromophenol blue, trace) for 15 minutes at room temperature. Afterwards the strips were equilibrated in a second solution of DTT (10 mg/mL; 65 mM) for 15 minutes at room temperature. The equilibrated strips were loaded on to a 4-15% gradient polyacrylamide gels and electrophoresed in Tris-glycine-SDS buffer. The gels are stained with ProtoBlue safe with identify protein spots.

To perform the electro-elution, the gel spots are cut out with a scalpel and destained in water or another appropriate destaining buffer. Next, the gel slices are placed in sample tubes (Millipore) and placed in a electro-eluter (Millipore) with the appropriate molecular weight cut off filter. For example, EA1 runs on a gel at approximately 100 kDa so a 100 kDa molecular weight filter would be used to capture the protein and still allow the degassed Tris-glycine buffer to run through. The protein samples are electro-eluted at 100 Vh for 22-24 hours depending upon the specific protein being electro-eluted (smaller proteins require less time). Finally, the protein samples are washed in their filter with ddH₂O three times and centrifuged at 5,000 rpm for 5 minute intervals until the desired volume is reached. Verification of a successful electro-elution can be done by re-running the electro-eluted sample on a one dimensional gel electrophoresis mini-gel system.

### Example 4

### Lectin Purification of Glycoprotein Complexes After Anthrax Spores have Been Lysed

The glycoproteins on the exosporium of the anthrax spore form complexes with other protein, glycoproteins, oligosaccarides, lipids, or phospholipids and can be isolated by first lysing the spores by urea extraction buffer or anther lysis method then purify the complexes by lectins. The lectins bind to sugars and should therefore bind to BclA of the exosporium of the B. anthracis spore. The BclA is also bound to other substances that should stay attached to it when it is bound to the lectin. The glycoprotein complexes can then be unbound to the lectin by washing the lectin with sugars that it can bind to stronger than the glycoproteins therefore the sugars will out compete the glycoproteins for binding space on the lectin leaving a mixture of glycoprotein complexes and sugar that did not bind to the lectin. The sugar can be washed away with a low molecular weight cut off filter leaving the purified glycoprotein complexes. Potential lectins that could be used for this procedure include but are not limited to SBA (E-Y laboratories), APA (E-Y laboratories), GSA-1 (E-Y laboratories), RCA-I (E-Y laboratories), RCA-II (E-Y laboratories), the L-rhamnose-binding lectins STL1, STL2, and STL3 (Tateno et al., 1998). These lectins can come in many forms such as but not limited to a gel or on a bead. Using Anthrax as a novel system there are many other microorganisms that may be purified using lectin technology (Table 1).

### Example 5

### Size Exclusion Chromatography

Lysed spores can be ran through a size exclusion column such as, but not limited to, a sephacyl column. In this technique, substances with a molecular weight that is within the range of the column will be trapped inside the column but any substance outside of the mass range will go through the column therefore sorting the substance by size.

### Example 6

### Spore Carbohydrate Complexes: Antigenic Determinants Provide Immunity Against Infection in a Guinea Pig Model

The B. anthracis spore, like those of its closely related species, appear to contain a carbohydrate component. It has also been shown that a complete immunity to anthrax requires a spore component to the vaccine, in addition to protective antigen.

(a) Protection Against Anthrax Infection with Lectin Purified Glycoprotein Complexes and Their Antibody Response

Groups of five guinea pigs (half male and half female) and groups of three rabbits (half male and half female) will be immunized intramuscularly with 100 μl to 2 mL volumes of the following 1) the animal current animal vaccine from Colorado Serum Co. (positive control); 2) an adjuvant only plus PBS (negative control); 3) lectin purified glycoprotein complexes with an adjuvant. Booster immunizations will be given at 4, 5, 6, 7, 9, 10, 11, 12, 13, 14, 15, 16, 17, 18, 19 or 20 weeks. The animals will be bled via the Saphenous vein or anther bleeding method at two and four weeks and tested for antibody response by an ELISA procedure. The guinea pigs will be challenged intramuscularly at week 20 with 100 time LD₅₀ Bacillus anthracis Ames or anther strain. The rabbits will be challenged inhalationally at week 20 with 100 time LD₅₀ Bacillus anthracis Vollum, Ames or anther strain or Bacillus cereus G9241 or another strain that can cause an anthrax like infection. Spore preparations diluted in PBS will be applied to Maxisorp ELISA plates. After overnight incubation at 4° C., the coated wells will be washed with wash buffer (PBS [pH 7.4], 0.1% Tween 20, 0.001% thimerosal). The plates will then be reacted with dilutions ofthe rabbit or guinea pig antiserum. Dilutions will be made in ELISA dilution buffer (PBS [pH 7.4], 5% dry skim milk, 0.001% thimerosal). The secondary antibody will be goat anti-rabbit horseradish peroxidase conjugate. Plates will be incubated at 37° C. for 1 hr and then washed six times with wash buffer. The substrate, 2,2′-azinobis (3-ethylbenzthiazolinesulfonic acid) will be added and the plates will be read at 405 nm after incubation at room temperature for 15 minutes with a microtiter plate reader (Dynex). The ELISA procedure will also be utilized to determine if reactivity exists against vegetative cells of Δ Sterne-1, Sterne 34F2, or any other suitable strain from anthrax. If such activity is found, it will be removed by an absorption procedure. Vegetative cells of Δ Sterne-1, Sterne 34F2, or other suitable strain from anthrax will repeatedly be subcultured to eliminate spores from the population and then grown in nutrient broth to mid-logarithmic phase, harvested by centrifugation, washed in PBS, fixed in formalin, and washed extensively in PBS. The fixed cells will be added to an aliquot of the antiserum and antibodies against vegetative cell antigens allowed to bind at 4° C. The bacteria and the bound antibodies will then be removed from the serum by centrifugation. This will be repeated until no vegetative cell reactivity is detected by ELISA. Antibodies from the antisera will be purified using a protein A-agarose affinity column (Pierce Chemical Co.). Western blot analysis will be carried out to determine if an antibody response to the exosporium glycoprotein complexes occurs and antigenic epitopes defined.

This protocol will determine if lectin purified glycoprotein spore complexes can provide protection against Ames strain of B. anthracis both cutaneously and inhalationally. Furthermore, this experiment expresses the individual antigens within the glycoprotein complex that are immunogenic and what types of antibodies are formed to these glycoprotein complexes.

(b) Protection Against Several Strains of Anthrax and Other Anthrax Like Infections

Groups of ten guinea pigs (half male and half female) and groups of six rabbits (half male and half female) will be immunized intradermally with 100 μl to 2 mL volumes of the following 1) the current animal vaccine made by Colorado Serum Co. (positive control); 2) an adjuvant only plus PBS (negative control); 3) lectin purified glycoprotein complexes with an adjuvant. Booster immunizations can be given at 4, 5, 6, 7, 9, 10, 11, 12, 13, 14, 15, 16, 17, 18, 19 or 20 weeks. The animals can be bled via Saphenous vein or anther bleeding method at two and four weeks and tested for antibody response by an ELISA procedure. The guinea pigs will be broken up into three sub groups in each of the above groups and challenged cutaneously at week 20 with 100 time LD₅₀ Bacillus anthracis 1) Vollum or other anthrax strain, 2) Ames or another strain or 3) Bacillus cereus G9241 or another strain that can cause an anthrax like infection. The rabbits will be broken up into three sub groups within each group and challenged inhalationally at week 20 with 100 time LD₅₀ Bacillus anthracis 1) Vollum or other anthrax strain, 2) Ames or anther strain or 3) Bacillus cereus G9241 or another strain that can cause an anthrax like infection. The above protocol will determine if lectin purified glycoprotein spore complexes will provide protection against B. anthracis and other bacteria that cause anthrax like infections both cutaneously and inhalationally.

### Example 7

### One Dimensional Gel of Lectin Purified Complexes from B. anthracis

FIG. 3 is a one-dimensional SDS gel that contains both urea extracted spores and lectin purified complexes. Sterne 34F2 spores were obtained from Colorado Serum Co. The spores were grown on nutrient agar plates (Difco, Detroit, Mich.) for one week when sporulation was complete for most of the bacterium (>95%). The spores were harvest from the plates using milliQ water set to 18.2 milliOhms. The spores were frozen at −80 degrees C. overnight. The next day, the spores were allowed to thaw at room temperature to lyse any of the remaining vegetative cells (approximately 3 hours). Next, the spores were washed centrifuging at 10,000 rpm for 10 minutes at 4 degrees C. The water on top of the spores was decanted off and new water was added on top to wash the spores. The amount of water added was equal to the volume of spores in the tube. The tube was vortexed and spun again 10,000 rpm for 10 minutes at 4 degrees C. The wash procedure just described was repeated three times until the water on the top of the spores was clear. The final volume of water added was equal to the volume of centrifuged spores in the tube. The spores were counted an analyzed for purity using phase contrast microscopy. Next, the spores were urea extracted. For urea extracted spores 1000 uL of concentrated B. anthracis suspension (1.27×10̂7 spores per microliter at 99.76% pure spore) was centrifuged at 10,000 rpm for 10 minutes. Then, the liquid on top was decanted off. Next, 300 microliters of urea extract buffer (50 mM Tris-HCl, ph 10, 8 M urea, 2% 2-mercaptoethanol) (Fisher Scientific) was added to the spores and vortexed until all the spores were dissolved in the solution. The urea solution was heated to 90 degrees C. for 15 minutes. Then, the urea extracted spores were centrifuged at 10,000 rpm for 10 minutes. The supernatant was removed and the particulate at the bottom was thrown away. Half the supernatant was used in the urea extracted lanes of the gel shown in this figure. The other half of the supernatant was used for lectin purification. Two mL of SBA lectin bound to agrose beads was placed in a gravity column (Fisher Scientific). The SBA lectin was washed using 4 mL of water. Next, 150 microliters of urea extracted spores was placed on the column and allowed to sit for 1 hour. Then, the excess unbound material was allowed to drain off into a waste container. Next, 1.2 mL of 0.1M D-galactose was added to the column and allowed to sit for 1 hour. Then, the column was allowed to drain and small samples of the bound material were collected (about 300 microliters). The bound samples were then run on an SDS page gel described below. The urea extracted spores (the supernatant) or lectin treated urea extracted spores was added to twice the volume of sample buffer (50 mM Tris-HCl, pH 6.8, 4% sodium dodecyl sulfate (SDS), 10% glycerol, 5% 2-mercaptoethanol, 0.02% bromophenol blue) (Fisher Scientific) and heated to 95 degrees C. for 4 minutes. Fifteen microliters of a kaleidoscope Prestained Standard (BioRad) was used in one lane. The prestained standard was, also, heated at 95 degrees C. for 4 minutes prior to being loaded onto the gel. Fifteen microliters of the urea extracted spores plus sample buffer or 15 microliters of lectin treated urea extracted spores plus sample buffer was loaded on to a 4-15% polyacrylamide minigel system (BioRad). The sample was electrophoresed using Tris-Glycine-SDS Buffer (Fisher Scientific). The gel was ran at 100V for 2 hours. The gel was washed three times with milliQ water set to 18.2 milliOhms for 15 minutes three times before staining. The gel was stained using gel code blue comassee stain overnight (Pierce, Rockford, Ill.). Finally, the gel was washed three times for 15 minutes to remove any excess stain. Lanes A, C, and E are all urea extracted spores. Lane B is the lectin isolated urea extracted spores. There are 7 bands in this lane. One band contains EA1. Lane D is the kaleidoscope prestained standard.

### Example 8

### Urea Extracted Spores Before Lectin Treatment

FIG. 4 shows urea extracted spores before lectin treatment. Sterne 34F2 spores were obtained from Colorado Serum Co. The spores were grown on nutrient agar plates (Difco, Detroit, Mich.) for one week when sporulation was complete for most of the bacterium (>95%). The spores were harvest from the plates using milliQ water set to 18.2 milliOhms. The spores were frozen at −80 degrees C. overnight. The next day, the spores were allowed to thaw at room temperature to lyse any of the remaining vegetative cells (approximately 3 hours). Next, the spores were washed centrifuging at 10,000 rpm for 10 minutes at 4 degrees C. The water on top of the spores was decanted off and new water was added on top to wash the spores. The amount of water added was equal to the volume of spores in the tube. The tube was vortexed and spun again 10,000 rpm for 10 minutes at 4 degrees C. The wash procedure just described was repeated three times until the water on the top of the spores was clear. The final volume of water added was equal to the volume of centrifuged spores in the tube. The spores were counted an analyzed for purity using phase contrast microscopy. Next, the spores were urea extracted. For urea extracted spores 1000 uL of concentrated B. anthracis suspension (1.27×10̂7 spores per microliter at 99.76% pure spore) was centrifuged at 10,000 rpm for 10 minutes. Then, the liquid on top was decanted off. Next, 300 microliters of urea extract buffer (50 mM Tris-HCl, ph 10, 8 M urea, 2% 2-mercaptoethanol) (Fisher Scientific) was added to the spores and vortexed until all the spores were dissolved in the solution. The urea solution was heated to 90 degrees C. for 15 minutes. Then, the urea extracted spores were centrifuged at 10,000 rpm for 10 minutes. The supernatant was removed and the particulate at the bottom was thrown away.

The urea extracted spore protein extract (the supernatant) was combined with loading buffer and loaded onto IPG strips (pH 3-10) using the multiphor II electrophoresis system (Amersham) or other appropriate piece of equipment. Next, the strips are rehydrated for focusing at 23,000 Vh for 24 hours. Then, the strips were equilibrated immediately in SDS equilibrium buffer (50 mM Tris-HCl, pH 8.8, 6M Urea, 30% (v/v) glycerol, 2% (w/v) SDS, bromophenol blue, trace) for 15 minutes at room temperature. Afterwards the strips were equilibrated in a second solution of DTT (10 mg/mL; 65 mM) for 15 minutes at room temperature. The equilibrated strips were loaded on to a 4-15% gradient polyacrylamide gels and electrophoresed in Tris-glycine-SDS buffer. The gel was stained for glycoproteins with ECL glycoprotein detection system (Amersham Biosciences) according to the manufacturer's description. The urea extracted spores reveal two glycoproteins.

### Example 9

### MALDI TOF MS Spectrum of an Anthrax Glycoprotein

FIG. 5 show a matrix-assisted laser desorption/ionization (MALDI) time-of-flight (TOF) mass spectrum of a gel slice obtained from a one dimensional gel, which is shown in FIG. 3. The protein was identified as B. anthracis S-layer protein EA1 pre-cursor (EA1 ID) from Swiss-Prot database, P94217, and with a MOWSE score of 7.39×10⁺¹⁴. With a score this high the probability that this is any other protein is almost zero. Additionally, 46.1% coverage of the protein was achieved with a mean ppm error of only 6.3. All of the masses above a signal-to-noise threshold of 10:1 were applied to data analyze, which generated the above identification. The MADLI TOF MS used in this experiment was a Applied Biosystems 4700 Protein Identification system. To generate this spectrum the following protocol was employed. After staining of the gel several spots of interest were selected for MS analysis. These spots were excised using a cleaned autoclaved razor blade and added to a 1.5 mL centrifuge tube. The gel slices were then de-stained for 45 min with 200 uL of 100 mM solution of ammonium bicarbonate in 50% acetonitrile. The tubes are then vacuum dried at 37 C until they are dry. Next, the samples are reduced by adding 100 uL of 2 mM TCEP (Tris(2-carboxyethyl)phosphine, in 25 nM ammonium bicarbonate (pH 8.0) and allowed to incubate for 15 minutes at 37 C with slight agitation. The supernatant is removed and 100 uL of 20 mM iodoacetamide in 25 mM ammonium bicarbonate (pH8.0) is added and allowed to sit in the dark for 15 minutes. The gels are then washed three times with 200 uL of 25 mM ammonium bicarbonate for 15 minutes, then dried with vacuum centrifugation. The gels are re-hydrated with 20 uL of 0.02 ug/uL of sequencing grade modified trypsin in 10% acetonitrile, with 40 mM ammonium bicarbonate (pH 8.0) and 0.1% n-octylgucoside for one hour at room temperature. Next, 50 uL of 10% acetonitrile with 40 mM ammonium bicarbonate) pH 8.0) is added to the tubes and allowed to sit for 5 minutes. The supernatant is removed placed into a fresh 1.5 mL centrifuge tube and vacuum centrifuged to dryness. Next, 200 uL of pure water is added and then spun to dryness again. This is repeated three times. Finally, on the forth re-suspension the solution is dried until only 10 uL of sample remains. This remaining solution is then ready for MALDI TOF MS analysis. For MS analysis 1 uL of sample is mixed with 1 uL of matrix and spotted until the stainless steel probe for analysis. The matrix used is 2,5 di-hydroxybenzoic acid (DHB) in 80/20 methanol water matrix with a saturated solution of DHB. After the spot dries the sample is running using a standard conditions with an Applied Biosystems 4700 Protein Analyzer MS.

While the invention has been described and illustrated with reference to certain embodiments thereof, those skilled in the art will appreciate that various changes, modifications and substitutions can be made therein without departing from the spirit and scope of the invention. For example, effective dosages other than the dosages as set forth herein may be applicable as a consequence of variations in the responsiveness insect population being treated. Likewise, the specific biochemical responses observed may vary according to and depending on the particular active compound selected or whether there are present pharmaceutical carriers, as well as the type of formulation and mode of administration employed, and such expected variations or differences in the results are contemplated in accordance with the objects and practices of the present invention. All references referred to herein are incorporated by reference in their entireties. The disclosures of the publications, patents, or patent applications referred to herein are hereby incorporated by reference in their entireties.

### APPENDIX

!List of Amino Acid and Nucleotide Sequence for Surface Proteins from? ?!Bacillus anthracis

B. anthracis CotS - (Q81XP5)

| | |
|---|---|
| 1. SQ Sequence 1002 BP; 340 A; 178 C; 196 G; 288 T; 0 other; 1100207923 CRC32; | |
| atgattcatc atatttatga gcattatcac atgcatgtta aagaattaat cccccttggc | 60 |
| ccctataaaa gcttttggat tcgcaacaaa atttatgtac ttgttccaat tggagaaatg | 120 |
| gaggaagaag tacttgtaga gatgaaaaag ctcagtgact atatgaacca gcaaggggat | 180 |
| ataactgtag cgactttcgt tccaactata catggctact atgtaagtga gatagaagaa | 240 |
| caaaattact gcttattaaa aggtatgcga gcgttagaac gacatgctat atcattaggt | 300 |
| agtgagcttt ctatattcca taaacgaggt gcattctttc cagaagaaat tgagcaacta | 360 |
| agccgcattg gtgaatggaa agcattatgg gaaaaaaggc tcgatcaatt agaaaagttt | 420 |
| tggcaatcac aagtgatgaa ccaccctaca gacgtattcg atcaattgtt tattgaatcc | 480 |
| ttcccgtatt acttaggggt tgcagaaaat gccattcaat atgttgttga tacagaaatg | 540 |
| gatgatacgc cgcaactaac tgatgcagca acaatttgcc aagaacgatt cacaccttta | 600 |
| ttatggcatc aaacgaagcg tctcaaactc ccttttgatt gggtgtatga tcacccaact | 660 |
| cgagatatag cagaattaat ccgttatatg atgattgaaa aaaagaaaga ctgggagaaa | 720 |
| acaatcgttc aatttgttac agattacgaa cgaaattatt cgctatcctc atttggttgg | 780 |
| cgcctattat ttgcaaggct cttgttcccg cttcactatt ttgaaacagt tgaacggtac | 840 |
| taccaaacag gaaacgaaga acaaaaaagc atatatagag atcgcttaga agccatttta | 900 |
| cacgatgtga accgctcaga gcaatttatg aagcattttt atagctcact tcgtttacca | 960 |
| gttgataagc tcgggattag aaaattagat tggttatctt aa | 1002 |
| 2. SQ SEQUENCE 333 AA; 40117 MW; 647CA3BA3D96DE6B CRC64; | |
| MIHHIYEHYH MHVKELIPLG PYKSFWIRNK IYVLVPIGEM EEEVLVEMKK LSDYMNQQGD | |
| ITVATFVPTI HGYYVSEIEE QNYCLLKGMR ALERHAISLG SELSIFHKRG AFFPEEIEQL | |
| SRIGEWKALW EKRLDQLEKF WQSQVMNHPT DVFDQLFIES FPYYLGVAEN AIQYVVDTEM | |
| DDTPQLTDAA TICQERFTPL LWHQTKRLKL PFDWVYDHPT RDIAELIRYM MIEKKKDWEK | |
| TIVQFVTDYE RNYSLSSFGW RLLFARLLFP LHYFETVERY YQTGNEEQKS IYRDRLEAIL | |
| HDVNRSEQFM KHFYSSLRLP VDKLGIRKLD WLS | |

B. anthracis CotJA - (Q81UQ8)

| | |
|---|---|
| 3. SQ Sequence 216 BP; 74 A; 44 C; 30 G; 68 T; 0 other; 4140865594 CRC32; | |
| atggataaat atatgaaatc atatgtgcca taccatagtc ctcaagatcc ttgtcctcct | 60 |
| attggtaaaa aatattactc tacccctcct aatttatatt taggttttca accgccaaat | 120 |
| ttaccacagt tctcaccgaa agaagcacta caaaaaggaa ctttatggcc tgttttttat | 180 |
| gattattacg aaaatcctta taaaaaaggg cggtga | |
| 4. SQ SEQUENCE 71 AA; 8410 MW; 448E6A60505B68D2 CRC64; | |
| MDKYMKSYVP YHSPQDPCPP IGKKYYSTPP NLYLGFQPPN LPQFSPKEAL QKGTLWPVFY | |
| DYYENPYKKG R | |
| 216 | |

B. anthracis CotJB - (Q81UQ9)

| | |
|---|---|
| 6. SQ SEQUENCE 91 AA; 10946 MW; 5FC13598D8DB7048 CRC64; | |
| MTTDVNQPLP EEYYRLLENL QELDFVLVEL TLYLDTHPDD TAAINQFNDF SYKRRVLKQQ | |
| MEEKYGPLQQ YGNSYSNAPW EWSKGPWPWQ I | |
| 5. SQ Sequence 276 BP; 101 A; 59 C; 50 G; 66 T; 0 other; 2169401454 CRC32; | |
| gtgacgactg acgtgaacca gccactacca gaagaatatt atcgactttt agagaatctc | 60 |
| caagaattag actttgtact agtcgaacta acgctttact tagacaccca cccagacgat | 120 |
| acagcagcta ttaatcaatt taatgacttt tcctataaac gaagagtact aaaacaacag | 180 |
| atggaagaaa aatatggacc acttcaacag tacggaaata gctattctaa tgccccttgg | 240 |
| gaatggagca aaggtccttg gccatggcaa atataa | 276 |

B. anthracis CotJC - (Q81UR0)

| | |
|---|---|
| 8. SQ SEQUENCE 189 AA; 21651 MW; 13F8D803CC0BEA83 CRC64; | |
| MWIYEKKLQY PVKVGTCNPA LAKLLIEQYG GADGELAAAL RYLNQRYTIP DKVIGLLTDI | |
| GTEEFAHLEM IATMVYKLTK DATPEQMKAA GLDPHYVDHD SALHYHNAAG VPFTATYIQA | |
| KGDPIADLYE DIAAEEKARA TYQWLINQSD DPDINDSLRF LREREIVHSQ RFREAVEILK | |
| EERDRKIYF | |
| 7. SQ Sequence 570 BP; 189 A; 113 C; 110 G; 158 T; 0 other; 3739425362 CRC32; | |
| atgtggattt atgaaaaaaa attacaatac cctgttaaag taggaacttg caatccagca | 60 |
| cttgcaaaat tattgattga acaatatggc ggtgcagatg gagagttagc tgctgcactc | 120 |
| cgttacttaa atcagcgtta tacaattcct gataaagtca ttggccttct taccgatatt | 180 |
| ggtacagaag aatttgcgca tcttgaaatg attgctacga tggtttataa actaacaaaa | 240 |
| gatgcgactc ctgaacagat gaaggcagcc ggtcttgatc ctcattacgt cgaccatgac | 300 |
| agcgcacttc attaccataa cgcggctggt gttccattta ctgcaaccta tatacaagct | 360 |
| aaaggtgatc caattgctga tttatacgaa gatattgccg ctgaagaaaa agcacgtgcc | 420 |
| acatatcaat ggcttattaa ccaatcagac gatcccgaca taaatgacag cttacgcttt | 480 |
| ttacgtgaac gagaaattgt ccattcacaa cgtttccgag aagcagttga aattttaaaa | 540 |
| gaagaacgcg atcgaaagat ttatttttaa | 570 |

B. anthracis CotM - (Q6HVHO/Q81Y76, Q6KPPO)

| | |
|---|---|
| 10. SQ SEQUENCE 131 AA; 15228 MW; 05D6AEAB8009D73C CRC64; | |
| MSYMGKKKKD CLFHVDGFEE WMDQFCSDSC SNFSFPNQIH IDLCETEQEY ILETDVPNVT | |
| EQNVVIKKME TGLNICILHK NISLQRNIPL PTTIIYKKML ACLENGFLAI HISKNEVANK | |
| HEEKVLFQIE N | |
| 9. SQ Sequence 396 BP; 141 A; 55 C; 68 G; 132 T; 0 other; 1286526549 CRC32; | |
| gtgtcttaca tgggcaagaa aaagaaggat tgtctttttc atgttgatgg ttttgaagaa | 60 |
| tggatggatc aattttgttc tgattcttgt agtaacttta gtttcccaaa tcaaattcat | 120 |
| attgatcttt gtgaaactga acaagaatac attttggaaa cagatgtacc aaatgtaact | 180 |
| gaacaaaatg tagttattaa aaagatggag acaggcctaa acatttgcat acttcataaa | 240 |
| aatatttctt tgcagcggaa cattccttta cccactacta ttatttataa gaagatgcta | 300 |
| gcctgcttag agaatggatt tttagccatt catatttcca aaaacgaagt agctaataaa | 360 |
| catgaagaga aagttctttt tcaaattgag aattaa | 396 |
| 12. SQ SEQUENCE 128 AA; 14846 MW; C091E32736F9AC79 CRC64; | |
| MGKKKKDCLF HVDGFEEWMD QFCSDSCSNF SFPNQIHIDL CETEQEYILE TDVPNVTEQN | |
| VVIKKMETGL NICILHKNIS LQRNIPLPTT IIYKKMLACL ENGFLAIHIS KNEVANKHEE | |
| KVLFQIEN | |
| 11. SQ Sequence 387 BP; 140 A; 53 C; 66 G; 128 T; 0 other; 3474606372 CRC32; | |
| atgggcaaga aaaagaagga ttgtcttttt catgttgatg gttttgaaga atggatggat | 60 |
| caattttgtt ctgattcttg tagtaacttt agtttcccaa atcaaattca tattgatctt | 120 |
| tgtgaaactg aacaagaata cattttggaa acagatgtac caaatgtaac tgaacaaaat | 180 |
| gtagttatta aaaagatgga gacaggccta aacatttgca tacttcataa aaatatttct | 240 |
| ttgcagcgga acattccttt acccactact attatttata agaagatgct agcctgctta | 300 |
| gagaatggat ttttagccat tcatatttcc aaaaacgaag tagctaataa acatgaagag | 360 |
| aaagttcttt ttcaaattga gaattaa | 387 |

B. anthracis CotH - (Q6HZS5/Q81RJ8)

| | |
|---|---|
| 14. SQ SEQUENCE 368 AA; 43725 MW; 8F14571D4C809A4F CRC64; | |
| MKRTEKGCEN MLPSYDFFIH PMYVVELKKD IWSDSPVPAK LTYGKKKYDI DIVYRGAHIR | |
| EFEKKSYHVM FYKPKKFQGA KEFHLNSEFM DPSLIRNKLS LDFFHDIGVH SPKSQHVFIK | |
| INGQIQGVYL QLESVDENFL KNRGLPSGSI YYAIDDDANF SLMSERDKDV KTELFAGYEF | |
| KYSNEHSEEQ LSEFVFQANA LSREAYEKEI GKFLNVDKYL RWLAGVIFTQ NFDGFVHNYA | |
| LYHNDETNLF EVIPWDYDAT WGRDVQGRPL NHEYIRIQGY NTLSARLLDI PVFRKQYRSI | |
| LEEILEEQFT VSFMMPKVES LCEAIRPYLL QDPYMKEKLE TFDQEPGVIE EYINKRRKYI | |
| QDHLHELD | |
| 13. SQ Sequence 1107 BP; 403 A; 125 C; 218 G; 361 T; 0 other; 1333935843 CRC32; | |
| atgaagagaa ctgagaaggg atgtgaaaat atgctacctt catatgattt ttttattcat | 60 |
| ccaatgtacg tagtggaatt gaaaaaagac atttggtcag acagtccagt accagcaaaa | 120 |
| ttaacttatg gaaaaaagaa gtatgatatt gatatcgtat atcggggtgc tcatattcgt | 180 |
| gaatttgaga aaaagtctta tcatgttatg ttttataagc caaaaaaatt tcaaggtgcg | 240 |
| aaagagtttc atttaaattc tgagtttatg gatccgtctc tcatacgaaa taaattatct | 300 |
| ttagattttt ttcatgatat tggtgtacat tcaccaaaat cacaacatgt atttataaaa | 360 |
| attaatggtc aaattcaagg agtatattta cagttagaat cagttgatga aaactttttg | 420 |
| aaaaatagag gattacctag tggttctatt tattatgcga tagatgatga tgcgaatttc | 480 |
| tctttaatga gtgaaagaga taaagatgtt aagactgagc tttttgcggg ttatgaattt | 540 |
| aaatattcga atgaacatag tgaagaacaa ttgagtgaat ttgtatttca agcgaacgct | 600 |
| ttgtcgaggg aagcgtatga aaaagaaatt gggaagtttc taaatgttga taagtattta | 660 |
| cgatggttag caggcgttat ttttacacaa aactttgatg gttttgttca taactatgca | 720 |
| ttataccata acgatgaaac aaatttattt gaagtgatac cgtgggatta tgatgcgact | 780 |
| tgggggcgtg atgtacaagg gagaccgctt aatcatgaat atattcgtat tcaaggttat | 840 |
| aacacgttaa gtgcaagatt gttagatata cctgtattta gaaaacaata ccgaagtatt | 900 |
| ttggaagaaa tattagaaga acaatttacg gtttcattta tgatgccgaa agtagaaagt | 960 |
| ttatgtgaag caatacgtcc ttatttacta caagatccat atatgaaaga aaaattagaa | 1020 |
| acctttgatc aagaacctgg tgtgattgag gaatatataa ataaaagaag aaagtatata | 1080 |
| caagatcatt tacatgaatt ggattaa | 1107 |
| 16. SQ SEQUENCE 358 AA; 42547 MW; 8269D4EDA237D846 CRC64; | |
| MLPSYDFFIH PMYVVELKKD IWSDSPVPAK LTYGKKKYDI DIVYRGAHIR EFEKKSYHVM | |
| FYKPKKFQGA KEFHLNSEFM DPSLIRNKLS LDFFHDIGVH SPKSQHVFIK INGQIQGVYL | |
| QLESVDENFL KNRGLPSGSI YYAIDDDANF SLMSERDKDV KTELFAGYEF KYSNEHSEEQ | |
| LSEFVFQANA LSREAYEKEI GKFLNVDKYL RWLAGVIFTQ NFDGFVHNYA LYHNDETNLF | |
| EVIPWDYDAT WGRDVQGRPL NHEYIRIQGY NTLSARLLDI PVFRKQYRSI LEEILEEQFT | |
| VSFMMPKVES LCEAIRPYLL QDPYMKEKLE TFDQEPGVIE EYINKRRKYI QDHLHELD | |
| 15. SQ Sequence 1077 BP; 389 A; 124 C; 208 G; 356 T; 0 other; 1858172502 CRC32; | |
| atgctacctt catatgattt ttttattcat ccaatgtacg tagtggaatt gaaaaaagac | 60 |
| atttggtcag acagtccagt accagcaaaa ttaacttatg gaaaaaagaa gtatgatatt | 120 |
| gatatcgtat atcggggtgc tcatattcgt gaatttgaga aaaagtctta tcatgttatg | 180 |
| ttttataagc caaaaaaatt tcaaggtgcg aaagagtttc atttaaattc tgagtttatg | 240 |
| gatccgtctc tcatacgaaa taaattatct ttagattttt ttcatgatat tggtgtacat | 300 |
| tcaccaaaat cacaacatgt atttataaaa attaatggtc aaattcaagg agtatattta | 360 |
| cagttagaat cagttgatga aaactttttg aaaaatagag gattacctag tggttctatt | 420 |
| tattatgcga tagatgatga tgcgaatttc tctttaatga gtgaaagaga taaagatgtt | 480 |
| aagactgagc tttttgcggg ttatgaattt aaatattcga atgaacatag tgaagaacaa | 540 |
| ttgagtgaat ttgtatttca agcgaacgct ttgtcgaggg aagcgtatga aaaagaaatt | 600 |
| gggaagtttc taaatgttga taagtattta cgatggttag caggcgttat ttttacacaa | 660 |
| aactttgatg gttttgttca taactatgca ttataccata acgatgaaac aaatttattt | 720 |
| gaagtgatac cgtgggatta tgatgcgact tgggggcgtg atgtacaagg gagaccgctt | 780 |
| aatcatgaat atattcgtat tcaaggttat aacacgttaa gtgcaagatt gttagatata | 840 |
| cctgtattta gaaaacaata ccgaagtatt ttggaagaaa tattagaaga acaatttacg | 900 |
| gtttcattta tgatgccgaa agtagaaagt ttatgtgaag caatacgtcc ttatttacta | 960 |
| caagatccat atatgaaaga aaaattagaa acctttgatc aagaacctgg tgtgattgag | 1020 |
| gaatatataa ataaaagaag aaagtatata caagatcatt tacatgaatt ggattaa | 1077 |

B. anthracis CotC - (Q81L62, Q6HSL4, Q6KLV8)

| | |
|---|---|
| 18. SQ SEQUENCE 110 AA; 12476 MW; A6E3127040680A6F CRC64; | |
| MNTKNKKIAL GTILLTSIIG VISVSLYFTY YGTPWGKQAA ITESKEYITK YFNLDAEVKN | |
| TSYDAKMNSY AIAFDTNKDG EFTIEYKSPN NFNISPEVQA YLSKHSKFTE | |
| 17. SQ Sequence 333 BP; 131 A; 51 C; 48 G; 103 T; 0 other; 1167375996 CRC32; | |
| ttgaatacaa agaataaaaa aatagctcta ggaactattt tattaacttc tattattgga | 60 |
| gttattagtg tatctcttta tttcacctat tatggtaccc cttggggaaa acaagcagca | 120 |
| attacggaat caaaagagta tattacaaaa tattttaatc tagatgcaga agtcaaaaac | 180 |
| acttcttacg atgctaaaat gaatagctat gcaatcgcct ttgacacaaa taaagacgga | 240 |
| gagtttacta tcgaatataa aagtcctaat aactttaata tttctccaga agtacaagcg | 300 |
| tatttaagta aacactctaa atttacagag tag | 333 |

B. anthracis CotAlpha - (Q81MI2, Q6HTY1, Q6KN63, Q6RVB2)

| | |
|---|---|
| 20. SQ SEQUENCE 120 AA; 13421 MW; 285E193D12756C12 CRC64; | |
| MFGSFGCCDN FRDCHHHERE RDHREKEREV KPQQPAVCNV LASISVGTEL SLLSVKGVGS | |
| FNNVIFEGFC NGVALFSALA RNNNDKDNKD NNKDDKHNQN RNTFTGILRV CPTDIVAIAI | |
| 19. SQ Sequence 363 BP; 117 A; 62 C; 73 G; 111 T; 0 other; 2762494190 CRC32; | |
| atgtttggat catttggatg ctgtgataac tttagagact gtcatcatca tgaaagagag | 60 |
| cgcgaccatc gtgagaaaga gagagaggtt aaaccacaac aaccagctgt atgtaacgta | 120 |
| cttgctagca tttcagttgg aacagagctt tctctattaa gcgttaaagg tgttggatct | 180 |
| ttcaacaatg taatttttga aggtttctgt aacggtgttg ctcttttctc tgctttagct | 240 |
| cgtaataaca atgacaaaga taacaaagat aacaacaaag atgataagca caatcaaaac | 300 |
| cgaaatactt ttactggtat tttacgtgta tgcccaactg atattgttgc gatcgctatc | 360 |
| taa | 363 |

B. anthracis CotF - (Q81NQ7, Q6HWX7, Q6KR09)

| | |
|---|---|
| 22. SQ SEQUENCE 159 AA; 18279 MW; 1B70A754AC5ED043 CRC64; | |
| MSYPNQLAWH ETLELHELVA FQANGLIKLK KSVRNVPDQA LQSLYIKAIN AIQNNLQELV | |
| QFYPYAPGFQ AQHRDDTGFY AGDLLGLAKT SVRNYAIAIT ETATPRLREV LTRQINGAIQ | |
| LHAQVFNFMY ERGYYPAYDL KELLKNDVQN VQKAIQMQY | |
| 21. SQ Sequence 480 BP; 173 A; 83 C; 82 G; 142 T; 0 other; 1446972935 CRC32; | |
| atgtcttatc ctaatcagct agcttggcat gaaacattgg agttacatga attagtagca | 60 |
| tttcaagcaa acggtttaat caaattaaaa aaatcagtta ggaatgtacc tgatcaagca | 120 |
| cttcaatcgt tatatattaa agctataaat gccatccaaa acaatctaca agagttagta | 180 |
| caattttatc cttatgctcc tggatttcaa gcgcagcatc gtgatgacac tggattttac | 240 |
| gctggagatt tacttggatt agcaaagaca tctgttcgaa actatgcaat agcgattacc | 300 |
| gaaactgcaa cgccgcgact tagagaagtt ttaacccgtc aaataaatgg agctatacaa | 360 |
| ttacatgcac aggtttttaa ctttatgtat gaacgtggtt actatccagc ttatgattta | 420 |
| aaggaactat taaaaaatga tgttcaaaat gtgcaaaagg caatacaaat gcaatattaa | 480 |

B. anthracis CotD - (Q81SR5, Q6I0Z7, Q6KUV1)

| | |
|---|---|
| 24. SQ SEQUENCE 140 AA; 14867 MW; 164F4228BBD63157 CRC64; | |
| MHHCHPCFGG HKPTGPICTT APVIHPTKQC VTHSFSTTVV PHIFPTHTTH VHHQQIKNQN | |
| FFPQTNSNVN VVDPIDPGFG GCGPCGHGHH HHHGHQISPF GPGPNVSPFG PGPNVSPFLP | |
| NNVSPVGPNI GPNVGGIFKK | |
| 23. SQ Sequence 423 BP; 134 A; 109 C; 74 G; 106 T; 0 other; 3067299696 CRC32; | |
| atgcatcatt gtcatccttg ctttggaggg cataagccta caggacctat ttgtacaact | 60 |
| gctcctgtca ttcatccgac gaaacaatgc gtaacacatt ctttttcaac aacggtggtg | 120 |
| ccacacattt tcccgacgca tacaacacat gtacatcatc aacaaattaa aaaccaaaac | 180 |
| ttcttcccgc aaacaaattc aaatgtaaat gttgtagacc caatcgatcc aggattcggc | 240 |
| ggatgtggac catgtggcca tggtcatcac caccaccacg gtcatcaaat atccccattc | 300 |
| ggaccaggac cgaatgtatc accgtttgga ccaggaccaa atgtatcgcc atttttacca | 360 |
| aacaatgtat caccagtagg tccgaatatt ggaccaaacg ttggtggaat atttaaaaag | 420 |
| taa | 423 |

B. anthracis CotZ - (Q81TN3, Q6I1W3, Q6KVQ5/Q81TN7, Q6I1W7, Q6KVQ9)

| | |
|---|---|
| 26. SQ SEQUENCE 156 AA; 16842 MW; 4AE98760DFB6BAB8 CRC64; | |
| MSCNCNEDHH HHDCDFNCVS NVVRFIHELQ ECATTTCGSG CEVPFLGAHN SASVANTRPF | |
| ILYTKAGAPF EAFAPSANLT SCRSPIFRVE SIDDDDCAVL RVLSVVLGDT SPVPPTDDPI | |
| CTFLAVPNAR LISTNTCLTV DLSCFCAIQC LRDVTI | |
| 25. SQ Sequence 471 BP; 127 A; 100 C; 90 G; 154 T; 0 other; 2646187239 CRC32; | |
| atgagctgca attgtaacga agaccatcat caccatgatt gtgatttcaa ctgtgtatca | 60 |
| aatgtcgttc gttttataca tgaattacaa gaatgcgcaa ctacaacatg cggatctggt | 120 |
| tgcgaagttc cctttttagg agcacataat agcgcatccg tagcaaatac gcgtcctttt | 180 |
| attttataca caaaagctgg cgcacctttt gaagcatttg caccttctgc aaaccttact | 240 |
| agctgccgat ctccaatttt ccgtgtcgag agtatagatg atgatgattg cgctgtattg | 300 |
| cgtgtattaa gtgtagtatt aggtgatact tctcctgtac cacctaccga cgatccaatc | 360 |
| tgtacattcc tagctgtacc aaatgcaaga ttaatatcga ctaacacttg tcttactgtt | 420 |
| gatttaagtt gcttctgtgc gattcaatgc ttgcgtgatg ttacgattta a | 471 |
| 28. SQ SEQUENCE 152 AA; 16146 MW; EB6C8561080FD288 CRC64; | |
| MSCNENKHHG SSHCVVDVVK FINELQDCST TTCGSGCEIP FLGAHNTASV ANTRPFILYT | |
| KAGAPFEAFA PSANLTSCRS PIFRVESVDD DSCAVLRVLS VVLGDSSPVP PTDDPICTFL | |
| AVPNARLVST STCITVDLSC FCAIQCLRDV TI | |
| 27. SQ Sequence 459 BP; 129 A; 93 C; 85 G; 152 T; 0 other; 2977073396 CRC32; | |
| atgagttgta acgaaaataa acaccatggc tcttctcatt gtgtagttga cgttgtaaaa | 60 |
| ttcatcaatg aattacaaga ttgttctaca acaacatgtg gatctggttg tgaaattcca | 120 |
| tttttaggcg cacacaatac tgcatcagta gcaaatacac gcccttttat tttatacaca | 180 |
| aaagctggcg caccttttga agcatttgca ccttctgcaa accttactag ctgccgatct | 240 |
| ccaattttcc gtgtggaaag tgtagatgat gatagctgtg ctgtactacg tgtattaagt | 300 |
| gtagtattag gtgatagctc tcctgtacca cctactgatg acccaatttg tacgttttta | 360 |
| gctgtaccaa atgcaagact agtatcgaca tctacttgta ttactgtaga tttaagctgt | 420 |
| ttctgtgcga ttcaatgctt acgcgacgtt actatctaa | 459 |

B. anthracis Cot(Putative 1) - (Q611R6)

| | |
|---|---|
| 30. SQ SEQUENCE 199 AA; 21922 MW; DD5A437A2CDDE9FC CRC64; | |
| MIVSLKKKLG MGVASAALGL SLIGGGTFAY FSDKEVSNNT FAAGTLDLTL DPKTLVDIKD | |
| LKPGDSVKKE FLLKNSGSLT IKDVKLATKY TVKDVKGDNA GEDFGKHVKV KFLWNWDKQS | |
| EPVYETTLAD LQKTDPDLLA QDIFAPEWGE KGGLEAGTED YLWVQFEFVD DGKDQNIFQG | |
| DSLNLEWTFN ANQEAGEEK | |
| 29. SQ Sequence 600 BP; 216 A; 76 C; 138 G; 170 T; 0 other; 217524501 CRC32; | |
| ttgattgtga gtctgaaaaa gaaattaggt atgggagttg catcagcagc attggggtta | 60 |
| tctttaattg gtggaggaac atttgcttac tttagcgata aagaagtatc gaacaataca | 120 |
| tttgcagctg ggacgttaga tcttacatta gaccctaaaa cgcttgtaga tattaaagat | 180 |
| ttaaaaccag gggattctgt taagaaagag ttcttattaa agaatagcgg ttcattaaca | 240 |
| attaaagacg ttaaactagc aacaaagtat actgtgaaag atgtaaaagg tgataatgct | 300 |
| ggtgaagact ttggtaagca cgttaaagtg aaattccttt ggaactggga taaacaaagt | 360 |
| gagcctgtat atgaaacaac tttagcagac ttacaaaaaa ctgatccaga tcttttagct | 420 |
| caagacattt ttgctcctga gtggggggaa aagggtggat tagaagctgg taccgaggat | 480 |
| tatttatggg tacaatttga atttgtagat gatggaaaag accaaaatat cttccaaggt | 540 |
| gattcattga atttagaatg gacattcaat gctaaccaag aagctggaga agaaaaataa | 600 |

B. anthracis Cot(Putative 2) - (Q6HYG8)

| | |
|---|---|
| 32. SQ SEQUENCE 135 AA; 15486 MW; 22A7318D9304ADA3 CRC64; | |
| MKGMNNAVDQ ANKGIQQMLN IKFPNSYHWF LKQYGSGGLD GMDIHGCETT AADSSVVYHT | |
| KSYRETYNLP EQYIVLNDID GTMTCLDTNQ MKDGECPVVF WSRFSKELYA ITYENFGDYL | |
| LDCLQESVDN LYDED | |
| 31. SQ Sequence 408 BP; 135 A; 62 C; 83 G; 128 T; 0 other; 443956393 CRC32; | |
| atgaagggca tgaataatgc agttgaccag gccaataaag gcatacaaca aatgctaaac | 60 |
| attaaattcc caaatagtta tcattggttt ttaaaacagt atggtagcgg cggactggat | 120 |
| ggtatggata ttcatggttg tgagacaaca gctgcagatt cttccgttgt ttaccacacc | 180 |
| aagtcatata gagaaacata taaccttcct gaacaataca ttgttttaaa tgatattgat | 240 |
| ggtactatga catgtttaga taccaatcaa atgaaagatg gcgagtgtcc tgttgtcttt | 300 |
| tggagtcgtt tttcaaagga actgtatgcc attacttatg aaaacttcgg cgactatcta | 360 |
| ttagattgtt tacaagaatc tgtagataat ttgtatgatg aggattaa | 408 |

B. anthracis Cot(Putative 3) - (Q81Q97, Q6KSH6)

| | |
|---|---|
| 34. SQ SEQUENCE 132 AA; 15170 MW; 0A9E664E548D0B19 CRC64; | |
| MNNAVDQANK GIQQMLNIKF PNSYHWFLKQ YGSGGLDGMD IHGCETTAAD SSVVYHTKSY | |
| RETYNLPEQY IVLNDIDGTM TCLDTNQMKD GECPVVFWSR FSKELYAITY ENFGDYLLDC | |
| LQESVDNLYD ED | |
| 33. SQ Sequence 399 BP; 132 A; 61 C; 79 G; 127 T; 0 other; 2816972438 CRC32; | |
| atgaataatg cagttgacca ggccaataaa ggcatacaac aaatgctaaa cattaaattc | 60 |
| ccaaatagtt atcattggtt tttaaaacag tatggtagcg gcggactgga tggtatggat | 120 |
| attcatggtt gtgagacaac agctgcagat tcttccgttg tttaccacac caagtcatat | 180 |
| agagaaacat ataaccttcc tgaacaatac attgttttaa atgatattga tggtactatg | 240 |
| acatgtttag ataccaatca aatgaaagat ggcgagtgtc ctgttgtctt ttggagtcgt | 300 |
| ttttcaaagg aactgtatgc cattacttat gaaaacttcg gcgactatct attagattgt | 360 |
| ttacaagaat ctgtagataa tttgtatgat gaggattaa | 399 |

B. anthracis Cot(Putative 4) - (Q81TI4, Q6I1R8, Q6KVK7)

| | |
|---|---|
| 36. SQ SEQUENCE 195 AA; 21542 MW; D49780F43EEF8198 CRC64; | |
| MTLKKKLGMG IASAVLGAAL VGGGTFAFFS DKEVSNNTFA TGTLDLALNP STVVNVSNLK | |
| PGDTVEKEFK LENKGTLDIK KVLLKTDYNV EDVKKDNKDD FGKHIKVTFL KNVDKHETIV | |
| KETALDKLKG DTLTAVNNDL AAWFWDEKGI SAGKSDKFKV KFEFVDNKKD QNEFQGDKLQ | |
| LTWTFDAQQG DGETK | |
| 35. SQ Sequence 588 BP; 241 A; 68 C; 119 G; 160 T; 0 other; 1741221389 CRC32; | |
| atgactttaa agaaaaaatt aggaatgggt atcgcatcag cagtattagg ggctgcatta | 60 |
| gttggcggag gaacatttgc atttttcagt gataaagaag tgtcaaacaa tacatttgcg | 120 |
| actggtacgc ttgatttagc attaaatcca tcaacagttg ttaatgtatc gaatttaaaa | 180 |
| cctggtgata cagttgaaaa agaatttaaa ttagaaaata aagggacatt agatattaaa | 240 |
| aaagtactac taaaaacaga ttacaatgta gaagatgtga agaaagataa taaagatgat | 300 |
| tttggtaaac atattaaagt aacattctta aaaaatgtag acaagcatga aacaatcgta | 360 |
| aaagaaacag cgcttgataa attgaagggt gacacactta ctgcggtaaa taacgattta | 420 |
| gctgcttggt tctgggatga aaaaggtatt tcagcaggta aatctgataa attcaaagtg | 480 |
| aaatttgaat tcgttgataa taaaaaagat caaaatgaat tccaaggcga taagttacaa | 540 |
| ttaacttgga cgtttgatgc acagcaaggc gatggtgaaa caaaataa | 588 |

B. anthracis CotHypoAlpha - (Q81MI2, Q6HTY1, Q6KN63, Q6RVB2)

| | |
|---|---|
| 38. SQ SEQUENCE 120 AA; 13421 MW; 285E193D12756C12 CRC64; | |
| MFGSFGCCDN FRDCHHHERE RDHREKEREV KPQQPAVCNV LASISVGTEL SLLSVKGVGS | |
| FNNVIFEGFC NGVALFSALA RNNNDKDNKD NNKDDKHNQN RNTFTGILRV CPTDIVAIAI | |
| 37. SQ Sequence 363 BP; 117 A; 62 C; 73 G; 111 T; 0 other; 2762494190 CRC32; | |
| atgtttggat catttggatg ctgtgataac tttagagact gtcatcatca tgaaagagag | 60 |
| cgcgaccatc gtgagaaaga gagagaggtt aaaccacaac aaccagctgt atgtaacgta | 120 |
| cttgctagca tttcagttgg aacagagctt tctctattaa gcgttaaagg tgttggatct | 180 |
| ttcaacaatg taatttttga aggtttctgt aacggtgttg ctcttttctc tgctttagct | 240 |
| cgtaataaca atgacaaaga taacaaagat aacaacaaag atgataagca caatcaaaac | 300 |
| cgaaatactt ttactggtat tttacgtgta tgcccaactg atattgttgc gatcgctatc | 360 |
| taa | 363 |

B. anthracis CotE - (Q81WR2, Q6HUW6, Q6KP42)

| | |
|---|---|
| 40. SQ SEQUENCE 180 AA; 20400 MW; CB4802E18F49BBD1 CRC64; | |
| MSEFREIITK AVVGKGRKYT KSTHTCESNN EPTSILGCWV INHSYEARKN GKHVEIEGFY | |
| DVNTWYSFDG NTKTEVVTER VNYTDEVSIG YRDKNFSGDD LEIIARVIQP PNCLEALVSP | |
| NGNKIVVTVE REFVTEVVGE TKICVSVNPE GCVESDEDFQ IDDDEFEELD PNFIVDAEEE | |
| 39. SQ Sequence 543 BP; 197 A; 67 C; 128 G; 151 T; 0 other; 764211315 CRC32; | |
| atgtccgaat ttagagagat tattacaaaa gcagtggttg gaaaaggacg taagtataca | 60 |
| aagtcaacgc atacatgtga atcgaataat gagccaacaa gtattttagg gtgctgggta | 120 |
| attaaccact cgtacgaagc aagaaagaat ggaaaacatg tggaaattga aggtttctat | 180 |
| gatgtgaaca cttggtattc atttgatggc aatacaaaga cagaagttgt aacagaacgt | 240 |
| gtgaactaca cggatgaagt aagtattggc tatcgtgata aaaacttttc aggtgatgat | 300 |
| ttagaaatta ttgctcgtgt cattcagcca ccaaattgtt tagaagctct tgtatcacca | 360 |
| aatggtaata aaattgttgt aacggtagaa cgtgaatttg taacagaagt agttggtgaa | 420 |
| acgaaaattt gtgtaagtgt aaatccggaa ggttgtgtag aatcagacga agatttccaa | 480 |
| atcgatgatg atgagtttga agagttagat ccaaacttta tcgttgatgc agaagaagag | 540 |
| taa | 543 |

B. anthracis CotF(Related) - (Q81XJ6, Q6HRC6, Q6KKP5)

| | |
|---|---|
| 42. SQ SEQUENCE 82 AA; 9519 MW; 9C64A6F847B2672F CRC64; | |
| MNEKDMVNDY LAGLNASLTS YANYIAQSDN EQLHQTLIQI RNQDEMRQRN MYEYAKQKSY | |
| YKPAAPANPM IVQQLKSQLS AE | |
| 41. SQ Sequence 249 BP; 102 A; 36 C; 46 G; 65 T; 0 other; 118011809 CRC32; | |
| atgaatgaaa aagatatggt aaatgattat ttagcaggat tgaatgcaag tttaacaagt | 60 |
| tatgcaaatt atattgctca gtctgataat gaacagttac accaaacgtt aatccaaatt | 120 |
| cgtaatcaag atgaaatgcg tcaacgtaat atgtatgagt atgcaaagca aaagagttat | 180 |
| tacaagccag cggcacctgc gaatccaatg attgtacaac aattaaaaag ccaattaagt | 240 |
| gcggaataa | 249 |

B. anthracis BclA (40048) - (Q52NY8)

| | |
|---|---|
| 44. SQ SEQUENCE 322 AA; 30133 MW; B036C1F1F4432E02 CRC64; | |
| MSNNNYSNGL NPDESLSASA FDPNLVGPTL PPIPPFTLPT GPTGPTGPTG PTGPTGPTGP | |
| TGPTGPTGPT GDTGTTGPTG PTGPTGPTGP TGPTGPTGPT GPTGPTGPTG PTGPTGDTGT | |
| TGPTGPTGPT GPTGPTGDTG TTGPTGPTGP TGPTGPTGPT GPTGPTGPTG PTGATGLTGP | |
| TGPTGPSGLG LPAGLYAFNS GGISLDLGIN DPVPFNTVGS QFGTAISQLD ADTFVISETG | |
| FYKITVIANT ATASVLGGLT IQVNGVPVPG TGSSLISLGA PIVIQAITQI TTTPSLVEVI | |
| VTGLGLSLAL GTSASIIIEK VA | |
| 43. SQ Sequence 969 BP; 265 A; 247 C; 231 G; 226 T; 0 other; 3713744812 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccaacc ggagacaccg gtactactgg accaactggg | 240 |
| ccaactggac caactgggcc gactgggcca actggaccaa ctgggccgac tgggccaact | 300 |
| ggaccaactg ggccgactgg gccaactgga ccaactgggc caactggaga cactggtact | 360 |
| actggaccaa ctgggccaac tggaccaact ggaccaactg ggccaactgg agacactggt | 420 |
| actactggac caaccgggcc aactggacca actggaccaa ctgggccgac tggaccgact | 480 |
| gggccgactg ggccaactgg gccaactggg ccaactggtg ctaccggact gactggaccg | 540 |
| actggaccga ctgggccatc cggactagga cttccagcag gactatatgc atttaactcc | 600 |
| ggtgggattt ctttagattc aggaattaat gatccagtac catttaatac cgttggatct | 660 |
| cagtttggta cagcaatttc tcaactagat gctgatactt tcgtaattag tgaaactgga | 720 |
| ttctataaaa ttactgttac cgctaacact gcaacagcaa gtgtattagg aggtcttaca | 780 |
| atccaagtga atggagtacc tgtaccaggt actggatcaa gtttgatttc actcggagca | 840 |
| cccatcgcta ctcaagcaat tacgcaaatt acgacaactc catcactagt cgaagcaatc | 900 |
| gttacagggc ttggaccatc actagccctt ggcacgagtg catccattat tattgaaaaa | 960 |
| gttgcttaa | 969 |

B. anthracis BclA (A16R) - (Q52NZ0)

| | |
|---|---|
| 46. SQ SEQUENCE 388 AA; 35793 MW; 50767CAB307A5A7F CRC64; | |
| MSNNNYSNGL NPDESLSASA FDPNLVGPTL PPIPPFTLPT GPTGPTGPTG PTGPTGPTGP | |
| TGPTGPTGPT GDTGTTGPTG PTGPTGPTGP TGDTGTTGPT GPTGPTGPTG PTGPTGPTGP | |
| TGPTGPTGPT GPTGPTGPTG PTGPTGDTGT TGPTGPTGPT GPTGPTGPTG PTGPTGPTGP | |
| TGPTGPTGPT GDTGTTGPTG PTGPTGPTGP TGDTGTTGPT GPTGPTGPTG PTGPTGPTGA | |
| TGLTGPTGPT GPSGLGLPAG LYAFNSGGIS LDLGINDPVP FNTVGSQFGT AISQLDADTF | |
| VISETGFYKI TVIANTATAS VLGGLTIQVN GVPVPGTGSS LISLGAPIVI QAITQITTTP | |
| SLVEVIVTGL GLSLALGTSA SIIIEKVA | |
| 45. SQ Sequence 1167 BP; 321 A; 309 C; 285 G; 252 T; 0 other; 3217654551 CRC32; | |
| atgtcaaata acaattattc aaatggatta aaccccgatg aatctttatc agctagtgca | 60 |
| tttgacccta atcctgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccaact ggagacaccg gtactactgg accaactggg | 240 |
| ccgactgggc caactggacc aactgggcca actggagaca ctggtactac tggaccaact | 300 |
| gggccaaccg gaccaactgg gccgactggg ccaactggac caactgggcc gactgggcca | 360 |
| actggaccaa ctgggccaac tggaccaact ggaccaaccg ggccaactgg accaactgga | 420 |
| ccaactgggc caactggaga cactggtact accggaccaa ctgggccaac tggaccaacc | 480 |
| ggaccaactg ggccgactgg accgactggg ccgactgggc caactggacc aactgggccg | 540 |
| accgggccaa ctggaccaac cgggccaact ggagacaccg gcactactgg accaactggg | 600 |
| ccaactggac caactggacc aactgggcca actggagaca ctggtactac tggaccaacc | 660 |
| gggccaactg gaccaactgg accaactggg ccaactggac caactgggcc aactggtgcc | 720 |
| accggactga ctggaccgac tggaccgact gggccatccg gactaggact tccagcagga | 780 |
| ctatatgcat ttaactccgg tgggatttct ttagatttag gaattaatga tccagtacca | 840 |
| tttaatactg ttggatctca gtttggtaca gcaatttctc aattagatgc tgatactttc | 900 |
| gtaattagtg aaactggatt ctataaaatt actgttatcg ccaatactgc aacagcaagt | 960 |
| gtattaggag gcctcacaat ccaagtgaat ggagtacctg taccaggtac tggatcaagt | 1020 |
| ttgatttcac tcggagcacc tatcgttatt caagcaatta cgcaaattac gacaactcca | 1080 |
| tcattagttg aagcaattgc cacagggctt ggactatcac tagctcttgg cacgagtgca | 1140 |
| tccattatta ttgaaaaagt tgcttaa | 1167 |

B. anthracis BclA (CIPA2) - (Q83TL0)

| | |
|---|---|
| 48. SQ SEQUENCE 262 AA; 25006 MW; CB03E1E413646488 CRC64; | |
| MSNNNYSNGL NPDESLSASA FDPNLVGPTL PPIPPFTLPT GPTGPTGPTG PTGPTGPTGP | |
| TGPTGPTGPT GDTGTTGPTG PTGPTGPTGP TGDTGTTGPT GPTGPTGPTG PTGATGLTGP | |
| TGPTGPSGLG LPAGLYAFNS GGISLDLGIN DPVPFNTVGS QFGTAISQLD ADTFVISETG | |
| FYKITVIANT ATASVLGGLT IQVNGVPVPG TGSSLISLGA PIVIQAITQI TTTPSLVEVI | |
| VTGLGLSLAL GTSASIIIEK VA | |
| 47. SQ Sequence 789 BP; 223 A; 189 C; 173 G; 204 T; 0 other; 668699339 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccaact ggagacactg gtactactgg accaactggg | 240 |
| ccaactggac caactgggcc aactgggcca actggagaca ctggtactac tggaccaact | 300 |
| gggccaactg gaccaactgg accaactggg ccaactggtg ctaccggact gactggaccg | 360 |
| actggaccga ctgggccatc cggactagga cttccagcag gactatatgc atttaactcc | 420 |
| ggtgggattt ctttagattt aggaattaat gatccagtac catttaatac tgttggatct | 480 |
| cagtttggta cagcaatttc tcaattagat gctgatactt tcgtaattag tgaaactgga | 540 |
| ttctataaaa ttactgttat cgctaatact gcaacagcaa gtgtattagg aggtcttaca | 600 |
| atccaagtga atggagtacc tgtaccaggt actggatcaa gtttgatttc actcggagca | 660 |
| cctatcgtta ttcaagcaat tacgcaaatt acgacaactc catcattagt tgaagtaatt | 720 |
| gttacagggc ttggactatc actagctctt ggcacgagtg catccattat tattgaaaaa | 780 |
| gttgcttaa | 789 |

B. anthracis BclA (7611) - (Q83UV2)

| | |
|---|---|
| 50. SQ SEQUENCE 253 AA; 24218 MW; 10231F93AD9A1385 CRC64; | |
| MSNNNYSNGL NPDESLSASA FDPNLVGPTL PPIPPFTLPT GPTGPTGPTG PTGPTGPTGP | |
| TGPTGPTGPT GPTGPTGPTG PTGDTGTTGP TGPTGPTGPT GPTGATGLTG PTGPTGPSGL | |
| GLPAGLYAFN SGGISLDLGI NDPVPFNTVG SQFGTAISQL DADTFVISET GFYKITVIAN | |
| TATASVLGGL TIQVNGVPVP GTGSSLISLG APIVIQAITQ ITTTPSLVEV IVTGLGLSLA | |
| LGTSASIIIE KVA | |
| 49. SQ Sequence 762 BP; 216 A; 182 C; 165 G; 199 T; 0 other; 3124681291 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tggaccaact gggccaactg gaccaactgg gccaactggg | 240 |
| ccaactggag acactggtac tactggacca actgggccaa ctggaccaac tggaccaact | 300 |
| gggccaactg gtgctaccgg actgactgga ccgactggac cgactgggcc atccggacta | 360 |
| ggacttccag caggactata tgcatttaac tccggtggga tttctttaga tttaggaatt | 420 |
| aatgatccag taccatttaa tactgttgga tctcagtttg gtacagcaat ttctcaatta | 480 |
| gatgctgata ctttcgtaat tagtgaaact ggattctata aaattactgt tatcgctaat | 540 |
| actgcaacag caagtgtatt aggaggtctt acaatccaag tgaatggagt acctgtacca | 600 |
| ggtactggat caagtttgat ttcactcgga gcacctatcg ttattcaagc aattacgcaa | 660 |
| attacgacaa ctccatcatt agttgaagta attgttacag ggcttggact atcactagct | 720 |
| cttggcacga gtgcatccat tattattgaa aaagttgctt aa | 762 |

B. anthracis BclA (ATCC4229) - (Q83WA5)

| | |
|---|---|
| 52. SQ SEQUENCE 223 AA; 21665 MW; 450F8ECB33FBC58E CRC64; | |
| MSNNNYSNGL NPDESLSASA FDPNLVGPTL PPIPPFTLPT GPTGPTGPTG PTGPTGDTGT | |
| TGPTGPTGPT GPTGATGLTG PTGPTGPSGL GLPAGLYAFN SGGISLDLGI NDPVPFNTVG | |
| SQFGTAISQL DADTFVISET GFYKITVIAN TATASVLGGL TIQVNGVPVP GTGSSLISLG | |
| APIVIQAITQ ITTTPSLVEV IVTGLGLSLA LGTSASIIIE KVA | |
| 51. SQ Sequence 672 BP; 195 A; 152 C; 136 G; 189 T; 0 other; 1857948650 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccaactgg accaactggg ccaactgggc caactggaga cactggtact | 180 |
| actggaccaa ctgggccaac tggaccaact gggccaactg gtgctaccgg actgactgga | 240 |
| ccgactggac cgactgggcc atccggacta ggacttccag caggactata tgcatttaac | 300 |
| tccggtggga tttctttaga tttaggaatt aatgatccag taccatttaa tactgttgga | 360 |
| tctcagtttg gtacagcaat ttctcaatta gatgctgata ctttcgtaat tagtgaaact | 420 |
| ggattctata aaattactgt tatcgctaat actgcaacag caagtgtatt aggaggtctt | 480 |
| acaatccaag tgaatggagt acctgtacca ggtactggat caagtttgat ttcactcgga | 540 |
| gcacctatcg ttattcaagc aattacgcaa attacgacaa ctccatcatt agttgaagta | 600 |
| attgttacag ggcttggact atcactagct cttggcacga gtgcatccat tattattgaa | 660 |
| aaagttgctt aa | 672 |

B. anthracis BclA (CIP5725) - (Q83WA6)

| | |
|---|---|
| 54. SQ SEQUENCE 244 AA; 23452 MW; AC95F5F306ACD892 CRC64; | |
| MSNNNYSNGL NPDESLSASA FDPNLVGPTL PPIPPFTLPT GPTGPTGPTG PTGPTGPTGP | |
| TGPTGPTGPT GPTGDTGTTG PTGPTGPTGP TGPTGATGLT GPTGPTGPSG LGLPAGLYAF | |
| NSGGISLDLG INDPVPFNTV GSQFGTAISQ LDADTFVISE TGFYKITVIA NTATASVLGG | |
| LTIQVNGVPV PGTGSSLISL GAPIVIQAIT QITTTPSLVE VIVTGLGLSL ALGTSASIII | |
| EKVA | |
| 53. SQ Sequence 735 BP; 210 A; 173 C; 156 G; 196 T; 0 other; 1433959005 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactggacca | 180 |
| actgggccaa ctggaccaac tgggccaact gggccaactg gagacactgg tactactgga | 240 |
| ccaactgggc caactggacc aactggacca actgggccaa ctggtgctac cggactgact | 300 |
| ggaccgactg gaccgactgg gccatccgga ctaggacttc cagcaggact atatgcattt | 360 |
| aactccggtg ggatttcttt agatttagga attaatgatc cagtaccatt taatactgtt | 420 |
| ggatctcagt ttggtacagc aatttctcaa ttagatgctg atactttcgt aattagtgaa | 480 |
| actggattct ataaaattac tgttatcgct aatactgcaa cagcaagtgt attaggaggt | 540 |
| cttacaatcc aagtgaatgg agtacctgta ccaggtactg gatcaagttt gatttcactc | 600 |
| ggagcaccta tcgttattca agcaattacg caaattacga caactccatc attagttgaa | 660 |
| gtaattgtta cagggcttgg actatcacta gctcttggca cgagtgcatc cattattatt | 720 |
| gaaaaagttg cttaa | 735 |

B. anthracis BclA (ATCC6602) - (Q83WA7)

| | |
|---|---|
| 56. SQ SEQUENCE 253 AA; 24208 MW; 01293B56EDB92731 CRC64; | |
| MSNNNYSNGL NPDESLSASA FDPNLVGPTL PPIPPFTLPT GPTGPTGPTG PTGPTGPTGP | |
| TGPTGPTGPT GPTGDTGTTG PTGPTGPTGP TGPTGPTGPT GPTGATGLTG PTGPTGPSGL | |
| GLPAGLYAFN SGGISLDLGI NDPVPFNTVG SQFGTAISQL DADTFVISET GFYKITVIAN | |
| TATASVLGGL TIQVNGVPVP GTGSSLISLG APIVIQAITQ ITTTSSLVEV IVTGLGLSLA | |
| LGTSASIIIE KVA | |
| 55. SQ Sequence 762 BP; 216 A; 182 C; 164 G; 200 T; 0 other; 645088734 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tggaccaact gggccaactg gagacactgg tactactgga | 240 |
| ccaactgggc caactggacc aactggacca actgggccaa ctggaccaac tggaccaact | 300 |
| gggccaactg gtgctaccgg actgactgga ccgactggac cgactgggcc atccggacta | 360 |
| ggacttccag caggactata tgcatttaac tccggtggga tttctttaga tttaggaatt | 420 |
| aatgatccag taccatttaa tactgttgga tctcagtttg gtacagcaat ttctcaatta | 480 |
| gatgctgata ctttcgtaat tagtgaaact ggattctata aaattactgt tatcgctaat | 540 |
| actgcaacag caagtgtatt aggaggtctt acaatccaag tgaatggagt acctgtacca | 600 |
| ggtactggat caagtttgat ttcactcgga gcacctatcg ttattcaagc aattacgcaa | 660 |
| attacgacaa cttcctcatt agttgaagta attgttacag ggcttggact atcactagct | 720 |
| cttggcacga gtgcatccat tattattgaa aaagttgctt aa | 762 |

B. anthracis BclA (CIP53169) - (Q83WA8)

| | |
|---|---|
| 58. SQ SEQUENCE 370 AA; 34262 MW; 064CEDCEF0EBB127 CRC64; | |
| MSNNNYSNGL NPDESLSASA FDPNLVGPTL PPIPPFTLPT GPTGPTGPTG PTGPTGPTGP | |
| TGPTGPTGPT GDTGTTGPTG PTGPTGPTGP TGPTGPTGPT GPTGPTGDTG TTGPTGPTGP | |
| TGPTGPTGDT GTTGPTGPTG PTGPTGPTGP TGPTGPTGPT GPTGPTGPTG PTGDTGTTGP | |
| TGPTGPTGPT GPTGDTGTTG PTGPTGPTGP TGPTGPTGPT GATGLTGPTG PTGPSGLGLP | |
| AGLYAFNSGG ISLDLGINDP VPFNTVGSQF GTAISQLDAD TFVISETGFY KITVIANTAT | |
| ASVLGGLTIQ VNGVPVPGTG SSLISLGAPI VIQAITQITT TPSLVEVIVT GLGLSLALGT | |
| SASIIIEKVA | |
| 57. SQ Sequence 1113 BP; 307 A; 291 C; 269 G; 246 T; 0 other; 2173493146 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccaact ggagacactg gtactactgg accaactggg | 240 |
| ccaactggac caactgggcc gactgggcca actggaccaa ctgggccgac tgggccaact | 300 |
| ggaccaactg ggccaactgg agacactggt actactggac caactgggcc aactggacca | 360 |
| actggaccaa ctgggccaac tggagacact ggtactactg gaccaactgg gccaactgga | 420 |
| ccaactggac caactgggcc gactggaccg actgggccga ctgggccaac tggaccaact | 480 |
| gggccgactg ggccaactgg accaactggg ccaactggag acactggtac tactggacca | 540 |
| actgggccaa ctggaccaac tggaccaact gggccaactg gagacactgg tactactgga | 600 |
| ccaactgggc caactggacc aactggacca actgggccaa ctggaccaac tgggccaact | 660 |
| ggtgctaccg gactgactgg accgactgga ccgactgggc catccggact aggacttcca | 720 |
| gcaggactat atgcatttaa ctccggtggg atttctttag atttaggaat taatgatcca | 780 |
| gtaccattta atactgttgg atctcagttt ggtacagcaa tttctcaatt agatgctgat | 840 |
| actttcgtaa ttagtgaaac tggattctat aaaattactg ttatcgctaa tactgcaaca | 900 |
| gcaagtgtat taggaggtct tacaatccaa gtgaatggag tacctgtacc aggtactgga | 960 |
| tcaagtttga tttcactcgg agcacctatc gttattcaag caattacgca aattacgaca | 1020 |
| actccatcat tagttgaagt aattgttaca gggcttggac tatcactagc tcttggcacg | 1080 |
| agtgcatcca ttattattga aaaagttgct taa | 1113 |

B. anthracis BclA (CIP8189) - (Q83WA9)

| | |
|---|---|
| 60. SQ SEQUENCE 391 AA; 36071 MW; E8B7B61480FD9DB9 CRC64; | |
| MSNNNYSNGL NPDESLSASA FDPNLVGPTL PPIPPFTLPT GPTGPTGPTG PTGPTGPTGP | |
| TGPTGPTGPT GDTGTTGPTG PTGPTGPTGP TGDTGTTGPT GPTGPTGPTG PTGPTGPTGP | |
| TGPTGPTGDT GTTGPTGPTG PTGPTGPTGD TGTTGPTGPT GPTGPTGPTG PTGPTGPTGP | |
| TGPTGPTGPT GPTGDTGTTG PTGPTGPTGP TGPTGDTGTT GPTGPTGPTG PTGPTGPTGP | |
| TGATGLTGPT GPTGPSGLGL PAGLYAFNSG GISLDLGIND PVPFNTVGSQ FGTAISQLDA | |
| DTFVISETGF YKITVIANTA TASVLGGLTI QVNGVPVPGT GSSLISLGAP IVIQAITQIT | |
| TTPSLVEVIV TGLGLSLALG TSASIIIEKV A | |
| 59. SQ Sequence 1176 BP; 323 A; 310 C; 288 G; 255 T; 0 other; 1987561614 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccaact ggagacactg gtactactgg accaactggg | 240 |
| ccgactgggc caactggacc aactgggcca actggagaca ctggtactac tggaccaact | 300 |
| gggccaactg gaccaactgg gccgactggg ccaactggac caactgggcc gactgggcca | 360 |
| actggaccaa ctgggccaac tggagacact ggtactactg gaccaactgg gccaactgga | 420 |
| ccaactggac caactgggcc aactggagac actggtacta ctggaccaac tgggccaact | 480 |
| ggaccaactg gaccaactgg gccgactgga ccgactgggc cgactgggcc aactggacca | 540 |
| actgggccga ctgggccaac tggaccaact gggccaactg gagacactgg tactactgga | 600 |
| ccaactgggc caactggacc aactggacca actgggccaa ctggagacac tggtactact | 660 |
| ggaccaactg ggccaactgg accaactgga ccaactgggc caactggacc aactgggcca | 720 |
| actggtgcta ccggactgac tggaccgact ggaccgactg ggccatccgg actaggactt | 780 |
| ccagcaggac tatatgcatt taactccggt gggatttctt tagatttagg aattaatgat | 840 |
| ccagtaccat ttaatactgt tggatctcag tttggtacag caatttctca attagatgct | 900 |
| gatactttcg taattagtga aactggattc tataaaatta ctgttatcgc taatactgca | 960 |
| acagcaagtg tattaggagg tcttacaatc caagtgaatg gagtacctgt accaggtact | 1020 |
| ggatcaagtt tgatttcact cggagcacct atcgttattc aagcaattac gcaaattacg | 1080 |
| acaactccat cattagttga agtaattgtt acagggcttg gactatcact agctcttggc | 1140 |
| acgagtgcat ccattattat tgaaaaagtt gcttaa | 1176 |

B. anthracis BclA (Sterne CIP7702) - (Q83WB0)

| | |
|---|---|
| 62. SQ SEQUENCE 445 AA; 40709 MW; DAF461B2B6FFA247 CRC64; | |
| MSNNNYSNGL NPDESLSASA FDPNLVGPTL PPIPPFTLPT GPTGPTGPTG PTGPTGPTGP | |
| TGPTGPTGPT GPTGPTGPTG DTGTTGPTGP TGPTGPTGPT GDTGTTGPTG PTGPTGPTGP | |
| TGPTGPTGPT GPTGPTGDTG TTGPTGPTGP TGPTGPTGDT GTTGPTGPTG PTGPTGPTGP | |
| TGPTGPTGPT GPTGPTGPTG PTGDTGTTGP TGPTGPTGPT GPTGDTGTTG PTGPTGPTGP | |
| TGPTGDTGTT GPTGPTGPTG PTGPTGDTGT TGPTGPTGPT GPTGPTGPTG PTGPTGATGL | |
| TGPTGPTGPS GLGLPAGLYA FNSGGISLDL GINDPVPFNT VGSQFGTAIS QLDADTFVIS | |
| ETGFYKITVI ANTATASVLG GLTIQVNGVP VPGTGSSLIS LGAPIVIQAI TQITTTPSLV | |
| EVIVTGLGLS LALGTSASII IEKVA | |
| 61. SQ Sequence 1338 BP; 368 A; 360 C; 333 G; 277 T; 0 other; 688694428 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccgact gggccaactg gaccaactgg gccaactgga | 240 |
| gacactggta ctactggacc aactgggccg actgggccaa ctggaccaac tgggccaact | 300 |
| ggagacactg gtactactgg accaactggg ccaactggac caactgggcc gactgggcca | 360 |
| actggaccaa ctgggccgac tgggccaact ggaccaactg ggccaactgg agacactggt | 420 |
| actactggac caactgggcc aactggacca actggaccaa ctgggccaac tggagacact | 480 |
| ggtactactg gaccaactgg gccaactgga ccaactggac caactgggcc gactggaccg | 540 |
| actgggccga ctgggccaac tggaccaact gggccgactg ggccaactgg accaactggg | 600 |
| ccaactggag acactggtac tactggacca actgggccaa ctggaccaac tggaccaact | 660 |
| gggccaactg gagacactgg tactactgga ccaactgggc caactggacc aactggacca | 720 |
| actgggccaa ctggagacac tggtactact ggaccaactg ggccaactgg accaactgga | 780 |
| ccaactgggc caactggaga cactggtact actggaccaa ctgggccaac tggaccaact | 840 |
| ggaccaactg ggccaactgg accaactgga ccaactgggc caactggtgc taccggactg | 900 |
| actggaccga ctggaccgac tgggccatcc ggactaggac ttccagcagg actatatgca | 960 |
| tttaactccg gcgggatttc tttagattta ggaattaatg atccagtacc atttaatact | 1020 |
| gttggatctc agtttggtac agcaatttct caattagatg ctgatacttt cgtaattagt | 1080 |
| gaaactggat tctataaaat tactgttatc gctaatactg caacagcaag tgtattagga | 1140 |
| ggtcttacaa tccaagtgaa tggagtacct gtaccaggta ctggatcaag tttgatttca | 1200 |
| ctcggagcac ctatcgttat tcaagcaatt acgcaaatta cgacaactcc atcattagtt | 1260 |
| gaagtaattg ttacagggct tggactatca ctagctcttg gcacgagtgc atccattatt | 1320 |
| attgaaaaag ttgcttaa | 1338 |

B. anthracis BclA (Ames) - (Q81JD7, Q6KVS0, Q7BYA5)

| | |
|---|---|
| 64. SQ SEQUENCE 382 AA; 35305 MW; 1DB4ED430DA07037 CRC64; | |
| MSNNNYSNGL NPDESLSASA FDPNLVGPTL PPIPPFTLPT GPTGPTGPTG PTGPTGPTGP | |
| TGPTGPTGPT GDTGTTGPTG PTGPTGPTGP TGDTGTTGPT GPTGPTGPTG PTGPTGPTGD | |
| TGTTGPTGPT GPTGPTGPTG DTGTTGPTGP TGPTGPTGPT GPTGPTGPTG PTGPTGPTGP | |
| TGPTGDTGTT GPTGPTGPTG PTGPTGDTGT TGPTGPTGPT GPTGPTGPTG PTGATGLTGP | |
| TGPTGPSGLG LPAGLYAFNS GGISLDLGIN DPVPFNTVGS QFGTAISQLD ADTFVISETG | |
| FYKITVIANT ATASVLGGLT IQVNGVPVPG TGSSLISLGA PIVIQAITQI TTTPSLVEVI | |
| VTGLGLSLAL GTSASIIIEK VA | |
| 63. SQ Sequence 1149 BP; 317 A; 301 C; 279 G; 252 T; 0 other; 3918642356 CRC32; | |
| atgtcaaata ataattattc aaatggatta aaccccgatg aatctttatc agctagtgca | 60 |
| tttgacccta atcttgtagg acctacatta ccaccgatac caccatttac ccttcctacc | 120 |
| ggaccaactg ggccgactgg accgactggg ccgactgggc caactggacc aactgggccg | 180 |
| actgggccaa ctggaccaac tgggccaact ggagacactg gtactactgg accaactggg | 240 |
| ccgactgggc caactggacc aactgggcca actggagaca ctggtactac tggaccaact | 300 |
| gggccaactg gaccaactgg gccgactggg ccaactggac caactgggcc aactggagac | 360 |
| actggtacta ctggaccaac tgggccaact ggaccaactg gaccaactgg gccaactgga | 420 |
| gacactggta ctactggacc aactgggcca actggaccaa ctggaccaac tgggccgact | 480 |
| ggaccgactg ggccgactgg gccaactgga ccaactgggc cgactgggcc aactggacca | 540 |
| actgggccaa ctggagacac tggtactact ggaccaactg ggccaactgg accaactgga | 600 |
| ccaactgggc caactggaga cactggtact actggaccaa ctgggccaac tggaccaact | 660 |
| ggaccaactg ggccaactgg accaactggg ccaactggtg ctaccggact gactggaccg | 720 |
| actggaccga ctgggccatc cggactagga cttccagcag gactatatgc atttaactcc | 780 |
| ggtgggattt ctttagattt aggaattaat gatccagtac catttaatac tgttggatct | 840 |
| cagtttggta cagcaatttc tcaattagat gctgatactt tcgtaattag tgaaactgga | 900 |
| ttctataaaa ttactgttat cgctaatact gcaacagcaa gtgtattagg aggtcttaca | 960 |
| atccaagtga atggagtacc tgtaccaggt actggatcaa gtttgatttc actcggagca | 1020 |
| cctatcgtta ttcaagcaat tacgcaaatt acgacaactc catcattagt tgaagtaatt | 1080 |
| gttacagggc ttggactatc actagctctt ggcacgagtg catccattat tattgaaaaa | 1140 |
| gttgcttaa | 1149 |

B. anthracis EA1 - (P94217, Q6I2R2, Q6KWJ3)

| | |
|---|---|
| 70. SQ SEQUENCE 862 AA; 91362 MW; CB16B202F62CCCA0 CRC64; | |
| MAKTNSYKKV IAGTMTAAMV AGIVSPVAAA GKSFPDVPAG HWAEGSINYL VDKGAITGKP | |
| DGTYGPTESI DRASAAVIFT KILNLPVDEN AQPSFKDAKN IWSSKYIAAV EKAGVVKGDG | |
| KENFYPEGKI DRASFASMLV SAYNLKDKVN GELVTTFEDL LDHWGEEKAN ILINLGISVG | |
| TGGKWEPNKS VSRAEAAQFI ALTDKKYGKK DNAQAYVTDV KVSEPTKLTL TGTGLDKLSA | |
| DDVTLEGDKA VAIEASTDGT SAVVTLGGKV APNKDLTVKV KNQSFVTKFV YEVKKLAVEK | |
| LTFDDDRAGQ AIAFKLNDEK GNADVEYLNL ANHDVKFVAN NLDGSPANIF EGGEATSTTG | |
| KLAVGIKQGD YKVEVQVTKR GGLTVSNTGI ITVKNLDTPA SAIKNVVFAL DADNDGVVNY | |
| GSKLSGKDFA LNSQNLVVGE KASLNKLVAT IAGEDKVVDP GSISIKSSNH GIISVVNNYI | |
| TAEAAGEATL TIKVGDVTKD VKFKVTTDSR KLVSVKANPD KLQVVQNKTL PVTFVTTDQY | |
| GDPFGANTAA IKEVLPKTGV VAEGGLDVVT TDSGSIGTKT IGVTGNDVGE GTVHFQNGNG | |
| ATLGSLYVNV TEGNVAFKNF ELVSKVGQYG QSPDTKLDLN VSTTVEYQLS KYTSDRVYSD | |
| PENLEGYEVE SKNLAVADAK IVGNKVVVTG KTPGKVDIHL TKNGATAGKA TVEIVQETIA | |
| IKSVNFKPVQ TENFVEKKIN IGTVLELEKS NLDDIVKGIN LTKETQHKVR VVKSGAEQGK | |
| LYLDRNGDAV FNAGDVKLGD VTVSQTSDSA LPNFKADLYD TLTTKYTDKG TLVFKVLKDK | |
| DVITSEIGSQ AVHVNVLNNP NL | |
| 69. SQ Sequence 2589 BP; 926 A; 421 C; 515 G; 727 T; 0 other; 2474321808 CRC32; | |
| atggcaaaga ctaactctta caaaaaagta atcgcaggta caatgacagc agcaatggta | 60 |
| gcaggtattg tatctccagt agcagcagca ggtaaatcat tcccagacgt tccagctgga | 120 |
| cattgggcag aaggttctat taattactta gtagataaag gtgcaattac aggtaagcca | 180 |
| gacggtacat atggtccaac cgaatcaatc gatcgtgctt ctgcagctgt aatcttcact | 240 |
| aaaattttaa atttaccagt tgatgaaaat gctcagcctt ctttcaaaga tgctaaaaat | 300 |
| atttggtctt caaaatatat tgcagcagtt gaaaaagctg gcgttgttaa aggtgatggc | 360 |
| aaagaaaact tctatccaga aggaaagatt gaccgtgctt catttgcttc tatgttagta | 420 |
| agtgcttata acttaaaaga taaagttaac ggcgagttag ttacgacatt tgaagattta | 480 |
| ttagatcatt ggggtgaaga gaaagcaaac atcctaatta accttggaat ctctgtaggt | 540 |
| actggtggta aatgggagcc aaataaatct gtatctcgtg cagaagcagc tcaatttatc | 600 |
| gcattaacag ataaaaaata tggaaaaaaa gataatgcac aagcgtatgt aactgatgtg | 660 |
| aaagtttctg agccaacgaa attaacatta acaggtactg gcttagacaa actttctgct | 720 |
| gatgatgtaa ctcttgaagg agacaaagca gttgcaatcg aagcaagtac tgatggtact | 780 |
| tctgcagttg taacacttgg tggcaaagta gctccaaata aagaccttac tgtaaaagtg | 840 |
| aaaaatcaat cattcgtaac gaaattcgta tacgaagtga aaaaattagc agtagaaaaa | 900 |
| cttacatttg atgatgatcg cgctggtcaa gcaattgctt tcaaattaaa cgatgaaaaa | 960 |
| ggtaacgctg atgttgagta cttaaactta gcaaaccatg acgtcaaatt tgtagcgaat | 1020 |
| aacttagacg gttcaccagc aaacatcttt gaaggtggag aagctacttc tactacaggt | 1080 |
| aaactagctg ttggcattaa gcagggtgac tacaaagtag aagtacaagt tacaaaacgc | 1140 |
| ggtggtttaa cagtttctaa cactggtatt attacagtga aaaaccttga tacaccagct | 1200 |
| tctgcaatta aaaatgttgt atttgcatta gatgctgata atgatggtgt tgtaaactat | 1260 |
| ggcagcaagc tttctggtaa agactttgct ttaaatagcc aaaacttagt tgttggtgaa | 1320 |
| aaagcatctc ttaataaatt agttgctaca attgctggag aagataaagt agttgatcca | 1380 |
| ggatcaatta gcattaaatc ttcaaaccac ggtattattt ctgtagtaaa taactacatt | 1440 |
| actgctgagg ctgctggtga agctacactt actattaaag taggtgacgt tacaaaagac | 1500 |
| gttaaattta aagtaacgac tgattctcgt aaattagtat cagtaaaagc taacccagat | 1560 |
| aaattacaag ttgttcaaaa taaaacatta cctgttacat tcgtaacaac tgaccaatat | 1620 |
| ggcgatccat ttggtgctaa cacagctgca attaaagaag ttcttccgaa aacaggtgta | 1680 |
| gttgcagaag gtggattaga tgtagtaacg actgactctg gttcaatcgg tacaaaaaca | 1740 |
| attggtgtta caggtaatga cgtaggcgaa ggtacagttc acttccaaaa cggtaatggt | 1800 |
| gctactttag gttcattata tgtgaacgta acagagggta acgttgcatt taaaaacttt | 1860 |
| gaacttgtat ctaaagtagg tcaatatggc caatcacctg atacaaaact tgacttaaat | 1920 |
| gtttcaacta ctgttgaata tcaattatct aagtacactt cagatcgcgt atactctgat | 1980 |
| cctgaaaact tagaaggtta tgaagttgaa tctaaaaatc tagctgtagc tgacgctaaa | 2040 |
| attgttggaa ataaagttgt tgttacaggt aaaactccag gtaaagttga tatccactta | 2100 |
| acgaaaaatg gtgcaactgc tggtaaagcg acagtcgaaa tcgttcaaga gacaattgct | 2160 |
| attaaatctg taaacttcaa accagttcaa acagaaaact ttgttgagaa gaaaatcaac | 2220 |
| atcggtactg tattagagct tgagaagagt aacctggatg atatcgtaaa aggtattaac | 2280 |
| ttaacgaaag aaacacaaca taaagtacgt gttgtgaaat ctggtgcaga gcaaggtaaa | 2340 |
| ctttacttag atagaaacgg tgatgctgta tttaacgctg gcgatgtaaa acttggcgat | 2400 |
| gtaacagtat ctcaaacaag tgattctgca cttccaaact tcaaggcaga tctttatgat | 2460 |
| actttaacta ctaagtacac tgacaaaggt acattagtat tcaaagtatt aaaagataaa | 2520 |
| gatgttatta caagcgaaat cggttcacaa gctgtacacg tgaacgttct taataaccca | 2580 |
| aatctataa | 2589 |

B. anthracis EA2 - (P49051, Q6I2R3, Q6KWJ4)

| | |
|---|---|
| 72. SQ SEQUENCE 814 AA; 86621 MW; C1638D26A1C6B101 CRC64; | |
| MAKTNSYKKV IAGTMTAAMV AGVVSPVAAA GKTFPDVPAD HWGIDSINYL VEKGAVKGND | |
| KGMFEPGKEL TRAEAATMMA QILNLPIDKD AKPSFADSQG QWYTPFIAAV EKAGVIKGTG | |
| NGFEPNGKID RVSMASLLVE AYKLDTKVNG TPATKFKDLE TLNWGKEKAN ILVELGISVG | |
| TGDQWEPKKT VTKAEAAQFI AKTDKQFGTE AAKVESAKAV TTQKVEVKFS KAVEKLTKED | |
| IKVTNKANND KVLVKEVTLS EDKKSATVEL YSNLAAKQTY TVDVNKVGKT EVAVGSLEAK | |
| TIEMADQTVV ADEPTALQFT VKDENGTEVV SPEGIEFVTP AAEKINAKGE ITLAKGTSTT | |
| VKAVYKKDGK VVAESKEVKV SAEGAAVASI SNWTVAEQNK ADFTSKDFKQ NNKVYEGDNA | |
| YVQVELKDQF NAVTTGKVEY ESLNTEVAVV DKATGKVTVL SAGKAPVKVT VKDSKGKELV | |
| SKTVEIEAFA QKAMKEIKLE KTNVALSTKD VTDLKVKAPV LDQYGKEFTA PVTVKVLDKD | |
| GKELKEQKLE AKYVNKELVL NAAGQEAGNY TVVLTAKSGE KEAKATLALE LKAPGAFSKF | |
| EVRGLEKELD KYVTEENQKN AMTVSVLPVD ANGLVLKGAE AAELKVTTTN KEGKEVDATD | |
| AQVTVQNNSV ITVGQGAKAG ETYKVTVVLD GKLITTHSFK VVDTAPTAKG LAVEFTSTSL | |
| KEVAPNADLK AALLNILSVD GVPATTAKAT VSNVEFVSAD TNVVAENGTV GAKGATSIYV | |
| KNLTVVKDGK EQKVEFDKAV QVAVSIKEAK PATK | |
| 71. SQ Sequence 2445 BP; 974 A; 381 C; 479 G; 611 T; 0 other; 1260040913 CRC32; | |
| atggcaaaga ctaactctta caaaaaagta atcgctggta caatgacagc agcaatggta | 60 |
| gcaggtgttg tttctccagt agcagcagca ggtaaaacat tcccagacgt tcctgctgat | 120 |
| cactggggaa ttgattctat taactactta gtagaaaaag gcgcagttaa aggtaacgac | 180 |
| aaaggaatgt tcgagcctgg aaaagaatta actcgtgcag aagcagctac aatgatggct | 240 |
| caaatcttaa acttaccaat cgataaagat gctaaaccat ctttcgctga ctctcaaggc | 300 |
| caatggtaca ctccattcat cgcagctgta gaaaaagctg gcgttattaa aggtacagga | 360 |
| aacggctttg agccaaacgg aaaaatcgac cgcgtttcta tggcatctct tcttgtagaa | 420 |
| gcttacaaat tagatactaa agtaaacggt actccagcaa ctaaattcaa agatttagaa | 480 |
| acattaaact ggggtaaaga aaaagctaac atcttagttg aattaggaat ctctgttggt | 540 |
| actggtgatc aatgggagcc taagaaaact gtaactaaag cagaagctgc tcaattcatt | 600 |
| gctaagactg acaagcagtt cggtacagaa gcagcaaaag ttgaatctgc aaaagctgtt | 660 |
| acaactcaaa aagtagaagt taaattcagc aaagctgttg aaaaattaac taaagaagat | 720 |
| atcaaagtaa ctaacaaagc taacaacgat aaagtactag ttaaagaggt aactttatca | 780 |
| gaagataaaa aatctgctac agttgaatta tatagtaact tagcagctaa acaaacttac | 840 |
| actgtagatg taaacaaagt tggtaaaaca gaagtagctg taggttcttt agaagcaaaa | 900 |
| acaatcgaaa tggctgacca aacagttgta gctgatgagc caacagcatt acaattcaca | 960 |
| gttaaagatg aaaacggtac tgaagttgtt tcaccagagg gtattgaatt tgtaacgcca | 1020 |
| gctgcagaaa aaattaatgc aaaaggtgaa atcactttag caaaaggtac ttcaactact | 1080 |
| gtaaaagctg tttataaaaa agacggtaaa gtagtagctg aaagtaaaga agtaaaagtt | 1140 |
| tctgctgaag gtgctgcagt agcttcaatc tctaactgga cagttgcaga acaaaataaa | 1200 |
| gctgacttta cttctaaaga tttcaaacaa aacaataaag tttacgaagg cgacaacgct | 1260 |
| tacgttcaag tagaattgaa agatcaattt aacgcagtaa caactggaaa agttgaatat | 1320 |
| gagtcgttaa acacagaagt tgctgtagta gataaagcta ctggtaaagt aactgtatta | 1380 |
| tctgcaggaa aagcaccagt aaaagtaact gtaaaagatt caaaaggtaa agaacttgtt | 1440 |
| tcaaaaacag ttgaaattga agctttcgct caaaaagcaa tgaaagaaat taaattagaa | 1500 |
| aaaactaacg tagcgctttc tacaaaagat gtaacagatt taaaagtaaa agctccagta | 1560 |
| ctagatcaat acggtaaaga gtttacagct cctgtaacag tgaaagtact tgataaagat | 1620 |
| ggtaaagaat taaaagaaca aaaattagaa gctaaatatg tgaacaaaga attagttctg | 1680 |
| aatgcagcag gtcaagaagc tggtaattat acagttgtat taactgcaaa atctggtgaa | 1740 |
| aaagaagcaa aagctacatt agctctagaa ttaaaagctc caggtgcatt ctctaaattt | 1800 |
| gaagttcgtg gtttagaaaa agaattagat aaatatgtta ctgaggaaaa ccaaaagaat | 1860 |
| gcaatgactg tttcagttct tcctgtagat gcaaatggat tagtattaaa aggtgcagaa | 1920 |
| gcagctgaac taaaagtaac aacaacaaac aaagaaggta aagaagtaga cgcaactgat | 1980 |
| gcacaagtta ctgtacaaaa taacagtgta attactgttg gtcaaggtgc aaaagctggt | 2040 |
| gaaacttata aagtaacagt tgtactagat ggtaaattaa tcacaactca ttcattcaaa | 2100 |
| gttgttgata cagcaccaac tgctaaagga ttagcagtag aatttacaag cacatctctt | 2160 |
| aaagaagtag ctccaaatgc tgatttaaaa gctgcacttt taaatatctt atctgttgat | 2220 |
| ggtgtacctg cgactacagc aaaagcaaca gtttctaatg tagaatttgt ttctgctgac | 2280 |
| acaaatgttg tagctgaaaa tggtacagtt ggtgcaaaag gtgcaacatc tatctatgtg | 2340 |
| aaaaacctga cagttgtaaa agatggaaaa gagcaaaaag tagaatttga taaagctgta | 2400 |
| caagttgcag tttctattaa agaagcaaaa cctgcaacaa aataa | 2445 |

B. anthracis SSPH1 - (Q81V87, Q6I3H4, Q6KX87)

| | |
|---|---|
| 74. SQ SEQUENCE 59 AA; 6545 MW; 314122FF7D3D7C55 CRC64; | |
| MDVKRVKQIL SSSSRIDVTY EGVPVWIESC DEQSGVAQVY DVSNPGESVH VHVNALEEK | |
| 73. SQ Sequence 180 BP; 55 A; 26 C; 50 G; 49 T; 0 other; 1292079022 CRC32; | |
| atggatgtaa aacgtgtgaa acaaatttta tcttcttcaa gtagaatcga cgttacatat | 60 |
| gaaggcgtac cggtatggat tgagagctgt gacgagcaga gtggggttgc tcaagtgtat | 120 |
| gatgtatcta atcctggaga aagcgttcac gttcacgtga acgctttaga ggagaagtaa | 180 |

B. anthracis SSPH2 - (Q81SD1, Q6KUH6)

| | |
|---|---|
| 76. SQ SEQUENCE 59 AA; 6628 MW; 562A5659E736BF4E CRC64; | |
| MNIQRAKELS VSAEQANVSF QGMPVMIQHV DESNETARIY EVKNPGRELT VPVNSLEEI | |
| 75. SQ Sequence 180 BP; 65 A; 34 C; 39 G; 42 T; 0 other; 2333600548 CRC32; | |
| atgaatattc aacgtgcaaa agagctttct gtgtcagcgg agcaagcgaa tgttagtttt | 60 |
| caaggcatgc ctgttatgat tcaacacgtc gacgaaagca atgaaaccgc ccgcatatat | 120 |
| gaagtaaaaa acccaggacg cgaattaaca gttccagtta atagcttaga ggaaatataa | 180 |

B. anthracis SSPI - (Q81L28, Q6HSI3, Q6KLS8)

| | |
|---|---|
| 78. SQ SEQUENCE 69 AA; 7687 MW; 3F5D0398D7D57A8C CRC64; | |
| MSFNLRGAVL ANVSGNTQDQ LQETIVDAIQ SGEEKMLPGL GVLFEVIWKN ADENEKHEML | |
| ETLEQGLKK | |
| 77. SQ Sequence 210 BP; 85 A; 24 C; 42 G; 59 T; 0 other; 1796731092 CRC32; | |
| atgagtttta atttacgcgg tgctgtatta gcaaatgtat ctggtaatac acaagatcaa | 60 |
| ttacaagaaa caattgttga tgcaattcaa agcggcgaag aaaaaatgct tccaggtctt | 120 |
| ggtgttttat ttgaagtcat ttggaaaaat gctgatgaaa atgaaaaaca cgaaatgtta | 180 |
| gaaacattag agcaaggatt aaaaaaataa | 210 |

B. anthracis SSPK - (Q81YW1, Q6KXH4)

| | |
|---|---|
| 80. SQ SEQUENCE 52 AA; 5946 MW; F92BD3CD5A408831 CRC64; | |
| MGKQAEFWSE SKNNSKIDGQ PKAKSRFASK RPNGTINTHP QERMRAANQQ EE | |
| 79. SQ Sequence 159 BP; 59 A; 39 C; 36 G; 25 T; 0 other; 4133010666 CRC32; | |
| atgggtaaac aagccgaatt ttggtctgag tcaaaaaaca acagcaaaat cgacggtcaa | 60 |
| ccgaaagcga aatcacgctt cgcttcgaag cgacctaacg gcacaattaa cacgcaccca | 120 |
| caagaacgta tgcgtgctgc aaatcagcag gaagagtag | 159 |

B. anthracis SSPN - (Q81Y87, Q6KPQ0)

| | |
|---|---|
| 82. SQ SEQUENCE 44 AA; 4681 MW; 1FCF20594230E137 CRC64; | |
| MGNPKKNSKD FAPNHIGTQS KKAGGNKGKQ MQDQTGKQPI VDNG | |
| 81. SQ Sequence 135 BP; 59 A; 22 C; 29 G; 25 T; 0 other; 547647061 CRC32; | |
| atgggtaatc cgaaaaagaa ttcaaaagac tttgcaccga atcatattgg aacacaatca | 60 |
| aaaaaagctg gtggcaataa agggaagcaa atgcaagacc aaacgggtaa acaaccgatt | 120 |
| gttgataacg gttaa | 135 |

B. anthracis SSPO - (Q81Y79, Q6HVH3, Q6KPP3)

| | |
|---|---|
| 84. SQ SEQUENCE 49 AA; 5390 MW; 5AE1415CB5B9B969 CRC64; | |
| MGKRKANHTI SGMNAASAQG QGAGYNEEFA NENLTPAERQ NNKKRKKNQ | |
| 83. SQ Sequence 150 BP; 67 A; 24 C; 31 G; 28 T; 0 other; 1440840437 CRC32; | |
| atgggtaaaa gaaaagcaaa tcatactatt tcaggaatga atgcggcatc tgcacaagga | 60 |
| caaggtgctg gttataacga agagtttgca aatgaaaact taactcctgc agaacgacaa | 120 |
| aataataaga aacgcaaaaa gaaccagtaa | 150 |

B. anthracis TLP - (Q81Y88, Q6HVH9, Q6KPQ1)

| | |
|---|---|
| 86. SQ SEQUENCE 65 AA; 7466 MW; 374CA2594D11E319 CRC64; | |
| MPNPDNRSDN AEKLQEMVQN TIDNFNEAKE TAELSNEKDR SAIEAKNQRR LESIDSLKSE | |
| IKDES | |
| 85. SQ Sequence 198 BP; 90 A; 27 C; 35 G; 46 T; 0 other; 39596844 CRC32; | |
| atgccaaatc cagataatcg aagtgataac gctgaaaagt tacaagaaat ggtgcaaaat | 60 |
| acaattgata actttaatga agcaaaagaa acagcggagc tttctaatga aaaagaccgt | 120 |
| tctgctattg aagcaaaaaa tcaaagacgt ttagaaagta ttgactcatt aaaaagtgaa | 180 |
| atcaaagatg aatcttaa | 198 |

B. anthracis SSPB - (Q81KU1, Q6HS97, Q6KLJ4)

| | |
|---|---|
| 88. SQ SEQUENCE 65 AA; 6810 MW; 79E631D24389825C CRC64; | |
| MARSTNKLAV PGAESALDQM KYEIAQEFGV QLGADATARA NGSVGGEITK RLVSLAEQQL | |
| GGFQK | |
| 87. SQ Sequence 198 BP; 62 A; 40 C; 46 G; 50 T; 0 other; 1091854369 CRC32; | |
| atggcacgta gcacaaataa attagcggtt cctggtgctg aatcagcatt agaccaaatg | 60 |
| aaatacgaaa tcgctcaaga gtttggtgtt caacttggag ctgatgcaac agctcgcgct | 120 |
| aacggttctg ttggtggcga aatcactaaa cgtctagttt cactagctga gcaacaatta | 180 |
| ggcggtttcc aaaaataa | 198 |

B. anthracis SSPalpha/beta-1 - (Q6HZY0)

| | |
|---|---|
| 90. SQ SEQUENCE 70 AA; 7442 MW; CD58D47B19F50683 CRC64; | |
| MVMARNRNSN QLASHGAQAA LDQMKYEIAQ EFGVQLGADT SSRANGSVGG EITKRLVAMA | |
| EQQLGGGYTR | |
| 89. SQ Sequence 213 BP; 68 A; 39 C; 50 G; 56 T; 0 other; 2897992167 CRC32; | |
| ttggtaatgg ctagaaatcg taattctaat caattagcat cacatggagc acaagcggct | 60 |
| ttagatcaaa tgaaatatga aattgcacaa gagtttggtg tacaacttgg cgctgatact | 120 |
| tcttcacgtg caaacggttc tgtaggcggt gaaattacaa aacgcctagt agcgatggca | 180 |
| gaacaacaac ttggtggcgg ttatactcgc taa | 213 |

B. anthracis SSPalpha/beta-2 - (Q81NQ2, Q6HWX2, Q6XR04)

| | |
|---|---|
| 92. SQ SEQUENCE 70 AA; 7294 MW; 5AE19EBFE3CAFA8F CRC64; | |
| MSNNNSGSSN QLLVRGAEQA LDQMKYEIAQ EFGVQLGADA TARANGSVGG EITKRLVSLA | |
| EQQLGGGVTR | |
| 91. SQ Sequence 213 BP; 68 A; 38 C; 51 G; 56 T; 0 other; 2311515668 CRC32; | |
| atgtcaaaca ataacagtgg aagcagcaat caattattag tacgtggcgc tgaacaagct | 60 |
| cttgatcaaa tgaaatatga aattgctcaa gaatttggcg tacaacttgg tgcagatgca | 120 |
| acagctcgtg caaacggatc tgttggtggt gaaattacga aacgtcttgt atcattagct | 180 |
| gagcaacaac ttggcggtgg cgttactcgt taa | 213 |

B. anthracis SSPalpha/beta-3 - (Q81RQ3, Q6KTV9)

| | |
|---|---|
| 94. SQ SEQUENCE 68 AA; 7212 MW; 3EB0ED7B6B413001 CRC64; | |
| MARNRNSNQL ASHGAQAALD QMKYEIAQEF GVQLGADTSS RANGSVGGEI TKRLVAMAEQ | |
| QLGGGYTR | |
| 93. SQ Sequence 207 BP; 67 A; 39 C; 48 G; 53 T; 0 other; 2919363707 CRC32; | |
| atggctagaa atcgtaattc taatcaatta gcatcacatg gagcacaagc ggctttagat | 60 |
| caaatgaaat atgaaattgc acaagagttt ggtgtacaac ttggcgctga tacttcttca | 120 |
| cgtgcaaacg gttctgtagg cggtgaaatt acaaaacgcc tagtagcgat ggcagaacaa | 180 |
| caacttggtg gcggttatac tcgctaa | 207 |

B. anthracis SSPalpha/beta-4 - (Q81TF3, Q6I1N6, Q6KVH8)

| | |
|---|---|
| 96. SQ SEQUENCE 61 AA; 6506 MW; 0EE8D71944105E23 CRC64; | |
| MVKTNKLLVP GAEQALEQFK YEIAQEFGVS LGSNTASRSN GSVGGEVTKR LVALAQQQLR | |
| G | |
| 95. SQ Sequence 186 BP; 67 A; 34 C; 36 G; 49 T; 0 other; 1601000462 CRC32; | |
| atggtaaaaa caaacaaatt actagttcct ggtgctgaac aagcacttga acaatttaaa | 60 |
| tatgaaattg cacaagaatt cggcgtaagc ttaggatcta atacagcatc tcgttctaac | 120 |
| ggatcagttg gcggtgaagt aacaaaacgt cttgtcgctt tagctcaaca acaattacgt | 180 |
| ggataa | 186 |

B. anthracis SASP-2 - (Q81NP9, Q6HWW9, Q6KR01)

| | |
|---|---|
| 98. SQ SEQUENCE 70 AA; 7480 MW; 7CEFC287FE699BD2 CRC64; | |
| MANNNSGSRN ELLVRGAEQA LDQMKYEIAQ EFGVQLGADT TARSNGSVGG EITKRLVAMA | |
| EQQLGGRANR | |
| 97. SQ Sequence 213 BP; 74 A; 32 C; 51 G; 56 T; 0 other; 2532906473 CRC32; | |
| atggcaaaca acaatagtgg aagtcgtaat gaattattag ttcgaggtgc tgaacaagct | 60 |
| cttgatcaaa tgaaatatga aattgcacaa gagtttggtg tacaacttgg tgcagataca | 120 |
| acagctcgtt caaatggatc tgttggtggt gaaattacaa aacgtttagt agcaatggct | 180 |
| gaacaacaac ttggtggtag agctaaccgc taa | 213 |

B. anthracis SSPF - (Q81VZ7, Q6I500, Q6KYP4)

| | |
|---|---|
| 100. SQ SEQUENCE 59 AA; 6800 MW; 4ABE95C3C32776CF CRC64; | |
| MSRRRGVMSN QFKEELAKEL GFYDVVQKEG WGGIRAKDAG NMVKRAIEIA EQQLMKQNQ | |
| 99. SQ Sequence 180 BP; 67 A; 22 C; 49 G; 42 T; 0 other; 3510911733 CRC32; | |
| ttgagtagac gaagaggtgt catgtcaaat caatttaaag aagagcttgc aaaagagcta | 60 |
| ggcttttatg atgttgttca gaaagaagga tggggcggaa ttcgtgcgaa agatgctggt | 120 |
| aacatggtga aacgtgctat agaaattgca gaacagcaat taatgaaaca aaaccagtag | 180 |

B. anthracis SASP-1 - (Q81UL0, Q6I2T9, Q6KWL8)

| | |
|---|---|
| 102. SQ SEQUENCE 67 AA; 6966 MW; 758493D3DD9ECB85 CRC64; | |
| MANQNSSNQL VVPGATAAID QMKYEIAQEF GVQLGADSTA RANGSVGGEI TKRLVAMAEQ | |
| SLGGFHK | |
| 101. SQ Sequence 204 BP; 70 A; 42 C; 45 G; 47 T; 0 other; 735920664 CRC32; | |
| atggcaaacc aaaattcttc aaatcaatta gtagtaccag gagcaacagc tgcaatcgac | 60 |
| caaatgaagt acgaaatcgc tcaagaattt ggtgtacaat taggagcaga ttctacagct | 120 |
| cgtgctaacg gttctgttgg tggcgaaatc acaaaacgtc tagttgcaat ggctgagcaa | 180 |
| agccttggcg gattccacaa ataa | 204 |

B. anthracis SSPE - (Q81YV6, Q6I3Q7, Q6KXG9, Q84DX8)

| | |
|---|---|
| 104. SQ SEQUENCE 95 AA; 9869 MW; F7A807EF8B845C4B CRC64; | |
| MSKKQQGYNK ATSGASIQST NASYGTEFAT ETNVQAVKQA NAQSEAKKAQ ASGASIQSTN | |
| ASYGTEFATE TDVHAVKKQN AQSAAKQSQS SSSNQ | |
| 103. SQ Sequence 288 BP; 119 A; 55 C; 54 G; 60 T; 0 other; 875991772 CRC32; | |
| atgagtaaaa aacaacaagg ttataacaag gcaacttctg gtgctagcat tcaaagcaca | 60 |
| aatgctagtt atggtacaga gtttgcgact gaaacaaatg tacaagcagt aaaacaagca | 120 |
| aacgcacaat cagaagctaa gaaagcgcaa gcttctggtg ctagcattca aagcacaaat | 180 |
| gctagttatg gtacagaatt tgcaactgaa acagacgtgc atgctgtgaa aaaacaaaat | 240 |
| gcacaatcag ctgcaaaaca atcacaatct tctagttcaa atcagtaa | 288 |

B. anthracis ExsB - (Q81TC7)

| | |
|---|---|
| 106. SQ SEQUENCE 220 AA; 24541 MW; B6DFE2417ECE0E63 CRC64; | |
| MKKEKAVVVF SGGQDSTTCL FWAIEQFAEV EAVTFNYNQR HKLEIDCAVE IAKELGIKHT | |
| VLDMSLLNQL APNALTRTDM EITHEEGELP STFVDGRNLL FLSFAAVLAK QVGARHIVTG | |
| VCETDFSGYP DCRDVFVKSL NVTLNLSMDY PFVIHTPLMW IDKAETWKLS DELGAFEFVR | |
| EKTLTCYNGI IGDGCGECPA CQLRKAGLDT YLQEREGASN | |
| 105. SQ Sequence 663 BP; 222 A; 89 C; 156 G; 196 T; 0 other; 1478510222 CRC32; | |
| atgaaaaaag aaaaggcagt tgttgttttt agtggaggac aagatagtac gacatgttta | 60 |
| ttttgggcaa tagagcagtt tgcagaagta gaggctgtaa cgtttaatta caatcaacgt | 120 |
| cataagctag aaattgattg tgcagtggaa attgcaaaag agctaggaat taaacatacg | 180 |
| gtactagata tgagtctatt aaatcaactt gctccaaatg cgttaacgag aacggatatg | 240 |
| gagattacac atgaagaagg tgaattacca tcgacgtttg tagatggacg aaatttacta | 300 |
| ttcttatcat ttgctgctgt attagcaaaa caagttggag cacgtcatat tgtaacgggt | 360 |
| gtatgtgaaa ctgattttag tggttatcca gattgccgtg acgtgtttgt gaaatcgtta | 420 |
| aacgttactt taaatttatc gatggattat ccgtttgtga ttcatacacc acttatgtgg | 480 |
| attgataaag cggaaacatg gaaattatca gatgaacttg gagcattcga gtttgttaga | 540 |
| gagaaaacat taacatgtta taacggaatc attggtgatg gttgcggtga atgtccagca | 600 |
| tgtcaacttc gtaaagcagg attagatacg tatctacaag aacgcgaagg agcgagtaac | 660 |
| taa | 663 |

B. anthracis cspA - (Q81TW8, Q6I254, Q6KVZ0)

| | |
|---|---|
| 108. SQ SEQUENCE 67 AA; 7475 MW; 2852D8BDA939823F CRC64; | |
| MAVTGQVKWF NNEKGFGFIE VPGENDVFVH FSAIETDGFK SLEEGQKVSF EIEEGNRGPQ | |
| AKNVIKL | |
| 107. SQ Sequence 204 BP; 78 A; 38 C; 42 G; 46 T; 0 other; 814803456 CRC32; | |
| atggcagtaa caggacaagt aaaatggttt aacaacgaaa aaggcttcgg tttcatcgaa | 60 |
| gttccaggcg aaaacgacgt attcgtacat ttctctgcaa tcgaaactga cggtttcaaa | 120 |
| tctctagaag aaggtcaaaa agttagcttc gaaatcgaag aaggtaaccg tggacctcaa | 180 |
| gctaaaaacg taatcaaact ataa | 204 |

B. anthracis cspB-1 - (Q81SL9, Q6I0V2, Q6KUQ7)

| | |
|---|---|
| 110. SQ SEQUENCE 65 AA; 7196 MW; EFACACA4C1B04DB0 CRC64; | |
| MQGKVKWFNN EKGFGFIEME GADDVFVHFS AIQGEGYKAL EEGQEVSFDI TEGNRGPQAA | |
| NVVKL | |
| 109. SQ Sequence 198 BP; 71 A; 32 C; 46 G; 49 T; 0 other; 319593732 CRC32; | |
| atgcaaggaa aagtaaaatg gtttaacaac gaaaaaggtt ttggatttat cgaaatggaa | 60 |
| ggcgctgacg atgtattcgt acatttctct gcgattcaag gcgaaggcta caaagcttta | 120 |
| gaagaaggtc aagaagtatc tttcgatatc actgaaggaa accgcggacc tcaagctgct | 180 |
| aacgtagtaa aactttaa | 198 |

B. anthracis cspB-2 - (Q81YF5, Q6HVP8, Q6KPW5)

| | |
|---|---|
| 112. SQ SEQUENCE 66 AA; 7366 MW; 2901135CCE1111DB CRC64; | |
| MQNGKVKWFN SEKGFGFIEV EGGEDVFVHF SAIQGEGFKT LEEGQEVTFE VEQGNRGPQA | |
| TNVNKK | |
| 111. SQ Sequence 201 BP; 76 A; 32 C; 46 G; 47 T; 0 other; 1261403496 CRC32; | |
| atgcaaaacg gtaaagtaaa atggtttaac tcagaaaaag gtttcggatt catcgaagtt | 60 |
| gaaggcggag aagacgtatt cgttcatttc tcagctatcc aaggcgaagg tttcaaaact | 120 |
| ttagaagaag gtcaagaagt tactttcgaa gtagaacaag gtaaccgtgg acctcaagct | 180 |
| acaaacgtta acaagaagta a | 201 |

B. anthracis cspC - (P62169, Q45098, Q6HQV9, Q6KK79)

| | |
|---|---|
| 114. SQ SEQUENCE 65 AA; 7305 MW; 0B6EE9EDDE1F7A21 CRC64; | |
| MQGRVKWFNA EKGFGFIERE DGDDVFVHFS AIQQDGYKSL EEGQQVEFDI VDGARGPQAA | |
| NVVKL | |
| 113. SQ Sequence 198 BP; 64 A; 19 C; 56 G; 59 T; 0 other; 1665891028 CRC32; | |
| atgcaaggaa gagtgaaatg gtttaatgca gaaaagggat ttgggtttat tgagcgtgaa | 60 |
| gatggtgatg atgtgtttgt tcatttttct gctattcaac aagatggata taagtcatta | 120 |
| gaagaagggc aacaagttga gtttgatatt gtagatggag cacgtggacc acaagcagct | 180 |
| aatgttgtaa aactgtag | 198 |

B. anthracis cspD - (Q81K90, Q6HRP0, Q6KL07)

| | |
|---|---|
| 116. SQ SEQUENCE 66 AA; 7239 MW; CDF117183B093356 CRC64; | |
| MQTGKVKWFN SEKGFGFIEV EGGDDVFVHF SAIQGDGFKT LEEGQEVSFE IVEGNRGPQA | |
| ANVTKN | |
| 115. SQ Sequence 201 BP; 70 A; 33 C; 46 G; 52 T; 0 other; 306020295 CRC32; | |
| atgcaaacag gtaaagttaa atggtttaac agcgaaaaag gtttcggttt catcgaagtt | 60 |
| gaaggtggag acgatgtatt cgttcacttc tcagctatcc aaggtgacgg attcaaaact | 120 |
| ttagaagaag gtcaagaagt ttctttcgaa atcgttgaag gtaaccgtgg accacaagct | 180 |
| gctaacgtta caaaaaacta a | 201 |

B. anthracis cspE - (Q81QK2, Q6HYS0, Q6KSS3)

| | |
|---|---|
| 118. SQ SEQUENCE 67 AA; 7325 MW; 35A0CBE7E8352721 CRC64; | |
| MTLTGKVKWF NSEKGFGFIE VEGGNDVFVH FSAITGDGFK SLDEGQEVSF EVEDGNRGPQ | |
| AKNVVKL | |
| 117. SQ Sequence 204 BP; 67 A; 36 C; 48 G; 53 T; 0 other; 3616195753 CRC32; | |
| atgacattaa caggtaaagt aaaatggttt aacagcgaaa aaggtttcgg tttcatcgaa | 60 |
| gttgaaggcg gtaacgacgt attcgttcac ttctcagcta tcactggcga cggtttcaaa | 120 |
| tctcttgacg aaggtcaaga agttagcttc gaagttgaag acggtaaccg tggacctcaa | 180 |
| gctaaaaacg ttgtaaagct ataa | 204 |

B. anthracis NDK - (Q81SV8, Q6I137, Q6KVZ1)

| | |
|---|---|
| 120. SQ SEQUENCE 148 AA; 16601 MW; 35756A25423B8551 CRC64; | |
| MEKTFLMVKP DGVQRAFIGE IVARFEKKGF QLVGAKLMQV TPEIAGQHYA EHTEKPFFGE | |
| LVDFITSGPV FAMVWQGEGV VDTARNMMGK TRPHEAAPGT IRGDFGVTVA KNIIHGSDSL | |
| ESAEREIGIF FKEEELVDYS KLMNEWIY | |
| 119. SQ Sequence 447 BP; 146 A; 70 C; 104 G; 127 T; 0 other; 4071309316 CRC32; | |
| atggaaaaaa catttctaat ggttaaacca gacggtgtac aacgtgcctt cattggggaa | 60 |
| attgtagctc gttttgagaa gaagggcttt caattagttg gtgcaaaatt aatgcaagtc | 120 |
| actccagaaa tcgctggaca acactatgct gagcacacag aaaaaccttt ctttggtgaa | 180 |
| ttagtagact ttattacatc tggtcctgta ttcgcaatgg tatggcaagg tgaaggtgta | 240 |
| gtagatacag ctcgtaacat gatgggtaaa acaagaccac atgaagcagc tcctggaaca | 300 |
| attcgtggag atttcggtgt aactgttgcg aaaaacatta tccatggttc tgattcgtta | 360 |
| gaaagtgcag agcgcgagat tggtattttc tttaaggaag aagaattagt tgactactca | 420 |
| aaattaatga atgaatggat ttactaa | 447 |

B. anthracis NupC-1 - (Q81P28, Q6HXA0, Q6KR2C)

| | |
|---|---|
| 122. SQ SEQUENCE 397 AA; 43837 MW; 36A752FE1AB6CF94 CRC64; | |
| MYFILNMLGI FVVILIVYLC SPNKKHIKWR PIVILIILEL FITWFMLGTK LGSIIINKIA | |
| SFFSWLLACA NEGIRFAFPS AMENQTIDFF FSALLPIIFV ITFFDILSYF GILTWIIDKV | |
| GAVISKISRL PKLESFFSIQ MMFLGNTEAL AVVRDQLSVL KENRLLTFGI MSMSSVSGSI | |
| LGAYLSMVPA TYIFSAIPLN CINALILANV LNPVEVSKEE DVVYTPSKHE KKDFFSTISN | |
| SMLVGMNMVI VILAMVIGYV ALTACLNGIL GFFVTGLTIQ KIFSIIFSPF AFLLGLSGSD | |
| AMYVAELMGI KITTNEFVAM MDLKSNLKSL QPHTVAVATT FLASFANFST VGMIYGTYNS | |
| LFGGEKSSVI GKNVWKLLVS GMAVSLLSAM LVGLFVW | |
| 121. SQ Sequence 1194 BP; 339 A; 176 C; 222 G; 457 T; 0 other; 1884235346 CRC32; | |
| atgtatttca tattgaatat gttagggatt ttcgttgtca tattaattgt ttacttatgt | 60 |
| tcgcctaata aaaaacatat aaaatggaga ccaattgtaa ttctcatcat attagagctt | 120 |
| tttattacgt ggtttatgtt aggcacaaag ctaggcagta ttatcattaa taaaattgct | 180 |
| tcatttttca gttggctact ggcatgtgcg aatgaaggaa ttcgatttgc atttccttct | 240 |
| gctatggaaa atcagacaat tgatttcttc tttagcgcat tactacctat catttttgtt | 300 |
| atcacgttct ttgatattct ttcttacttt ggaatcttaa cttggattat tgataaagta | 360 |
| ggtgcagtta tttcaaagat ttctcgttta ccaaagttag aaagtttctt ttcgattcaa | 420 |
| atgatgtttt taggaaacac tgaagcactt gcggttgttc gtgatcaatt atctgtttta | 480 |
| aaagaaaacc gtttgctgac ttttggaatt atgagtatga gtagcgtcag cggttccatt | 540 |
| cttggtgctt atttatcaat ggttccagca acatatattt tcagcgcaat cccattaaat | 600 |
| tgtattaacg cattaatttt agccaatgta ttaaatcctg tggaagtttc gaaagaagaa | 660 |
| gatgttgttt acacaccttc caaacatgaa aaaaaggatt tcttttctac tatttcaaac | 720 |
| agcatgttag tcgggatgaa tatggttatc gttattttag ctatggtaat tggttatgta | 780 |
| gctttaactg catgtttaaa tgggatttta ggattttttg taacggggtt aacaattcaa | 840 |
| aaaatcttct ccattatctt tagtcctttc gcttttttac tcggtttatc gggcagtgat | 900 |
| gctatgtatg tagctgaatt aatggggatc aaaataacga cgaatgaatt tgttgcaatg | 960 |
| atggatttaa aatcaaactt aaagtcttta caaccgcata cggttgcggt tgccacaaca | 1020 |
| tttctagctt cttttgctaa ctttagtaca gtaggtatga tttatggaac ttacaattca | 1080 |
| ttatttggcg gcgaaaaatc atcagtcatc ggtaaaaatg tttggaagct tcttgtgagc | 1140 |
| ggaatggctg tttccttatt aagcgctatg cttgttgggc tttttgtatg gtaa | 1194 |

B. anthracis NupC-2 - (Q81RZ2, Q6I069, Q6KU46)

| | |
|---|---|
| 124. SQ SEQUENCE 393 AA; 42491 MW; E735B5BB5BA11A5F CRC64; | |
| MKYLIGVFGL VLILGIAWLA SNDRKKVKYR PIITMVILQF ILGFLLLNTS VGNILISGIA | |
| DGFGELLKYA ADGVNFVFGG LVNQKEFSFF LGVLMPIVFI SALIGILQHI KVLPIIVKSI | |
| GLALSKVNGM GKLESYNAVA SAILGQSEVF ISVKKQLGLL PEKRMYTLCA SAMSTVSMSI | |
| VGSYMVLLKP QYVVTALVLN LFGGFIIASI INPYEVTEEE DMLEVQEEEK KTFFEVLGEY | |
| IIDGFKVAIT VAAMLIGFVA LIAFINAVFK GVIGISFQEI LGYAFAPFAF IMGVPWHEAV | |
| NAGNIMATKL VSNEFVAMTD LAQGNFNFSD RTTAIISVFL VSFANFSSIG IIAGAVKSLN | |
| EKQGNVVARF GLKLLFGATL VSFLSATIVG LLF | |
| 123. SQ Sequence 1182 BP; 362 A; 160 C; 241 G; 419 T; 0 other; 2336716326 CRC32; | |
| atgaaatact taatcggtgt ttttggcctc gtattgattt taggtatcgc ttggcttgct | 60 |
| agtaatgata gaaagaaagt caaatatcgc ccaatcataa cgatggttat attacaattc | 120 |
| attttggggt ttctattatt aaatacaagt gtcgggaata tattaattag cggaatagca | 180 |
| gatggttttg gagagctgtt aaaatatgcc gctgacggtg tgaatttcgt atttggtgga | 240 |
| ttagtaaatc aaaaagagtt ttcattcttt ttaggtgtat taatgccaat tgtatttatt | 300 |
| tcagctttaa tcggtatttt gcagcacatt aaagtattac ctattattgt gaaatctatc | 360 |
| ggtctagcat taagtaaagt aaatggaatg gggaaactag aatcatacaa tgctgttgct | 420 |
| tccgcgattt taggacaatc tgaagtgttt atttcagtta agaagcaact agggttattg | 480 |
| ccagagaaaa gaatgtatac attatgtgca tctgcaatgt ctaccgtttc catgtctatc | 540 |
| gttggatcat acatggtctt attaaaaccg caatatgttg taaccgcttt agtgcttaac | 600 |
| ttattcggtg gttttattat tgcttctatc attaatcctt atgaagttac ggaagaagaa | 660 |
| gatatgttag aagtacaaga agaagagaaa aagactttct ttgaagtatt aggggaatac | 720 |
| attattgatg gatttaaagt tgcgattaca gtagcagcta tgttaattgg tttcgttgct | 780 |
| cttatcgcat tcattaatgc cgtatttaaa ggtgtaatcg gtatttcatt ccaagaaatt | 840 |
| ctcggttatg catttgcacc atttgcattt attatgggtg taccttggca tgaagcagtt | 900 |
| aatgccggaa atattatggc aacaaaatta gtatcgaatg aatttgtcgc tatgacagat | 960 |
| ttagcacaag gaaactttaa tttctcagat agaacgacag cgattatatc tgtattctta | 1020 |
| gtttcatttg caaacttctc ttcaattgga attattgcag gggcagtgaa gagtttaaat | 1080 |
| gaaaagcaag ggaatgtagt cgcaagattt ggtttgaagt tacttttcgg tgcaacatta | 1140 |
| gtaagtttct tatcagcaac aatcgtaggc ttattatttt aa | 1182 |

B. anthracis NupC-3 - (Q81V93, Q6I3I0, Q6KX93)

| | |
|---|---|
| 126. SQ SEQUENCE 392 AA; 43087 MW; 37D7C8E9294BB526 CRC64; | |
| MKFITFFLGL IVVFFLAYIA SNNKKHIKFK PIFIMLLIQL ILTYLLLNTE IGLILIRVIS | |
| SLFTKLLEYA ADGINFVFGG LANKGEMPFF LTVLLPIVFI SVLIGILQHF KILPFFIHWI | |
| GYFLSKINGL GKLESYNAIA SAIVGQSEVF ITVKKQLAQI PKHRLYTLCA SAMSTVSMSI | |
| VGAYMTMIEP KYVVTALVLN LFSGFIIVLI INPYDVKDDE DILEIKGEKQ SFFEMLGEYI | |
| LDGFRVAIVV GAMLIGFVAL ISCINDLFLI IFGITFQQLI GYVFAPIAFL IGVPSSEIVA | |
| AGSIMATKLV TNEFVAMMDL SKISNSLSPR TVGIISVFLV SFANFSSIGI ISGAVKGLNE | |
| EQGNVVARFG LKLLYGATLV SILSAIIVSI ML | |
| 125. SQ Sequence 1179 BP; 325 A; 225 C; 197 G; 432 T; 0 other; 3533660419 CRC32; | |
| atgaaattta ttactttttt cttaggactt atcgtcgtct tcttccttgc ttatatcgct | 60 |
| agtaacaata agaagcatat taaatttaaa cctattttca tcatgcttct tatacagtta | 120 |
| attttaacct atttattatt gaatacagaa atcggtctca tacttattcg ggtcatctcc | 180 |
| agtttgttta caaagctact cgagtatgct gctgatggta taaacttcgt atttggcggc | 240 |
| cttgccaata aaggtgaaat gccctttttc cttactgtct tattaccaat tgtcttcatt | 300 |
| tccgtcttaa ttggtatact acaacatttc aaaatactac catttttcat tcattggatc | 360 |
| ggttacttcc tgagcaaaat aaatggtctt gggaaattag aatcttataa tgctatcgcc | 420 |
| tctgccattg tcggccaatc agaagttttt attacagtca aaaaacaatt agctcaaatt | 480 |
| ccaaaacacc gtctttatac actttgtgca tctgccatgt caaccgtatc tatgtctatc | 540 |
| gtaggtgcct atatgacaat gattgaacct aaatatgtag taaccgcact cgttctcaat | 600 |
| ttatttagcg gttttattat cgtacttatc attaaccctt acgacgttaa agatgacgaa | 660 |
| gatattttag agattaaagg cgaaaagcaa agcttttttg aaatgcttgg agaatacatt | 720 |
| ttagatggct ttcgcgtagc tatcgttgtc ggggctatgc ttatcggatt cgtcgcatta | 780 |
| attagctgca ttaatgatct attcctcatt atattcggca ttactttcca acaattaatc | 840 |
| ggctacgtct ttgcgcctat tgcattcctt atcggtgtac caagttctga aattgtcgcg | 900 |
| gctggtagca ttatggcaac gaagcttgta acgaatgaat ttgtagcaat gatggacctt | 960 |
| agtaaaatct ctaatagcct ttctccccgt acagttggta ttatttctgt tttcctcgtt | 1020 |
| tcttttgcca acttttcttc tatcggcatt atttcaggtg cggtaaaagg attaaacgaa | 1080 |
| gaacaaggaa acgttgttgc aaggtttggc cttaaattac tatatggagc tactctcgtt | 1140 |
| agtattttat ctgcaattat cgtaagcatt atgttgtaa | 1179 |

B. anthracis NupC-4 - (Q81XE1, Q6HR75, Q6KKJ3)

| | |
|---|---|
| 128. SQ SEQUENCE 393 AA; 42210 MW; AFBFB9D59447CD8A CRC64; | |
| MKFVMFLVGL LVVFVLGFLI SSDRKKIKYK PIALMLVIQL VLAYFLLNTK VGFVLVKGIA | |
| DGFGAILKFA EAGVNFVFGG LANDGQAPFF LTVLLPIIFL AVLIGILQHI KILPIIIRAV | |
| GFLLSKVNGL GKLESYNAVA AAIVGQGEVF ITVKDQLSKL PKNRLYTLCA SSMSTVSMSI | |
| VGSYMKMIDP KYVVTALVLN LFSGFIIVHI INPYDVKEED DILELQEDKK QTFFEMLGEY | |
| IMLGFSIAVT VAAMLIGFVA LITAINGVFD SIFGITFQSI LGYIFSPLAF VMGIPTSEML | |
| TAGQVMATKL VTNEFVAMLD LGKVAGDLSA RTVGILSIFL VSFANFSSIG IIAGATKSID | |
| GKQANVVSSF GLKLVYGATL VSILSAVIVG VML | |
| 127. SQ Sequence 1182 BP; 374 A; 187 C; 227 G; 394 T; 0 other; 1670261224 CRC32; | |
| atgaagttcg taatgtttct agtcggttta cttgtagtat ttgtactagg gttccttatc | 60 |
| agttcagatc gtaaaaagat taaatataaa ccaattgcac ttatgcttgt cattcaattg | 120 |
| gtacttgcgt atttcttact aaatacaaag gtcggatttg tattagtaaa agggattgca | 180 |
| gatggatttg gggctatttt aaaatttgcg gaagcagggg ttaatttcgt atttggtggt | 240 |
| ctagcaaatg atggacaagc accattcttc ttaacagtat tattaccaat tattttctta | 300 |
| gcagtactaa ttgggatctt acaacatatt aaaattttac cgattatcat tcgtgcagtc | 360 |
| ggtttcctat taagcaaagt taacggttta ggaaaactag aatcatataa tgcggtagca | 420 |
| gctgcaatcg ttggtcaagg ggaagtattc attacagtaa aagatcaatt aagcaaacta | 480 |
| ccgaaaaatc gtttatacac actttgtgca tcttctatgt caacggtatc gatgtcaatc | 540 |
| gtcggttctt atatgaaaat gattgatcca aaatatgtag taacagcact tgtactaaac | 600 |
| ttattcagtg gatttattat cgttcatatt attaatccat atgacgtaaa agaagaagac | 660 |
| gatattttag aattacaaga agataaaaaa caaacattct ttgaaatgtt aggcgaatat | 720 |
| attatgcttg gtttctctat cgctgtaaca gtagcggcga tgttaatcgg tttcgtagca | 780 |
| ttaattacag caattaacgg tgtattcgat tcaattttcg gaatcacatt ccaaagcatt | 840 |
| ttaggataca ttttctcacc attagcattc gtaatgggta tcccaacatc agagatgcta | 900 |
| acagcaggac aagttatggc aacgaaatta gtaacgaacg aatttgttgc aatgcttgac | 960 |
| cttggaaaag tagctggcga tttatcagct cgtacagtag gtattttatc tatcttcctt | 1020 |
| gtatcatttg cgaacttctc atcaatcgga attatcgcag gtgcaacgaa gagtatcgat | 1080 |
| ggcaaacaag caaacgttgt atcatcattc ggcttaaaac ttgtatacgg tgcaacgtta | 1140 |
| gtaagtatat tatcagcggt tatcgttggg gttatgcttt aa | 1182 |

B. anthracis NupC-5 - (Q81K60, Q6HQR4, Q6KK34)

| | |
|---|---|
| 130. SQ SEQUENCE 403 AA; 42528 MW; C4DAB3827B2E9F7E CRC64; | |
| MNLLWGIGGV IGVLAIAFLL SSNRKAINWR TILIALALQM SFSFIVLRWD AGKAGLKHAA | |
| DGVQGLINFS YEGIKFVAGD LVNAKGPWGF VFFIQALLPI VFISSLVAIL YHFGIMQRFV | |
| SVVGGALSKL LGTSKAESLN SVTTVFLGQT EAPILIKPYL ARLTNSEFFA IMVSGMTAVA | |
| GSVLVGYAAM GIPLEHLLAA AIMAAPSSLL IAKLIMPETE KVDNNVELST EREDANVIDA | |
| AARGASEGMQ LVINVAAMLM AFIALIALLN GLLGLIGSLF DIKLSLDLIF GYLLSPFAIL | |
| IGVSPGEAVQ AASFIGQKLA INEFVAYANL GPHMAEFSDK TNLILTFAIC GFANFSSIAI | |
| QLGVTGTLAP TRRKQIAQLG IKAVIAGTLA NFLNAAVAGM MFL | |
| 129. SQ Sequence 1212 BP; 360 A; 218 C; 236 G; 398 T; 0 other; 1175765933 CRC32; | |
| atgaatcttt tatggggaat tggcggcgtg attggagtat tagcaatcgc ttttttacta | 60 |
| tcttccaacc gcaaagctat taattggcgc acaattttaa tcgcgctagc attacaaatg | 120 |
| tcattttcat ttatcgtatt acgctgggat gccggaaaag caggtttaaa acacgctgca | 180 |
| gatggcgttc aaggattaat taatttttct tacgagggaa ttaagttcgt tgctggggat | 240 |
| ttagtcaacg caaaaggccc ttggggattt gttttcttca ttcaagcact acttccaatc | 300 |
| gtatttatta gttcattagt agcaatctta tatcatttcg gtattatgca aagatttgtt | 360 |
| agtgtcgttg gtggcgcatt aagtaaactt cttggaactt ctaaagcaga aagtttaaac | 420 |
| tcagtaacaa ctgtattttt aggacaaact gaagctccaa tcttaatcaa accttactta | 480 |
| gcacgtttaa caaatagtga attcttcgct attatggtaa gcggtatgac agctgttgct | 540 |
| ggatcagttc ttgtcggtta tgcagcaatg ggtattccgt tagaacactt attagcagca | 600 |
| gcaattatgg cagctccatc aagtttatta attgcaaaat taattatgcc agaaacagaa | 660 |
| aaagtagata ataacgttga actttctaca gaacgtgaag atgcaaacgt tattgacgct | 720 |
| gcggcacgtg gtgcatctga aggtatgcaa cttgttatta acgtagcagc aatgttaatg | 780 |
| gcttttatcg cattaatcgc tttactaaac ggtttattag gattaattgg ctctctgttt | 840 |
| gatattaaac ttagtcttga tttaatcttc ggttatttac tatcaccatt tgcaatttta | 900 |
| atcggggttt ctcctggtga agctgtacaa gcagcaagct ttatcggtca aaaacttgca | 960 |
| atcaacgaat tcgttgcata cgcaaactta ggaccacaca tggcagagtt ctctgacaaa | 1020 |
| acaaatttaa ttttaacatt cgcaatctgt ggattcgcaa acttctcttc tatcgcaatt | 1080 |
| caattaggtg taacaggaac attggctcct actcgccgta aacaaattgc acaattaggg | 1140 |
| attaaagcag ttatcgctgg tacattagca aacttcttaa atgcagcagt tgcaggtatg | 1200 |
| atgttcctat aa | 1212 |

B. anthracis NupC-6 - (Q81XE0, Q6HR74, Q6KKJ2)

| | |
|---|---|
| 132. SQ SEQUENCE 393 AA; 42471 MW; 0C976432FE2524C1 CRC64; | |
| MKFVMFLVGL LVVFVLGFLI SADRKKIKYK PIAIMLVIQL ALSYFLLNTQ VGYILVKGIS | |
| DGFGALLGYA EAGIVFVFGG LVNKGEVSFF LTALLPIVFF AVLIGILQHF KILPIFIRAI | |
| GTLLSKVNGL GKLESYNAVA AAIVGQAEVF ITVKDQLSKI PKHRLYTLCA SSMSTVSMSI | |
| VGSYMKMIEP KYVVTALVLN LFSGFIIIHI INPYDITEEE DTLKLENKKK QSFFEMLSEY | |
| IMLGFTIAIT VAAMLLGFVA LITAINSLFD SMFGITFQAI LGYIFSPLAF VMGIPQAEMV | |
| TAGQIMATKL VSNEFVAMLD LGKVAGDLSA RTVGILSVFL VSFANFSSIG IIAGATKGID | |
| ENQSNVVSSF GLRLVYGATL VSILSAIIVG VML | |
| 131. SQ Sequence 1182 BP; 355 A; 183 C; 230 G; 414 T; 0 other; 3621345752 CRC32; | |
| atgaagtttg ttatgtttct tgtaggatta ctcgttgtat ttgtactcgg ttttcttata | 60 |
| agtgccgatc gaaagaagat taagtataaa ccaatcgcaa ttatgcttgt tattcagtta | 120 |
| gcgttatctt atttcttatt aaatacgcaa gttggttata ttttagtaaa aggaatttca | 180 |
| gatggatttg gcgcgcttct tggatatgca gaagctggaa tcgttttcgt atttggtggc | 240 |
| cttgttaata aaggagaggt ttcattcttc ttaacagcgt tattaccaat cgtattcttt | 300 |
| gccgttttaa tcggaattct gcaacacttt aaaattttac cgatatttat tcgtgctatt | 360 |
| ggtactttgt taagtaaagt aaatggtcta ggaaaactag aatcatataa cgcagtagca | 420 |
| gctgctattg ttgggcaagc ggaagtattt attacagtaa aagatcaatt aagtaaaatc | 480 |
| ccaaaacatc gtttatatac attatgtgca tcttccatgt cgacagtatc gatgtcaatc | 540 |
| gtcggttctt acatgaaaat gatcgaacca aaatatgtag taacagcact tgtattaaat | 600 |
| ttatttagtg gtttcattat tattcatatt attaacccgt acgatattac agaagaagaa | 660 |
| gatacactga aattagaaaa taagaaaaaa cagtcattct ttgaaatgtt aagtgaatat | 720 |
| attatgcttg gtttcacaat cgcgattaca gtagcagcga tgttacttgg tttcgtagcg | 780 |
| ttaattacag caatcaatag cttgtttgat tccatgttcg gtattacatt ccaagcgatt | 840 |
| ttaggatata ttttctcccc attagcattc gtaatgggta tcccgcaagc agagatggta | 900 |
| acagcgggac aaattatggc aacgaaatta gtatcaaacg aatttgttgc gatgcttgat | 960 |
| cttggaaaag tagctggtga tttatcagct cgtacagttg gtatcctttc tgtattcctt | 1020 |
| gtatcatttg cgaacttctc atcaatcgga attatcgcag gtgcaacgaa aggtatcgat | 1080 |
| gagaaccaat caaatgtagt atcatcattc ggtctacgcc ttgtgtacgg tgcgacatta | 1140 |
| gtaagtattc tatcagcgat tatcgttggt gttatgttat ag | 1182 |

B. anthracis NupC-7 - (Q81ZD7, Q6I483, Q6KXY9)

| | |
|---|---|
| 134. SQ SEQUENCE 398 AA; 42688 MW; 35AC4C1C565F88F4 CRC64; | |
| MQYVMSIIGI LVVLGLCFAL SNNKSKINFR AIAIMIGFQI LIGWFMFGTK IGQQIIIFIS | |
| KVFNKLIKLG TTGVDFLFNG IQRDFVFFLN VLLIIVFFSA LLSIFSYLGV LPFIVRVVGG | |
| AISKVTGLPR VESFHAVNSV FFGSSEALIV IKNDLQHFNK NRMFIICCSA MSSVSASVTA | |
| SYVMMLDAKY VLAALPLNLF SSLIVCSLLT PVDTKKEDEV VQKFDRTVFG DSFIGAMING | |
| ALDGLKVAGI VAALMIAFIG VMEVVNYVIS AASGAMGHAV TLQQIFGYVL APFAFLMGIP | |
| AQDIIPAGGI MGTKIVLNEF VAILDLKGVA ATLSPRTVGI VTVFLISFAS ISQIGAIVGT | |
| IRALSEKQGS IVSKFGWKML FASTLASILS ATIAGLFI | |
| 133. SQ Sequence 1197 BP; 334 A; 205 C; 229 G; 429 T; 0 other; 1867719549 CRC32; | |
| atgcaatatg taatgagcat tatcggtatt cttgtcgttt taggtttatg ttttgctttg | 60 |
| tcaaacaaca aaagtaaaat caacttccgt gcaattgcaa ttatgattgg tttccaaatt | 120 |
| ttaatcggtt ggtttatgtt tggcacaaaa attggtcaac aaattatcat cttcattagt | 180 |
| aaagttttca acaaactaat taaacttggt acgacaggcg tcgattttct ctttaatgga | 240 |
| attcaaagag attttgtctt tttcttaaac gtattattaa ttatcgtatt tttctcagca | 300 |
| ctactttcta tctttagtta tttaggtgtt ttaccattca tcgttcgcgt tgtcggcggt | 360 |
| gccatttcaa aagttactgg tttaccacgc gttgaatcat tccacgcagt aaactctgta | 420 |
| ttcttcggtt caagtgaagc tttaatcgtt attaaaaatg atttacagca ttttaacaaa | 480 |
| aaccgtatgt ttatcatttg ttgttctgcg atgagctcag tttctgcttc tgttacagca | 540 |
| tcatacgtaa tgatgttaga tgcaaaatat gtattagcag ctcttccatt aaacttattc | 600 |
| tcaagcttaa tcgtttgttc gttattaaca cctgttgata cgaaaaagga agacgaagta | 660 |
| gttcagaaat ttgaccgaac tgtattcggg gacagcttta tcggtgcaat gattaacggt | 720 |
| gcgcttgacg gtttaaaagt agcaggtatc gttgccgcat taatgatcgc tttcatcggt | 780 |
| gtgatggaag ttgtaaacta cgtaattagc gcagcttcag gtgcaatggg acatgccgtt | 840 |
| acgttacaac aaatctttgg ttacgtactt gctccatttg cattcttaat gggtattcca | 900 |
| gctcaagata ttatcccagc tggcggaatt atgggtacga agattgtatt aaacgagttt | 960 |
| gtagcaatcc ttgatttaaa aggtgttgca gcaacattat ctccacgtac agttggaatc | 1020 |
| gttacagtat tcttaattag cttcgcaagt attagccaaa ttggagcgat cgttggtaca | 1080 |
| attcgtgctc tttctgagaa acaaggaagc atcgtatcga aatttggttg gaaaatgcta | 1140 |
| tttgcatcaa cacttgcttc tattttatct gcgacaatcg ctggattgtt tatttaa | 1197 |

B. anthracis PnuC - (Q81VJ8, Q6I4J6, Q6KY98)

| | |
|---|---|
| 136. SQ SEQUENCE 216 AA; 25000 MW; E5C21CD80DE9F357 CRC64; | |
| MIRSPLFLLI TSIICVLVGL YIQSSYIEIF ASVMGIINVW LLAREKVSNF LFGMITVAVF | |
| LYIFITQGLY AMAVLAAFQF IFNVYGWYHW IARSGEEEVK ATVRLDLKGW IFYIIFILVA | |
| WIGWGYYQVH YLESTSPYLD ALNAVLGLVA QFMLSRKILE NWHLWILYNV VSISIYISTG | |
| LYVMLILAVI NLFICVAGLL EWKNNYKGQK HTNNYI | |
| 135. SQ Sequence 651 BP; 196 A; 74 C; 128 G; 253 T; 0 other; 931887757 CRC32; | |
| atgattagaa gtccgctctt tttactcatt actagtatta tttgtgtatt ggttggactg | 60 |
| tatattcaat cgagctatat tgaaatcttt gcatcggtca tgggaattat taatgtttgg | 120 |
| ctattagcaa gagaaaaagt atccaacttt ttattcggta tgattaccgt tgcggtattt | 180 |
| ctatatattt ttattacaca aggtttatat gcaatggcag tattggcagc ctttcaattt | 240 |
| atatttaatg tatatggttg gtatcattgg attgcacgta gtggggagga agaggtaaaa | 300 |
| gcaacagttc gtttagattt gaaaggttgg attttttata taatctttat tttagttgca | 360 |
| tggattggtt gggggtatta tcaagtccat tacttagaat caacaagtcc atatttagac | 420 |
| gctttaaatg ctgtactagg attagtagct caatttatgt taagtcgaaa aatcttagaa | 480 |
| aactggcatt tatggatttt atataatgta gttagtattt caatttatat ttccactggg | 540 |
| ttatacgtta tgctaatatt agctgttatt aatctcttta tatgtgtagc gggtttgcta | 600 |
| gagtggaaga ataattataa gggacaaaaa catacaaata attatatcta g | 651 |

B. anthracis Alanine racemase - (Q81RG8, Q6HZP3, Q6KTN0)

| | |
|---|---|
| 138. SQ SEQUENCE 391 AA; 43372 MW; F8AA173912483DF4 CRC64; | |
| MSLKYGRDTI VEVDLNAVKH NVKEFKKRVN DENIAMMAAV KANGYGHGAV EVAKAAIEAG | |
| INQLAIAFVD EAIELREAGI NVPILILGYT SVAAAEEAIQ YDVMMTVYRS EDLQGINEIA | |
| NRLQKKAQIQ VKIDTGMSRI GLQEEEVKPF LEELKRMEYV EVVGMFTHYS TADEIDKSYT | |
| NMQTSLFEKA VNTAKELGIH IPYIHSSNSA GSMEPSNTFQ NMVRVGIGIY GMYPSKEVNH | |
| SVVSLQPALS LKSKVAHIKH AKKNRGVSYG NTYVTTGEEW IATVPIGYAD GYNRQLSNKG | |
| HALINGVRVP VIGRVCMDQL MLDVSKAMPV QVGDEVVFYG KQGEENIAVE EIADMLGTIN | |
| YEVTCMLDRR IPRVYKENNE TTAVVNILRK N | |
| 137. SQ Sequence 1176 BP; 430 A; 149 C; 272 G; 325 T; 0 other; 1685638731 CRC32; | |
| atgagtttga aatatggaag agatacaatt gttgaagttg acttaaatgc agtaaaacat | 60 |
| aatgtaaaag aatttaaaaa acgtgtgaat gatgaaaata ttgcaatgat ggctgctgta | 120 |
| aaagcgaatg ggtatggtca tggggcagtt gaagttgcaa aagctgctat tgaagcagga | 180 |
| ataaatcagc ttgcaattgc atttgtagat gaagcgatag agttaagaga agcaggaatt | 240 |
| aacgtgccga ttttaatttt aggctataca tcagtagcgg ctgcggaaga agcaattcaa | 300 |
| tatgacgtta tgatgaccgt ttatagaagt gaagatttac aaggtataaa tgaaatcgca | 360 |
| aaccgtcttc aaaagaaagc gcaaattcag gtgaaaattg atacaggaat gagtcgcatt | 420 |
| ggtttacagg aagaagaggt taaaccattt ttagaggaat taaaacgtat ggagtatgta | 480 |
| gaggtagtgg gaatgtttac acattactct acggcagatg aaatcgataa atcatatacg | 540 |
| aatatgcaaa caagtttatt tgagaaagct gtcaatacag caaaagaatt aggaattcat | 600 |
| attccatata ttcatagttc aaatagtgca ggttcaatgg aacctagcaa tacatttcaa | 660 |
| aatatggttc gtgtaggtat cggaatttat ggaatgtatc cttcaaaaga ggtaaatcat | 720 |
| tcagttgttt cgttacagcc tgcgttgtcg ttaaaatcaa aagtagccca tattaagcat | 780 |
| gcgaagaaaa atcgcggtgt aagttatggg aatacgtatg taacgactgg tgaagaatgg | 840 |
| attgccaccg taccgattgg ttatgctgat ggttataatc gtcagttgtc taataaaggg | 900 |
| catgcattaa taaatggagt tcgagtacct gttattggcc gtgtttgtat ggatcagctc | 960 |
| atgttagacg tttcaaaagc aatgccagta caagtgggag acgaagtagt attctacggt | 1020 |
| aaacaaggcg aagaaaacat cgcagtagaa gaaatagcgg atatgttagg tacaattaac | 1080 |
| tatgaagtta catgtatgtt agatagaaga attccacgtg tgtataaaga aaataatgaa | 1140 |
| acaactgctg ttgtaaatat actaagaaaa aactga | 1176 |

B. anthracis Alanine dehydrogenase - (Q81VA6, Q6I3J2, Q6KXA6)

| | |
|---|---|
| 140. SQ SEQUENCE 377 AA; 40234 MW; 5ED5B3B2F858EBAE CRC64; | |
| MRIGVPAEIK NNENRVAMTP AGVVHLIRNN HEVFIQKGAG LGSGFTDAQY VEAGAKIVDT | |
| AEEAWNMEMV MKVKEPIESE YKHFSEGLIL FTYLHLAPEP ELTKALIEKK VVSIAYETVQ | |
| LENRSLPLLA PMSEVAGRMA AQIGAQFLEK NKGGKGILLA GVPGVKRGKV TIIGGGQAGT | |
| NAAKIAVGLG ADVTIIDLSA ERLRQLDDIF GNQVKTLMSN PYNIAEAVKE SDLVIGAVLI | |
| PGAKAPKLVT EEMIKSMEPG SVVVDIAIDQ GGIFETTDRI TTHDNPTYEK HGVVHYAVAN | |
| MPGAVPRTST LALTNVTVPY AVQIANKGYK EACLGNSALL KGINTLDGYV TFEAVAEAHG | |
| VEYKGAKELL EAETVSC | |
| 139. SQ Sequence 1134 BP; 395 A; 201 C; 242 G; 296 T; 0 other; 3241826283 CRC32; | |
| atgcgtattg gggtaccagc agaaattaaa aacaacgaaa accgtgtggc aatgacacca | 60 |
| gcaggtgttg tacatttaat tcgtaacaat cacgaagtat tcattcaaaa gggtgcaggt | 120 |
| ttaggatctg gtttcacaga tgctcagtat gttgaagcag gagcgaaaat tgttgataca | 180 |
| gctgaagaag cttggaacat ggaaatggtt atgaaagtta aggaaccaat tgaaagcgaa | 240 |
| tacaaacact tcagcgaagg tttgatctta ttcacatact tacacttagc tccagaacca | 300 |
| gaattaacaa aagcattaat cgaaaagaaa gttgtttcta ttgcatatga aacagtacaa | 360 |
| ttagaaaacc gttctctacc attacttgca cctatgagtg aagtagctgg tcgtatggct | 420 |
| gcacaaattg gtgcacaatt ccttgagaaa aacaaaggcg gtaaaggtat cttacttgca | 480 |
| ggtgttccag gggttaaacg tggtaaagta acaatcatcg gtggtggaca agctggtaca | 540 |
| aatgctgcta aaatcgcagt tggactaggt gcggatgtaa caatcatcga cttaagtgca | 600 |
| gaacgtcttc gtcaattaga tgatattttc ggaaatcaag taaaaacttt aatgtctaat | 660 |
| ccttacaata ttgcagaagc tgtaaaagag tctgatcttg taatcggtgc agtattaatc | 720 |
| ccaggtgcaa aagctccaaa acttgtaaca gaagaaatga ttaaatcaat ggaaccaggt | 780 |
| tctgttgttg tagatatcgc gattgaccaa ggtggtattt tcgaaacaac tgaccgtatt | 840 |
| acaactcatg ataacccaac ttacgaaaaa cacggcgttg ttcattatgc agttgcaaac | 900 |
| atgccaggtg cggttccacg tacatcaact cttgcattaa caaacgtaac agtaccatat | 960 |
| gcagtgcaaa ttgctaacaa aggctacaaa gaagcttgcc taggcaactc tgcattacta | 1020 |
| aaaggtatta acacattaga tggctatgta acattcgaag cagttgcaga agctcacggt | 1080 |
| gtagagtaca aaggtgctaa agaattatta gaagcagaaa cagtatcttg ctaa | 1134 |

B. anthracis Nucleoside hydrolase - (Q81YE3, Q6KPV2)

| | |
|---|---|
| 142. SQ SEQUENCE 310 AA; 34464 MW; 3F5DD1D3C7E8AEB4 CRC64; | |
| MKKVLFLGDP GIDDSLAIMY GLLHPDIDIV GVVTGYGNVT QEKATSNAAY LLQLAGREDI | |
| PIINGAKIPL SGDITTYYPE IHGAEGLGPI RPPKNLSPNI RPFCEFFDIL EKYKGELIIV | |
| DAGRSTTLAT AFILEKPLMK YVKEYYIMGG AFLMPGNVTP VAEANFHGDP IASQLVMQNA | |
| KNVTLVPLNV TSEAIITPEM VKYITKHSKT SFNKLIEPIF TYYYKAYRKL NPKITGSPVH | |
| DVVTMMVAAN PSILDYVYRR VDVDTVGIAK GESIADFRPQ PDAKALKNWV RIGWSLHYKK | |
| FLEDFVKIMT | |
| 141. SQ Sequence 933 BP; 314 A; 137 C; 201 G; 281 T; 0 other; 2650827719 CRC32; | |
| atgaaaaaag tattattttt aggagaccca ggaattgatg actctttagc aattatgtat | 60 |
| ggattgttgc atcctgatat tgatattgtt ggtgtagtaa ctggatatgg aaatgtaacg | 120 |
| caagaaaagg cgacaagtaa tgcggcatat ttattgcaac tggcaggacg ggaagatata | 180 |
| cctattatta atggtgcgaa aatcccttta tctggagata ttacaacgta ttatccagaa | 240 |
| attcatgggg cggaaggctt aggaccaatt cgaccgccga aaaatctttc tccaaatata | 300 |
| aggccttttt gtgagttttt tgacattctt gaaaaatata aaggagaatt aattatagtt | 360 |
| gatgctggga ggtcaacgac acttgcaaca gcatttattt tagaaaaacc attgatgaag | 420 |
| tatgtgaaag aatattatat aatgggcggt gcttttttaa tgcctggaaa tgttacacca | 480 |
| gtcgcagaag cgaattttca tggtgaccct attgcatcac aattagtcat gcaaaatgcc | 540 |
| aagaatgtga cgttggtgcc gctgaatgtt acatctgaag ctataatcac gccagagatg | 600 |
| gtaaagtaca ttacgaaaca ttctaaaacg agttttaata aattaattga accgattttt | 660 |
| acgtattatt ataaagctta tagaaagtta aatccgaaaa taacaggaag tccagtacat | 720 |
| gacgttgtta caatgatggt cgcggcgaat ccttcaatac tggattatgt gtatcgtcgt | 780 |
| gtagatgtag atacagtggg gattgcaaaa ggagaaagta ttgcagattt ccgtcctcaa | 840 |
| cctgatgcaa aagccttaaa aaattgggta cgaattggtt ggtcattaca ttataaaaaa | 900 |
| ttccttgagg attttgtgaa aatcatgacg tag | 933 |

Staphylococcus aureus (MRSA252) srtA - (Q6GDS0)

| | |
|---|---|
| 144. SQ SEQUENCE 206 AA; 23599 MW; 5EE14FC04E42BA9B CRC64; | |
| MKKWTNRLMT IAGVVLILVA AYLFAKPHID NYLHDKDKDE KIEQYDKNVK EQASKDNKQQ | |
| AKPQIPKDKS KVAGYIEIPD ADIKEPVYPG PATPEQLNRG VSFAEENESL DDQNISIAGH | |
| TFIDRPNYQF TNLKAAKKGS MVYFKVGNET RKYKMTSIRD VKPTDVEVLD EQKGKDKQLT | |
| LITCDDYNEK TGVWEKRKIF VATEVK | |
| 143. SQ Sequence 621 BP; 264 A; 89 C; 113 G; 155 T; 0 other; 1991146918 CRC32; | |
| atgaaaaaat ggacaaatcg attaatgaca atcgctggtg tagtacttat cctagtggca | 60 |
| gcatatttgt ttgctaaacc acatatcgat aattatcttc acgataaaga taaagatgaa | 120 |
| aagattgaac aatatgataa aaatgtaaaa gaacaggcga gtaaagacaa taagcagcaa | 180 |
| gctaaacctc agattccgaa agataaatca aaagtggcag gctatattga aattccagat | 240 |
| gctgatatta aagaaccagt atatccagga ccagcaacac ctgaacaatt aaatagaggt | 300 |
| gtaagctttg cagaagaaaa tgaatcacta gatgatcaaa atatttcaat tgcaggacac | 360 |
| actttcattg accgtccgaa ctatcaattt acaaatctta aagcagccaa aaaaggtagt | 420 |
| atggtttact ttaaagttgg taatgaaaca cgtaagtata aaatgacaag tataagagat | 480 |
| gttaagccta cagatgtaga agttctagat gaacaaaaag gtaaagataa acaattaaca | 540 |
| ttaattactt gtgatgatta caatgaaaag acaggcgttt gggaaaaacg taaaatcttt | 600 |
| gtagctacag aagtcaaata a | 621 |

Document C: List of Amino Acid and Nucleotide Sequence for Surface
Proteins from Bacillus subtilis that are predicted to be included in Bacillus
anthracis

B. subtilis, CotA - (P07788)

| | |
|---|---|
| 146. SQ SEQUENCE 513 AA; 58499 MW; 836B83B458D75F87 CRC64; | |
| MTLEKFVDAL PIPDTLKPVQ QSKEKTYYEV TMEECTHQLH RDLPPTRLWG YNGLFPGPTI | |
| EVKRNENVYV KWMNNLPSTH FLPIDHTIHH SDSQHEEPEV KTVVHLHGGV TPDDSDGYPE | |
| AWFSKDFEQT GPYFKREVYH YPNQQRGAIL WYHDHAMALT RLNVYAGLVG AYIIHDPKEK | |
| RLKLPSDEYD VPLLITDRTI NEDGSLFYPS APENPSPSLP NPSIVPAFCG ETILVNGKVW | |
| PYLEVEPRKY RFRVINASNT RTYNLSLDNG GDFIQIGSDG GLLPRSVKLN SFSLAPAERY | |
| DIIIDFTAYE GESIILANSA GCGGDVNPET DANIMQFRVT KPLAQKDESR KPKYLASYPS | |
| VQHERIQNIR TLKLAGTQDE YGRPVLLLNN KRWHDPVTET PKVGTTEIWS IINPTRGTHP | |
| IHLHLVSFRV LDRRPFDIAR YQESGELSYT GPAVPPPPSE KGWKDTIQAH AGEVLRIAAT | |
| FGPYSGRYVW HCHILEHEDY DMMRPMDITD PHK | |
| 145. SQ Sequence 1536 BP; 457 A; 396 C; 337 G; 346 T; 0 other; 2755677677 CRC32; | |
| atgacacttg aaaaatttgt ggatgctctc ccaatcccag atacactaaa gccagtacag | 60 |
| caatcaaaag aaaaaacata ctacgaagtc accatggagg aatgcactca tcagctccat | 120 |
| cgcgatctcc ctccaacccg cctgtggggc tacaacggct tatttccggg accgaccatt | 180 |
| gaggttaaaa gaaatgaaaa cgtatatgta aaatggatga ataaccttcc ttccacgcat | 240 |
| ttccttccga ttgatcacac cattcatcac agtgacagcc agcatgaaga gcccgaggta | 300 |
| aagactgttg ttcatttaca cggcggcgtc acgccagatg atagtgacgg gtatccggag | 360 |
| gcttggtttt ccaaagactt tgaacaaaca ggaccttatt tcaaaagaga ggtttatcat | 420 |
| tatccaaacc agcagcgcgg ggctatattg tggtatcacg atcacgccat ggcgctcacc | 480 |
| aggctaaatg tctatgccgg acttgtcggt gcatatatca ttcatgaccc aaaggaaaaa | 540 |
| cgcttaaaac tgccttcaga cgaatacgat gtgccgcttc ttatcacaga ccgcacgatc | 600 |
| aatgaggatg gttctttgtt ttatccgagc gcaccggaaa acccttctcc gtcactgcct | 660 |
| aatccttcaa tcgttccggc tttttgcgga gaaaccatac tcgtcaacgg gaaggtatgg | 720 |
| ccatacttgg aagtcgagcc aaggaaatac cgattccgtg tcatcaacgc ctccaataca | 780 |
| agaacctata acctgtcact cgataatggc ggagatttta ttcagattgg ttcagatgga | 840 |
| gggctcctgc cgcgatctgt taaactgaat tctttcagcc ttgcgcctgc tgaacgttac | 900 |
| gatatcatca ttgacttcac agcatatgaa ggagaatcga tcattttggc aaacagcgcg | 960 |
| ggctgcggcg gtgacgtcaa tcctgaaaca gatgcgaata tcatgcaatt cagagtcaca | 1020 |
| aaaccattgg cacaaaagac gaaagcagaa agccgaagta cctcgcctca tacccttcgg | 1080 |
| tacagcatga aagatacaaa catcagaacg ttaaaactgg caggcaccca ggacgaatac | 1140 |
| ggcagacccg tccttctgct taataacaaa cgctggcacg atcccgtcac agaaacacca | 1200 |
| aaagtcggca caactgaaat atggtccatt atcaaccgac acgcggaaca catcctgatc | 1260 |
| cacctgcatc tagtctcctt ccgtgtatta gaccggcggc cgtttgatat cgcccgttat | 1320 |
| caagaaagcg gggaattgtc ctatacagtc cgctgtcccg ccgccgcaag tgaaaagggc | 1380 |
| tggaaagaca ccattcaagc gcatgcaggt gaagtcctga gaatcgcggc gacattcggt | 1440 |
| ccgtacagcg gacgatacgt atggcattgc catattctag agcatgaaga ctatgacatg | 1500 |
| atgagaccga tggatataac tgatccccat aaataa | 1536 |

B. subtilis, CotB - (P07789)

| | |
|---|---|
| 148. SQ SEQUENCE 380 AA; 42971 MW; A42451945976CC79 CRC64; | |
| MSKRRMKYHS NNEISYYNFL HSMKDKIVTV YRGGPESKKG KLTAVKSDYI ALQAEKKIIY | |
| YQLEHVKSIT EDTNNSTTTI ETEEMLDADD FHSLIGHLIN QSVQFNQGGP ESKKGRLVWL | |
| GDDYAALNTN EDGVVYFNIH HIKSISKHEP DLKIEEQTPV GVLEADDLSE VFKSLTHKWV | |
| SINRGGPEAI EGILVDNADG HYTIVKNQEV LRIYPFHIKS ISLGPKGSYK KEDQKNEQNQ | |
| EDNNDKDSNS FISSKSYSSS KSSKRSLKSS DDQSSKSGRS SRSKSSSKSS KRSLKSSDYQ | |
| SSKSGRSSRS KSSSKSSKRS LKSSDYQSSK SSKRSPRSSD YQSSRSPGYS SSIKSSGKQK | |
| EDYSYETIVR TIDYHWKRKF | |
| 147. SQ Sequence 1143 BP; 441 A; 191 C; 204 G; 307 T; 0 other; 464288522 CRC32; | |
| atgagcaaga ggagaatgaa atatcattca aataatgaaa tatcgtatta taactttttg | 60 |
| cactcaatga aagataaaat tgttactgta tatcgtggag gtccggaatc taaaaaagga | 120 |
| aaattaacag ctgtaaaatc agattatata gctttacaag ctgaaaaaaa aataatttat | 180 |
| tatcagttgg agcatgtgaa aagtattact gaggatacca ataatagcac cacaacaatt | 240 |
| gagactgagg aaatgctcga tgctgatgat tttcatagct taatcggaca tttaataaac | 300 |
| caatcagttc aatttaacca agggggtccg gaatctaaaa aaggaagatt ggtctggctg | 360 |
| ggagatgatt acgctgcgtt aaacacaaat gaggatgggg tagtgtattt taatatccat | 420 |
| cacatcaaaa gtataagtaa acacgagcct gatttgaaaa tagaagagca gacgccagtt | 480 |
| ggagttttgg aagctgatga tttaagcgag gtttttaaga gtctgactca taaatgggtt | 540 |
| tcaattaatc gtggaggtcc ggaagccatt gagggtatcc ttgtagataa tgccgacggc | 600 |
| cattatacta tagtgaaaaa tcaagaggtg cttcgcatct atccttttca cataaaaagc | 660 |
| atcagcttag gtccaaaagg gtcgtacaaa aaagaggatc aaaaaaatga acaaaaccag | 720 |
| gaagacaata atgataagga cagcaattcg ttcatttctt caaaatcata tagctcatca | 780 |
| aaatcatcta aacgatcact aaaatcttca gatgatcaat catccaaatc tggtcgttcg | 840 |
| tcacgttcaa aaagttcttc aaaatcatct aaacgatcac taaaatcttc ggattatcaa | 900 |
| tcatccaaat ctggccgttc gtcacgttca aaaagttctt caaaatcatc taaacgatca | 960 |
| ttaaaatctt cagattatca atcatcaaaa tcatctaaac gatcaccaag atcttcagat | 1020 |
| tatcaatcat caagatcacc aggctattca agttcaataa aaagttcagg aaaacaaaag | 1080 |
| gaagattata gctatgaaac gattgtcaga acgatagact atcactggaa acgtaaattt | 1140 |
| taa | 1143 |

B. subtilis CotC - (P07790)

| | |
|---|---|
| 150. SQ SEQUENCE 66 AA; 8817 MW; 61739934006450AC CRC64; | |
| MGYYKKYKEE YYTVKKTYYK KYYEYDKKDY DCDYDKKYDD YDKKYYDHDK KDYDYVVEYK | |
| KHKKHY | |
| 149. SQ Sequence 201 BP; 101 A; 17 C; 30 G; 53 T; 0 other; 1456660706 CRC32; | |
| atgggttatt acaaaaaata caaagaagag tattatacgg tcaaaaaaac gtattataag | 60 |
| aagtattacg aatatgataa aaaagattat gactgtgatt acgacaaaaa atatgatgac | 120 |
| tatgataaaa aatattatga tcacgataaa aaagactatg attatgttgt agagtataaa | 180 |
| aagcataaaa aacactacta a | 201 |

B. subtilis CotD - (P07791)

| | |
|---|---|
| 152. SQ SEQUENCE 75 AA; 8840 MW; A5019889CA6CC0EA CRC64; | |
| MHHCRPHMMA PIVHPTHCCE HHTFSKTIVP HIHPQHTTNV NHQHFQHVHY FPHTFSNVDP | |
| ATHQHFQAGK PCCDY | |
| 151. SQ Sequence 228 BP; 65 A; 71 C; 36 G; 56 T; 0 other; 1875148613 CRC32; | |
| atgcatcact gcagaccgca tatgatggcg ccaattgtcc atcctactca ttgctgtgaa | 60 |
| caccatacgt tttcgaagac tatcgtgccg cacattcacc cacagcatac aacaaacgta | 120 |
| aaccaccagc attttcagca cgttcactac tttccacaca ctttctcaaa tgttgacccg | 180 |
| gctacgcatc agcattttca agcaggaaaa ccttgctgcg actactag | 228 |

B. subtilis CotE - (P14016)

| | |
|---|---|
| 154. SQ SEQUENCE 181 AA; 20977 MW; 6E9FBAE3E059BFC2 CRC64; | |
| MSEYREIITK AVVAKGRKFT QCTNTISPEK KPSSILGGWI INHKYDAEKI GKTVEIEGYY | |
| DINVWYSYAD NTKTEVVTER VKYVDVIKLR YRDNNYLDDE HEVIAKVLQQ PNCLEVTISP | |
| NGNKIVVQAE REFLAEVVGE TKVVVEVNPD WEEDDEEDWE DELDEELEDI NPEFLVGDPE | |
| E | |
| 153. SQ Sequence 546 BP; 196 A; 84 C; 144 G; 122 T; 0 other; 715049785 CRC32; | |
| atgtctgaat acagggaaat tattacgaag gcagtagtag cgaaaggccg aaaattcacc | 60 |
| caatgcacca acaccatctc gcctgagaaa aaaccgagca gcattttggg tggttggatt | 120 |
| attaaccaca agtatgacgc tgaaaaaatt ggaaaaacgg tagaaattga agggtattat | 180 |
| gatataaacg tatggtactc ttacgcggac aacacaaaga cagaggttgt cacagaacgg | 240 |
| gtaaaatatg tagatgtcat taaactcaga tacagagaca ataattactt agatgatgag | 300 |
| catgaagtga ttgccaaagt gcttcagcag ccaaactgcc ttgaagtgac catttcgccg | 360 |
| aatggaaata aaatcgttgt gcaggcagaa agagaatttt tggcggaagt ggtaggggaa | 420 |
| acaaaggtag ttgttgaggt caatcctgac tgggaagagg atgacgagga agattgggaa | 480 |
| gatgagcttg atgaagagct tgaagacatc aacccggagt ttttagtggg agatcctgaa | 540 |
| gaataa | 546 |

B. subtilis CotF - (P23261)

| | |
|---|---|
| 156. SQ SEQUENCE 160 AA; 18725 MW; F3F7869A26D56916 CRC64; | |
| MDERRTLAWH ETLEMHELVA FQSNGLIKLK KMIREVKDPQ LRQLYNVSIQ GVEQNLRELL | |
| PFFPQAPHRE DEEEERADNP FYSGDLLGFA KTSVRSYAIA ITETATPQLR NVLVKQLNAA | |
| IQLHAQVYRY MYQHGYYPSY NLSELLKNDV RNANRAISMK | |
| 155. SQ Sequence 483 BP; 160 A; 109 C; 100 G; 114 T; 0 other; 1161608513 CRC32; | |
| atggatgaac gcagaacatt ggcttggcat gaaacattag aaatgcacga gctggttgct | 60 |
| tttcaatcaa acggactcat taaactgaag aaaatgataa gagaagtaaa agaccctcag | 120 |
| ctcagacagc tttataacgt gtctattcag ggtgttgagc aaaatttgag agagcttctt | 180 |
| ccgttctttc cacaggctcc gcacagagag gatgaggaag aagaacgcgc agataaccca | 240 |
| ttttacagcg gtgacctgct cggttttgcc aaaacatctg tccgcagcta tgccatcgca | 300 |
| attacagaaa cagcaacacc tcaattaaga aacgtactgg tcaaacagct gaatgctgcc | 360 |
| atccagctgc acgcccaagt ttatcgatac atgtatcagc atggatatta tccgtcttac | 420 |
| aacctttctg aactgttgaa aaacgatgtc agaaacgcca acagagccat ttcaatgaaa | 480 |
| taa | 483 |

B. subtilis CotG - (P39801)

| | |
|---|---|
| 158. SQ SEQUENCE 195 AA; 23957 MW; FDAF2D58595D7082 CRC64; | |
| MGHYSHSDIE EAVKSAKKEG LKDYLYQEPH GKKRSHKKSH RTHKKSRSHK KSYCSHKKSR | |
| SHKKSFCSHK KSRSHKKSYC SHKKSRSHKK SYRSHKKSRS YKKSYRSYKK SRSYKKSCRS | |
| YKKSRSYKKS YCSHKKKSRS YKKSCRTHKK SYRSHKKYYK KPHHHCDDYK RHDDYDSKKE | |
| YWKDGNCWVV KKKYK | |
| 157. SQ Sequence 588 BP; 246 A; 141 C; 80 G; 121 T; 0 other; 1703511360 CRC32; | |
| ttgggccact attcccattc tgacatcgaa gaagcggtga aatccgcaaa aaaagaaggt | 60 |
| ttaaaggatt atttatacca agagcctcat ggaaaaaaac gcagtcataa aaagtcgcac | 120 |
| cgcactcaca aaaaatctcg cagccataaa aaatcatact gctctcacaa aaaatctcgc | 180 |
| agtcacaaaa aatcattctg ttctcacaaa aaatctcgca gccacaaaaa atcatactgc | 240 |
| tctcacaaga aatctcgcag ccacaaaaaa tcgtaccgtt ctcacaaaaa atctcgcagc | 300 |
| tataaaaaat cttaccgttc ttacaaaaaa tctcgtagct ataaaaaatc ttgccgttct | 360 |
| tacaaaaaat ctcgcagcta caaaaagtct tactgttctc acaagaaaaa atctcgcagc | 420 |
| tataagaagt catgccgcac acacaaaaaa tcttatcgtt cccataagaa atactacaaa | 480 |
| aaaccgcacc accactgcga cgactacaaa agacacgatg attatgacag caaaaaagaa | 540 |
| tactggaaag acggcaattg ctgggtagtc aaaaagaaat acaaataa | 588 |

B. subtilis CotH - (Q45535)

| | |
|---|---|
| 160. SQ SEQUENCE 362 AA; 42813 MW; 79C5E30BA01B3311 CRC64; | |
| MKNQSNLPLY QLFVHPKDLR ELKKDIWDDD PVPAVMKVNQ KRLDIDIAYR GSHIRDFKKK | |
| SYHISFYQPK TFRGAREIHL NAEYKDPSLM RNKLSLDFFS ELGTLSPKAE FAFVKMNGKN | |
| EGVYLELESV DEYYLAKRKL ADGAIFYAVD DDANFSLMSD LERETKTSLE LGYEKKTGTE | |
| EDDFYLQDMI FKINTVPKAQ FKSEVTKHVD VDKYLRWLAG IVFTSNYDGF VHNYALYRSG | |
| ETGLFEVIPW DYDATWGRDI HGERMAADYV RIQGFNTLTA RILDESEFRK SYKRLLEKTL | |
| QSLFTIEYME PKIMAMYERI RPFVLMDPYK KNDIERFDRE PDVICEYIKN RSQYLKDHLS | |
| IL | |
| 159. SQ Sequence 1089 BP; 340 A; 184 C; 260 G; 305 T; 0 other; 437598408 CRC32; | |
| atgaagaatc aatccaattt accgctttat cagctgtttg ttcatccaaa agacttgcgt | 60 |
| gaattaaaaa aggatatatg ggacgatgat ccggtgccag ctgtgatgaa ggtaaatcaa | 120 |
| aaaaggctgg atattgatat cgcttatcgg ggatcacata tcagagactt caaaaagaag | 180 |
| tcataccata tttcctttta tcagccgaaa acattccgcg gcggccgaga gattcactta | 240 |
| aatgcggagt ataaagaccc ttccttgatg agaaacaaat tgtctctgga ttttttctcg | 300 |
| gagctaggga cactgtctcc aaaggcagag tttgcgtttg taaagatgaa tgggaagaat | 360 |
| gaaggggttt atcttgaact tgaatccgta gatgaatatt atttggcgaa aaggaagctg | 420 |
| gctgatggcg cgatttttta tgcggtggat gatgatgcca acttttctct gatgagcgat | 480 |
| ttagaaaggg aaacgaaaac atcgctggag cttggatatg aaaagaaaac agggactgag | 540 |
| gaagatgatt tttatttaca agatatgatt tttaaaatta atacggtccc taaagctcag | 600 |
| tttaagtcag aagtgacaaa acacgtggat gtcgataagt atttgcgctg gcttgctggt | 660 |
| attgtattca cctcgaacta tgacgggttt gtccacaact acgcattata cagaagcggg | 720 |
| gaaaccggat tatttgaggt gattccttgg gattatgatg cgacttgggg cagggatatc | 780 |
| catggagagc ggatggctgc cgattatgta agaattcaag gatttaatac actaaccgcc | 840 |
| cggatattgg atgaatccga gtttcgcaag tcctacaagc gcctgttaga aaaaacgctc | 900 |
| caatctcttt ttacaataga atatatggaa ccgaaaatca tggcgatgta tgaacggatt | 960 |
| aggccgtttg tcctcatgga cccgtataaa aagaatgata ttgagcgttt tgaccgtgag | 1020 |
| ccggatgtga tctgcgagta tattaaaaac cgttcacaat acctcaaaga tcatttaagt | 1080 |
| attttatga | 1089 |

B. subtilis CotJA - (Q45536)

| | |
|---|---|
| 162. SQ SEQUENCE 82 AA; 9739 MW; 405E8CDCEA23A3EF CRC64; | |
| MKDMQPFTPV KSYTPFHSRF DPCPPIGKKY YRTPPNLYMT FQPEHMEQFS PMEALRKGTL | |
| WKDLYDFYEN PYRGGDAHGK KG | |
| 161. SQ Sequence 249 BP; 69 A; 59 C; 58 G; 63 T; 0 other; 3568063845 CRC32; | |
| atgaaggata tgcagccgtt tacgcctgtc aaatcatata cgccctttca cagccgtttt | 60 |
| gatccctgtc cgcccatagg gaagaaatat tacagaacgc cccctaacct ttatatgacc | 120 |
| tttcagcctg agcacatgga gcagttttcg ccgatggagg ctttgaggaa aggcaccctt | 180 |
| tggaaggatc tctatgattt ttatgaaaac ccttatcgag ggggagacgc acatggcaaa | 240 |
| aaaggttga | 249 |

B. subtilis CotJB - (Q45537)

| | |
|---|---|
| 164. SQ SEQUENCE 100 AA; 11752 MW; 0392E266020495E0 CRC64; | |
| MIFMKTLIEG ETHMAKKVDA EYYRQLEQIQ AADFVLVELS LYLNTHPHDE DALKQFNQYS | |
| GYSRHLKRQF ESSYGPLLQF GNSPAGKDWD WGKGPWPWQV | |
| 163. SQ Sequence 303 BP; 89 A; 61 C; 76 G; 77 T; 0 other; 3529835581 CRC32; | |
| atgattttta tgaaaaccct tatcgagggg gagacgcaca tggcaaaaaa ggttgacgcc | 60 |
| gaatattatc gtcagctaga gcaaatacag gctgctgatt ttgtgcttgt tgagctgagt | 120 |
| ctttatttaa atacacatcc tcatgatgaa gatgcgttga agcaattcaa tcaatattcc | 180 |
| ggctattcaa ggcacttaaa aagacagttc gaatcctctt acggaccgct tctgcagttc | 240 |
| ggcaacagcc ccgcgggcaa ggattgggat tggggaaaag ggccatggcc gtggcaagta | 300 |
| taa | 303 |

B. subtilis CotJC - (Q25538)

| | |
|---|---|
| 166. SQ SEQUENCE 189 AA; 21696 MW; 8EB66EFABE66BC65 CRC64; | |
| MWVYEKKLQY PVKVSTCNPT LAKYLIEQYG GADGELAAAL RYLNQRYTIP DKVIGLLTDI | |
| GTEEFAHLEM IATMVYKLTK DATPEQLREA GLGDHYVNHD SALFYHNAAG VPFTASYIQA | |
| KGDPIADLYE DIAAEEKARA TYQWLIDISD DPDLNDSLRF LREREIVHSM RFREAVEILK | |
| EERDKKKIF | |
| 165. SQ Sequence 570 BP; 153 A; 119 C; 159 G; 139 T; 0 other; 2983140167 CRC32; | |
| atgtgggtgt atgaaaagaa gctgcaatac cctgtcaagg tcagtacgtg caacccgacg | 60 |
| ctggcgaagt atttgattga gcagtatggc ggagcggacg gcgagctggc cgcggctctc | 120 |
| cggtatttga accagcgtta tacgattcct gataaggtca tcggcctttt aacagatatc | 180 |
| ggcacggagg agtttgccca tttggaaatg attgcgacca tggtctataa gttaacgaaa | 240 |
| gacgccacac cggagcagct gcgtgaagct gggcttggcg atcattacgt caatcacgac | 300 |
| agcgcgcttt tttatcataa tgcggcgggc gtgccgttta ccgcgagcta tatccaagcg | 360 |
| aagggcgatc cgattgccga tttatacgaa gatattgccg cagaagaaaa ggcgcgggcg | 420 |
| acgtatcaat ggctgattga tatttcggat gatcctgatt taaacgattc cctgcgtttt | 480 |
| ttgcgtgagc gagaaatcgt acactctatg cgcttcagag aagcggttga aattttaaaa | 540 |
| gaagaacggg ataaaaagaa gattttctaa | 570 |

B. subtilis CotM - (Q45058)

| | |
|---|---|
| 168. SQ SEQUENCE 130 AA; 15222 MW; 6EB9D44CBD0126A7 CRC64; | |
| MWRNASMNHS KRNDANDFDS MDEWLRQFFE DPFAWYDETL PIDLYETSQQ YIIEADLTFL | |
| QPTQVTVTLS GCEFILTVKS SGQTFEKQMM LPFYFNDKNI QVECENQILT VAVNKETEDG | |
| SSFSLQFPLS | |
| 167. SQ Sequence 375 BP; 122 A; 77 C; 63 G; 113 T; 0 other; 2212745149 CRC32; | |
| atgaaccatt caaaacgcaa cgatgcgaat gatttcgata gtatggatga atggcttcgg | 60 |
| caattttttg aagacccctt cgcctggtac gacgaaacat tgcctattga tttatatgaa | 120 |
| acaagtcagc agtatattat agaagcggat ctgacttttt tacagcctac acaagtaaca | 180 |
| gttacccttt ctggatgcga gttcatctta actgtcaaat cgtcaggaca gacttttgaa | 240 |
| aaacaaatga tgcttccttt ttattttaat gacaaaaaca ttcaagtcga atgcgaaaat | 300 |
| caaatactca cagtcgccgt caataaagaa acagaagatg gctcttcttt ttctcttcaa | 360 |
| tttcctctca gctaa | 375 |

B. subtilis CotR - (Unavailable)
B. subtilis CotSA - (P46915)

| | |
|---|---|
| 170. SQ SEQUENCE 377 AA; 42912 MW; 1F978E1B79F9E660 CRC64; | |
| MKIALIATEK LPVPSVRGGA IQIYLEAVAP LIAKKHEVTV FSIKDPNLAD REKVDGVHYV | |
| HLDEDRYEEA VGAELKKSRF DLVHVCNRPS WVPKLKKQAP DAVFILSVHN EMFAYDKISQ | |
| AEGEICIDSV AQIVTVSDYI GQTITSRFPS ARSKTKTVYS GVDLKTYHPR WTNEGQRARE | |
| EMRSELGLHG KKIVLFVGRL SKVKGPHILL QALPDIIEEH PDVMMVFIGS KWFGDNELNN | |
| YVKHLHTLGA MQKDHVTFIQ FVKPKDIPRL YTMSDVFVCS SQWQEPLARV HYEAMAAGLP | |
| IITSNRGGNP EVIEEGKNGY IIHDFENPKQ YAERINDLLS SSEKRERLGK YSRREAESNF | |
| GWQRVAENLL SVYEKNR | |
| 169. SQ Sequence 1134 BP; 332 A; 231 C; 294 G; 277 T; 0 other; 2322560928 CRC32; | |
| atgaaaatag cactgatcgc cacagagaag cttcctgtcc catcggttcg aggaggcgcc | 60 |
| attcaaatct acctcgaagc ggttgcccct ttaattgcaa aaaaacatga ggtgactgtg | 120 |
| ttttctatta aagatccgaa tctcgctgat agagagaagg tagacggtgt ccattatgtg | 180 |
| catttggatg aagaccgtta tgaagaagcc gttggagcag agctgaaaaa gagccgtttt | 240 |
| gatcttgtgc atgtttgtaa tcgcccaagc tgggttccga aattgaagaa acaggcgccg | 300 |
| gatgctgttt ttattttaag cgttcacaat gaaatgttcg cttacgataa aatcagccag | 360 |
| gcggaaggcg agatttgcat cgactccgta gcgcagattg ttacggtcag cgattatatc | 420 |
| ggacagacga tcacaagccg ttttccgtca gcacgatcaa aaacaaaaac ggtgtattct | 480 |
| ggtgtggatt taaaaacgta ccaccctcgc tggacgaatg aagggcagcg agctcgcgaa | 540 |
| gagatgcgaa gcgagctggg gcttcacggc aaaaaaatcg tcttgtttgt cggccggctt | 600 |
| agcaaagtca aaggcccgca catattattg caggctttgc cggacatcat tgaggagcac | 660 |
| cccgatgtca tgatggtgtt tatcgggtca aaatggttcg gagataatga attaaataac | 720 |
| tatgtcaaac atcttcatac ccttggtgcg atgcaaaagg atcatgtcac atttattcaa | 780 |
| tttgtgaagc caaaggacat tccgcgcctt tataccatgt cagatgtatt tgtatgctct | 840 |
| tcgcaatggc aggagccttt agcaagggtg cattatgaag cgatggctgc gggacttcct | 900 |
| attattacaa gcaatcgggg aggcaatcca gaggtcatag aggaagggaa aaacggctac | 960 |
| atcattcatg actttgaaaa tcctaaacaa tatgccgaac gtatcaatga tttgctgagc | 1020 |
| agctcggaaa agcgggaacg gcttgggaaa tacagccgcc gtgaggcaga aagcaatttt | 1080 |
| ggctggcaga gggtggctga aaatctgctc agcgtctatg aaaagaacag atag | 1134 |

B. subtilis CotS - (P46914)

| | |
|---|---|
| 172. SQ SEQUENCE 351 AA; 41084 MW; 7F6DEF041417B26D CRC64; | |
| MYQKEHEEQI VSEILSYYPF HIDHVALKSN KSGRKIWEVE TDHGPKLLKE AQMKPERMLF | |
| ITQAHAHLQE KGLPIAPIHQ TKNGGSCLGT DQVSYSLYDK VTGKEMIYYD AEQMKKVMSF | |
| AGHFHHASKG YVCTDESKKR SRLGKWHKLY RWKLQELEGN MQIAASYPDD VFSQTFLKHA | |
| DKMLARGKEA LRALDDSEYE TWTKETLEHG GFCFQDFTLA RLTEIEGEPF LKELHSITYD | |
| LPSRDLRILL NKVMVKLSVW DTDFMVALLA AYDAVYPLTE KQYEVLWIDL AFPHLFCAIG | |
| HKYYLKQKKT WSDEKYNWAL QNMISVEESK DSFLDKLPEL YKKIKAYREA N | |
| 171. SQ Sequence 1056 BP; 338 A; 198 C; 257 G; 263 T; 0 other; 1829510316 CRC32; | |
| gtgtaccaaa aagagcatga agaacagatt gtgtccgaaa ttctcagtta ttatccgttt | 60 |
| catatcgacc atgtggcgct gaaatcgaac aaaagcgggc gcaaaatctg ggaagtcgaa | 120 |
| actgatcatg gcccaaagct gctaaaagaa gcgcaaatga aaccggagcg gatgcttttt | 180 |
| atcactcagg cacacgccca tttacaagag aaagggctgc cgatagcgcc gattcatcaa | 240 |
| acaaaaaatg gcggtagctg cttgggcacg gatcaggttt cttacagttt atatgacaaa | 300 |
| gtgacaggaa aagaaatgat ttactatgat gcagagcaaa tgaaaaaagt catgtcattt | 360 |
| gccggccatt ttcatcatgc ctcaaaagga tatgtttgca cagatgaaag caagaagaga | 420 |
| agcaggctgg gaaaatggca caaattgtac cgttggaagc tgcaggaact tgaagggaat | 480 |
| atgcagatcg cagcatccta tcctgatgac gtattttcgc aaactttctt aaaacatgct | 540 |
| gataaaatgc tggcaagagg aaaagaagct ctcagagcgc ttgatgactc agaatacgaa | 600 |
| acctggacaa aagagacact cgagcatggc ggattctgtt ttcaggattt tacattggca | 660 |
| cgtttgactg agatcgaagg ggagcctttt ttaaaggagc ttcactcgat tacctacgat | 720 |
| ttgccgtcaa gagaccttcg tattctgctg aataaagtga tggttaagct ttctgtatgg | 780 |
| gatactgatt tcatggttgc actgcttgcg gcctacgacg cagtgtatcc gctcacagaa | 840 |
| aaacagtacg aggtactttg gattgatctc gcgtttccgc atttgttctg tgcaatcggg | 900 |
| cacaaatatt atttgaagca aaagaaaacg tggtcagatg agaagtataa ctgggcgctg | 960 |
| caaaacatga tttctgttga agaatctaaa gattcgtttt tggataaact gccggaactg | 1020 |
| tataaaaaga taaaagcgta tcgggaggcg aattga | 1056 |

B. subtilis CotT - (P11863)

| | |
|---|---|
| 174. SQ SEQUENCE 82 AA; 10131 MW; E2E9C3B9E0B7FCCE CRC64; | |
| MDYPLNEQSF EQITPYDERQ PYYYPRPRPP FYPPYYYPRP YYPFYPFYPR PPYYYPRPRP | |
| PYYPWYGYGG GYGGGYGGGY GY | |
| 173. SQ Sequence 324 BP; 86 A; 79 C; 69 G; 90 T; 0 other; 2507283673 CRC32; | |
| atgaatgtac atacacccaa cttaagcatc aggaatatgg taaaaggaat aaaaaaagct | 60 |
| agggaggttt tcctcttgga ttaccctttg aatgaacagt catttgaaca aattacccct | 120 |
| tatgatgaaa gacagcctta ttattatccg cgtccgagac cgccatttta tccgccttat | 180 |
| tattatccaa gaccgtatta tccgttctac ccgttttatc cgcgcccgcc ttattactac | 240 |
| ccgcgcccgc gaccgcctta ctacccttgg tacggttacg gcggaggtta tggcggagga | 300 |
| tatgggggag gttacggtta ctag | 324 |

B. subtilis CotV - (Q08309)

| | |
|---|---|
| 176. SQ SEQUENCE 128 AA; 14227 MW; E72A503E516B4DED CRC64; | |
| MSFEEKVESL HPAIFEQLSS EFEQQIEVID CENITIDTSH ITAALSIQAF VTTMIIVATQ | |
| LVIADEDLAD AVASEILILD SSQIKKRTII KIINSRNIKI TLSADEIITF VQILLQVLNS | |
| ILSELDVL | |
| 175. SQ Sequence 387 BP; 127 A; 87 C; 68 G; 105 T; 0 other; 586070402 CRC32; | |
| atgtcatttg aagaaaaagt cgaatccctg caccctgcaa tatttgagca attatcaagc | 60 |
| gaattcgaac agcagatcga agtgattgat tgcgaaaata tcacaattga cacgtcacat | 120 |
| ataacagctg ccctttctat acaagccttt gtgacaacca tgattatcgt ggcgactcag | 180 |
| ctcgtcatcg ccgacgagga tttggctgac gcagtggcaa gtgaaattct tattctcgat | 240 |
| agctcccaaa tcaaaaaaag aaccatcatt aaaattatca acagccgcaa catcaaaatt | 300 |
| actttgtctg ccgacgagat aataaccttt gtacaaatct tgcttcaggt gttaaacagc | 360 |
| attcttagtg aacttgacgt cctttaa | 387 |

B. subtilis CotW - (Q08310)

| | |
|---|---|
| 178. SQ SEQUENCE 105 AA; 12336 MW; 2044C2885C63F7D4 CRC64; | |
| MSDNDKFKEE LAKLPEVDPM TKMLVQNIFS KHGVTKDKMK KVSDEEKEML LNLVKDLQAK | |
| SQALIENQKK KKEEAAAQEQ KNTKPLSRRE QLIEQIRQRR KNDNN | |
| 177. SQ Sequence 318 BP; 152 A; 55 C; 59 G; 52 T; 0 other; 3742021663 CRC32; | |
| atgtcagata acgataaatt caaagaagag cttgcaaagc ttccagaagt tgatccaatg | 60 |
| acgaaaatgc tggtccaaaa tatattttct aaacatgggg tcacaaaaga caaaatgaaa | 120 |
| aaagtatcag acgaagaaaa agaaatgctc ttaaatcttg taaaagactt acaagctaaa | 180 |
| tcacaagcgc taatagaaaa ccaaaagaag aaaaaagaag aagcagccgc acaagagcaa | 240 |
| aagaacacaa aaccgttaag ccgcagagag cagctcattg aacagatcag acaaagacgg | 300 |
| aaaaacgata acaattag | 318 |

B. subtilis CotY - (Q08311)

| | |
|---|---|
| 180. SQ SEQUENCE 162 AA; 17884 MW; E468C15B22A9E99B CRC64; | |
| MSCGKTHGRH ENCVCDAVEK ILAEQEAVEE QCPTGCYTNL LNPTIAGKDT IPFLVFDKKG | |
| GLFSTFGNVG GFVDDMQCFE SIFFRVEKLC DCCATLSILR PVDVKGDTLS VCHPCDPDFF | |
| GLEKTDFCIE VDLGCFCAIQ CLSPELVDRT SPHKDKKHHH NG | |
| 179. SQ Sequence 489 BP; 138 A; 105 C; 117 G; 129 T; 0 other; 3120539689 CRC32; | |
| atgagctgcg gaaaaaccca tggccggcat gagaactgtg tatgcgatgc agtggaaaag | 60 |
| attttagcag agcaggaggc agttgaagaa cagtgtccga ctggctgcta taccaacctt | 120 |
| ttaaacccta cgattgctgg aaaagacaca attccgtttc tcgtttttga taaaaaaggc | 180 |
| ggattgttct ccacattcgg aaacgtaggg ggatttgtgg atgatatgca atgctttgaa | 240 |
| tccattttct tccgcgtcga aaaattatgc gattgctgtg caacactgtc tattttacgc | 300 |
| ccggtcgatg tcaaaggcga taccttaagt gtttgccacc cttgcgaccc ggatttcttc | 360 |
| gggctagaaa aaacagattt ctgcattgaa gtggatctcg gatgcttctg cgcgattcag | 420 |
| tgcctgtcac cagagctagt tgacagaaca tcgcctcaca aagataaaaa gcatcatcac | 480 |
| aatggataa | 489 |

B. subtilis CotZ - (Q08312)

| | |
|---|---|
| 182. SQ SEQUENCE 148 AA; 16534 MW; 90429FFB0550896E CRC64; | |
| MSQKTSSCVR EAVENIEDLQ NAVEEDCPTG CHSKLLSVSH SLGDTVPFAI FTSKSTPLVA | |
| FGNVGELDNG PCFNTVFFRV ERVHGSCATL SLLIAFDEHK HILDFTDKDT VCEVFRLEKT | |
| NYCIEVDLDC FCAINCLNPR LINRTHHH | |
| 181. SQ Sequence 447 BP; 138 A; 99 C; 90 G; 120 T; 0 other; 2177378295 CRC32; | |
| atgagccaga aaacatcaag ctgcgtgcgt gaagctgtag aaaatattga agatctgcaa | 60 |
| aacgctgttg aagaagactg cccgaccggc tgccactcta agcttttatc tgtaagccat | 120 |
| tcgttaggcg acacagtgcc ttttgcaata tttacatcaa aatcaacgcc attagtcgcc | 180 |
| ttcggaaatg tcggcgaact cgataacggc ccttgcttta atacagtatt tttcagggtc | 240 |
| gaaagagtgc atggaagctg tgcaacactg tcattattaa tcgcatttga cgaacacaaa | 300 |
| cacattttgg acttcaccga taaagatacg gtgtgtgaag tgttccgact cgaaaaaacg | 360 |
| aactactgta ttgaagttga cttagactgc ttctgcgcaa tcaactgctt aaatcctcga | 420 |
| ttaatcaatc gtacacatca tcattaa | 447 |

B. subtilis GerPA - (O06721)

| | |
|---|---|
| 184. SQ SEQUENCE 73 AA; 7541 MW; 8D9EE207B2FC4864 CRC64; | |
| MPAIVGAFKI NAIGTSGVVH IGDCITISPQ AQVRTFAGAG SFNTGDSLKV MNYQNATNVY | |
| DNDAVDQPIV ANA | |
| 183. SQ Sequence 222 BP; 55 A; 49 C; 63 G; 55 T; 0 other; 290912503 CRC32; | |
| atgccggcca ttgtcggagc gtttaaaatt aatgcgattg gtacgagcgg agtcgttcac | 60 |
| atcggggact gcattacgat ttctcctcag gctcaggtca gaacgtttgc cggtgctggc | 120 |
| agctttaata ccggcgacag cctcaaggtg atgaattatc aaaacgcgac gaatgtgtat | 180 |
| gacaatgatg cggttgatca gccgatcgtg gccaatgcgt aa | 222 |

B. subtilis GerPB - (O06720)

| | |
|---|---|
| 186. SQ SEQUENCE 77 AA; 8280 MW; 5A8A8E71836ADC34 CRC64; | |
| MNFYINQTIQ INYLRLESIS NSSILQIGSA GSIKSLSNLY NTGSYVEPAP EVSGSGQPLQ | |
| LQEPDTGSLV PLQPPGR | |
| 185. SQ Sequence 234 BP; 65 A; 67 C; 48 G; 54 T; 0 other; 851474871 CRC32; | |
| atgaacttct atattaatca aaccattcaa atcaactatc tccggctgga atcaatcagc | 60 |
| aactcctcca ttctgcaaat cgggagcgcc ggatcaatca agtcactgtc aaatttgtat | 120 |
| aatacaggaa gctatgtaga gccggcacca gaagtttctg gctcagggca accgctccag | 180 |
| ctgcaggagc ccgacacagg ttcattggtc ccgctccagc ctcctggccg ttaa | 234 |

B. subtilis GerPC - (O06719)

| | |
|---|---|
| 188. SQ SEQUENCE 205 AA; 24240 MW; C5060B92C8CB0021 CRC64; | |
| MYDQSVSSYL QNLNSFVQQQ AIHIQQLERQ LKEIQTEMNT MKQRPATTIE RVEYKFDQLK | |
| IERLDGTLNI GLNPTDPNSV QNFDVSQSTP QIGMMQQEES AQLMQQIRQN VDMYLTEEIP | |
| DILEQLENQY DSRLDDTNRH HVIEDIRKQM DSRIHYYMSH IKKEENTPPA QYAEHIAEHV | |
| KRDVIRAVEH FLEHIPSEMK GDEQA | |
| 189. SQ Sequence 618 BP; 211 A; 137 C; 135 G; 135 T; 0 other; 3299727878 CRC32; | |
| atgtatgatc aatctgtttc ctcttacctg caaaacttga attccttcgt tcagcagcag | 60 |
| gcgattcaca ttcagcagct cgaacgtcag ctgaaagaga ttcaaactga aatgaatacg | 120 |
| atgaaacagc ggccggccac taccattgag cgtgtggagt ataaatttga tcagctgaaa | 180 |
| atcgaaaggc tcgacgggac tttgaatatc ggtttaaatc cgactgaccc gaacagcgtc | 240 |
| caaaactttg acgtcagcca gtcgacaccg caaatcggga tgatgcagca ggaagagagc | 300 |
| gctcagctca tgcagcagat ccgccagaat gtcgacatgt acttaaccga ggaaatccca | 360 |
| gatattttgg aacagcttga aaatcaatat gacagcagac ttgacgatac aaacagacat | 420 |
| catgttattg aagacatcag aaaacaaatg gacagcagga ttcactatta tatgtcccat | 480 |
| atcaaaaaag aagaaaatac accgcctgca caatatgcag aacatatcgc tgagcatgtg | 540 |
| aagcgtgatg tcatccgcgc tgtagaacac tttctggagc atattccatc agaaatgaaa | 600 |
| ggagatgagc aagcatga | 618 |

B. subtilis GerPD - (O06718)

| | |
|---|---|
| 190. SQ SEQUENCE 58 AA; 6269 MW; 8A5141328C155920 CRC64; | |
| MIFTVINRSL EVGDIRMNGV SSSSVFHIGD TESIYLSSIF DTPPESLIIG PFAPLAPE | |
| 189. SQ Sequence 177 BP; 38 A; 46 C; 37 G; 56 T; 0 other; 1494235746 CRC32; | |
| atgatcttta cagtcatcaa ccgcagcttg gaagtcgggg atattcggat gaacggtgtg | 60 |
| tccagttcct ccgttttcca catcggagac actgaatcca tctacctgtc ttctattttt | 120 |
| gatacaccgc ctgaatctct tattattggg ccgtttgctc cgcttgcgcc agaataa | 177 |

B. subtilis GerPE - (O06717)

| | |
|---|---|
| 192. SQ SEQUENCE 133 AA; 14814 MW; EAB9E097F2FA202D CRC64; | |
| MLKRISRIRL VKFNSLGIAS VFQVGDTNEI DMSVKVFAVQ RSLSTFYHNE GSFNKKEYQI | |
| FQQQAVKPLP ETGVQSAFCH EVPAIYVRSI KIQGVSASSV LHAGSASLIR GDARLKHIRQ | |
| IQSPRSQSPA KNI | |
| 191. SQ Sequence 402 BP; 110 A; 96 C; 89 G; 107 T; 0 other; 1911633807 CRC32; | |
| atgcttaaac gcatatcgcg catcagacta gttaagttta attctctcgg gatcgcaagt | 60 |
| gtgtttcaag ttggcgacac aaatgaaatc gatatgagtg taaaagtatt tgctgtgcag | 120 |
| cgttctctgt ccacgtttta ccataatgaa ggctcattta acaaaaagga gtatcagatc | 180 |
| tttcagcagc aggccgtgaa gccgctcccc gaaacaggtg tacaaagcgc gttttgccac | 240 |
| gaggtgccgg ctatttatgt tcgcagcatc aaaattcaag gggtctcagc ctcttctgtt | 300 |
| ttacatgccg gatcagcttc gcttattcgc ggtgatgcga gactcaaaca tatcagacag | 360 |
| attcagtctc cgcgctcaca atcgcccgcc aagaacatat aa | 402 |

B. subtilis GerPF - (O06716)

| | |
|---|---|
| 194. SQ SEQUENCE 72 AA; 7248 MW; BAA1C310EB022486 CRC64; | |
| MPAIVGPIAI NSISGGVVNF GDSFYLSPKS SSKSALGSGA GNTGDFLLLN NAVNATNYID | |
| PDVNDQDMVG NG | |
| 193. SQ Sequence 228 BP; 63 A; 49 C; 52 G; 64 T; 0 other; 3534675991 CRC32; | |
| gtgtcgttta tgccagcaat tgtcgggcct atagctatca attccatatc gggcggagtc | 60 |
| gtaaactttg gtgattcctt ttacctttct ccgaaaagct cttcaaaatc tgcgctcggt | 120 |
| tcgggagcag gaaacacggg agatttcctt ctattaaata atgcagtcaa cgcgacaaat | 180 |
| tatatagacc ccgatgtcaa cgatcaggat atggttggaa acggataa | 228 |

B. subtilis YaaH - (P37531)

| | |
|---|---|
| 196. SQ SEQUENCE 427 AA; 48637 MW; 77FEF6AB327379A3 CRC64; | |
| MVKQGDTLSA IASQYRTTTN DITETNEIPN PDSLVVGQTI VIPIAGQFYD VKRGDTLTSI | |
| ARQFNTTAAE LARVNRIQLN TVLQIGFRLY IPPAPKRDIE SNAYLEPRGN QVSENLQQAA | |
| REASPYLTYL GAFSFQAQRN GTLVAPPLTN LRSITESQNT TLMMIITNLE NQAFSDELGR | |
| ILLNDETVKR RLLNEIVENA RRYGFRDIHF DFEYLRPQDR EAYNQFLREA RDLFHREGLE | |
| ISTALAPKTS ATQQGRWYEA HDYRAHGEIV DFVVLMTYEW GYSGGPPQAV SPIGPVRDVI | |
| EYALTEMPAN KIVMGQNLYG YDWTLPYTAG GTPARAVSPQ QAIVIADQNN ASIQYDQTAQ | |
| APFFRYTDAE NRRHEVWFED ARSIQAKFNL IKELNLRGIS YWKLGLSFPQ NWLLLSDQFN | |
| VVKKTFR | |
| 195. SQ Sequence 1284 BP; 385 A; 305 C; 285 G; 309 T; 0 other; 2121106037 CRC32; | |
| gtggtaaaac aaggcgacac tctttctgct atcgcttcac aatacagaac aaccacaaat | 60 |
| gacatcactg aaacgaatga aataccgaat cccgacagcc ttgttgtcgg acaaaccatt | 120 |
| gtcattccaa tagctggcca gttctatgat gtgaagcgag gtgataccct gacatccatc | 180 |
| gcccggcagt tcaatacaac agcagccgag ctcgcaaggg ttaaccgcat ccagttaaat | 240 |
| accgtgcttc agattggttt ccgtttatac atccctccag ctcctaaacg agacatcgaa | 300 |
| tcaaatgctt atttggagcc ccgaggaaat caagtcagcg aaaatctcca gcaggcggcc | 360 |
| agagaagcgt cgccctactt aacttacctt ggcgcattca gcttccaggc acagcggaac | 420 |
| ggaaccttag tcgcaccgcc tttaacgaat ttaaggagca ttacagaaag tcaaaataca | 480 |
| acattgatga tgattataac gaacctagaa aaccaggcat tcagcgatga acttggccgg | 540 |
| atccttttga acgacgaaac tgtaaaaaga cggcttctaa atgaaatagt cgagaatgcc | 600 |
| agaagatatg gcttccgtga cattcatttc gactttgaat atttgcggcc ccaggataga | 660 |
| gaggcctata atcaattcct ccgcgaagca agggatcttt tccatcgaga gggcttagaa | 720 |
| atttctacgg ctcttgctcc taaaacaagt gcaacacagc agggcaggtg gtatgaagct | 780 |
| catgattaca gggcacatgg cgaaattgtc gactttgttg ttctcatgac atatgaatgg | 840 |
| ggctatagcg gcggaccgcc tcaagcggtt tctccaattg gacctgtccg tgatgtcata | 900 |
| gaatatgctt tgactgaaat gcctgcgaac aaaattgtca tgggccagaa tttatatgga | 960 |
| tatgactgga cgctgccata tacagcaggg ggaactccag caagagcagt aagccctcag | 1020 |
| caagccattg tcatagctga tcagaacaat gcttccattc agtatgacca aaccgctcaa | 1080 |
| gctcctttct tccgctatac tgatgcagaa aacagaaggc acgaggtatg gttcgaggat | 1140 |
| gcccgctcga ttcaagcaaa attcaatctg attaaagagc tgaatttaag aggcatcagc | 1200 |
| tattggaagc tgggtctttc ctttccacaa aactggctgc tgctgtctga tcaatttaat | 1260 |
| gttgtcaaaa agacgtttcg ataa | 1284 |

B. subtilis YabG - (P37548)

| | |
|---|---|
| 198. SQ SEQUENCE 290 AA; 33318 MW; B60A5B9F9D3209BB CRC64; | |
| MQFQIGDMVA RKSYQMDVLF RIIGIEQTSK GNSIAILHGD EVRLIADSDF SDLVAVKKDE | |
| QMMRKKKDES RMNESLELLR QDYKLLREKQ EYYATSQYQH QEHYFHMPGK VLHLDGDEAY | |
| LKKCLNVYKK IGVPVYGIHC HEKKMSASIE VLLDKYRPDI LVITGHDAYS KQKGGIDDLN | |
| AYRHSKHFVE TVQTARKKIP HLDQLVIFAG ACQSHFESLI RAGANFASSP SRVNIHALDP | |
| VYIVAKISFT PFMERINVWE VLRNTLTREK GLGGIETRGV LRIGMPYKSN | |
| 197. SQ Sequence 873 BP; 275 A; 153 C; 216 G; 229 T; 0 other; 2281252163 CRC32; | |
| gtgcaatttc aaatagggga tatggtagcc agaaaatcct atcagatgga tgttttgttt | 60 |
| cgaattatag gaatagagca aacaagcaaa ggaaattcaa ttgccatttt gcatggagat | 120 |
| gaagtcaggc tgattgctga ttcggatttt tctgatctgg tggcagtgaa aaaggatgag | 180 |
| cagatgatgc ggaaaaagaa agatgagagc agaatgaatg agtcgctcga attgctccgc | 240 |
| caagattata agctgctcag agaaaagcag gagtactatg cgacaagcca atatcagcat | 300 |
| caggagcatt atttccatat gccgggcaaa gtgcttcatc tggatggtga cgaagcatat | 360 |
| ttgaaaaaat gcctgaatgt ctataaaaaa attggagtgc cggtctatgg catccattgc | 420 |
| catgaaaaga aaatgtctgc ttctattgaa gtattgctcg acaaatatcg acctgacatc | 480 |
| ctggtgatca cagggcatga tgcgtactcg aagcaaaagg gcggtattga tgatttgaat | 540 |
| gcgtacagac attctaagca ctttgttgaa acagttcaaa cagcccgaaa aaagatccct | 600 |
| cacttagatc agcttgttat ttttgcgggg gcctgccaat cccattttga atcactcatc | 660 |
| agagcgggtg cgaattttgc aagttcaccg tcaagagtca atattcatgc gcttgatccg | 720 |
| gtatatatcg tcgcgaagat cagctttacg ccgtttatgg aacggattaa tgtatgggaa | 780 |
| gtgctccgta atacgctgac aagagagaaa gggcttggag gtattgaaac aagaggagtt | 840 |

B. subtilis YrbA/SafA - (O32062/Q799D6)

| | |
|---|---|
| 200. SQ SEQUENCE 387 AA; 43229 MW; CE619293E809E5D4 CRC64; | |
| MKIHIVQKGD SLWKIAEKYG VDVEEVKKLN TQLSNPDLIM PGMKIKVPSE GVPVRKEPKA | |
| GKSPAAGSVK QEHPYAKEKP KSVVDVEDTK PKEKKSMPYV PPMPNLQENV YPEADVNDYY | |
| DMKQLFQPWS PPKPEEPKKH HDGNMDHMYH MQDQFPQQEA MSNMENANYP NMPNMPKAPE | |
| VGGIEEENVH HTVPNMPMPA VQPYYHYPAH FVPCPVPVSP ILPGSGLCYP YYPAQAYPMH | |
| PMHGYQPGFV SPQYDPGYEN QHHENSHHGH YGSYGAPQYA SPAYGSPYGH MPYGPYYGTP | |
| QVMGAYQPAA AHGYMPYKDH DDCGCDGDHQ PYFSAPGHSG MGAYGSPNMP YGTANPNPNP | |
| YSAGVSMPMT NQPSVNQMFG RPEEENE | |
| 199. SQ Sequence 1164 BP; 357 A; 274 C; 270 G; 263 T; 0 other; 2380158318 CRC32; | |
| ttgaaaatcc atatcgttca aaaaggcgat tcgctctgga aaatagctga aaagtacgga | 60 |
| gtcgatgttg aggaagtgaa aaaactcaat acacagctta gcaatccaga cttaatcatg | 120 |
| cctggaatga aaataaaagt gccgtcagaa ggagtcccgg tcagaaaaga gccaaaagcg | 180 |
| ggcaaaagtc ctgcggccgg gagtgtgaag caagaacatc catatgcgaa agagaagcct | 240 |
| aaatccgttg tcgatgtaga agacacaaag ccgaaagaaa agaagtccat gccgtatgtc | 300 |
| ccgccgatgc ctaatttgca ggaaaatgtg taccctgaag ctgatgtgaa cgattattat | 360 |
| gatatgaaac agcttttcca gccttggtcg cctcctaaac cggaggagcc gaaaaaacat | 420 |
| catgacggaa atatggatca tatgtatcat atgcaagacc aatttccaca acaggaggct | 480 |
| atgagtaata tggaaaatgc aaattatccg aatatgccta atatgccaaa ggcgccagag | 540 |
| gtaggcggta tagaagagga aaacgttcat cacacagttc cgaatatgcc gatgccggct | 600 |
| gttcagcctt attatcatta tccggctcat ttcgtaccgt gtccggtgcc tgtttcgcca | 660 |
| attcttccag gatcaggatt atgctatccg tactatccgg cacaagctta tccaatgcat | 720 |
| ccgatgcatg gataccagcc aggctttgta tcgcctcagt atgacccggg ttatgaaaac | 780 |
| cagcatcatg aaaacagcca tcacggacat tacggttcat acggtgcgcc gcaatacgca | 840 |
| tctccggctt atggatctcc gtatggacat atgccgtatg gcccttatta cggcactccc | 900 |
| caagtaatgg gagcatacca gcctgctgcg gctcatggtt acatgccata caaagatcat | 960 |
| gacgactgcg gctgtgacgg tgatcatcag ccatatttct ctgcacctgg ccattcggga | 1020 |
| atgggagctt atggaagccc taatatgcca tatggcacag ctaacccaaa tccaaaccca | 1080 |
| tattcggcag gagtttctat gccaatgacg aaccagcctt ctgtaaacca aatgtttggc | 1140 |
| cgtccggaag aagaaaatga gtga | 1164 |

B. subtilis CotQ/YvdP - (O06997/Q795H3)

| | |
|---|---|
| 202. SQ SEQUENCE 447 AA; 50085 MW; 1096092D325229DB CRC64; | |
| MGSTQLTGRV IFKGDPGYTE AIKNWNPYVD VYPLVFVFAQ NSYDVSNAIK WARENKVPLR | |
| VRSGRHALDK NLSVVSGGIV IDVSDMNKVF LDEENAIATV QTGIPVGPLV KGLARDGFMA | |
| PFGDSPTVGI GGITMGGGFG VLSRSIGLIS DNLLALKTVD AKGRIIHADQ SHNEDLLWAS | |
| RGGGGGNFGY NTQYTFKVHR APKTATVFNI IWPWEQLETV FKAWQKWAPF VDERLGCYLE | |
| IYSKINGLCH AEGIFLGSKT ELIRLLKPLL HAGTPTEADI KTLYYPDAID FLDPDEPIPG | |
| RNDQSVKFSS AWGHDFWSDE PISIMRKFLE DATGTEANFF FINWGGAISR VPKDETAFFW | |
| RHPLFYTEWT ASWKNKSQED SNLASVERVR QLMQPYVAGS YVNVPDQNIE NFGKEYYGAN | |
| FARLREIKAK YDPENVFRFP QSIPPSR | |
| 201. SQ Sequence 1344 BP; 408 A; 250 C; 306 G; 380 T; 0 other; 1853373320 CRC32; | |
| atgggatcaa cacagttgac agggcgtgta atcttcaaag gagaccccgg ctatacagag | 60 |
| gctattaaga attggaaccc ttatgtggat gtctatcctc ttgtctttgt ttttgcgcaa | 120 |
| aattcatacg atgtaagtaa tgccattaaa tgggctcgtg agaataaagt gcccttacgt | 180 |
| gtcagaagcg gtcgccatgc tttagataag aacctttcag tagtaagtgg aggaattgtt | 240 |
| attgatgtga gtgacatgaa taaagttttc ttagatgaag aaaacgctat tgcaaccgtt | 300 |
| caaactggta ttcccgttgg cccgcttgta aagggattag ctcgagacgg ttttatggct | 360 |
| ccgtttggag atagcccaac agttggaatc gggggaatta cgatgggcgg cggatttggt | 420 |
| gtactctcac gatcgattgg ccttataagt gataaccttc tcgcgctgaa aacggtagat | 480 |
| gcaaaaggaa ggattattca cgcagatcaa tctcacaatg aggatttgct atgggcttct | 540 |
| agaggcggag gaggaggtaa ctttggatat aatacccaat atacattcaa agttcatcgt | 600 |
| gcccctaaaa ctgcaaccgt cttcaatatt atctggccgt gggaacaatt agaaacggta | 660 |
| tttaaagctt ggcagaaatg ggctccgttt gtagatgaac gattaggatg ctaccttgaa | 720 |
| atttacagca aaataaatgg tttgtgtcat gcagaaggaa ttttcctcgg ttcgaaaact | 780 |
| gaattgattc gattattaaa acctttatta catgcgggaa ctccaacaga agcagatatc | 840 |
| aaaacattat actatccaga tgctatagat ttcttagacc ctgacgaacc catccctggc | 900 |
| agaaatgatc agagtgttaa attctcctcg gcatggggtc atgatttttg gtctgacgaa | 960 |
| cccatttcaa tcatgagaaa atttttggaa gatgctactg gaacagaagc caatttcttt | 1020 |
| tttatcaatt ggggtggtgc tataagcaga gtccctaaag acgaaactgc ctttttttgg | 1080 |
| cgccatccat tattttatac ggaatggacg gctagttgga aaaataaatc acaagaagat | 1140 |
| tcaaatcttg catcagttga aagagtgcgt cagctgatgc aaccatatgt agcaggttca | 1200 |
| tatgttaatg ttccagatca aaacattgaa aacttcggaa aagaatatta tggcgcaaac | 1260 |
| tttgcgcggc ttcgagaaat aaaggcgaaa tatgaccccg aaaatgtatt tcgttttccg | 1320 |
| caaagcatcc cgccatctcg ttaa | 1344 |

B. subtilis CotU/YnzH - (O31802)

| | |
|---|---|
| 204. SQ SEQUENCE 86 AA; 11562 MW; D5E8AE82B09A9BF6 CRC64; | |
| MGYYKKYKEE YYTWKKTYYK KYYDNDKKHY DCDKYYDHDK KHYDYDKKYD DHDKKYYDDH | |
| DYHYEKKYYD DDDHYYDFVE SYKKHH | |
| 203. SQ Sequence 261 BP; 120 A; 26 C; 38 G; 77 T; 0 other; 2555772873 CRC32; | |
| ttgggttatt ataaaaaata taaagaagag tattatactt ggaaaaaaac atattacaaa | 60 |
| aagtattacg acaatgataa gaagcattat gattgcgaca agtattatga tcatgataaa | 120 |
| aaacattatg attacgacaa aaagtatgat gaccatgata aaaagtatta cgatgatcac | 180 |
| gattatcatt acgaaaaaaa gtattatgat gacgatgatc attattatga ttttgtcgaa | 240 |
| tcatataaaa aacatcacta a | 261 |

B. subtilis CotI/YtaA - (O34656/Q7BVVO)

| | |
|---|---|
| 206. SQ SEQUENCE 357 AA; 41245 MW; ED6C7BA6BC3FBFEA CRC64; | |
| MCPLMAENHE VIEEGNSSEL PLSAEDAKKL TELAENVLQG WDVQAEKIDV IQGNQMALVW | |
| KVHTDSGAVC LKRIHRPEKK ALFSIFAQDY LAKKGMNVPG ILPNKKGSLY SKHGSFLFVV | |
| YDWIEGRPFE LTVKQDLEFI MKGLADFHTA SVGYQPPNGV PIFTKLGRWP NHYTKRCKQM | |
| ETWKLMAEAE KEDPFSQLYL QEIDGFIEDG LRIKDRLLQS TYVPWTEQLK KSPNLCHQDY | |
| GTGNTLLGEN EQIWVIDLDT VSFDLPIRDL RKMIIPLLDT TGVWDDETFN VMLNAYESRA | |
| PLTEEQKQVM FIDMLFPYEL YDVIREKYVR KSALPKEELE SAFEYERIKA NALRQLI | |
| 205. SQ Sequence 1074 BP; 334 A; 219 C; 249 G; 272 T; 0 other; 244379893 CRC32; | |
| atgtgtcctt taatggcaga aaaccatgaa gtcattgagg aggggaattc atcagagctt | 60 |
| cctttatcag cagaagatgc aaaaaaatta acggagctgg ctgaaaatgt gcttcaagga | 120 |
| tgggatgtgc aggctgaaaa aatagacgtc attcagggaa accagatggc gcttgtctgg | 180 |
| aaggtccaca cagactccgg cgcggtttgt ctaaaacgaa tacacaggcc agaaaagaaa | 240 |
| gcgttgtttt ccattttcgc gcaggactat ttagcaaaaa aaggcatgaa tgttcctggc | 300 |
| atactcccaa acaaaaaagg cagcctatat tctaagcacg gctcatttct atttgtcgta | 360 |
| tatgactgga tcgaaggaag accgtttgag ctgactgtaa agcaggactt ggagtttatc | 420 |
| atgaaaggcc ttgctgattt tcatacagct tccgtcggat atcagccgcc aaatggcgtt | 480 |
| cccatattta ccaaattagg tcgctggccg aatcactaca cgaaacgatg caaacagatg | 540 |
| gaaacgtgga agctgatggc ggaggcggaa aaagaagatc ctttctcaca gctttatctt | 600 |
| caggagatag atggctttat tgaagacggg ctgcgcatca aagaccggct tttgcaatcg | 660 |
| acctatgttc catggactga acagctgaaa aaaagcccta acctttgcca ccaggattac | 720 |
| ggaaccggga atacactctt aggagaaaat gaacagattt gggtcatcga cttagatacc | 780 |
| gtatcatttg atctgcctat tcgcgatttg cgcaaaatga ttattccgct tttggatacg | 840 |
| acgggtgttt gggatgacga aacatttaat gtcatgctga acgcatacga atccagagcc | 900 |
| ccattaactg aagaacaaaa acaagtcatg tttattgata tgctgtttcc ttacgagctt | 960 |
| tacgatgtca ttcgcgaaaa atacgtccgc aagtctgctt taccgaagga agaattagaa | 1020 |
| tcagcttttg aatatgaacg cattaaagca aacgcattgc ggcagcttat ttaa | 1074 |

B. subtilis YckK - (P42199/P94402)

| | |
|---|---|
| 208. SQ SEQUENCE 268 AA; 29470 MW; 6F513D0E05E6DCCA CRC64; | |
| MKKALLALFM VVSIAALAAC GAGNDNQSKD NAKDGDLWAS IKKKGVLTVG TEGTYEPFTY | |
| HDKDTDKLTG YDVEVITEVA KRLGLKVDFK ETQWGSMFAG LNSKRFDVVA NQVGKTDRED | |
| KYDFSDKYTT SRAVVVTKKD NNDIKSEADV KGKTSAQSLT SNYNKLATNA GAKVEGVEGM | |
| AQALQMIQQA RVDMTYNDKL AVLNYLKTSG NKNVKIAFET GEPQSTYFTF RKGSGEVVDQ | |
| VNKALKEMKE DGTLSKISKK WFGEDVSK | |
| 207. SQ Sequence 807 BP; 292 A; 156 C; 180 G; 179 T; 0 other; 1942198485 CRC32; | |
| atgaaaaaag cattattggc tttattcatg gtcgtaagta ttgcagctct tgcagcttgc | 60 |
| ggagcaggaa atgacaatca gtcaaaagat aatgccaaag atggcgatct ttgggcttca | 120 |
| attaagaaaa aaggtgtgct cacagtcgga acggaaggaa catatgagcc gttcacttac | 180 |
| cacgacaaag acactgataa actgactggc tatgatgtcg aagttatcac agaagtcgca | 240 |
| aacagcctcg ggcttaaagt cgactttaag gaaacacagt gggacagcat gtttgccggc | 300 |
| ctgaattcca aacggtttga cgttgttgcc aaccaagtcg gaaaaacaga tcgtgaaaat | 360 |
| caatatgatt tctcagataa atacacaaca tcaagagccg ttgtcgtaac gaaaaaagac | 420 |
| aacaacgata ttaagtctga agcagatgta aaaggaaaaa cgtcagctca atcactgaca | 480 |
| agcaactaca acaaattagc tacaaatgcc ggcgctaaag tagaaggcgt tgaaggcatg | 540 |
| gcgcaggccc ttcaaatgat ccagcaaggc cgcgtcgata tgacatacaa cgataagctt | 600 |
| gccgtattga actacttaaa aacatctggc aataaaaacg tgaaaatcgc gtttgaaaca | 660 |
| ggtgagcctc agtcaacata tttcacgttc cgtaaaggaa gcggcgaggt tgttgatcaa | 720 |
| gtcaacaaag cattaaaaga aatgaaagag gacgggactc tttctaaaat ttctaaaaaa | 780 |
| tggttcggcg aagatgtttc taaataa | 807 |

B. subtilis YdhD - (O05495/Q797E3)

| | |
|---|---|
| 210. SQ SEQUENCE 439 AA; 48964 MW; F260CE0D32C73966 CRC64; | |
| MFIHIVGPGD SLFSIGRRYG ASVDQIRGVN GLDETNIVPG QALLIPLYVY TVQPRDTLTA | |
| IAAKAFVPLE RLRAANPGIS PNALQAGAKI TIPSISNYIA GTLSFYVLRN PDLDRELIND | |
| YAPYSSSISI FEYHIAPNGD IANQLNDAAA IETTWQRRVT PLATITNLTS GGFSTEIVHQ | |
| VLNNPTARTN LVNNIYDLVS TRGYGGVTID FEQVSAADRD LFTGFLRQLR DRLQAGGYVL | |
| TIAVPAKTSD NIPWLRGYDY GGIGAVVNYM FIMAYDWHHA GSEPGPVAPI TEIRRTIEFT | |
| IAQVPSRKII IGVPLYGYDW IIPYQPGTVA SAISNQNAIE RAMRYQAPIQ YSAEYQSPFF | |
| RYSDQQGRTH EVWFEDVRSM SRKMQIVREY RLQAIGAWQL TLALRRAHGF CGNFLRSEKC | |
| KKRHQSLGVF FLIKSRAAE | |
| 209. SQ Sequence 1320 BP; 358 A; 288 C; 343 G; 331 T; 0 other; 2682817624 CRC32; | |
| atgtttatcc atatcgtcgg gcctggtgat tctttgtttt cgataggcag aagatacggt | 60 |
| gcttctgttg atcaaatacg gggtgtgaat ggtttagatg aaacgaatat cgtgccgggg | 120 |
| caggctctgc ttatccctct ttatgtatat acagtccagc cgagagatac gcttaccgcc | 180 |
| attgcagcta aagcgtttgt gccattagag cgactgcgag cggccaatcc gggcatcagc | 240 |
| ccaaatgctt tacaagcggg agcaaaaata acgattcctt cgatctcaaa ttacattgcg | 300 |
| ggaacgttaa gtttttatgt gctccgaaac ccagacctcg atcgggaatt aatcaatgat | 360 |
| tatgcgccat actcgtcttc gatttcaatt ttcgaatacc atattgcacc gaacggcgac | 420 |
| attgcaaacc aattgaatga tgcggccgct attgagacaa cttggcaaag acgagtcacg | 480 |
| ccgctggcaa caataacgaa ccttacatca ggaggcttca gtacggagat tgttcaccaa | 540 |
| gtgctaaaca atccgacagc gagaaccaat ctggtcaaca acatttatga cttagtttcc | 600 |
| acaaggggat atggcggtgt cacaatcgat tttgagcagg tgagcgccgc ggatcgcgat | 660 |
| cttttcactg gatttttacg ccagctgaga gatcgacttc aggcgggagg gtatgtgctg | 720 |
| acgatagctg ttcctgcaaa aacaagtgat aatatcccat ggctgagggg ctacgattac | 780 |
| ggggggatag gagcggttgt caattatatg tttatcatgg cttatgattg gcatcatgcg | 840 |
| ggaagtgagc cgggtcctgt agcgccgatt actgaaataa ggagaaccat tgagtttacg | 900 |
| attgcgcagg tgccgagcag aaaaatcatt atcggagtcc cgctctacgg gtacgactgg | 960 |
| atcatcccgt accagccggg cacagttgct tcagcgattt caaatcaaaa cgcaatcgaa | 1020 |
| agagcgatga ggtaccaagc cccgatacaa tattcagccg aatatcaatc accgtttttc | 1080 |
| cggtacagtg atcagcaggg gcggacgcat gaggtatggt ttgaggatgt cagaagcatg | 1140 |
| agccggaaga tgcagatcgt ccgtgaatac agattgcagg ctattggcgc ttggcagtta | 1200 |
| acgctggctt tacgccgggc ccatggcttc tgcggaaatt ttttacgatc agaaaagtgt | 1260 |
| aaaaaaagac accagagctt gggtgtcttt tttttgatta agtccagagc agcagaatag | 1320 |

B. subtilis YhdA - (P97030/Q796Y4)

| | |
|---|---|
| 212. SQ SEQUENCE 435 AA; 48534 MW; 5E0C6194BA0CD275 CRC64; | |
| MTAAACKPAA RSVITESSLI FTSIHSSYVI STYYKRCVVL SQRKEAVQNM NVYQLKEELI | |
| EYAKSIGVDK IGFTTADTFD SLKDRLILQE SLGYLSGFEE PDIEKRVTPK LLLPKAKSIV | |
| AIALAYPSRM KDAPRSTRTE RRGIFCRASW GKDYHDVLRE KLDLLEDFLK SKHEDIRTKS | |
| MVDTGELSDR AVAERAGIGF SAKNCMITTP EYGSYVYLAE MITNIPFEPD VPIEDMCGSC | |
| TKCLDACPTG ALVNPGQLNA QRCISFLTQT KGFLPDEFRT KIGNRLYGCD TCQTVCPLNK | |
| GKDFHLHPEM EPDPEIAKPL LKPLLAISNR EFKEKFGHVS GSWRGKKPIQ RNAILALAHF | |
| KDASALPELT ELMHKDPRPV IRGTAAWAIG KIGDPAYAEE LEKALEKEKD EEAKLEIEKG | |
| IELLKASGMT KQGLS | |
| 211. SQ Sequence 1308 BP; 386 A; 281 C; 333 G; 308 T; 0 other; 3960484223 CRC32; | |
| atgacagcag ctgcatgtaa gccggccgca cgttcagtaa taacagaatc aagtttgata | 60 |
| ttcactagca ttcactccag ttacgtgata tcaacctatt ataaacgctg tgtcgtttta | 120 |
| tcacaaagaa aggaggctgt gcaaaacatg aacgtttatc agctcaaaga agaattaatt | 180 |
| gaatacgcga aaagcattgg cgtagacaag attggtttta cgaccgctga tacttttgac | 240 |
| agtttaaaag accgtttgat tcttcaagaa tcactcggct atctctccgg ctttgaagag | 300 |
| ccagatatcg aaaaaagggt gacgccgaag cttcttttgc cgaaagcgaa atcaatagtg | 360 |
| gcaattgctc tcgcatatcc ttccagaatg aaggatgcgc cgagaagcac gagaactgag | 420 |
| cgcaggggca ttttttgcag agcttcctgg ggaaaagact atcatgatgt gctgagggaa | 480 |
| aagcttgatc tgctggagga ttttctaaaa agcaagcatg aggatatcag aacgaagtca | 540 |
| atggttgata caggtgaatt gtctgatcgc gccgttgcgg aacgtgccgg aatcggattc | 600 |
| agtgcgaaaa actgtatgat cacaacaccc gagtatggct cttatgtgta tttggcggaa | 660 |
| atgatcacaa atatcccttt tgagcctgat gtgccgattg aagatatgtg cgggtcctgc | 720 |
| acgaaatgct tggacgcctg cccaacggga gcactggtta atcccgggca gcttaatgcg | 780 |
| cagcgctgca tctcttttct gacccagaca aaaggatttt tgcctgatga attccggaca | 840 |
| aaaatcggaa accgcctgta cgggtgcgat acgtgccaaa cggtatgccc tctcaataaa | 900 |
| gggaaggatt ttcatcttca tccggaaatg gagcctgatc ctgagattgc caaaccgtta | 960 |
| ttgaagccgc ttttggccat cagcaatcgg gaatttaagg agaaattcgg gcatgtctca | 1020 |
| ggttcttggc gcggaaaaaa accgattcag cgaaacgcca ttctcgcgct tgcccatttt | 1080 |
| aaggatgctt ccgcactgcc tgaattgacg gaactgatgc acaaggatcc gcgtcctgtc | 1140 |
| atcaggggga cagccgcatg ggcaatcgga aaaatcggag accccgccta cgcggaagag | 1200 |
| cttgaaaaag cgctggaaaa agagaaggat gaagaggcaa agctggaaat tgaaaaagga | 1260 |
| attgagttgc taaaagcttc aggcatgact aaacaaggcc tgtcctga | 1308 |

B. subtilis YhdE - (O07573)

| | |
|---|---|
| 214. SQ SEQUENCE 146 AA; 16609 MW; 02C519057F1A3A9C CRC64; | |
| MKLTNYTDYS LRVLIFLAAE RPGELSNIKQ IAETYSISKN HLMKVIYRLG QLGYVETIRG | |
| RGGGIRLGMD PEDINIGEVV RKTEDDFNIV ECFDANKNLC VISPVCGLKH VLNEALLAYL | |
| AVLDKYTLRD LVKNKEDIMK LLKMKE | |
| 213. SQ Sequence 441 BP; 143 A; 84 C; 98 G; 116 T; 0 other; 3020939562 CRC32; | |
| atgaagttaa ccaattatac agattattca ttaagagtgt tgatttttct ggctgcagag | 60 |
| cgtcccggag aactttcaaa tataaaacag attgccgaaa cgtattctat ttcaaaaaat | 120 |
| catctcatga aagtcatata caggctcggc cagctcggct acgtagaaac gatacgcgga | 180 |
| cggggcggcg gcatacgatt aggcatggac cctgaagaca tcaacatcgg tgaggttgtc | 240 |
| agaaaaacgg aggacgattt taatattgtt gaatgttttg atgcgaacaa gaatctctgt | 300 |
| gttatttccc cggtttgcgg cttaaaacat gtgctgaatg aagcgctttt agcctacctc | 360 |
| gcagttttag acaaatacac actgcgcgac ctcgtcaaaa acaaagaaga tatcatgaag | 420 |
| cttttaaaaa tgaaggaata g | 441 |

B. subtilis YirY - (O06712, O06713, O06714)

| | |
|---|---|
| 216. SQ SEQUENCE 1130 AA; 128918 MW; E35A8293631B4835 CRC64; | |
| MKPIALSIKG LHSFREEQTI DFEGLSGAGV FGIFGPTGSG KSSILDAMTL ALYGKVERAA | |
| NNTHGILNHA EDTLSVSFTF ALQTNHQISY KVERVFKRTD EMKVKTALCR FIEIKDEHTV | |
| LADKASEVNK RVEELLGLTI DDFTRAVVLP QGKFAEFLSL KGAERRHMLQ RLFNLEQYGD | |
| RLVKKLRRQA QEANARKNEM LAEQSGLGEA SSEAVEQAEK VLEQAEVRLE AMRKNRDQAK | |
| ERFTEHQEIW NVQKEKSTYE EEEKRLAEEQ PHIDSMQKRL LEAETAAALK PYADRYAEAI | |
| QHEEQAEKEQ TLAQKDLADR TAFFQQKHEE YEAWRQHKSE KEPELLAKQE QLSRLQEIEI | |
| KLSEAKQEEE RKKADLRQKE EALQSVMNEL ETVTDRLTRG QNRQTELKQQ LKSLQVTSDE | |
| RKSCQQAAEM ALRIRQTEEQ IKKEKKRSEE LNLVLQKMNE EKNTLVQKTE AEENNIIQAY | |
| EAVQTVYHLV CETERSLTRM TEEARKSQHT LHLQREKARV ALLTKELAQK LTAGKPCPVC | |
| GSTDHDPSAS VHETYEADSH LEEDIKRTDV LLTEAAALSQ EILSAKIMLE EQSARFIEQC | |
| PFLQTIQAQN LEAAASFEHQ PVYEAFETAK FEWKRIKQDI LSVKTRMAQM IGAYQESLKK | |
| AEQLNEKIGF EKREADRIES IISELQSSMD SSLNMFKEAF QNQSVDEAEK WQQAIEEKDR | |
| AAEECEKRIE KSIAFLAEHE AQKEKLRESG HRLEREKLEL HYAAERIKSV IADYEHELGD | |
| YAKGDSIPIQ LRSVQQDLKL LKEKEQSLYE ELQSAQMKLN QAKSRASASE LTLQEAKGRL | |
| EKAKAAWLEH TKNTSITRTE EVEQSLIPAD ELEKMKTGID QFMDKLKQNA ANLKRVAEIL | |
| AGRALSESEW NETVAALQEA EDAFGAAIEE KGAAAKALAV IRDHHKRFNE IEAELKKWQM | |
| HIDRLDKLQA VFKGNTFVEF LAEEQLESVA RDASARLSML TRQRYAIEVD SEGGFVMRDD | |
| ANGGVRRPVS SLSGGETFLT SLSLALALSA QIQLRGEYPL QFFFLDEGFG TLDQDLLDTV | |
| VTALEKLQSD NLAVGVISHV QELRARLPKK LIVHPAEPSG RGTRVSLELM | |
| 215. SQ Sequence 3393 BP; 1091 A; 727 C; 905 G; 670 T; 0 other; 1438739044 CRC32; | |
| atgaagccga tcgccttaag cattaagggg ctccacagcc ttagagagga gcagacgata | 60 |
| gattttgaag gcctttccgg tgccggtgtt ttcggcattt tcggcccgac aggaagcggt | 120 |
| aaatcctcta tactcgacgc aatgacgctt gctttatacg gaaaggtgga acgggcggcg | 180 |
| aataatacgc acggaatctt aaatcacgcc gaagatacgc tgtctgtgtc ctttaccttt | 240 |
| gcgcttcaga cgaatcacca aatctcatac aaagtcgagc gtgtgtttaa gagaacggat | 300 |
| gaaatgaagg taaaaacggc actttgccgc ttcatcgaaa tcaaggacga gcatacggtg | 360 |
| ctggctgata aagccagcga agtgaataaa agagtggagg agctcttagg gctgacgatc | 420 |
| gacgatttta cgagagcggt ggtgctgccc caagggaaat ttgctgaatt tctgtcttta | 480 |
| aaaggggcag agcgcaggca tatgcttcag cgtttattta atttggagca atatggagac | 540 |
| aggcttgtga aaaagctgag acggcaggcg caggaagcca atgcgagaaa aaatgaaatg | 600 |
| cttgctgaac agtccggtct cggtgaggcg agctcagagg cagtggagca ggctgaaaag | 660 |
| gttctcgaac aagctgaagt ccggctggaa gcgatgagga agaaccgtga tcaggcgaag | 720 |
| gagcggttta cagagcatca ggagatatgg aatgtccaaa aggaaaaatc cacttatgaa | 780 |
| gaagaggaaa aacgtctcgc agaagaacag ccgcatatag acagcatgca aaaacgcctg | 840 |
| ctggaagcag aaacagcagc agcccttaag ccctatgcgg accggtacgc agaagcgatc | 900 |
| cagcatgagg agcaagctga aaaggaacaa acgctagccc aaaaggattt agcagaccgg | 960 |
| acagctttct ttcagcaaaa acatgaagag tatgaagcgt ggcgccagca taaaagcgag | 1020 |
| aaagagcctg agcttttagc caaacaggaa cagctttcac gcttgcagga aatcgaaatc | 1080 |
| aaactgagtg aggccaagca agaggaagag cgcaaaaagg ctgacctccg gcagaaagaa | 1140 |
| gaggctcttc aatctgtcat gaatgaatta gagaccgtaa cagaccgcct gacacgaggg | 1200 |
| caaaacagac agacagaatt gaagcagcag ctcaaatccc tgcaggtgac atccgatgag | 1260 |
| cgaaaaagct gccagcaggc cgcagagatg gcattgcgca tcagacaaac cgaggaacaa | 1320 |
| atcaaaaaag agaaaaaacg aagtgaagaa ttgaacctcg tgctgcagaa gatgaatgaa | 1380 |
| gagaagaata cactcgttca aaagacggaa gcggaagaaa acaacatcat tcaggcatat | 1440 |
| gaggcagttc aaactgtgta ccatttggtg tgcgaaacgg aacgctcatt aacacgtatg | 1500 |
| acggaagagg ctagaaagag tcaacacacg cttcacttac agcgtgaaaa agcaagggtg | 1560 |
| gcactgctga caaaagagtt agcccaaaag ctgactgccg gaaagccttg cccggtatgc | 1620 |
| ggttcaaccg atcatgatcc atctgcctcg gtacatgaaa cgtatgaagc cgacagccat | 1680 |
| cttgaagagg acatcaaacg gacagatgtg ttattgacgg aagctgcagc tctcagccag | 1740 |
| gagattcttt cagccaaaat tatgcttgaa gaacagtccg cgcgctttat tgaacagtgt | 1800 |
| ccgtttttgc agacaattca agcacagaac cttgaagcgg cagcttcctt cgaacatcag | 1860 |
| ccggtgtatg aagcatttga aactgccaaa tttgaatgga aacgaatcaa gcaggacatt | 1920 |
| ctttctgtta agacacgaat ggcacaaatg attggcgcct atcaggagtc tttaaaaaag | 1980 |
| gccgagcagc ttaatgaaaa aatcggtttt gaaaaaagag aagccgaccg tattgaaagc | 2040 |
| atcatcagtg agcttcaatc ctcaatggac agcagtctga acatgtttaa agaagcattt | 2100 |
| cagaatcaat ctgtggacga agcagaaaaa tggcagcaag ccattgaaga aaaggaccgg | 2160 |
| gctgcagaag aatgtgaaaa acgaattgag aagagtatcg cgtttcttgc tgagcatgaa | 2220 |
| gcacaaaagg aaaaactgcg ggaatcggga caccggcttg agcgggaaaa gctggagctt | 2280 |
| cattatgcgg ctgaacgcat caagagcgtg atagctgatt atgagcacga actcggagat | 2340 |
| tatgcaaaag gagattcgat tccaatccaa ctccgctctg tccagcagga tctaaagctg | 2400 |
| ttaaaggaaa aagaacaatc tttatatgaa gaactgcaaa gcgcccaaat gaagctcaac | 2460 |
| caagcgaaaa gccgcgcttc tgcaagcgag ctcactcttc aagaggcgaa gggcagattg | 2520 |
| gaaaaagcaa aagctgcttg gcttgagcat acaaaaaaca cctccattac ccggactgag | 2580 |
| gaggttgaac aaagtctcat cccagctgat gaacttgaaa agatgaaaac cggcatagac | 2640 |
| cagtttatgg ataaactgaa gcaaaatgct gcaaacttaa aacgagtagc agagatactt | 2700 |
| gccggcagag cattatcaga gagcgaatgg aacgaaaccg ttgcagcatt acaagaagct | 2760 |
| gaggacgcat ttggcgctgc tatagaggaa aaaggcgcgg ccgcaaaagc actggctgtc | 2820 |
| attcgcgacc atcataaacg gtttaatgaa attgaagctg aactgaaaaa atggcagatg | 2880 |
| catatcgaca ggctggacaa gctgcaagct gtgtttaaag gcaatacctt cgtcgaattt | 2940 |
| ttagctgagg agcagcttga aagcgttgcg agggacgcct cagcaagact cagtatgctg | 3000 |
| acaagacagc gctatgccat cgaagtagat tctgagggcg gcttcgtgat gcgggatgac | 3060 |
| gcgaatggag gcgtacgacg cccggtttcc agtttgtctg gaggagagac cttcctcacc | 3120 |
| tcgctttcac ttgctcttgc gctgtctgcg cagattcagc ttcgggggga atacccgctg | 3180 |
| cagttctttt tcttagatga aggcttcggc acactggatc aagatctgct tgatacggtt | 3240 |
| gtaacggcct tggaaaaact tcagtcagac aacctggctg tcggtgtcat cagccatgtg | 3300 |
| caggaactgc gtgcacggct tccgaaaaag ctgatcgtcc atccggctga accgagcggc | 3360 |
| cgcggtacgc gggtatcact tgagttgatg taa | 3393 |

B. subtilis YisY - (O06734/Q796Q4)

| | |
|---|---|
| 218. SQ SEQUENCE 268 AA; 30559 MW; E0B0B2490CE28E38 CRC64; | |
| MGHYIKTEEH VTLFVEDIGH GRPIIFLHGW PLNHKMFEYQ MNELPKRGFR FIGVDLRGYG | |
| QSDRPWEGYD YDTMADDVKA VIYTLQLENA ILAGFSMGGA IAIRYMARHE GADVDKLILL | |
| SAAAPAFTKR PGYPYGMRKQ DIDDMIELFK ADRPKTLADL GKQFFEKKVS PELRQWFLNL | |
| MLEASSYGTI HSGIALRDED LRKELAAIKV PTLILHGRKD RIAPFDFAKE LKRGIKQSEL | |
| VPFANSGHGA FYEEKEKINS LIAQFSNS | |
| 217. SQ Sequence 807 BP; 220 A; 157 C; 226 G; 204 T; 0 other; 2218419891 CRC32; | |
| atggggcatt acatcaaaac cgaggagcat gtgacactgt ttgtagagga tatcggacat | 60 |
| ggaaggccga tcatcttttt gcacgggtgg ccgttgaatc ataagatgtt tgaatatcaa | 120 |
| atgaatgagc ttccgaaaag gggatttcgt tttatcggcg ttgatttgcg gggatatggg | 180 |
| caatctgacc gcccttggga aggctacgat tatgacacga tggccgatga tgtgaaagca | 240 |
| gtcatttata cgctgcagct tgagaatgcg attcttgccg gtttttcaat gggcggcgca | 300 |
| attgcaatcc gttatatggc aaggcatgaa ggagccgatg ttgataagct gattttactg | 360 |
| tctgcggcgg cccccgcgtt tacaaaacgc ccgggttatc cgcacgggat gaggaagcag | 420 |
| gatattgacg atatgattga attgttcaaa gctgatcggc ccaaaacact ggctgattta | 480 |
| gggaaacagt tttttgagaa aaaagtgtct ccagagctta ggcagtggtt tctcaatctg | 540 |
| atgctggagg cttcctccta cgggacgatc cactcgggca tcgcattaag agacgaagat | 600 |
| ctcagaaagg aacttgctgc aatcaaggtg ccgacgctga tcctgcacgg gagaaaggat | 660 |
| agaattgcgc cgtttgattt tgcgaaagaa ttgaagcgcg gcatcaaaca gtcggaattg | 720 |
| gttccgtttg caaacagcgg gcacggagca ttttatgagg aaaaagagaa gatcaacagt | 780 |
| ttgattgcgc agttctccaa ctcataa | 807 |

B. subtilis YodI - (O34654)

| | |
|---|---|
| 220. SQ SEQUENCE 83 AA; 9194 MW; 99F58EA2F0F36A43 CRC64; | |
| MERYYHLCKN HQGKVVRITE RGGRVHVGRI TRVTRDRVFI APVGGGPRGF GYGYWGGYWG | |
| YGAAYGISLG LIAGVALAGL FFW | |
| 219. SQ Sequence 252 BP; 62 A; 42 C; 79 G; 69 T; 0 other; 4000863713 CRC32; | |
| ttggagagat attatcatct ttgcaaaaac catcaaggta aagtcgtcag aattacagag | 60 |
| agaggcggga gagttcacgt cggcagaatt acccgtgtaa caagagacag agtttttata | 120 |
| gctccggtcg gcggagggcc aagaggtttc ggttacggat attggggcgg ttattgggga | 180 |
| tatggagcgg cttacgggat ttccctcggt ttaattgcag gagtggctct ggctggttta | 240 |
| ttcttctggt aa | 252 |

B. subtilis YopQ - (O34448)

| | |
|---|---|
| 222. SQ SEQUENCE 460 AA; 53504 MW; A986850A734D97CD CRC64; | |
| MTVIFDQSAN EKLLSEMKDA ISKNKHIRSF INDIQLEMAK NKITPGTTQK LIYDIENPEV | |
| EISKEYMYFL AKSLYSVLES ERFNPRNYFT ETDMREIETL WEGSVEEDIK FPYTFKQVVK | |
| YSDDNYFFPI TAKELFMLFE NKLLHYNPNA QRTNKTKKLE GSDIEIPVPQ LNKQSVEEIK | |
| ELFLDGKLIK SVFTFNARVG SASCGEELKY DDDTMSLTVT EDTILDVLDG YHRLIGITMA | |
| IRQHPELDHL FEETFKVDIY NYTQKRAREH FGQQNTINPV KKSKVAEMSQ NVYSNKIVKF | |
| IQDNSIIGDY IKTNGDWINQ NQNLLITFSD FKKAIERSYS KKDFSTQADI LKTARYLTSF | |
| FDALATQYVD EFLGDIAKER KRSFVNNYLF FNGYVGLAKK LQLDGVSLDE LESKITDVLG | |
| SIDFSKKNKL WDELGVVDKN GNAKSPQKIW NFFNNLKIDE | |
| 221. SQ Sequence 1383 BP; 533 A; 191 C; 257 G; 402 T; 0 other; 1098563836 CRC32; | |
| atgacagtga tctttgatca gtctgcaaat gagaaactgc tttcagaaat gaaagatgct | 60 |
| atctcgaaaa ataaacacat aagatctttt attaacgata ttcaattaga gatggctaaa | 120 |
| aataaaatta ctccagggac aacacaaaaa ttaatttatg atatagaaaa tccagaagtc | 180 |
| gaaatttcta aagaatatat gtacttttta gccaagtccc tatactcagt tcttgaaagt | 240 |
| gaaaggttta atccacgaaa ttacttcaca gaaacggata tgagagaaat tgaaacgtta | 300 |
| tgggaaggat ctgtggagga agatataaaa tttccgtata cattcaaaca agttgtaaag | 360 |
| tattcggatg ataattattt cttccccatc actgctaaag agttgtttat gctatttgaa | 420 |
| aataagttat tgcactataa tcctaatgct caaagaacga acaaaacgaa aaaactagag | 480 |
| ggctcagata ttgagatacc tgtaccgcag ctcaataaac aatcggttga agaaataaag | 540 |
| gaactgttct tagatgggaa attaattaaa tcagttttta cgtttaatgc acgtgttgga | 600 |
| agcgcaagtt gtggcgaaga attaaaatat gatgacgaca ctatgtctct tacagtgact | 660 |
| gaagacacca ttttagacgt tttagacggg tatcaccggc taataggcat tactatggct | 720 |
| ataagacaac atcctgagtt agatcatttg tttgaagaaa cctttaaagt ggacatctat | 780 |
| aactacactc aaaaaagggc gagagagcat tttgggcaac aaaacacaat aaacccagtt | 840 |
| aaaaaatcta aagtggctga gatgagtcaa aatgtttatt caaataaaat tgttaagttt | 900 |
| attcaggaca atagcataat tggtgattat ataaagacaa atggagactg gataaatcag | 960 |
| aatcaaaact tacttataac tttttctgac ttcaaaaagg caattgaaag aagctattct | 1020 |
| aaaaaagatt tttctactca agcagacatc ttaaaaactg caagatacct tacatctttc | 1080 |
| tttgatgctt tagctacaca atatgtagat gagttcttag gtgatatagc aaaagaacgg | 1140 |
| aagagaagtt ttgtaaacaa ctatttgttc tttaatggtt atgtgggatt agctaagaaa | 1200 |
| ttgcaattag atggggtaag cctagacgag ttggaaagta agattactga tgttttaggc | 1260 |
| tctatagatt ttagtaagaa aaataagttg tgggatgaat taggtgtagt agacaagaat | 1320 |
| ggaaatgcta aatcaccaca aaagatatgg aatttcttca ataatttaaa aatagacgag | 1380 |
| taa | 1383 |

B. subtilis YpeP/YpeB - (P54164/P38490, P40774)

| | |
|---|---|
| 224. SQ SEQUENCE 120 AA; 13720 MW; D3F4FFA765E0A867 CRC64; | |
| MRKNKSFRLK TNNEAEYAAL YEAIREVREL GASRNSITIK GDSLVVLNQL DGSWPCYDPS | |
| HNEWLDKIEA LLESLKLTPT YETIQRKDNQ EADGLAKKIL SHQFVESHTK LDRNGDDDIG | |
| 223. SQ Sequence 363 BP; 135 A; 73 C; 78 G; 77 T; 0 other; 1949058336 CRC32; | |
| ttgagaaaaa ataaaagctt ccggctgaaa accaataatg aagctgaata cgcagcgctt | 60 |
| tatgaagcaa taagagaagt aagagagctt ggggcaagca gaaattcaat tacaatcaaa | 120 |
| ggggactcgc ttgttgtgct gaatcagctt gacggcagct ggccttgtta tgatccatct | 180 |
| cataatgaat ggctggacaa aatagaagca ctccttgaat cgctgaagct tactccaacc | 240 |
| tacgaaacaa tacaacgaaa agacaatcag gaagctgacg gcctcgctaa aaaaattcta | 300 |
| tcccatcaat tcgtagaaag ccacacgaaa ttagaccgta acggagatga cgatattgga | 360 |
| taa | 363 |
| 226. SQ SEQUENCE 450 AA; 51185 MW; 8B4A7E479C088E6B CRC64; | |
| MIRGILIAVL GIAIVGTGYW GYKEHQEKDA VLLHAENNYQ RAFHELTYQV DQLHDKIGTT | |
| LAMNSQKSLS PALIDVWRIT SEAHNSVSQL PLTLMPFNKT EELLSKIGDF SYKTSVRDLD | |
| QKPLDKNEYT SLNKLYQQSE DIQNELRHVQ HLVMSKNLRW MDVEMALASD EKQSDNTIIN | |
| SFKTVEKNVG AFSTGTDLGP SFTSTKKEEK GFSHLKGKQI SEQEAKQIAE RFAPDDNYSI | |
| KVVKSGKKTN RDVYSISMKD PDHKAVIYMD ITKKGGHPVY LIQNREVKDQ KISLNDGSNR | |
| ALAFLKKNGF ETDDLEIDES AQYDKIGVFS YVPVENKVRM YPEAIRMKVA LDDGEVVGFS | |
| ARDFLTSHRK RTIPKPAITE AEAKSKLNKN VQVRETRLAL ITNELGQEVL CYEMLGTIEN | |
| DTFRMYINAK DGSEEKVEKL KNAEPIYKDL | |
| 225. SQ Sequence 1353 BP; 497 A; 248 C; 290 G; 318 T; 0 other; 3710928530 CRC32; | |
| atgatcagag gaattttaat cgccgtgctt ggtattgcaa tagtcggtac aggctactgg | 60 |
| ggatacaaag aacaccagga aaaagacgca gttcttcttc atgctgaaaa taactatcag | 120 |
| cgggcgtttc atgagcttac ctatcaggtg gatcagcttc atgataaaat cggaacaaca | 180 |
| cttgccatga acagccaaaa atcactgtcg cctgcattga tcgatgtgtg gaggattaca | 240 |
| tcagaagctc ataacagcgt cagtcagctg ccgcttacat taatgccgtt taataaaact | 300 |
| gaagagctat tatcaaagat cggcgatttc agctataaaa cgtcagtcag agatttggac | 360 |
| caaaagccgc ttgataaaaa cgagtataca tcactaaata agctatatca gcagtccgaa | 420 |
| gatatacaaa atgaattgcg tcatgttcag caccttgtca tgagcaaaaa ccttcgctgg | 480 |
| atggacgtag aaatggctct ggcttctgac gaaaaacaaa gtgataatac gattatcaac | 540 |
| agctttaaaa cagtcgaaaa aaatgttggt gcattctcca ctggcactga tcttggcccg | 600 |
| agtttcacca gtacgaaaaa agaagagaaa ggcttcagcc atctgaaggg aaaacaaatt | 660 |
| tccgaacagg aagcaaaaca aattgctgag cgctttgccc cagatgacaa ttattcaatt | 720 |
| aaagtggtaa agagcggaaa aaaaacaaat cgcgatgtat atagcatcag catgaaagac | 780 |
| ccagaccata aagcagtgat ttatatggat attacgaaga agggcgggca tccggtatac | 840 |
| ttgatccaaa acagagaagt gaaagatcag aaaatcagtt taaatgacgg atcgaaccga | 900 |
| gcgcttgcat ttttaaagaa aaacggattt gaaacagatg atttggaaat tgatgaaagt | 960 |
| gcccaatatg ataaaatcgg tgtattttca tatgttcctg ttgaaaataa agtccggatg | 1020 |
| taccccgagg caattcgtat gaaagtggcc ttggatgacg gtgaggttgt cggcttttca | 1080 |
| gcaagagact tcctcacatc tcacagaaaa agaaccatac ctaagcctgc aattactgaa | 1140 |
| gcagaggcaa agtctaaatt aaataaaaat gtacaagtga gagaaacaag gctcgctttg | 1200 |
| attacaaatg aactaggtca agaagtgtta tgctacgaaa tgcttgggac aattgaaaat | 1260 |
| gacacattca gaatgtatat caatgccaaa gacggatcgg aagaaaaggt tgaaaaacta | 1320 |
| aaaaatgcag aacctatata taaagaccta taa | 1353 |

B. subtilis YpzA - (O32007)

| | |
|---|---|
| 228. SQ SEQUENCE 89 AA; 10062 MW; AE0BB729F2323A7E CRC64; | |
| MTSEFHNEDQ TGFTDKRQLE LAVETAQKTT GAATRGQSKT LVDSAYQAIE DARELSQSEE | |
| LAALDDPEFV KQQQQLLDDS EHQLDEFKE | |
| 227. SQ Sequence 270 BP; 92 A; 58 C; 71 G; 49 T; 0 other; 2060329115 CRC32; | |
| gtgacttcag aatttcataa tgaggatcag accggcttta cggataagcg gcagctggaa | 60 |
| ctagcggtgg aaacagcgca gaaaacaaca ggagccgcga cgagaggcca aagcaaaaca | 120 |
| ttagtcgact ctgcatacca agccattgag gatgctagag aactgtcaca atctgaagag | 180 |
| ctggcagctc tcgatgatcc tgaatttgta aagcagcaac agcagctgct agatgacagc | 240 |
| gagcatcagc tggatgaatt caaagaataa | 270 |

B. subtilis YusA - (O32167)

| | |
|---|---|
| 230. SQ SEQUENCE 274 AA; 30355 MW; 3D40F949A1BFC73C CRC64; | |
| MKKLFLGALL LVFAGVMAAC GSNNGAESGK KEIVVAATKT PHAEILKEAE PLLKEKGYTL | |
| KVKVLSDYKM YNKALADKEV DANYFQHIPY LEQEMKENTD YKLVNAGAVH LEPFGIYSKT | |
| YKSLKDLPDG ATIILTNNVA EQGRMLAMLE NAGLITLDSK VETVDATLKD IKKNPKNLEF | |
| KKVAPELTAK AYENKEGDAV FINVNYAIQN KLNPKKDAIE VESTKNNPYA NIIAVRKGEE | |
| DSAKIKALME VLHSKKIKDF IEKKYDGAVL PVSE | |
| 229. SQ Sequence 825 BP; 316 A; 158 C; 165 G; 186 T; 0 other; 2582378374 CRC32; | |
| ttgaaaaagc tatttttggg tgcattactg cttgtatttg caggagttat ggctgcctgc | 60 |
| ggttcgaata acggcgctga atccggcaag aaagaaattg tcgttgcggc aacaaaaaca | 120 |
| ccgcatgcgg aaattttaaa agaagctgaa ccattgctga aagaaaaagg ctatacgctg | 180 |
| aaagtgaaag tgcttagtga ttacaaaatg tacaataaag ctttagctga taaagaagtg | 240 |
| gacgcgaact acttccagca cattccttac cttgagcaag aaatgaaaga aaacacagat | 300 |
| tacaaacttg tgaatgccgg cgctgttcac ttagagccat tcggtattta ctctaaaaca | 360 |
| tacaaatcac tgaaagacct tccagacggt gcgacaatca ttctgacaaa caacgttgct | 420 |
| gaacaaggcc gtatgcttgc aatgcttgaa aacgctggat taatcactct tgattctaaa | 480 |
| gtggaaacag ttgacgcaac attgaaagac attaagaaaa acccgaaaaa ccttgaattc | 540 |
| aaaaaagtag cgcctgaatt aacggcaaaa gcatatgaaa acaaagaagg agacgcggtc | 600 |
| ttcatcaatg taaactatgc gatccaaaat aaattaaatc ctaaaaaaga cgcaattgaa | 660 |
| gtagaatcaa cgaaaaacaa cccatacgct aacatcatcg cagtaagaaa aggcgaagaa | 720 |
| gattctgcaa aaatcaaagc gctgatggaa gttcttcact ctaaaaagat caaagacttc | 780 |
| atcgagaaaa aatacgacgg agctgtgctt cctgtatctg aataa | 825 |

B. subtilis YwqH - (P96720)

| | |
|---|---|
| 232. SQ SEQUENCE 140 AA; 15867 MW; 8FA05E8632B025B2 CRC64; | |
| MGYESMLADI KSSLNGKISD VEDKIEKLKK AKKDIDTLQE EAITEIKEIV KPELGKHWTG | |
| TKADDFDKGR EEAKSEASKI VNDKYNEYMA SINGKIFDLE WDKAKYASEL FIANGAADLL | |
| KKGEEFAEEV GNTISKLKWW | |
| 231. SQ Sequence 423 BP; 171 A; 55 C; 109 G; 88 T; 0 other; 1419947656 CRC32; | |
| atgggttatg aaagtatgct agcggatatc aaaagttcgc tcaacggaaa aatttcagac | 60 |
| gtggaagaca agatcgaaaa gctgaaaaaa gcaaaaaagg acatagacac actgcaagaa | 120 |
| gaggcaatca ctgaaatcaa agaaattgtg aaaccggaat tgggcaagca ttggacggga | 180 |
| acaaaagccg atgatttcga caagggaaga gaagaggcga aatcggaagc atctaagatt | 240 |
| gtgaatgata aatataacga gtatatggct tcgattaacg ggaaaatttt tgatcttgaa | 300 |
| tgggataaag ctaaatatgc atcggaattg ttcatagcaa atggtgcagc agatcttctt | 360 |
| aaaaagggag aagagttcgc ggaagaagtc ggaaatacaa ttagtaaact aaaatggtgg | 420 |
| tga | 423 |

B. subtilis YxeF - (P54945)

| | |
|---|---|
| 234. SQ SEQUENCE 144 AA; 16271 MW; D6320F00C082B969 CRC64; | |
| MVIPLRNKYG ILFLIAVCIM VSGCQQQKEE TPFYYGTWDE GRAPGPTDGV KSATVTFTED | |
| EVVETEVMEG RGEVQLPFMA YKVISQSTDG SIEIQYLGPY YPLKSTLKRG ENGTLIWEQN | |
| GQRKTMTRIE SKTGREEKDE KSKS | |
| 233. SQ Sequence 435 BP; 145 A; 80 C; 125 G; 85 T; 0 other; 276588478 CRC32; | |
| atggtgatcc ccttgagaaa caaatatggc attttgtttt taattgctgt atgcatcatg | 60 |
| gtatcgggct gccagcagca aaaagaagag acgccgtttt attacggaac gtgggatgag | 120 |
| gggcgtgccc ccgggccaac ggacggtgtg aaatcagcaa cagtcacatt taccgaagac | 180 |
| gaggttgtgg aaacggaagt gatggaagga agaggagagg tacagctgcc ttttatggca | 240 |
| tacaaggtga tttcccaaag cactgacggg tctatcgaga ttcagtacct cggcccttat | 300 |
| tatccgctca aaagcacgct gaaaagagga gaaaacggga cattgatatg ggagcaaaat | 360 |
| ggacagagaa aaacgatgac aagaatcgaa tcaaagaccg gcagggagga gaaagatgag | 420 |
| aaatcaaaaa gctga | 435 |

B. subtilis CspD - (P51777)

| | |
|---|---|
| 236. SQ SEQUENCE 66 AA; 7309 MW; 1A6CDA24E3A5AC58 CRC64; | |
| MQNGKVKWFN NEKGFGFIEV EGGDDVFVHF TAIEGDGYKS LEEGQEVSFE IVEGNRGPQA | |
| SNVVKL | |
| 235. SQ Sequence 201 BP; 73 A; 29 C; 46 G; 53 T; 0 other; 2696444462 CRC32; | |
| atgcaaaacg gtaaagtaaa atggttcaac aacgaaaaag gattcggctt cattgaagtt | 60 |
| gaaggcggag acgatgtatt tgttcacttc acagctatcg aaggagatgg atacaaatca | 120 |
| ttagaagaag gacaagaagt ttcttttgaa attgtcgaag gtaatcgtgg acctcaagct | 180 |
| tctaatgttg taaaactcta a | 201 |

B. subtilis Hsb - (Q5MCL3/Q9X3Z5)

| | |
|---|---|
| 238. SQ SEQUENCE 125 AA; 14560 MW; 377A6774F049CB6B CRC64; | |
| MSLVPYDPFR QLSNMRREFD RFFSELPISF DNEHGIGGIR VDVHETENEV VATCDLPGLE | |
| KKEDVDIDIQ NNRLSISGSI KRTNEIKEEN MLKKERYTGR FQRMITLPSP VSHDGVKSYV | |
| QKWNT | |
| 237. SQ Sequence 378 BP; 138 A; 52 C; 77 G; 111 T; 0 other; 1884122968 CRC32; | |
| atgtcattag taccttatga tccatttaga caattatcaa atatgagaag agaattcgat | 60 |
| cgtttctttt cggaattacc aatttcgttt gacaatgaac atggtatagg tgggattcga | 120 |
| gttgatgttc atgaaactga gaatgaggtt gtggcaacat gtgatttacc tggtcttgaa | 180 |
| aagaaagaag atgtagatat tgatatacaa aataacagat taagcattag tggttctatc | 240 |
| aagcgtacca atgaaataaa agaagaaaat atgttaaaaa aggaacgcta tacaggtcgt | 300 |
| tttcaacgta tgataacact tccaagcccc gtttcacatg atggggttaa aagctacgta | 360 |
| caaaaatgga atacttga | 378 |
| 240. SQ SEQUENCE 145 AA; 16701 MW; 821E4C9D66527563 CRC64; | |
| MSLVPYDPFR QLSNMRREFD RFFSELPISF DNEHGIGGIR VDVHETENEV VATCDLPGLE | |
| KKEDVDIDIQ NNRLSISGSI KRTNEIKEEN MLKKERYTGR FQRMITLPSP VSHDGVKATY | |
| KNGILEITMP KVAKDVKKKI DVSFQ | |
| 239. SQ Sequence 438 BP; 166 A; 59 C; 91 G; 122 T; 0 other; 776509077 CRC32; | |
| atgtcattag taccttatga tccatttaga caattatcaa atatgagaag agaattcgat | 60 |
| cgtttctttt cggaattacc aatttcgttt gacaatgaac atggtatagg tgggattcga | 120 |
| gttgatgttc atgaaactga gaatgaggtt gtggcaacat gtgatttacc tggtcttgaa | 180 |
| aagaaagaag atgtagatat tgatatacaa aataacagat taagcattag tggttctatc | 240 |
| aagcgtacca atgaaataaa agaagaaaat atgttaaaaa aggaacgcta tacaggtcgt | 300 |
| tttcaacgta tgataacact tccaagcccc gtttcacatg atggggttaa agctacgtac | 360 |
| aaaaatggaa tacttgaaat aacaatgcca aaagtggcga aggacgtaaa aaagaagata | 420 |
| gatgtaagtt tccagtaa | 438 |

B. subtilis PhoA - (P13792/O34804)

| | |
|---|---|
| 242. SQ SEQUENCE 240 AA; 27683 MW; 461A7CADB369C021 CRC64; | |
| MNKKILVVDD EESIVTLLQY NLERSGYDVI TASDGEEALK KAETEKPDLI VLDVMLPKLD | |
| GIEVCKQLRQ QKLMFPILML TAKDEEFDKV LGLELGADDY MTKPFSPREV NARVKAILRR | |
| SEIRAPSSEM KNDEMEGQIV IGDLKILPDH YEAYFKESQL ELTPKEFELL LYLGRHKGRV | |
| LTRDLLLSAV WNYDFAGDTR IVDVHISHLR DKIENNTKKP IYIKTIRGLG YKLEEPKMNE | |
| 241. SQ Sequence 723 BP; 244 A; 124 C; 181 G; 174 T; 0 other; 2080209762 CRC32; | |
| atgaacaaga aaattttagt tgtggatgat gaagaatcta ttgttactct tttacagtac | 60 |
| aatttggaac ggtcaggcta tgatgtcatt accgcctcgg atggggaaga agcactcaaa | 120 |
| aaagcggaaa cagagaaacc tgatttgatt gtgcttgatg tgatgcttcc aaaattggac | 180 |
| ggaatcgaag tatgcaagca gctgagacag caaaaactga tgtttcccat tttaatgctg | 240 |
| acagcgaagg atgaggaatt cgacaaagta ttagggctgg agctcggtgc tgatgattat | 300 |
| atgaccaagc cgttcagtcc aagggaagta aatgcgagag tcaaagcgat tttaaggcgt | 360 |
| tcggaaatag ctgcgccctc tagtgagatg aagaacgatg aaatggaagg ccagatcgta | 420 |
| atcggcgatc tgaaaatcct gcctgatcat tatgaagcgt actttaaaga aagtcagctt | 480 |
| gaactgacac cgaaagaatt cgaactgctg ctctatttag gcagacataa aggcagagtt | 540 |
| ctgacaagag acctgctgct gagcgcagtc tggaattatg attttgccgg agatacgaga | 600 |
| attgttgatg tgcacatcag ccatcttcgc gacaaaattg aaaacaatac caaaaaaccg | 660 |
| atctacatta aaacgattag gggattgggg tataaactgg aggagccaaa aatgaatgaa | 720 |
| taa | 723 |

B. subtilis SleB - (P50739)

| | |
|---|---|
| 244. SQ SEQUENCE 305 AA; 34002 MW; 9DF1305975F5BE16 CRC64; | |
| MKSKGSIMAC LILFSFTITT FINTETISAF SNQVIQRGAT GDDVVELQAR LQYNGYYNGK | |
| IDGVYGWGTY WAVRNFQDQF GLKEVDGLVG AKTKQTLICK SKYYREYVME QLNKGNTFTH | |
| YGKIPLKYQT KPSKAATQKA RQQAEARQKQ PAEKTTQKPK ANANKQQNNT PAKARKQDAV | |
| AANMPGGFSN NDIRLLAQAV YGEARGEPYE GQVAIAAVIL NRLNSPLFPN SVAGVIFEPL | |
| AFTAVADGQI YMQPNETARE AVLDAINGWD PSEEALYYFN PDTATSPWIW GRPQIKRIGK | |
| HIFCE | |
| 243. SQ Sequence 918 BP; 301 A; 189 C; 226 G; 202 T; 0 other; 3289157100 CRC32; | |
| atgaagtcca aaggatcgat tatggcatgt ctcatccttt tttcctttac aataacgacg | 60 |
| tttattaata ctgaaacgat ctctgccttt tcgaatcagg tcattcaaag aggggcaaca | 120 |
| ggggatgatg tggtcgagct tcaggcgcgt cttcaataca acggatatta taacggaaaa | 180 |
| attgacgggg tttatggatg ggggacgtac tgggcagttc gaaattttca ggatcaattc | 240 |
| gggttaaaag aggttgacgg ccttgtagga gctaaaacaa agcaaacctt aatatgtaaa | 300 |
| tcaaaatact atcgtgaata tgtcatggaa cagctcaata aagggaatac attcacgcat | 360 |
| tacggaaaaa ttccgctaaa gtatcagacg aaaccatcaa aagcagcaac acaaaaggca | 420 |
| agacaacaag cagaagcacg gcagaaacag cctgcggaaa aaacaacgca gaagcctaaa | 480 |
| gcgaatgcga ataaacagca aaacaataca ccagcaaaag caagaaaaca ggatgcggta | 540 |
| gcagcgaaca tgcctggtgg attttccaac aacgatatca ggctgcttgc tcaagcggtt | 600 |
| tatggcgaag cccggggcga gccgtacgag gggcaggttg ctattgcagc agtcatttta | 660 |
| aaccgtttga acagcccgtt atttccaaat tcagtagcgg gggttatttt tgagccgctt | 720 |
| gccttcacag cagtagccga cggacaaatt tacatgcagc cgaatgaaac ggcacgagaa | 780 |
| gcagtgctgg atgccatcaa tggctgggac ccatcagagg aagcacttta ctactttaat | 840 |
| ccggatacgg ctacaagtcc gtggatttgg gggcgtccgc agattaaaag aatcggtaaa | 900 |
| cacattttct gtgagtag | 918 |

B. subtilis SspA - (P04831)

| | |
|---|---|
| 246. SQ SEQUENCE 69 AA; 7071 MW; 270AC5260342C5D1 CRC64; | |
| MANNNSGNSN NLLVPGAAQA IDQMKLEIAS EFGVNLGADT TSRANGSVGG EITKRLVSFA | |
| QQNMGGGQF | |
| 245. SQ Sequence 210 BP; 69 A; 46 C; 45 G; 50 T; 0 other; 3172339658 CRC32; | |
| atggctaaca ataactcagg taacagcaac aaccttttag taccaggagc tgctcaagcg | 60 |
| atcgaccaaa tgaaattaga aatcgcttct gaattcggtg taaaccttgg agcagacaca | 120 |
| acttctcgcg ctaacggttc tgttggagga gagatcacaa aacgtcttgt atcttttgct | 180 |
| caacaaaaca tgggcggagg acaattctaa | 210 |

B. subtilis SspE - (P07784)

| | |
|---|---|
| 248. SQ SEQUENCE 84 AA; 9268 MW; 3C94015E1C0B237A CRC64; | |
| MANSNNFSKT NAQQVRKQNQ QSAAGQGQFG TEFASETNAQ QVRKQNQQSA GQQGQFGTEF | |
| ASETDAQQVR QQNQSAEQNK QQNS | |
| 247. SQ Sequence 255 BP; 110 A; 61 C; 44 G; 40 T; 0 other; 2461363522 CRC32; | |
| atggctaact caaataactt cagcaaaaca aacgctcaac aagttagaaa acaaaaccaa | 60 |
| caatcagctg ctggtcaagg tcaatttggc actgaatttg ctagcgaaac aaacgctcag | 120 |
| caagtcagaa aacaaaacca gcaatcagct ggacaacaag gtcaattcgg cactgaattc | 180 |
| gctagtgaaa ctgacgcaca gcaggtaaga cagcaaaacc aatctgctga acaaaacaaa | 240 |
| caacaaaaca gctaa | 255 |

B. subtilis YhcN - (P54598)

| | |
|---|---|
| 250. SQ SEQUENCE 189 AA; 20988 MW; 8C0BED95AC73E32D CRC64; | |
| MFGKKQVLAS VLLIPLLMTG CGVADQGEGR RDNNDVRNVN YRNPANDDMR NVNNRDNVDN | |
| NVNDNANNNR VNDDNNNDRK LEVADEAADK VTDLKEVKHA DIIVAGNQAY VAVVLTNGNK | |
| GAVENNLKKK IAKKVRSTDK NIDNVYVSAN PDFVERMQGY GKRIQNGDPI AGLFDEFTQT | |
| VQRVFPNAE | |
| 249. SQ Sequence 570 BP; 207 A; 97 C; 124 G; 142 T; 0 other; 1328369965 CRC32; | |
| atgtttggaa aaaaacaagt ccttgcgtct gtgcttctta tccctttgct tatgactggc | 60 |
| tgcggtgtag ccgaccaagg tgagggcaga cgtgataata atgatgtaag aaacgtaaat | 120 |
| tatcgaaatc cggccaatga cgatatgcgg aatgtaaaca atcgggataa cgttgacaac | 180 |
| aatgttaatg ataatgccaa taacaatcgt gtaaatgacg ataataacaa cgaccgaaaa | 240 |
| cttgaggttg ctgatgaagc agctgataaa gtaacagacc taaaagaagt aaagcatgcc | 300 |
| gatatcattg tggctggaaa tcaagcctac gttgcagtcg ttttaaccaa tggaaataaa | 360 |
| ggtgcagtag aaaacaatct gaagaaaaaa atagccaaaa aggtaagatc tactgacaaa | 420 |
| aacattgata atgtttacgt ttcagctaac cctgattttg tagagcgtat gcaaggatat | 480 |
| ggaaagcgta ttcaaaatgg tgacccaatc gccggattat ttgatgaatt tacacaaact | 540 |
| gtacagcgtg tattccctaa cgctgaataa | 570 |

B. subtilis YrbB(CoxA) - (P94446/O32061)

| | |
|---|---|
| 252. SQ SEQUENCE 172 AA; 19539 MW; 751B792B10F82D97 CRC64; | |
| MNDTRNNGNT RPIGYYTNEN DADRQGDGID HDGPVSELME DQNDGNRNTT NVNNRDRVTA | |
| DDRVPLATDG TYNNTNNRNM DRNAANNGYD NQENRRLAAK IANRVKQVKN VNDTQVMVSD | |
| DRVVIAVKSH REFTKSDRDN VVKAARNYAN GRDVQVSTDK GLFRKLHKMN NR | |
| 251. SQ Sequence 519 BP; 196 A; 110 C; 117 G; 96 T; 0 other; 4134087094 CRC32; | |
| atgaatgata cgcgcaataa cggcaatacc cgtccaatcg gatattatac aaatgaaaat | 60 |
| gacgccgata gacagggaga cggaatcgac cacgatggtc ctgtttctga attaatggag | 120 |
| gatcagaacg acggtaaccg aaacaccacg aatgtaaata accgtgaccg tgttactgct | 180 |
| gacgatcgtg ttcctttggc aactgacgga acatataaca acacgaataa ccgaaacatg | 240 |
| aatcggaatg cagcgaacaa cgggtatgac aaccaagaaa acagaagact ggctgcaaaa | 300 |
| attgccaacc gtgtgaaaca agtgaaaaac atcaatgaca cacaagttat ggtatcggat | 360 |
| gaccgagtag ttatcgcagt caaaagccac agagagttca caaagtctga cagagataat | 420 |
| gtcgtaaaag cagcgcgcaa ctatgcaaat ggccgtgacg ttcaagtatc aacagataaa | 480 |
| gggctgttca gaaaactcca taaaatgaac aaccgctag | 519 |

B. subtilis CggR - (O32253)

| | |
|---|---|
| 254. SQ SEQUENCE 340 AA; 37382 MW; 18C885966DDB42DB CRC64; | |
| MNQLIQAQKK LLPDLLLVMQ KRFEILQYIR LTEPIGRRSL SASLGISERV LRGEVQFLKE | |
| QNLVDIKTNG MTLTEEGYEL LSVLEDTMKD VLGLTLLEKT LKERLNLKDA IIVSGDSDQS | |
| PWVKKEMGRA AVACMKKRFS GKNIVAVTGG TTIEAVAEMM TPDSKNRELL FVPARGGLGE | |
| DVKNQANTIC AHMAEKASGT YRLLFVPGQL SQGAYSSIIE EPSVKEVLNT IKSASMLVHG | |
| IGEAKTMAQR RNTPLEDLKK IDDNDAVTEA FGYYFNADGE VVHKVHSVGM QLDDIDAIPD | |
| IIAVAGGSSK AEAIEAYFKK PRNTVLVTDE GAAKKLLRDE | |
| 253. SQ Sequence 1023 BP; 317 A; 203 C; 266 G; 237 T; 0 other; 1518175148 CRC32; | |
| atgaaccagt taatacaagc tcaaaaaaaa ttattgcctg atcttctgct cgttatgcaa | 60 |
| aagaggtttg aaatcttgca gtatatcagg ctgacagaac ccatcgggcg aagaagcctg | 120 |
| tctgccagtc tcggaatcag cgagcgtgtg ctgaggggcg aggttcagtt tttaaaggaa | 180 |
| cagaacctgg tcgatattaa gacaaacggc atgacattga cagaagaggg ctatgaactg | 240 |
| ctttctgttt tggaagatac gatgaaagat gttttaggtt tgactctttt ggaaaagaca | 300 |
| ttaaaagaac gtttaaatct aaaggatgcc attatcgtat ccggagacag cgatcaatcc | 360 |
| ccatgggtca aaaaagaaat gggaagagcg gctgtcgcat gtatgaaaaa aagattttca | 420 |
| ggcaaaaata tcgtcgctgt aactggcggt acgacaattg aagctgtcgc cgaaatgatg | 480 |
| acgccggatt ctaaaaaccg cgagcttttg tttgtgcctg caagaggcgg tttaggcgaa | 540 |
| gacgtgaaaa accaggcgaa caccatatgc gcgcatatgg cggagaaggc ttcaggcact | 600 |
| taccggcttt tgtttgttcc gggacagctg tcacaaggcg cctattcatc tattattgaa | 660 |
| gagccttctg tcaaagaggt gctgaacacc attaaatcag cgagtatgct ggttcacgga | 720 |
| atcggcgaag ctaaaactat ggctcagcgc agaaacacgc ctttagaaga tttaaagaaa | 780 |
| atagatgata acgacgcggt gacggaagcg ttcggctact attttaacgc ggacggcgaa | 840 |
| gtggttcaca aagtgcattc tgtcggaatg cagctggatg acatagacgc catccccgat | 900 |
| attattgcgg tagcgggcgg atcatcaaaa gccgaagcga tcgaggctta ctttaaaaag | 960 |
| ccacgcaaca cggttctcgt cacagacgaa ggagccgcaa agaagttatt aagggatgaa | 1020 |
| taa | 1023 |

B. subtilis CoxA - (P94446, O32061)

| | |
|---|---|
| 256. SQ SEQUENCE 172 AA; 19539 MW; 751B792B10F82D97 CRC64; | |
| MNDTRNNGNT RPIGYYTNEN DADRQGDGID HDGPVSELME DQNDGNRNTT NVNNRDRVTA | |
| DDRVPLATDG TYNNTNNRNM DRNAANNGYD NQENRRLAAK IANRVKQVKN VNDTQVMVSD | |
| DRVVIAVKSH REFTKSDRDN VVKAARNYAN GRDVQVSTDK GLFRKLHKMN NR | |
| 255. SQ Sequence 519 BP; 196 A; 110 C; 117 G; 96 T; 0 other; 4134087094 CRC32; | |
| atgaatgata cgcgcaataa cggcaatacc cgtccaatcg gatattatac aaatgaaaat | 60 |
| gacgccgata gacagggaga cggaatcgac cacgatggtc ctgtttctga attaatggag | 120 |
| gatcagaacg acggtaaccg aaacaccacg aatgtaaata accgtgaccg tgttactgct | 180 |
| gacgatcgtg ttcctttggc aactgacgga acatataaca acacgaataa ccgaaacatg | 240 |
| aatcggaatg cagcgaacaa cgggtatgac aaccaagaaa acagaagact ggctgcaaaa | 300 |
| attgccaacc gtgtgaaaca agtgaaaaac atcaatgaca cacaagttat ggtatcggat | 360 |
| gaccgagtag ttatcgcagt caaaagccac agagagttca caaagtctga cagagataat | 420 |
| gtcgtaaaag cagcgcgcaa ctatgcaaat ggccgtgacg ttcaagtatc aacagataaa | 480 |
| gggctgttca gaaaactcca taaaatgaac aaccgctag | 519 |

B. subtilis CwlJ - (P42249)

| | |
|---|---|
| 258. SQ SEQUENCE 142 AA; 16364 MW; 275A5BF1F6970912 CRC64; | |
| MAVVRATSAD VDLMARLLRA EAEGEGKQGM LLVGNVGINR LRANCSDFKG LRTIRQMIYQ | |
| PHAFEAVTHG YFYQRARDSE RALARGSING ERRWPAKFSL WYFRPQGDCP AQWYNQPFVA | |
| RFKSHCFYQP TAETCENVYN TF | |
| 257. SQ Sequence 429 BP; 104 A; 91 C; 133 G; 101 T; 0 other; 3513983261 CRC32; | |
| atggcggtcg tgagagcaac gagtgcggat gtcgatttga tggcaaggct gctcagagcg | 60 |
| gaagcggaag gcgaaggcaa gcaggggatg ctgcttgtcg gcaacgttgg aattaatcgg | 120 |
| ctgcgggcga attgctcaga ttttaaaggc ctccgcacca tcaggcagat gatttatcag | 180 |
| ccacacgcgt ttgaggctgt gactcatgga tatttttatc aaagggcgcg agatagcgag | 240 |
| cgtgcccttg cacgcggctc gattaatggt gaaaggcgct ggcctgcaaa atttagttta | 300 |
| tggtacttca ggccgcaggg ggactgtcca gcccagtggt ataaccagcc gtttgtggcc | 360 |
| agatttaagt cacactgctt ttatcagccg acggcggaga cgtgtgaaaa tgtatataac | 420 |
| acattttag | 429 |

B. subtilis SpoI VA - (P35149)

| | |
|---|---|
| 260. SQ SEQUENCE 492 AA; 55175 MW; 29EBA349DD18D12A CRC64; | |
| MEKVDIFKDI AERTGGDIYL GVVGAVRTGK STFIKKFMEL VVLPNISNEA DRARAQDELP | |
| QSAAGKTIMT TEPKFVPNQA MSVHVSDGLD VNIRLVDCVG YTVPGAKGYE DENGPRMINT | |
| PWYEEPIPFH EAAEIGTRKV IQEHSTIGVV ITTDGTIGDI ARSDYIEAEE RVIEELKEVG | |
| KPFIMVINSV RPYHPETEAM RQDLSEKYDI PVLAMSVESM RESDVLSVLR EALYEFPVLE | |
| VNVNLPSWVM VLKENHWLRE SYQESVKETV KDIKRLRDVD RVVGQFSEFE FIESAGLAGI | |
| ELGQGVAEID LYAPDHLYDQ ILKEVVGVEI RGRDHLLELM QDFAHAKTEY DQVSDALKMV | |
| KQTGYGIAAP ALADMSLDEP EIIRQGSRFG VRLKAVAPSI HMIKVDVESE FAPIIGTEKQ | |
| SEELVRYLMQ DFEDDPLSIW NSDIFGRSLS SIVREGIQAK LSLMPENARY KLKETLERII | |
| NEGSGGLIAI IL | |
| 259. SQ Sequence 1479 BP; 448 A; 293 C; 400 G; 338 T; 0 other; 2247466266 CRC32; | |
| ttggaaaagg tcgatatttt caaggatatc gctgaacgaa caggaggcga tatatactta | 60 |
| ggagtcgtag gtgctgtccg tacaggaaaa tccacgttca ttaaaaaatt tatggagctt | 120 |
| gtggtgctcc cgaatatcag taacgaagca gaccgggccc gagcgcagga tgaactgccg | 180 |
| cagagcgcag ccggcaaaac cattatgact acagagccta aatttgttcc gaatcaggcg | 240 |
| atgtctgttc atgtgtcaga cggactcgat gtgaatataa gattagtaga ttgtgtaggt | 300 |
| tacacagtgc ccggcgctaa aggatatgaa gatgaaaacg ggccgcggat gatcaatacg | 360 |
| ccttggtacg aagaaccgat cccatttcat gaggctgctg aaatcggcac acgaaaagtc | 420 |
| attcaagaac actcgaccat cggagttgtc attacgacag acggcaccat tggagatatc | 480 |
| gccagaagtg actatataga ggctgaagaa agagtcattg aagagctgaa agaggttggc | 540 |
| aaacctttta ttatggtcat caactcagtc aggccgtatc acccggaaac ggaagccatg | 600 |
| cgccaggatt taagcgaaaa atatgatatc ccggtattgg caatgagtgt agagagcatg | 660 |
| cgggaatcag atgtgctgag tgtgctcaga gaggccctct acgagtttcc ggtgctagaa | 720 |
| gtgaatgtca atctcccaag ctgggtaatg gtgctgaaag aaaaccattg gttgcgtgaa | 780 |
| agctatcagg agtccgtgaa ggaaacggtt aaggatatta aacggctccg ggacgtagac | 840 |
| agggttgtcg gccaattcag cgagtttgaa ttcattgaaa gtgccggatt agccggaatt | 900 |
| gagctgggcc aaggggtggc agaaattgat ttgtacgcgc ctgatcatct atatgatcaa | 960 |
| atcctaaaag aagttgtggg cgtcgaaatc agaggaagag accatctgct tgagctcatg | 1020 |
| caagacttcg cccatgcgaa aacagaatat gatcaagtgt ctgatgcctt aaaaatggtc | 1080 |
| aaacagacgg gatacggcat tgcagcgcct gctttagctg atatgagtct cgatgagccg | 1140 |
| gaaattataa ggcagggctc gcgattcggt gtgaggctga aagctgtcgc tccgtcgatc | 1200 |
| catatgatca aagtagatgt cgaaagcgaa ttcgccccga ttatcggaac ggaaaaacaa | 1260 |
| agtgaagagc ttgtacgcta tttaatgcag gactttgagg atgatccgct ctccatctgg | 1320 |
| aattccgata tcttcggaag gtcgctgagc tcaattgtga gagaagggat tcaggcaaag | 1380 |
| ctgtcattga tgcctgaaaa cgcacggtat aaattaaaag aaacattaga aagaatcata | 1440 |
| aacgaaggct ctggcggctt aatcgccatc atcctgtaa | 1479 |

B. subtilis SpoVM - (P37817)

| | |
|---|---|
| 262. SQ SEQUENCE 26 AA; 3018 MW; AC1BD750FCD420D5 CRC64; | |
| MKFYTIKLPK FLGGIVRAML GSFRKD | |
| 261. SQ Sequence 81 BP; 26 A; 11 C; 19 G; 25 T; 0 other; 1404161072 CRC32; | |
| atgaaatttt acaccattaa attgccgaag tttttaggag gaattgtccg ggcgatgctg | 60 |
| ggctcattta gaaaagatta a | 81 |

B. subtilis SpoVID - (P37963, O32062)

| | |
|---|---|
| 264. SQ SEQUENCE 575 AA; 64977 MW; 9A879AB16B18884F CRC64; | |
| MPQNHRLQFS VEESICFQKG QEVSELLSIS LDPDIRVQEV NDYVSIIGSL ELTGEYNIDQ | |
| NKHTEEIYTD KRFVEQVRKR EDGSAELTHC FPVDITIPKN KVSHLQDVFV FIDAFDYQLT | |
| DSRILTIQAD LAIEGLLDDT QDKEPEIPLY EAPAAFREEE LSEPPAHSVV EEPGASSAEE | |
| AVLQHEPPAE PPELFISKAG LREELETEKA ESEPPESVAS EPEAREDVKE EEESEELAVP | |
| ETEVRAESET EESEPEPDPS EIEIQEIVKA KKETAEPAAA IADVREEADS PAETELREHV | |
| GAEESPALEA ELHSETVIAK EKEETTVSPN HEYALRQEAQ NEEAAQSDQA DPALCQEEAE | |
| PDEALESVSE AALSIEDSRE TASAVYMEND NADLHFHFNQ KTSSEEASQE ELPEPAYRTF | |
| LPEQEEEDSF YSAPKLLEEE EQEEESFEIE VRKTPSAEEP KEETPFQSFQ LPESSETERK | |
| ETDAVPRVAP AAETKEPQTK ESDNSLYLTK LFTKEADEFS RMKICIVQQE DTIERLCERY | |
| EITSQQLIRM NSLALDDELK AGQILYIPQY KNSHA | |
| 263. SQ Sequence 1728 BP; 570 A; 334 C; 429 G; 395 T; 0 other; 1462811163 CRC32; | |
| ttgccgcaaa atcatcgatt gcaattttct gtagaagaat cgatctgttt ccaaaaagga | 60 |
| caggaagttt ctgaactgct ttctatttca ttagatcctg atattagggt tcaggaagta | 120 |
| aatgattatg tatcaatcat aggatcgctt gaacttacag gtgagtacaa catagatcaa | 180 |
| aacaaacata ccgaagagat ttatacagat aagcggtttg ttgaacaagt cagaaagaga | 240 |
| gaggatggaa gtgcggaact gactcactgt tttcctgtgg atattaccat tccgaaaaat | 300 |
| aaagtgagcc atttacagga tgtcttcgtc tttattgacg catttgacta tcaattgacg | 360 |
| gattcgcgca ttttaacaat tcaagctgat ttagcgatcg aagggctttt ggacgatacg | 420 |
| caagacaaag agccggagat acctttatat gaagctcctg cggcattcag ggaggaagag | 480 |
| ctttcagagc cgccggcaca tagtgtagta gaagaaccgg gtgcatcatc ggcagaggaa | 540 |
| gcagttcttc agcatgaacc gccagccgaa ccgccagaac tttttatctc gaaagcgggg | 600 |
| ctccgtgaag aactggagac agaaaaagca gaatctgagc cgcctgaatc ggttgcttca | 660 |
| gaaccagagg ccagagaaga tgtgaaagag gaagaagagt cagaagagct tgctgtgccg | 720 |
| gaaacggagg ttcgtgctga atcggaaaca gaagaatctg agccagaacc tgatccttca | 780 |
| gaaatagaga ttcaagagat cgttaaagca aaaaaagaaa cggcagagcc ggcagctgca | 840 |
| atagcggatg ttcgtgaaga agcagactct ccagcggaga ctgagcttcg tgaacacgtt | 900 |
| ggagcagaag aatcgcctgc tttggaagct gagcttcatt cagagactgt gattgcaaag | 960 |
| gaaaaagagg aaacaacagt gtctcctaat catgaatatg cgctgcgcca agaggctcaa | 1020 |
| aatgaagaag cagctcaatc ggatcaggct gatcctgcgc tttgccaaga agaggcggaa | 1080 |
| ccggatgaag ctttggagag tgtatcagag gccgctctct ccatagaaga tagcagagaa | 1140 |
| acagcttcag ctgtatatat ggagaatgac aatgccgatt tacatttcca tttcaatcaa | 1200 |
| aaaacaagct cggaggaggc atctcaagaa gaattgcctg aaccggcata ccgtaccttc | 1260 |
| ctgcctgaac aagaagagga ggattctttt tattcagcgc ctaagctgct ggaggaggaa | 1320 |
| gaacaagagg aagagagctt cgaaattgaa gtgagaaaaa caccatcagc tgaagagcct | 1380 |
| aaggaagaaa caccttttca atccttccag ctgcctgaat cttctgagac tgaaaggaag | 1440 |
| gaaacggatg ctgttcctag ggttgctcct gctgctgaaa cgaaggaacc tcaaacaaag | 1500 |
| gaaagtgata attctcttta tttaacaaaa ctctttacaa aagaagcgga tgagttttcg | 1560 |
| agaatgaaaa tttgtattgt gcagcaggaa gatacgatcg agcgtttatg cgaacggtat | 1620 |
| gaaattacat cccagcagct gatcaggatg aattctttag ccttggatga tgaattaaaa | 1680 |
| gcaggacaga ttctctatat tcctcaatat aaaaatagcc atgcgtaa | 1728 |

B. subtilis YhbA - (P97030, Q796Y4)

| | |
|---|---|
| 266. SQ SEQUENCE 435 AA; 48534 MW; 5E0C6194BA0CD275 CRC64; | |
| MTAAACKPAA RSVITESSLI FTSIHSSYVI STYYKRCVVL SQRKEAVQNM NVYQLKEELI | |
| EYAKSIGVDK IGFTTADTFD SLKDRLILQE SLGYLSGFEE PDIEKRVTPK LLLPKAKSIV | |
| AIALAYPSRM KDAPRSTRTE RRGIFCRASW GKDYHDVLRE KLDLLEDFLK SKHEDIRTKS | |
| MVDTGELSDR AVAERAGIGF SAKNCMITTP EYGSYVYLAE MITNIPFEPD VPIEDMCGSC | |
| TKCLDACPTG ALVNPGQLNA QRCISFLTQT KGFLPDEFRT KIGNRLYGCD TCQTVCPLNK | |
| GKDFHLHPEM EPDPEIAKPL LKPLLAISNR EFKEKFGHVS GSWRGKKPIQ RNAILALAHF | |
| KDASALPELT ELMHKDPRPV IRGTAAWAIG KIGDPAYAEE LEKALEKEKD EEAKLEIEKG | |
| IELLKASGMT KQGLS | |
| 265. SQ Sequence 1308 BP; 386 A; 281 C; 333 G; 308 T; 0 other; 3960484223 CRC32; | |
| atgacagcag ctgcatgtaa gccggccgca cgttcagtaa taacagaatc aagtttgata | 60 |
| ttcactagca ttcactccag ttacgtgata tcaacctatt ataaacgctg tgtcgtttta | 120 |
| tcacaaagaa aggaggctgt gcaaaacatg aacgtttatc agctcaaaga agaattaatt | 180 |
| gaatacgcga aaagcattgg cgtagacaag attggtttta cgaccgctga tacttttgac | 240 |
| agtttaaaag accgtttgat tcttcaagaa tcactcggct atctctccgg ctttgaagag | 300 |
| ccagatatcg aaaaaagggt gacgccgaag cttcttttgc cgaaagcgaa atcaatagtg | 360 |
| gcaattgctc tcgcatatcc ttccagaatg aaggatgcgc cgagaagcac gagaactgag | 420 |
| cgcaggggca ttttttgcag agcttcctgg ggaaaagact atcatgatgt gctgagggaa | 480 |
| aagcttgatc tgctggagga ttttctaaaa agcaagcatg aggatatcag aacgaagtca | 540 |
| atggttgata caggtgaatt gtctgatcgc gccgttgcgg aacgtgccgg aatcggattc | 600 |
| agtgcgaaaa actgtatgat cacaacaccc gagtatggct cttatgtgta tttggcggaa | 660 |
| atgatcacaa atatcccttt tgagcctgat gtgccgattg aagatatgtg cgggtcctgc | 720 |
| acgaaatgct tggacgcctg cccaacggga gcactggtta atcccgggca gcttaatgcg | 780 |
| cagcgctgca tctcttttct gacccagaca aaaggatttt tgcctgatga attccggaca | 840 |
| aaaatcggaa accgcctgta cgggtgcgat acgtgccaaa cggtatgccc tctcaataaa | 900 |
| gggaaggatt ttcatcttca tccggaaatg gagcctgatc ctgagattgc caaaccgtta | 960 |
| ttgaagccgc ttttggccat cagcaatcgg gaatttaagg agaaattcgg gcatgtctca | 1020 |
| ggttcttggc gcggaaaaaa accgattcag cgaaacgcca ttctcgcgct tgcccatttt | 1080 |
| aaggatgctt ccgcactgcc tgaattgacg gaactgatgc acaaggatcc gcgtcctgtc | 1140 |
| atcaggggga cagccgcatg ggcaatcgga aaaatcggag accccgccta cgcggaagag | 1200 |
| cttgaaaaag cgctggaaaa agagaaggat gaagaggcaa agctggaaat tgaaaaagga | 1260 |
| attgagttgc taaaagcttc aggcatgact aaacaaggcc tgtcctga | 1308 |

B. subtilis CSI5 - (P81095)

267. SQ SEQUENCE 11 AA; 1360 MW; 15F6ECEE6322C330 CRC64;
MRNIKVKPFL N
Nucleotide sequence not available

B. subtilis CspB - (P32081, P41017, Q45690)

| | |
|---|---|
| 270. SQ SEQUENCE 67 AA; 7365 MW; 1E7340FDB19E5BDC CRC64; | |
| MLEGKVKWFN SEKGFGFIEV EGQDDVFVHF SAIQGEGFKT LEEGQAVSFE IVEGNRGPQA | |
| ANVTKEA | |
| 269. SQ Sequence 204 BP; 69 A; 34 C; 47 G; 54 T; 0 other; 4076134933 CRC32; | |
| atgttagaag gtaaagtaaa atggttcaac tctgaaaaag gtttcggatt catcgaagta | 60 |
| gaaggtcaag acgatgtatt cgttcatttc tctgctattc aaggcgaagg cttcaaaact | 120 |
| ttagaagaag gccaagctgt ttcttttgaa atcgttgaag gaaaccgcgg accacaagct | 180 |
| gctaacgtta ctaaagaagc gtaa | 204 |

B. subtilis CspC - (P39158, Q79B46)

| | |
|---|---|
| 272. SQ SEQUENCE 66 AA; 7255 MW; C730336C131CB726 CRC64; | |
| MEQGTVKWFN AEKGFGFIER ENGDDVFVHF SAIQSDGFKS LDEGQKVSFD VEQGARGAQA | |
| ANVQKA | |
| 271. SQ Sequence 201 BP; 67 A; 32 C; 48 G; 54 T; 0 other; 1371678003 CRC32; | |
| atggaacaag gtacagttaa atggtttaat gcagaaaaag gttttggctt tatcgaacgc | 60 |
| gaaaatggag acgatgtatt cgtacacttt tctgcaatcc aaagtgacgg attcaaatct | 120 |
| ttagacgaag gtcaaaaagt atcgtttgac gttgagcaag gtgctcgtgg agctcaagct | 180 |
| gctaacgttc aaaaagctta a | 201 |

B. subtilis CspD - (P51777)

| | |
|---|---|
| 274. SQ SEQUENCE 66 AA; 7309 MW; 1A6CDA24E3A5AC58 CRC64; | |
| MQNGKVKWFN NEKGFGFIEV EGGDDVFVHF TAIEGDGYKS LEEGQEVSFE IVEGNRGPQA | |
| SNVVKL | |
| 273. SQ Sequence 201 BP; 73 A; 29 C; 46 G; 53 T; 0 other; 2696444462 CRC32; | |
| atgcaaaacg gtaaagtaaa atggttcaac aacgaaaaag gattcggctt cattgaagtt | 60 |
| gaaggcggag acgatgtatt tgttcacttc acagctatcg aaggagatgg atacaaatca | 120 |
| ttagaagaag gacaagaagt ttcttttgaa attgtcgaag gtaatcgtgg acctcaagct | 180 |
| tctaatgttg taaaactcta a | 201 |

B. subtilis DHBA - (P39071)

| | |
|---|---|
| 276. SQ SEQUENCE 261 AA; 27494 MW; 00B0EFBA53AB407C CRC64; | |
| MNAKGIEGKI AFITGAAQGI GEAVARTLAS QGAHIAAVDY NPEKLEKVVS SLKAEARHAE | |
| AFPADVRDSA AIDEITARIE REMGPIDILV NVAGVLRPGL IHSLSDEEWE ATFSVNSTGV | |
| FNASRSVSKY MMDRRSGSIV TVGSNPAGVP RTSMAAYASS KAAAVMFTKC LGLELAEYNI | |
| RCNIVSPGST ETDMQWSLWA DENGAEQVIK GSLETFKTGI PLKKLAKPSD IADAVLFLVS | |
| GQAGHITMHN LCVDGGATLG V | |
| 275. SQ Sequence 786 BP; 209 A; 164 C; 229 G; 184 T; 0 other; 475900199 CRC32; | |
| atgaatgcaa agggtataga gggaaaaatt gcttttataa caggggctgc ccaaggaata | 60 |
| ggcgaagctg ttgcgcggac gcttgccagt caaggcgcac atattgcggc agttgattat | 120 |
| aatcctgaaa agctggaaaa ggttgtgagc agcctcaaag cagaagcccg ccatgcagaa | 180 |
| gcttttcctg cggatgtgag agacagcgcg gcgattgacg agatcacggc gcgcatcgaa | 240 |
| cgtgaaatgg ggccgattga tattttagtg aatgtagcgg gtgtccttcg cccgggactg | 300 |
| atccattcgc ttagcgatga ggaatgggag gcgacgttct cagtgaattc gactggcgta | 360 |
| tttaacgcct cgcgttcagt cagcaaatat atgatggacc gaagatcggg ttcgattgta | 420 |
| acagtcggat cgaatcctgc cggtgtacca agaacatcta tggcggcata tgcgtcttca | 480 |
| aaggctgcgg ctgtgatgtt tacgaaatgc cttggccttg agcttgcaga atacaatatt | 540 |
| cgctgcaaca ttgtatctcc cggatcaacg gaaacagaca tgcagtggtc attatgggcc | 600 |
| gacgagaatg gagcggagca agtcataaaa ggatcacttg agacatttaa aacagggatc | 660 |
| ccgctcaaaa aactagccaa gccttcggat attgcggatg cggtgctctt tttggtttct | 720 |
| ggccaggcag ggcatattac gatgcataat ttatgcgtag atggcggggc gaccttaggc | 780 |
| gtgtaa | 786 |

B. subtilis FABI - (P54616, O31621)

| | |
|---|---|
| 278. SQ SEQUENCE 258 AA; 27874 MW; 097667168B3F0470 CRC64; | |
| MNFSLEGRNI VVMGVANKRS IAWGIARSLH EAGARLIFTY AGERLEKSVH ELAGTLDRND | |
| SIILPCDVTN DAEIETCFAS IKEQVGVIHG IAHCIAFANK EELVGEYLNT NRDGFLLAHN | |
| ISSYSLTAVV KAARPMMTEG GSIVTLTYLG GELVMPNYNV MGVAKASLDA SVKYLAADLG | |
| KENIRVNSIS AGPIRTLSAK GISDFNSILK DIEERAPLRR TTTPEEVGDT AAFLFSDMSR | |
| GITGENLHVD SGFHITAR | |
| 277. SQ Sequence 777 BP; 205 A; 187 C; 187 G; 198 T; 0 other; 4253509264 CRC32; | |
| atgaattttt cacttgaagg ccgtaacatt gttgtgatgg gggtagccaa caaacgcagc | 60 |
| atcgcctggg gcattgcgcg ttctttacat gaagcgggtg cacgtttgat tttcacatac | 120 |
| gctggtgaac gcctggagaa atccgttcac gagcttgccg gaacattaga ccgcaacgat | 180 |
| tccatcatcc tcccttgcga tgttacaaac gacgcagaaa tcgaaacttg cttcgcaagc | 240 |
| attaaggagc aggtcggtgt aatccacggt atcgcgcatt gtatcgcgtt tgccaacaaa | 300 |
| gaagagcttg tcggcgagta cttaaacaca aatcgtgacg gcttcctttt ggctcataac | 360 |
| atcagctcat attctctgac tgctgttgtc aaagcggcac gtccgatgat gactgaaggc | 420 |
| ggaagcattg tcactttgac gtaccttggc ggagagcttg tgatgccaaa ctacaacgtc | 480 |
| atgggtgtag caaaagcttc tcttgatgca agtgtgaaat atttagctgc tgacttagga | 540 |
| aaagaaaata tccgcgtcaa cagcatttct gccggcccga tcagaacatt atctgctaaa | 600 |
| ggcatcagcg atttcaactc tatcttaaaa gacatcgaag agcgtgcacc gcttcgccgc | 660 |
| acgacaacac ctgaagaagt gggcgataca gctgcgttct tgttcagcga tatgtcccgc | 720 |
| gggattacag gtgaaaatct tcacgttgat tctggtttcc atatcactgc ccgctaa | 777 |

B. subtilis RL10 - (P42923)

| | |
|---|---|
| 280. SQ SEQUENCE 165 AA; 17898 MW; 79AD7253D7EECDE5 CRC64; | |
| SSAIETKKVV VEEIASKLKE SKSTIIVDYR GLNVSEVTEL RKQLREANVE SKVYKNTMTR | |
| RAVEQAELNG LNDFLTGPNA IAFSTEDVVA PAKVLNDFAK NHEALEIKAG VIEGKVSTVE | |
| EVKALAELPP REGLLSMLLS VLKAPVRNLA LAAKAVAEQK EEQGA | |
| 279. SQ Sequence 501 BP; 158 A; 101 C; 110 G; 132 T; 0 other; 1367890263 CRC32; | |
| atgagcagcg caattgaaac aaaaaaagtt gttgttgaag aaattgcttc taaactgaaa | 60 |
| gaaagtaaat caacgatcat cgttgactat cgcggactta acgtttctga agtaactgaa | 120 |
| cttcgtaaac agcttcgcga agctaacgtt gagtccaaag tttacaaaaa tacgatgact | 180 |
| cgccgtgcgg ttgaacaagc tgagcttaat ggtttgaatg atttcttaac tggaccgaac | 240 |
| gcgatcgcat tcagcactga agatgttgtc gctccggcta aagttcttaa tgacttcgct | 300 |
| aaaaatcacg aagctcttga aatcaaagct ggcgttatcg aaggtaaagt ttctactgtt | 360 |
| gaagaagtga aagctcttgc tgaacttcca ccacgcgaag gcttgctttc tatgttgctt | 420 |
| agcgtactta aagctccagt tcgcaacctt gctcttgctg caaaagctgt ggcagaacaa | 480 |
| aaggaagaac aaggcgctta a | 501 |

B. subtilis SRFAD - (Q08788)

| | |
|---|---|
| 282. SQ SEQUENCE 241 AA; 27489 MW; 0333A4BDDE3B9682 CRC64; | |
| SQLFKSFDAS EKTQLICFPF AGGYSASFRP LHAFLQGECE MLAAEPPGHG TNQTSAIEDL | |
| EELTDLYKQE LNLRPDRPFV LFGHSMGGMI TFRLAQKLER EGIFPQAVII SAIQPPHIQR | |
| KKVSHLPDDQ FLDHIIQLGG MPAELVENKE VMSFFLPSFR SDYRALEQFE LYDLAQIQSP | |
| VHVFNGLDDK KCIRDAEGWK KWAKDITFHQ FDGGHMFLLS QTEEVAERIF AILNQHPIIQ | |
| P | |
| 281. SQ Sequence 729 BP; 177 A; 181 C; 184 G; 187 T; 0 other; 1087771314 CRC32; | |
| atgagccaac tcttcaaatc atttgatgcg tcggaaaaaa cacagctcat ctgttttccg | 60 |
| tttgccggcg gctattcggc gtcgtttcgc cctctccatg cttttttgca gggggagtgc | 120 |
| gagatgctcg ctgccgagcc gccgggacac ggcacgaatc aaacgtcagc cattgaggat | 180 |
| ctcgaagagc tgacggattt gtacaagcaa gaactgaacc ttcgccctga tcggccgttt | 240 |
| gtgctgttcg gacacagtat gggcggaatg atcaccttca ggctggcgca aaagcttgag | 300 |
| cgtgaaggca tctttccgca ggcggttatc atttctgcaa tccagccgcc tcatattcag | 360 |
| cggaagaaag tgtcccacct gcctgatgat cagtttctcg atcatattat ccaattaggc | 420 |
| ggaatgcccg cagagcttgt tgaaaataag gaggtcatgt cctttttcct gccttctttc | 480 |
| cgatcagatt accgggctct tgaacaattt gagctttacg atctggccca gatccagtcg | 540 |
| cctgttcatg tctttaacgg gcttgatgat aaaaaatgca tacgagatgc ggaagggtgg | 600 |
| aagaagtggg caaaagacat cacattccat caatttgacg gcgggcacat gttcctgctg | 660 |
| tcacaaacgg aagaagtcgc agaacggatt tttgcgatct tgaatcagca tccgatcatt | 720 |
| caaccgtga | 729 |

B. subtilis SAS1 - (P84583)

283. SQ SEQUENCE 69 AA; 7068 MW; 7F47C5761E50D440 CRC64;
PNQSGSNSSN QLLVPGAAQA IDQMKFEIAS EFGVNLGAET TSRANGSVGG EITKRLVSFA
QQQMGGGVQ
Nucleotide sequence not available

B. subtilis SAS2 - (P84584)

285. SQ SEQUENCE 70 AA; 7332 MW; D5BC83049D1CA815 CRC64;
AQNSQNGNSS NQLLVPGAAQ AIDQMKFEIA SEFGVNLGAE TTSRANGSVG GEITKRLVSF
AQQNMSGQQF
Nucleotide sequence not available

B. subtilis SASG - (P04585)

| | |
|---|---|
| 288. SQ SEQUENCE 1003 AA; 113780 MW; C426B37D23C5FA9F CRC64; | |
| FFREDLAFLQ GKAREFSSEQ TRANSPTRRE LQVWGRDNNS PSEAGADRQG TVSFNFPQVT | |
| LWQRPLVTIK IGGQLKEALL DTGADDTVLE EMSLPGRWKP KMIGGIGGFI KVRQYDQILI | |
| EICGHKAIGT VLVGPTPVNI IGRNLLTQIG CTLNFPISPI ETVPVKLKPG MDGPKVKQWP | |
| LTEEKIKALV EICTEMEKEG KISKIGPENP YNTPVFAIKK KDSTKWRKLV DFRELNKRTQ | |
| DFWEVQLGIP HPAGLKKKKS VTVLDVGDAY FSVPLDEDFR KYTAFTIPSI NNETPGIRYQ | |
| YNVLPQGWKG SPAIFQSSMT KILEPFRKQN PDIVIYQYMD DLYVGSDLEI GQHRTKIEEL | |
| RQHLLRWGLT TPDKKHQKEP PFLWMGYELH PDKWTVQPIV LPEKDSWTVN DIQKLVGKLN | |
| WASQIYPGIK VRQLCKLLRG TKALTEVIPL TEEAELELAE NREILKEPVH GVYYDPSKDL | |
| IAEIQKQGQG QWTYQIYQEP FKNLKTGKYA RMRGAHTNDV KQLTEAVQKI TTESIVIWGK | |
| TPKFKLPIQK ETWETWWTEY WQATWIPEWE FVNTPPLVKL WYQLEKEPIV GAETFYVDGA | |
| ANRETKLGKA GYVTNRGRQK VVTLTDTTNQ KTELQAIYLA LQDSGLEVNI VTDSQYALGI | |
| IQAQPDQSES ELVNQIIEQL IKKEKVYLAW VPAHKGIGGN EQVDKLVSAG IRKVLFLDGI | |
| DKAQDEHEKY HSNWRAMASD FNLPPVVAKE IVASCDKCQL KGEAMHGQVD CSPGIWQLDC | |
| THLEGKVILV AVHVASGYIE AEVIPAETGQ ETAYFLLKLA GRWPVKTIHT DNGSNFTGAT | |
| VRAACWWAGI KQEFGIPYNP QSQGVVESMN KELKKIIGQV RDQAEHLKTA VQMAVFIHNF | |
| KRKGGIGGYS AGERIVDIIA TDIQTKELQK QITKIQNFRV YYRDSRNPLW KGPAKLLWKG | |
| EGAVVIQDNS DIKVVPRRKA KIIRDYGKQM AGDDCVASRQ DED | |
| 287. SQ Sequence 2739 BP; 1084 A; 431 C; 619 G; 605 T; 0 other; 4122321072 CRC32; | |
| atgagtttgc caggaagatg gaaaccaaaa atgatagggg gaattggagg ttttatcaaa | 60 |
| gtaagacagt atgatcagat actcatagaa atctgtggac ataaagctat aggtacagta | 120 |
| ttagtaggac ctacacctgt caacataatt ggaagaaatc tgttgactca gattggttgc | 180 |
| actttaaatt ttcccattag ccctattgag actgtaccag taaaattaaa gccaggaatg | 240 |
| gatggcccaa aagttaaaca atggccattg acagaagaaa aaataaaagc attagtagaa | 300 |
| atttgtacag agatggaaaa ggaagggaaa atttcaaaaa ttgggcctga aaatccatac | 360 |
| aatactccag tatttgccat aaagaaaaaa gacagtacta aatggagaaa attagtagat | 420 |
| ttcagagaac ttaataagag aactcaagac ttctgggaag ttcaattagg aataccacat | 480 |
| cccgcagggt taaaaaagaa aaaatcagta acagtactgg atgtgggtga tgcatatttt | 540 |
| tcagttccct tagatgaaga cttcaggaag tatactgcat ttaccatacc tagtataaac | 600 |
| aatgagacac cagggattag atatcagtac aatgtgcttc cacagggatg gaaaggatca | 660 |
| ccagcaatat tccaaagtag catgacaaaa atcttagagc cttttagaaa acaaaatcca | 720 |
| gacatagtta tctatcaata catggatgat ttgtatgtag gatctgactt agaaataggg | 780 |
| cagcatagaa caaaaataga ggagctgaga caacatctgt tgaggtgggg acttaccaca | 840 |
| ccagacaaaa aacatcagaa agaacctcca ttcctttgga tgggttatga actccatcct | 900 |
| gataaatgga cagtacagcc tatagtgctg ccagaaaaag acagctggac tgtcaatgac | 960 |
| atacagaagt tagtggggaa attgaattgg gcaagtcaga tttacccagg gattaaagta | 1020 |
| aggcaattat gtaaactcct tagaggaacc aaagcactaa cagaagtaat accactaaca | 1080 |
| gaagaagcag agctagaact ggcagaaaac agagagattc taaaagaacc agtacatgga | 1140 |
| gtgtattatg acccatcaaa agacttaata gcagaaatac agaagcaggg gcaaggccaa | 1200 |
| tggacatatc aaatttatca agagccattt aaaaatctga aaacaggaaa atatgcaaga | 1260 |
| atgaggggtg cccacactaa tgatgtaaaa caattaacag aggcagtgca aaaaataacc | 1320 |
| acagaaagca tagtaatatg gggaaagact cctaaattta aactgcccat acaaaaggaa | 1380 |
| acatgggaaa catggtggac agagtattgg caagccacct ggattcctga gtgggagttt | 1440 |
| gttaataccc ctcccttagt gaaattatgg taccagttag agaaagaacc catagtagga | 1500 |
| gcagaaacct tctatgtaga tggggcagct aacagggaga ctaaattagg aaaagcagga | 1560 |
| tatgttacta atagaggaag acaaaaagtt gtcaccctaa ctgacacaac aaatcagaag | 1620 |
| actgagttac aagcaattta tctagctttg caggattcgg gattagaagt aaacatagta | 1680 |
| acagactcac aatatgcatt aggaatcatt caagcacaac cagatcaaag tgaatcagag | 1740 |
| ttagtcaatc aaataataga gcagttaata aaaaaggaaa aggtctatct ggcatgggta | 1800 |
| ccagcacaca aaggaattgg aggaaatgaa caagtagata aattagtcag tgctggaatc | 1860 |
| aggaaagtac tatttttaga tggaatagat aaggcccaag atgaacatga gaaatatcac | 1920 |
| agtaattgga gagcaatggc tagtgatttt aacctgccac ctgtagtagc aaaagaaata | 1980 |
| gtagccagct gtgataaatg tcagctaaaa ggagaagcca tgcatggaca agtagactgt | 2040 |
| agtccaggaa tatggcaact agattgtaca catttagaag gaaaagttat cctggtagca | 2100 |
| gttcatgtag ccagtggata tatagaagca gaagttattc cagcagaaac agggcaggaa | 2160 |
| acagcatatt ttcttttaaa attagcagga agatggccag taaaaacaat acatactgac | 2220 |
| aatggcagca atttcaccgg tgctacggtt agggccgcct gttggtgggc gggaatcaag | 2280 |
| caggaatttg gaattcccta caatccccaa agtcaaggag tagtagaatc tatgaataaa | 2340 |
| gaattaaaga aaattatagg acaggtaaga gatcaggctg aacatcttaa gacagcagta | 2400 |
| caaatggcag tattcatcca caattttaaa agaaaagggg ggattggggg gtacagtgca | 2460 |
| ggggaaagaa tagtagacat aatagcaaca gacatacaaa ctaaagaatt acaaaaacaa | 2520 |
| attacaaaaa ttcaaaattt tcgggtttat tacagggaca gcagaaatcc actttggaaa | 2580 |
| ggaccagcaa agctcctctg gaaaggtgaa ggggcagtag taatacaaga taatagtgac | 2640 |
| ataaaagtag tgccaagaag aaaagcaaag atcattaggg attatggaaa acagatggca | 2700 |
| ggtgatgatt gtgtggcaag tagacaggat gaggattag | 2739 |

B. subtilis SSPA - (P04831)

| | |
|---|---|
| 290. SQ SEQUENCE 69 AA; 7071 MW; 270AC5260342C5D1 CRC64; | |
| MANNNSGNSN NLLVPGAAQA IDQMKLEIAS EFGVNLGADT TSRANGSVGG EITKRLVSFA | |
| QQNMGGGQF | |
| 289. SQ Sequence 210 BP; 69 A; 46 C; 45 G; 50 T; 0 other; 3172339658 CRC32; | |
| atggctaaca ataactcagg taacagcaac aaccttttag taccaggagc tgctcaagcg | 60 |
| atcgaccaaa tgaaattaga aatcgcttct gaattcggtg taaaccttgg agcagacaca | 120 |
| acttctcgcg ctaacggttc tgttggagga gagatcacaa aacgtcttgt atcttttgct | 180 |
| caacaaaaca tgggcggagg acaattctaa | 210 |

B. subtilis SSPB - (P04832)

| | |
|---|---|
| 292. SQ SEQUENCE 67 AA; 6980 MW; 19A3972001E81621 CRC64; | |
| MANQNSSNDL LVPGAAQAID QMKLEIASEF GVNLGADTTS RANGSVGGEI TKRLVSFAQQ | |
| QMGGRVQ | |
| 291. SQ Sequence 204 BP; 60 A; 48 C; 45 G; 51 T; 0 other; 2069831197 CRC32; | |
| atggctaacc aaaactcttc aaatgactta ctagttcctg gcgcagctca ggctatcgat | 60 |
| caaatgaaac ttgaaatcgc ttctgaattc ggcgttaacc ttggagcgga cacaacttct | 120 |
| cgcgctaacg gttctgtcgg aggagaaatc acaaaacgtt tagtatcttt cgctcagcag | 180 |
| caaatgggcg gcagagttca ataa | 204 |

B. subtilis SSPC - (P02958)

| | |
|---|---|
| 294. SQ SEQUENCE 72 AA; 7758 MW; F1E1788E86F28F8D CRC64; | |
| MAQQSRSRSN NNNDLLIPQA ASAIEQMKLE IASEFGVQLG AETTSRANGS VGGEITKRLV | |
| RLAQQNMGGQ FH | |
| 293. SQ Sequence 219 BP; 75 A; 41 C; 42 G; 61 T; 0 other; 2865265306 CRC32; | |
| atggctcaac aaagtagatc aagatcaaac aacaataatg atttactaat tcctcaagca | 60 |
| gcttcagcta ttgaacaaat gaaacttgaa atagcttctg agtttggtgt tcaattaggc | 120 |
| gctgagacta catctcgtgc aaacggttca gttggtggag aaatcactaa acgtttagtt | 180 |
| cgcttagctc aacaaaacat gggcggtcaa tttcattaa | 219 |

B. subtilis SSPD - (P04833)

| | |
|---|---|
| 296. SQ SEQUENCE 63 AA; 6672 MW; ACBD22A3F707DC78 CRC64; | |
| ASRNKLVVPG VEQALDQFKL EVAQEFGVNL GSDTVARANG SVGGEMTKRL VQQAQSQLNG | |
| TTK | |
| 295. SQ Sequence 195 BP; 64 A; 41 C; 51 G; 39 T; 0 other; 392481711 CRC32; | |
| atggcgagca gaaataaact cgttgttcca ggggtggagc aggcactaga ccaatttaaa | 60 |
| ctcgaagtgg ctcaagaatt cggtgtgaac cttggttctg atacagtcgc acgcgctaac | 120 |
| ggctctgtag gcggagaaat gacaaagcgg ctggtacagc aagcacaatc acaattaaat | 180 |
| ggcacaacta aataa | 195 |

B. subtilis SSPE - (P07784)

| | |
|---|---|
| 298. SQ SEQUENCE 84 AA; 9268 MW; 3C94015E1C0B237A CRC64; | |
| MANSNNFSKT NAQQVRKQNQ QSAAGQGQFG TEFASETNAQ QVRKQNQQSA GQQGQFGTEF | |
| ASETDAQQVR QQNQSAEQNK QQNS | |
| 297. SQ Sequence 255 BP; 110 A; 61 C; 44 G; 40 T; 0 other; 2461363522 CRC32; | |
| atggctaact caaataactt cagcaaaaca aacgctcaac aagttagaaa acaaaaccaa | 60 |
| caatcagctg ctggtcaagg tcaatttggc actgaatttg ctagcgaaac aaacgctcag | 120 |
| caagtcagaa aacaaaacca gcaatcagct ggacaacaag gtcaattcgg cactgaattc | 180 |
| gctagtgaaa ctgacgcaca gcaggtaaga cagcaaaacc aatctgctga acaaaacaaa | 240 |
| caacaaaaca gctaa | 255 |

B. subtilis SSPG - (Q7WY59)

| | |
|---|---|
| 300. SQ SEQUENCE 47 AA; 5139 MW; 111336E247EEDD8C CRC64; | |
| SENRHENEEN RRDAAVAKVQ NSGNAKVVVS VNTDQDQAQA QSQDGED | |
| 299. SQ Sequence 147 BP; 58 A; 29 C; 42 G; 18 T; 0 other; 2452688163 CRC32; | |
| atgagcgaaa atcgtcatga aaatgaagaa aacagacgcg atgcggcagt ggcaaaagtc | 60 |
| caaaacagcg gtaatgcaaa agtcgtggtc agcgtgaaca cagatcagga tcaggcacag | 120 |
| gcgcagtcac aagacggaga agactaa | 147 |

B. subtilis SSPH - (O31552)

| | |
|---|---|
| 302. SQ SEQUENCE 59 AA; 6869 MW; E54FF9C14FDE96F1 CRC64; | |
| MNIQRAKEIV ESPDMKKVTY NGVPIYIQHV NEETGTARIY PLDEPQEEHE VQLNSLKED | |
| 301. SQ Sequence 180 BP; 72 A; 31 C; 40 G; 37 T; 0 other; 2308147894 CRC32; | |
| atgaatattc aaagggcgaa agaaattgta gaatctcccg acatgaagaa agtaacatat | 60 |
| aacggcgttc ctatttacat tcagcacgta aatgaagaaa ctggaacagc aagaatttat | 120 |
| ccgcttgacg aaccgcaaga ggagcatgaa gtgcagctga acagcttaaa agaggattaa | 180 |

B. subtilis SSPI - (P94537)

| | |
|---|---|
| 304. SQ SEQUENCE 71 AA; 7853 MW; 010361FF63A925B5 CRC64; | |
| MDLNLRHAVI ANVTGNNQEQ LEHTIVDAIQ SGEEKMLPGL GVLFEVIWQH ASESEKNEML | |
| KTLEGGLKPA E | |
| 303. SQ Sequence 216 BP; 71 A; 45 C; 52 G; 48 T; 0 other; 1669772580 CRC32; | |
| atggatctta atttacgtca tgccgtcatt gccaatgtca ccggcaataa tcaggagcag | 60 |
| cttgagcata caatcgtaga tgcgattcaa agcggtgaag aaaaaatgct tccagggctc | 120 |
| ggcgttttat tcgaagtcat ttggcagcac gcatccgaaa gtgagaaaaa cgaaatgctg | 180 |
| aaaacgcttg aaggcggatt aaaacccgcc gaataa | 216 |

B. subtilis SSPJ - (Q7WY58)

| | |
|---|---|
| 306. SQ SEQUENCE 45 AA; 5031 MW; 59F70296024A6EDD CRC64; | |
| GFFNKDKGKR SEKEKNVIQG ALEDAGSALK DDPLQEAVQK KKNNR | |
| 305. SQ Sequence 141 BP; 62 A; 20 C; 28 G; 31 T; 0 other; 99470552 CRC32; | |
| atgggtttct ttaataaaga taaaggaaaa cgttccgaaa aagaaaaaaa cgtaatccaa | 60 |
| ggagctcttg aagatgctgg ttcagctcta aaagatgatc cgcttcaaga agctgtgcaa | 120 |
| aaaaagaaaa ataatcgata a | 141 |

B. subtilis SSPK - (Q7WY75)

| | |
|---|---|
| 308. SQ SEQUENCE 49 AA; 5722 MW; 0272AD15F94BBA6C CRC64; | |
| VRNKEKGFPY ENENKFQGEP RAKDDYASKR ADGSINQHPQ ERMRASGKR | |
| 307. SQ Sequence 153 BP; 61 A; 30 C; 35 G; 27 T; 0 other; 2628757375 CRC32; | |
| atggtccgaa ataaagaaaa aggatttcct tacgaaaacg aaaacaaatt tcagggtgaa | 60 |
| ccgagagcaa aggacgacta tgcttcaaag cgtgctgacg gatctatcaa tcagcatcct | 120 |
| caagaaagaa tgagagcctc aggcaaacgg taa | 153 |

B. subtilis SSPL - (Q7WY66)

| | |
|---|---|
| 310. SQ SEQUENCE 42 AA; 4694 MW; 96CEA320BA4D180B CRC64; | |
| MKKKDKGRLT GGVTPQGDLE GNTHNDPKTE LEERAKKSNT KR | |
| 309. SQ Sequence 129 BP; 54 A; 26 C; 33 G; 16 T; 0 other; 2802479283 CRC32; | |
| atgaaaaaga aagataaagg ccggctgacc ggcggtgtta ctccgcaagg cgacctggaa | 60 |
| ggcaatacac ataatgaccc taaaacagag cttgaggaga gagcaaaaaa aagcaataca | 120 |
| aaacgctag | 129 |

B. subtilis SSPM - (Q7WY65)

| | |
|---|---|
| 312. SQ SEQUENCE 34 AA; 3725 MW; 890554D4C2BB9A42 CRC64; | |
| MKTRPKKAGQ QKKTESKAID SLDKKLGGPN RPST | |
| 311. SQ Sequence 105 BP; 45 A; 24 C; 20 G; 16 T; 0 other; 1126293400 CRC32; | |
| atgaaaacaa gaccgaaaaa agccggccag caaaaaaaga ctgaatcaaa ggcaatcgat | 60 |
| tctttagata aaaaattagg cggcccgaac cgcccttcta cgtaa | 105 |

B. subtilis SSPN - (Q7WY69)

| | |
|---|---|
| 314. SQ SEQUENCE 48 AA; 5353 MW; 283A62D662070859 CRC64; | |
| MGNNKKNGQP QYVPSHLGTK PVKYKANKGE KMHDTSGQRP IIMQTKGE | |
| 313. SQ Sequence 147 BP; 60 A; 28 C; 34 G; 25 T; 0 other; 3569110721 CRC32; | |
| atgggaaaca acaagaaaaa cggtcagcct caatatgttc caagccactt gggtacaaag | 60 |
| cctgtaaaat ataaagccaa taaaggggaa aaaatgcatg atacttcagg acagcggccg | 120 |
| attatcatgc agacaaaagg cgagtag | 147 |

B. subtilis SSPO - (P71031)

| | |
|---|---|
| 316. SQ SEQUENCE 47 AA; 5296 MW; E9C1A7B3F4759911 CRC64; | |
| VKRKANHVIN GMNDAKSQGK GAGYIENDQL VLTEAERQNN KKRKTNQ | |
| 315. SQ Sequence 147 BP; 69 A; 29 C; 29 G; 20 T; 0 other; 3053943211 CRC32; | |
| atggtcaaaa gaaaagcgaa tcacgtcatt aacggaatga atgacgcaaa aagccaaggc | 60 |
| aaaggcgccg gctatattga aaacgaccag cttgtactga ctgaagcaga acgccaaaat | 120 |
| aacaaaaaaa gaaaaaccaa tcaataa | 147 |

B. subtilis SSPP - (P71032)

| | |
|---|---|
| 318. SQ SEQUENCE 48 AA; 5431 MW; 95977382600C9217 CRC64; | |
| MTNKNTSKDM HKNAPKGHNP GQPEPLSGSK KVKNRNHTRQ KHNSSHDM | |
| 317. SQ Sequence 147 BP; 70 A; 36 C; 23 G; 18 T; 0 other; 3603452568 CRC32; | |
| atgaccaata agaatacaag taaagatatg cataaaaacg cccctaaagg acacaatccc | 60 |
| ggccaacccg agcctctaag cggaagcaaa aaagtaaaaa accgaaacca tacaagacaa | 120 |
| aagcacaact caagccatga tatgtaa | 147 |

B. subtilis TLP - (Q45060)

| | |
|---|---|
| 320. SQ SEQUENCE 82 AA; 9591 MW; 46760A24FC2F7766 CRC64; | |
| TKNQNQYQQP NPDDRSDNVE KLQDMVQNTI ENIEEAEASM EFASGEDKQR IKEKNARREQ | |
| SIEAFRNEIQ DESAARQNGY RS | |
| 319. SQ Sequence 252 BP; 105 A; 41 C; 55 G; 51 T; 0 other; 1430022231 CRC32; | |
| atgacaaaga accaaaatca atatcagcag cctaatcctg atgatcgttc tgacaatgtg | 60 |
| gaaaaattgc aggatatggt tcaaaataca attgaaaata tagaagaagc agaagcatca | 120 |
| atggagtttg cttcaggaga agataaacag cgtatcaaag aaaaaaatgc aaggcgcgaa | 180 |
| cagagcattg aagcgtttcg taatgaaata caggacgaat ctgcagcgag acaaaacgga | 240 |
| taccgttctt aa | 252 |

B. subtilis SSPG-1 - (Q9AH72)

| | |
|---|---|
| 322. SQ SEQUENCE 85 AA; 9339 MW; BCD55A8C95C66877 CRC64; | |
| MANSNNFSKT NAQQVRKQNQ QSAAGQGQFG TEFASETNAQ QVRKQNQQSA AGQQGQFGTE | |
| FASETDAQQV RQQNQSAEQN KQQNS | |
| 321. SQ Sequence 258 BP; 110 A; 64 C; 45 G; 39 T; 0 other; 3108717180 CRC32; | |
| atggctaact caaacaattt cagcaaaaca aacgcacaac aagttagaaa acaaaaccaa | 60 |
| caatcagctg ctggtcaagg tcaattcggc actgaatttg ctagcgaaac aaacgctcag | 120 |
| caagtcagaa aacaaaacca gcaatcagct gctggccaac aaggtcaatt cggcactgaa | 180 |
| ttcgctagtg aaactgacgc acagcaggta agacagcaaa accaatctgc tgaacaaaac | 240 |
| aaacaacaaa acagctaa | 258 |

B. subtilis SSPG-2 - (Q9AH73)

| | |
|---|---|
| 324. SQ SEQUENCE 85 AA; 9367 MW; BCD5423BC5C66877 CRC64; | |
| MANSNNFSKT NAQQVRKQNQ QSAAGQGQFG TEFASETNAQ QVRKQNQQSA AGQQGQFGTE | |
| FASETDVQQV RQQNQSAEQN KQQNS | |
| 323. SQ Sequence 258 BP; 110 A; 63 C; 45 G; 40 T; 0 other; 1588272575 CRC32; | |
| atggctaact caaacaattt cagcaaaaca aacgcacaac aagttagaaa acaaaaccaa | 60 |
| caatcagctg ctggtcaagg tcaattcggc actgaatttg ctagcgaaac aaacgctcag | 120 |
| caagtcagaa aacaaaacca gcaatcagct gctggccaac aaggtcaatt cggcactgaa | 180 |
| ttcgctagtg aaactgacgt acagcaggta agacagcaaa accaatctgc tgaacaaaac | 240 |
| aaacaacaaa acagctaa | 258 |

| Document D: List of Amino Acid and Nucleotide Sequence for Surface | | |
|---|---|---|
| Proteins from Bacillus cereus that are predicted to be included in | | |
| Bacillus anthracis | | |
| B. cereus ExsA-(Q6B4J5) | | |
| 326. | SQ SEQUENCE 643 AA; 72839 MW; 51BB9AC63021CFD9 CRC64; | |
| | MKIHIVQKGD TLWKIAKKYG VDFDTLKKTN TQLSNPDLIM PGMKIKVPSK SVHMKQQAGA | |
| | GSAPPKQYVK EVQQKEFAAT PTPLGIEDEE EVTYQSAPIT QQPAMQQTQK EVQIKPQKEM | |
| | QVKPQKEVQV KPQKEMQVKP QKEVQKEQPI QKEKPVEKPS VIQKPPVIEK QKPAEKENTK | |
| | FSVNVLPQPP QPPIKPKKEY KISDVIKKGS ELIAPQISKM KPNNIISPQT KKNNIISPQV | |
| | KKENVGNIVS PQVKKENVGN IVSPQVKKEN VGNIVSPQVK KENVGNIVSP QVKKENVGNI | |
| | VSPQVKKENV GNIVSPQVKK ENVGNIVSPN VSKENVVIPQ VIPPNIQMPN IMPIMDNNQP | |
| | PNIMPIMDNN QPPNIMPIMD NNQMPNMMPI MDNNQMPNMM PIMDNNQMPN MMPIMDNNQM | |
| | PNMMPIMDNN QMPNMMPIMD NNQMPNMMPI MDNNQMPNMM PIMDNNQMPN IMPIMDNNQM | |
| | PNMMPIMDNN QMPNIMPIMD NNQMPNMMPI MDNNQPPNMM PYQMPYQQPM MPPNPYYQQP | |
| | NPYQMPYQQG APFGPQHTSM PNQNMMPMDN NMPPLVQGEE DCGCGGESRL YSPQPGGPQY | |
| | ANPLYYQPTQ SAYAPQPGTM YYQPDPPNVF GEPVSEEEDE EEV | |
| 325. | SQ Sequence 1932 BP; 813 A; 355 C; 371 G; 393 T; 0 other; 206901513 | |
| | CRC32; | |
| | ttgaaaattc atatcgtgca aaaaggggat accctttgga aaattgcgaa aaagtacgga | 60 |
| | gtggattttg acacgttgaa aaaaacaaat acacaactta gtaatccaga tttaatcatg | 120 |
| | ccaggtatga aaattaaagt gccatcaaag agtgttcata tgaaacaaca ggctggagca | 180 |
| | ggttcagcgc ctccaaagca atacgtaaaa gaagtgcagc aaaaagaatt tgcagcaaca | 240 |
| | ccaactccgc ttggaataga agatgaggaa gaagttacgt atcaatcagc accaattaca | 300 |
| | cagcagccag ctatgcaaca aacacaaaaa gaagtgcaaa taaaaccgca gaaagagatg | 360 |
| | caagtaaagc cacaaaaaga agtacaggtg aaaccacaga aggagatgca ggtaaagccg | 420 |
| | caaaaagagg tgcaaaaaga acagccaatt caaaaagaaa aaccagttga aaaaccgtct | 480 |
| | gttattcaaa aaccacctgt gatagaaaaa caaaaaccgg cggaaaaaga aaacacgaag | 540 |
| | ttttcggtaa atgtattacc gcagccgcca caaccaccaa taaaaccgaa aaaagaatat | 600 |
| | aaaatttcag atgtaataaa aaaaggaagc gagttaattg ctcctcaaat tagtaaaatg | 660 |
| | aaacctaaca atatcatttc tccgcaaacg aaaaaaaata atataatatc gccgcaagtg | 720 |
| | aagaaagaga atgtagggaa tatagtgtca ccacaagtga aaaaagagaa tgtagggaat | 780 |
| | atagtgtcac cacaagtgaa aaaagaaaat gtaggaaata tagtgtcgcc gcaagtgaaa | 840 |
| | aaagaaaatg taggaaatat agtgtcgccg caagtgaaga aagagaatgt agggaatata | 900 |
| | gtgtcaccac aagtgaaaaa agaaaatgta ggaaatatag tgtcaccaca agtgaagaaa | 960 |
| | gaaaacgtag ggaatatagt atcgccaaat gtatcgaaag aaaatgtagt tattccacaa | 1020 |
| | gtcataccgc caaatattca aatgccgaat ataatgccaa ttatggataa caatcaacca | 1080 |
| | ccgaatataa tgccaattat ggataacaat caaccaccga atataatgcc aattatggat | 1140 |
| | aacaatcaaa tgccgaatat gatgccaatt atggataaca atcaaatgcc gaatatgatg | 1200 |
| | ccaattatgg ataacaatca aatgccgaat atgatgccaa ttatggataa caatcaaatg | 1260 |
| | ccgaatatga tgccaattat ggataacaat caaatgccga atatgatgcc aattatggat | 1320 |
| | aacaatcaaa tgccgaatat gatgccaatt atggataaca atcaaatgcc gaacatgatg | 1380 |
| | ccaattatgg ataacaatca aatgccgaat ataatgccga ttatggataa taaccaaatg | 1440 |
| | ccgaatatga tgccaatcat ggataacaat caaatgccga atataatgcc aattatggat | 1500 |
| | aacaatcaaa tgccgaatat gatgccgatt atggataaca atcaaccacc aaatatgatg | 1560 |
| | ccctatcaaa tgccgtatca acagcccatg atgccgccga atccgtatta tcaacaacca | 1620 |
| | aatccatatc aaatgccata tcagcaagga gcgccgtttg gaccgcaaca tacgtctatg | 1680 |
| | ccaaaccaga atatgatgcc aatggataat aacatgccgc cgcttgtgca gggtgaggaa | 1740 |
| | gattgtggat gcggaggaga aagtagacta tatagtccac aaccaggcgg tccgcaatat | 1800 |
| | gcgaatcctt tatattatca accaactcag tctgcatatg caccacagcc aggaacgatg | 1860 |
| | tattatcaac cagatccacc aaatgtattt ggagagcccg tttcagaaga agaggacgaa | 1920 |
| | gaagaagttt aa | 1932 |
| B. cereus ExsB-(Q7WTL9) | | |
| 328. | SQ SEQUENCE 192 AA; 22865 MW; B814643A401417A6 CRC64; | |
| | MKRDIRKAVE EIKSAGMEDF LHQDPSTFEC DDDKFTHHHC TTGCKCTTGG KCPRTRCTRV | |
| | KHCTFVTKCT HVKKWTFVTK CTRVRVQKWT FVTKVTRRKE CVLVTKRTRR KHCTFITKCI | |
| | RFEKKFFWTK RSFCKKCEFF PNRHGGSCDD SCDHGKDCHD SGHKWNDCKG GHKFPSCKNK | |
| | KFDHFWYKKR NC | |
| 327. | SQ Sequence 579 BP; 210 A; 96 C; 120 G; 153 T; 0 other; 3864053855 | |
| | CRC32; | |
| | atgaaacgtg atattagaaa agctgtcgaa gaaatcaaaa gtgctgggat ggaggatttc | 60 |
| | ttacaccaag atccaagtac ttttgaatgc gatgatgata aattcactca tcatcattgt | 120 |
| | acaactggat gtaaatgtac aactgggggt aaatgtccaa gaacaagatg tactcgcgtg | 180 |
| | aaacattgta cgttcgttac aaaatgtacg catgtgaaaa aatggacatt tgttacgaaa | 240 |
| | tgtactcgtg tacgtgttca aaaatggacg ttcgttacga aagtaacgcg tagaaaagaa | 300 |
| | tgcgtattag ttacgaaacg tactcgcaga aaacattgta cattcattac aaaatgcata | 360 |
| | cgctttgaaa agaaattttt ctggacaaaa cgaagtttct gtaaaaaatg cgaattcttc | 420 |
| | cctaacagac acggtggctc ttgcgatgat tcatgtgatc atggtaaaga ctgtcacgat | 480 |
| | agcggacaca aatggaatga ttgcaaaggc ggacataaat tcccatcttg caaaaataag | 540 |
| | aaattcgatc acttctggta taaaaaacgt aactgctag | 579 |
| B. cereus ExsC-(Q7WTL1) | | |
| 330. | SQ SEQUENCE 144 AA; 15774 MW; 1638897AB274F15E CRC64; | |
| | MTHIIDYQAT QPISKTGETT FAIPSSPNKA ILANLKLRIS SRDSRNNRVE LIATIGIEGI | |
| | TETSQVLFRI FRDNIEIFNA QVGIESTDSE QFYVQTFQAI DQNVSSGTHE YSLTVENLTS | |
| | GASAEVVGPL SFSALAIGQE RKCC | |
| 329. | SQ Sequence 435 BP; 153 A; 75 C; 72 G; 135 T; 0 other; 2869138336 | |
| | CRC32; | |
| | atgactcata tcattgatta ccaagctact caacctatta gtaaaactgg tgaaacaact | 60 |
| | tttgcaatcc catcttctcc aaataaagca attttagcaa atttgaaatt gcgaatttca | 120 |
| | agtagagatt cacgtaataa tcgagtagaa ttaatcgcta caattggtat agaaggtata | 180 |
| | actgagactt cacaagtttt attccgaatt ttccgtgata atattgaaat ttttaatgca | 240 |
| | caagtaggta ttgaatctac agattctgaa caattctatg tacaaacatt tcaagctata | 300 |
| | gatcaaaacg ttagcagtgg aacacacgaa tattcattaa ctgtagaaaa ccttactagt | 360 |
| | ggtgcaagcg cagaagttgt tggcccacta tcttttagcg ctttagctat tggacaagag | 420 |
| | cgtaaatgtt gctaa | 435 |
| B. cereus ExsD-(Q7WTL6) | | |
| 332. | SQ SEQUENCE 154 AA; 17458 MW; F31BC1243DA52C00 CRC64; | |
| | MADYFYKDGK KYYKNQSHSN DQKNNCFIET HTIAGSAENE NGNIPVSVFL ETTAPQTVFE | |
| | DFTNNHNKTL IQLFVVGMSA PVQVTILTRR SSVPITTTLQ PVQTKIFQVE DFQSLTLTKQ | |
| | EGSTSVVSLF VQKTFCICCK DNNDSCDEYY HECN | |
| 331. | SQ Sequence 465 BP; 174 A; 75 C; 68 G; 148 T; 0 other; 3005698428 | |
| | CRC32; | |
| | atggctgatt acttttataa agatggtaaa aaatattata aaaaccaatc gcattcgaac | 60 |
| | gaccaaaaaa acaactgttt tattgaaact catacgatag ctggttctgc agaaaatgaa | 120 |
| | aatggaaata tacctgtatc tgttttcctt gaaaccaccg ctccacaaac tgtatttgag | 180 |
| | gattttacaa acaatcataa taaaacatta attcagttat tcgttgtcgg tatgagtgca | 240 |
| | cctgttcaag taactattct aacaagaaga tctagcgtac caattactac tacattacaa | 300 |
| | cctgttcaaa caaaaatatt tcaagttgaa gattttcaaa gtcttactct tacaaagcag | 360 |
| | gaaggttcta ctagtgtagt tagtttattt gttcaaaaaa cattttgtat atgctgtaaa | 420 |
| | gataataacg attcatgtga tgaatattac cacgaatgta attga | 465 |
| B. cereus ExsE-(Q7WTK9) | | |
| 334. | SQ SEQUENCE 318 AA; 35841 MW; 1353B4C36124C986 CRC64; | |
| | MRTWRVGTFS MGLSIISLGC FLLFSVVKGI QVLDTLTAWW PVLLIILGAE VLLYLLFSKK | |
| | EQSFIKYDIF SIFFIGVLGS VGIAFYCLLS TGLLEEVRHS INTTRQTSNI PDGQFDIPES | |
| | IKKIVVDAGH QPLTIEGNNT NQIHLLGTYE MTTKANEKLN LKRDDFLSVQ TAGETMYITL | |
| | KSLPVQHTLF NSAPQVKPTL VLPQNKNVEI RASNNELSLY PGQLQNNWFV QESSRVSVHL | |
| | AKESDVSLTA VTNQKETHGS TPWEQVEDLT KNENTSSEEH PELNTQEHWY KNSIKTGNGT | |
| | YKLNIEKAYN LNMSVLEK | |
| 333. | SQ Sequence 957 BP; 348 A; 153 C; 166 G; 290 T; 0 other; 1357372653 | |
| | CRC32; | |
| | atgagaacat ggcgtgttgg aacattctca atggggcttt ctattatatc gttaggatgc | 60 |
| | tttttacttt tttcagtcgt aaaaggaatt caagtattag atacactaac tgcatggtgg | 120 |
| | ccagttttac ttatcatact tggagctgaa gttttactat accttctatt ctctaaaaaa | 180 |
| | gagcaatctt ttattaaata tgatattttt agtattttct ttatcggcgt tttaggaagt | 240 |
| | gtcggaattg ctttttactg tttattatca actggattac tagaagaagt tcgtcattct | 300 |
| | attaatacaa cgaggcaaac gagtaatatt ccagacggac aatttgatat acctgaatct | 360 |
| | atcaaaaaaa tcgtagtaga tgcaggacat cagcctctaa cgatagaggg aaataataca | 420 |
| | aatcaaattc atcttttggg aacttatgaa atgacaacga aagcaaatga aaaactcaat | 480 |
| | ttaaaacgag atgatttcct ttcagttcaa acggctggag aaacgatgta tatcacttta | 540 |
| | aaatcattac ctgttcagca tacgttattt aattcagcac cacaggtgaa accaacgctt | 600 |
| | gttcttccac aaaataaaaa tgtggaaatc cgtgcttcaa ataacgaact atctctttat | 660 |
| | ccaggtcaat tgcaaaataa ttggtttgta caggaaagct caagagtgtc tgtccatctt | 720 |
| | gcaaaagaga gtgatgtttc tttaacagca gtaacgaatc aaaaagaaac acatggaagt | 780 |
| | acaccttggg aacaagtaga agatttaacg aaaaacgaaa atacttcttc agaagaacat | 840 |
| | ccagaattaa acacccaaga acattggtat aaaaattcga ttaaaactgg aaatgggacg | 900 |
| | tacaagttaa atattgagaa agcttataat ttgaatatga gtgttctcga aaaataa | 957 |
| B. cereus ExsG-(Q7WTL4) | | |
| 336. | SQ SEQUENCE 50 AA; 5368 MW; 2DD07ADA453EE513 CRC64; | |
| | MEFQLLVTCI LQEGNAYFLV TKVDDVITLK VPITAGVAGL FLALGVPRCS | |
| 335. | SQ Sequence 153 BP; 46 A; 16 C; 34 G; 57 T; 0 other; 1457900509 | |
| | CRC32; | |
| | atggaatttc aattgttggt aacttgtata ttacaagaag gtaatgctta ctttttagta | 60 |
| | acgaaggtag atgatgttat tacgttaaaa gtaccgatta ctgcgggagt agcaggttta | 120 |
| | tttttagctt taggtgtacc aagatgttct taa | 153 |
| B. cereus ExsH-(Q7WTL0) | | |
| 338. | SQ SEQUENCE 425 AA; 40970 MW; 6318F1D1E210F6BE CRC64; | |
| | MTNNNCFGHN HCNNPIVFTP DCCNNPQTVP ITSEQLGRLI TLLNSLIAAI AAFFANPSDA | |
| | NRLALLNLFT QLLNLLNELA PSPEGNFLKQ LIQSIINLLQ SPNPNLSQLL SLLQQFYSAL | |
| | APFFFSLIID PASLQLLLNL LTQLIGATPG GGATGPTGPT GPGGGATGPT GPTGPGGGAT | |
| | GPTGPTGATG PAGTGGATGL TGATGLTGAT GLTGATGPTG ATGLTGATGL TGATGLTGAT | |
| | GPTGATGPTG ATGLTGATGA TGGGAIIPFA SGTTPSALVN ALIANTGTLL GFGFSQPGVA | |
| | LTGGTSITLA LGVGDYAFVA PRAGVITSLA GFFSATAALA PLSPVQVQIQ ILTAPAASNT | |
| | FTVQGAPLLL TPAFAAIAIG STASGIIPEA IPVAAGDKIL LYVSLTAASP IAAVAGFVSA | |
| | GINIV | |
| 337. | SQ Sequence 1278 BP; 397 A; 272 C; 262 G; 347 T; 0 other; 3047036472 | |
| | CRC32; | |
| | atgacaaaca ataattgttt tggtcataac cactgcaata atccgattgt tttcactcca | 60 |
| | gattgctgta acaatccaca aacagttcca attactagtg agcaattagg tagattaatt | 120 |
| | actttactaa actctttaat agcggctatt gcagcgtttt ttgcaaatcc aagtgatgca | 180 |
| | aacagattag ctttactcaa tttgtttact caactattga acttactaaa tgaattagca | 240 |
| | ccttccccag aagggaattt cttaaaacaa ttaattcaaa gtattattaa tttactacaa | 300 |
| | tctcctaacc caaatctaag tcaattactt tctttattac aacaattcta cagtgctctt | 360 |
| | gcaccattct tcttctcttt aattattgac cctgcaagtt tacaactttt attaaactta | 420 |
| | ttaactcaat taattggtgc tactccagga ggcggagcaa caggtccaac aggtccaaca | 480 |
| | ggtccaggag gcggagcaac aggtccaaca ggtccaacag gtccaggagg cggagcgaca | 540 |
| | ggtccaacag gcccaacagg agcgacaggt ccagcaggta ctggtggagc aacaggttta | 600 |
| | acaggagcaa caggtttaac aggagcaaca ggcttaacag gagcgacagg cccaacggga | 660 |
| | gcaacaggtt taacaggagc aacaggttta acaggagcaa caggcttaac aggagcgaca | 720 |
| | ggtccaacag gagcaacagg tccaacagga gcaacaggtt taacaggagc aactggtgca | 780 |
| | actggtggcg gagctattat tccatttgct tcaggtacaa caccatctgc gttagttaac | 840 |
| | gcgttaatag ctaatacagg aactcttctt ggatttggat ttagtcagcc tggtgtagct | 900 |
| | ttaactggtg gaacaagtat cacattagca ttaggtgtag gtgattatgc atttgtagca | 960 |
| | ccacgcgcag gggttattac gtcattagct ggtttcttta gtgcaacagc tgcattagct | 1020 |
| | ccattatcac ctgttcaagt gcaaatacaa atattaactg cacctgcagc aagcaatacg | 1080 |
| | tttacagtac aaggcgcacc tcttttatta acaccagcat ttgccgcaat agcgattggt | 1140 |
| | tctacagcat caggaatcat acctgaagct attccagtag cagctgggga taaaatactg | 1200 |
| | ttatatgttt cattaacagc agcaagtcca atagctgcag ttgctggatt tgtaagtgca | 1260 |
| | ggtattaata tcgtttaa | 1278 |
| B. cereus ExsY-(Q7WTL8) | | |
| 340. | SQ SEQUENCE 154 AA; 16419 MW; DB85816F3BE16D0F CRC64; | |
| | MSCNENKHHG SSHCVVDVVK FINELQDCST TTCGSGCEIP FLGAHNTASV ANTRPFILYT | |
| | KTGEPFEAFA PSASLTSCRS PIFRVESVDD DSCAVLRVLT VVLGDSSPVP PGDDPICTFL | |
| | AVPNARLIST TTCITVDLSC FCAIQCLRDV SIVK | |
| 339. | SQ Sequence 465 BP; 135 A; 92 C; 87 G; 151 T; 0 other; 3150213378 | |
| | CRC32; | |
| | atgagttgta acgaaaataa acaccatggc tcttctcatt gtgtagttga cgttgtaaaa | 60 |
| | ttcatcaatg aattacaaga ttgttctaca acaacatgtg gatctggttg tgaaatccca | 120 |
| | tttttaggtg cacacaatac tgcatcagta gcaaatacac gcccttttat tttatacaca | 180 |
| | aaaactggag aaccttttga agcattcgca ccatcagcaa gccttactag ctgccgatct | 240 |
| | ccaattttcc gtgtggaaag tgtagatgat gatagctgtg ctgtgctacg tgtattaact | 300 |
| | gtagtattag gtgacagttc tccagtacca cctggtgacg atccaatttg tacgttttta | 360 |
| | gctgtaccaa atgcaagatt aatatctaca actacttgca ttactgttga tttaagctgt | 420 |
| | ttctgtgcga ttcaatgctt acgcgacgtt tctatcgtaa agtaa | 465 |
| B. cereus ExsJ-(Q7WTL2) | | |
| 342. | SQ SEQUENCE 430 AA; 41701 MW; A78F8E86868AA69C CRC64; | |
| | MKHNDCFDHN NCNPIVFSAD CCKNPQSVPI TREQLSQLIT LLNSLVSAIS AFFANPSNAN | |
| | RLVLLDLFNQ FLIFLNSLLP SPEVNFLKQL TQSIIVLLQS PAPNLGQLST LLQQFYSALA | |
| | QFFFALDLIP ISCNSNVDSA TLQLLFNLLI QLINATPGAT GPTGPTGPTG PTGPAGTGAG | |
| | PTGATGATGA TGPTGATGPA GTGGATGATG ATGVTGATGA TGATGPTGPT GATGPTGATG | |
| | ATGATGPTGA TGPTGATGLT GATGAAGGGA IIPFASGTTP SALVNALVAN TGTLLGFGFS | |
| | QPGVALTGGT SITLALGVGD YAFVAPRAGT ITSLAGFFSA TAALAPISPV QVQIQILTAP | |
| | AASNTFTVQG APLLLTPAFA AIAIGSTASG IIAEAIPVAA GDKILLYVSL TAASPIAAVA | |
| | GFVSAGINIV | |
| 341. | SQ Sequence 1293 BP; 403 A; 274 C; 263 G; 353 T; 0 other; 1562486421 | |
| | CRC32; | |
| | atgaaacata atgattgttt tgatcataat aactgcaatc cgattgtttt ttcagcagat | 60 |
| | tgttgtaaaa atccacagtc agttcctatt actagggaac aattaagtca attaattact | 120 |
| | ttactaaact cattagtatc agctatttca gcattttttg caaatccaag taatgcaaac | 180 |
| | agattagtgt tactcgattt atttaatcaa tttttaattt tcttaaattc cttattacct | 240 |
| | tccccagaag ttaatttttt gaaacaatta actcaaagta ttatagtttt attacaatct | 300 |
| | ccagcaccta atttaggaca attgtcaaca ttattgcaac aattttatag cgcccttgca | 360 |
| | caattcttct tcgctttaga tcttatccct atatcctgca actcaaatgt tgattctgca | 420 |
| | actttacaac ttctttttaa tttattaatt caattaatca atgctactcc aggggcgaca | 480 |
| | ggtccaacag gtccaacagg tccaacaggt ccaacgggcc cagcaggaac cggagcaggt | 540 |
| | ccaacgggag caacgggagc aacaggagca acaggcccaa caggagcgac aggtccagca | 600 |
| | ggtactggtg gagcaacagg agcaacagga gcaacaggag taacaggagc aacaggggca | 660 |
| | acaggagcaa caggtccaac aggtccaaca ggggcaacag gtccaacagg ggcaacagga | 720 |
| | gcaacaggag caacaggtcc aacaggagca acaggtccaa caggggcaac gggcttaaca | 780 |
| | ggagcaactg gtgcagctgg tggcggagct attattccat ttgcttcagg tacaacacca | 840 |
| | tctgcgttag ttaacgcgtt agtagctaat acaggaactc ttcttggatt tggatttagt | 900 |
| | cagcctggtg tagcattaac aggtggaact agtatcacat tagcattagg tgtaggtgat | 960 |
| | tatgcatttg tagcaccacg tgcaggaact atcacgtcat tagcaggttt ctttagtgca | 1020 |
| | acagctgcat tagctccaat atcacctgtt caagtgcaaa tacaaatatt aactgcacct | 1080 |
| | gcagcaagca atacgtttac agtacaaggc gcacctcttt tattaacacc agcatttgcc | 1140 |
| | gcaatagcga ttggttctac agcatcaggt atcatagctg aagctattcc agtagctgct | 1200 |
| | ggagataaaa tactactgta tgtttcatta acagcagcaa gtccaatagc tgcagttgct | 1260 |
| | ggatttgtaa gtgcaggtat taatatcgtt taa | 1293 |
| B. cereus ExsF-(Q7WTL3) | | |
| 344. | SQ SEQUENCE 167AA; 17374MW; CB29A5CFBE9ABB33 CRC64; | |
| | MFSSDCEFTK IDCEAKPAST LPAFGFAFNA SAPQFASLFT PLLLPSVSPN PNITVPVIND | |
| | TVSVGDGIRI LRAGIYQISY TLTISLDNVP TAPEAGRFFL SLNTPANIIP GSGTAVRSNV | |
| | IGTGEVDVSS GVILINLNPG DLIQIVPVEL IGTVDIRAAA LTVAQIS | |
| 343. | SQ Sequence 504 BP; 142 A; 104 C; 90 G; 168 T; 0 other; 852047041 | |
| | CRC32; | |
| | atgttctctt ctgattgcga atttactaaa atcgattgcg aggcaaaacc agctagtaca | 60 |
| | ctacctgcct ttggttttgc tttcaatgct tctgcacctc agttcgcttc actatttaca | 120 |
| | ccactactat tacctagtgt aagtccaaac ccaaatatta ctgttcctgt aatcaacgat | 180 |
| | acagtaagtg tcggagatgg cattcgaatt ctacgagctg gtatttatca aattagttat | 240 |
| | acattaacaa ttagtcttga taacgtacct actgcaccag aagctggtcg tttcttctta | 300 |
| | tcattaaata caccagctaa tattattcct ggatcaggta cagcagttcg ttctaacgtt | 360 |
| | attggtactg gtgaagtgag tgtatccagt ggtgtcattc ttattaactt aaatcctggt | 420 |
| | gacttaattc aaattgtgcc agttgagtta attggaactg tagacatccg tgcggcagca | 480 |
| | ttaacagttg cacaaattag ctag | 504 |
| B. cereus YrbB-(Q6B4J4) | | |
| 346. | SQ SEQUENCE 213 AA; 24327 MW; 806E9ED79054A443 CRC64; | |
| | MNTKVKVIAA SLLVTSALAA CGTPKNNAMD GRNYNYERTS YNDTHQYRDN VTRNDRYTDY | |
| | VTYRNGRNDT GYNYYRDVNY NGQIANPHPT RNITMNNSYI NNDGKTAERI TNRVKRMNNV | |
| | DRVSTVVYGN DVAIAVKPRN TVTNETAMAN EIRQAVANEV GNRNVYVSVR NDMFTRVDAM | |
| | STRLRNGTVT NDFNRDIGNM FRDIRYGLTG TVR | |
| 345. | SQ Sequence 642 BP; 230 A; 101 C; 135 G; 176 T; 0 other; 1643929295 | |
| | CRC32; | |
| | ttgaatacga aagtaaaagt gattgctgct tctttgttag ttactagtgc attagctgca | 60 |
| | tgtggtacac caaaaaacaa tgcaatggat ggacgtaact acaattacga gcgtacatct | 120 |
| | tataatgata cacaccagta tcgtgataat gtgacgcgta atgatcgtta tacagattat | 180 |
| | gtaacatata gaaatggtcg taacgataca ggatacaatt attaccgtga tgtaaattac | 240 |
| | aatggacaaa ttgctaatcc gcatccaact cgtaatatta caatgaacaa ttcatacatt | 300 |
| | aacaatgatg gtaaaacagc tgaaagaata acaaatcgtg tgaaacgtat gaataacgta | 360 |
| | gaccgtgtgt ctacagttgt atatggaaac gatgtagcga ttgcggtaaa accacgtaac | 420 |
| | acagtgacaa atgaaacggc gatggcgaac gaaattcgtc aagctgttgc aaatgaagtt | 480 |
| | ggaaacagaa acgtatatgt ttctgtaaga aatgatatgt ttactcgtgt cgatgcaatg | 540 |
| | agtacgcgtc tacgtaacgg tacagttaca aacgatttta atcgtgatat aggaaatatg | 600 |
| | ttcagagaca ttcgttacgg tttaactggt acagtgcgat ag | 642 |
| B. cereus NadA-(Q6B4J6) | | |
| 348. | SQ SEQUENCE 186 AA; 21109 MW; 56DCC137D5363F80 CRC64; | |
| | PDQHLGRNTA YDLGIPLDKM AVWDPHTDSL EYDGDIEEIQ VILWKGHCSV HQNFTVKNIE | |
| | SVRKNHSNMN IIVHPECCYE VVAASDYAGS TKYIIDMIES APSGSKWAIG TEMNLVNRII | |
| | QQHPDKEIVS LNPFMCPCLT MNRIDLPHLL WTLETIERGE EINVISVDKQ VTAEAVLALN | |
| | RMLERV | |
| 347. | SQ Sequence 562 BP; 198 A; 83 C; 121 G; 160 T; 0 other; 2102162024 | |
| | CRC32; | |
| | accagaccaa catttaggga gaaatacagc gtacgatcta ggtatcccgt tagataaaat | 60 |
| | ggcagtatgg gacccgcaca cagattcatt agagtacgat ggggatatag aagaaattca | 120 |
| | agtgatttta tggaaaggac attgttctgt tcatcaaaat tttacagtga agaatattga | 180 |
| | gagtgtacga aaaaatcatt ctaatatgaa tattattgta catccagaat gttgctatga | 240 |
| | agttgtagct gcttcagatt atgcaggctc aacgaaatat attattgata tgattgaatc | 300 |
| | agcgccatct ggtagcaaat gggcgattgg tacagaaatg aatttagtga atcgaattat | 360 |
| | tcagcaacat ccagataaag aaattgtttc gcttaatcca tttatgtgtc cgtgcttaac | 420 |
| | gatgaatcga atagatctgc ctcacttatt atggacactt gaaacgatag aaagaggaga | 480 |
| | agaaattaac gttattagcg tagacaaaca agtaacggca gaagcagttc ttgcattaaa | 540 |
| | tcgtatgtta gagcgtgtgt aa | 562 |

1. A method for isolation of a glycoprotein complex from the exosporium of a Bacillus anthracis or an anthrax-like bacterium for use as a vaccine, the method comprising the steps of:
a) lysing said exosporium to form an extract;
b) isolating at least one glycoprotein complex comprising a glycoprotein and at least one other molecule from said extract by absorption of the extract to a lectin; and
c) administering to a subject an immunogenic amount of the at least one glycoprotein complex as a vaccine to induce an immune response, wherein the glycoprotein complex induces an antibody titer of at least 800 in the subject.

2. (canceled)

3. The method of claim 1, wherein said at least one other molecule is selected from the group consisting of a protein, an oligosaccharide, a lipid, and a phospholipid.

4. The method of claim 1, wherein said isolating step further comprises using at least one of size-exclusion chromatography or electro-elution.

5. (canceled)

6. The method of claim 29, wherein the glycoprotein comprises an amino acid sequence having at least 90% homology to at least one of SEQ ID. NOs: 44, 46, 48, 50, 52, 54, 56, 58, 60, 62, or 64.

7.-20. (canceled)

21. The method of claim 6, wherein the glycoprotein has at least 95% homology to at least one of SEQ ID. NOs: 44, 46, 48, 50, 52, 54, 56, 58, 60, 62, or 64.

22. The method of claim 6, wherein the glycoprotein has at least 99% homology to at least one of SEQ ID. NOs: 44, 46, 48, 50, 52, 54, 56, 58, 60, 62, or 64.

23. The method of claim 6, wherein the glycoprotein is at least one of SEQ ID. NOs: 44, 46, 48, 50, 52, 54, 56, 58, 60, 62, or 64.

24. A method for isolation of a glycoprotein complex from the exosporium of a Bacillus anthracis or an anthrax-like bacterium for use as a vaccine, the method comprising the steps of:
a) lysing said exosporium to form an extract;
b) isolating at least one glycoprotein complex comprising a glycoprotein and at least one other molecule from said extract by absorption of the extract to a lectin, wherein the glycoprotein comprises an amino acid sequence having at least 90% homology to at least one of SEQ ID. NOs: 44, 46, 48, 50, 52, 54, 56, 58, 60, 62, or 64, and wherein said amino acid sequence of the glycoprotein comprises SEQ ID NO:380; and
c) administering to a subject an immunogenic amount of the at least one glycoprotein complex as a vaccine to induce an immune response, wherein the glycoprotein complex induces an antibody titer of at least 800 in the subject.

25. The method of claim 24, wherein the glycoprotein has at least 90% homology to at least one of SEQ ID. NOs: 44, 46, 48, 50, 52, 54, 56, 58, 60, 62, or 64.

26. The method of claim 24, wherein the glycoprotein has at least 95% homology to at least one of SEQ ID. NOs: 44, 46, 48, 50, 52, 54, 56, 58, 60, 62, or 64.

27. The method of claim 24, wherein the glycoprotein has at least 99% homology to at least one of SEQ ID. NOs: 44, 46, 48, 50, 52, 54, 56, 58, 60, 62, or 64.

28. The method of claim 24, wherein the glycoprotein is at least one of SEQ ID. NOs: 44, 46, 48, 50, 52, 54, 56, 58, 60, 62, or 64.

29. The method of claim 1, wherein said glycoprotein comprises SEQ ID NO:380.
